(19) United States
(12) Patent Application Publication
(10) Pub. No.: US 2016/0346333 A1
(43) Pub. Date: Dec. 1, 2016

(54) PLACENTAL-DERIVED STEM CELLS AND USES THEREOF TO RESTORE THE REGENERATIVE ENGINE, CORRECT PROTEOMIC DEFECTS AND EXTEND LIFESPAN OF AGING SUBJECTS

(71) Applicant: Human Longevity, Inc., San Diego, CA (US)

(72) Inventor: Robert J. Hariri, Bernardsville, NJ (US)

(21) Appl. No.: 15/167,696

(22) Filed: May 27, 2016

Related U.S. Application Data
(60) Provisional application No. 62/167,786, filed on May 28, 2015.

(51) Int. Cl. A61K 35/545; C12Q 1/06; C12N 5/073

(52) U.S. Cl. CPC A61K 35/545; C12N 5/0605; C12Q 1/06

(57) ABSTRACT

The present invention relates, in part, to the use of stem cells, such as placental-derived stem cells (PDSC), to reduce the effects of aging by, for example, restoring the regenerative engine and extending the lifespan of aging subjects. Provided herein, for example, are methods for maintaining or increasing the ratio of the number of stem cells to the number of differentiated cells in a tissue of a subject over time, comprising administering to the subject an effective amount of a population of stem cells (e.g., PDSC), wherein the ratio is maintained or increased over time as compared to the ratio of the number of stem cells to the number of differentiated cells in a tissue of a control subject over time. Further provided are methods of maintaining or increasing the number of stem cells in a tissue of a subject over time, comprising administering to the subject an effective amount of a population of stem cells (e.g., PDSC), wherein the number of stem cells in the tissue of the subject is maintained or increased over time as compared to the number of stem cells in the same tissue of a control subject. Also provided herein are methods of altering the phenotype or proteome of an aging stem cell resident in a tissue of a subject, comprising administering to the subject an effective amount of a population of stem cells (e.g., PDSC), wherein the amount is effective to alter the environmental niche of the aging stem cell such that the phenotype or proteome of the stem cell is altered as compared to the phenotype of the stem cell resident in the tissue of a control subject.

### CROSS-REFERENCE TO RELATED APPLICATIONS

The present application claims priority to U.S. Provisional Patent Application No. 62/167,786 filed May 28, 2015, the entirety of which is incorporated herein by reference.

### 1. FIELD

The present invention relates, in part, to the use of stem cells, such as placental-derived stem cells (PDSC), to reduce the effects of aging by, for example, restoring the regenerative engine—the complex physiologic system driven by populations of stem and progenitor cells which remodel and renew damaged and diseased tissues and organs and restore the synthetic repertoire resident in those tissues and organs-thereby extending the lifespan and quality of life of aging subjects. Provided herein, for example are methods for maintaining or increasing the ratio of the number of stem cells to the number of differentiated cells in a tissue of a subject over time, comprising administering to the subject an effective amount of a population of stem cells (e.g., PDSC), wherein the ratio is maintained or increased over time as compared to the ratio of the number of stem cells to the number of differentiated cells in a tissue of a control subject over time. Further provided are methods of maintaining or increasing the number of stem cells in a tissue of a subject over time, comprising administering to the subject an effective amount of a population of stem cells (e.g., PDSC), wherein the number of stem cells in the tissue of the subject is maintained or increased over time as compared to the number of stem cells in the same tissue of a control subject. Also provided herein are methods of altering the phenotype or proteome of an aging stem cell resident in a tissue of a subject, comprising administering to the subject an effective amount of a population of stem cells (e.g., PDSC), wherein the amount is effective to alter the environmental niche of the aging stem cell such that the phenotype or proteome of the stem cell is altered as compared to the phenotype of the stem cell resident in the tissue of a control subject.

### 2. SUMMARY

The process of aging represents a complex decline in physio-anatomic quality and performance in the subject which is characterized by a reduced capacity to recover and repair following injury and/or disease. This results in the accumulation of molecular and microscopic defects which can be thought to coalesce into the macroscopic phenotypic changes seen in an aged subject. These changes are seen by example in the skin of an aged subject having less elasticity, diminished turgor, irregular texture and color and having a diminished ability to repair after injury while that of a newborn subject is highly elastic, has normal tissue turgor and is very consistent in color and texture and repairs quickly and functionally normally to injury. Varani et al. (Am J Pathol 2006, 168(6):1861-1868) reported that collagen production in chronologically aged skin is dramatically different from youthful skin.

These changes can also be seen in the tissue of the cartilage system which changes with age and produces a different range of proteins in aged versus younger subjects. It is proposed that this difference in the proteome is a principal causative factor in the biomechanical changes seen in aged cartilage tissue.

Manavalan et al. (Exp Mol Med, 2013, 45:e39) reported changes in the proteome of aging brains and postulated that of 950 proteins, 31 were significantly altered. Most of the differentially regulated proteins are involved in molecular transport, nervous system development, synaptic plasticity and apoptosis. Particularly, proteins such as Gelsolin (GSN), Tenascin-R (TNR) and AHNAK could potentially act as novel biomarkers of aging-related neurodegeneration and moreover, protein turnover dependent on protease systems could be responsible for the changes seen in age-related dementia.

Piec et al. (FASEB J 2005, 19:1143-1145) reported that differential protein expression occurs with age and it is proposed herein that progressive decline in skeletal muscle mass and function which occurs during aging can be reversed or ameliorated by introducing cells derived from a young source.

These changes occur in all tissues and reflect a quantitative change in the population of undamaged stem and progenitor cells resident in the tissue, the fuel of the “regenerative engine” of the tissue. It is the undamaged stem and progenitor cells which maintain the intact and complete synthetic repertoire of the complete transcribable genome and which are capable of proliferating and differentiating to repair, and renovate the tissues of the aged subject and restore a state of function consistent with youthful performance. Chaves et al. (J Proteome Res 2013, 12(10):4532-4546) showed through comparative proteomic analysis that aging muscles express a very different proteome than youthful muscles and that this is associated with a decline in certain performance attributes of the tissue.

Stem cells retain their unique regenerative abilities by being capable of differentiating or maturing into the various highly specialized cell types of the mature phenotype. This process of differentiating in a versatile manner is referred to as “pluripotency,” which refers to the ability of a stem cell to divide and produce progeny that are phenotypically different from the source. This division can occur either asymmetrically or symmetrically. Asymmetric division yields daughter cells which are different from one another. The process of differentiating or specializing is the result of very specific molecular signaling events and changes in the manner in which these differentiating cells read and transcribe regions on their DNA resulting in expression of specific gene products and an altered proteome. As used herein, the “proteome” is the entire set of proteins expressed by a genome, cell tissue or organism at a certain time. More specifically, the proteome is the set of expressed proteins in a given type of cell or organism, at a given time, under defined conditions.

Thus, over time, a differentiating cell specializes and loses the ability to produce the proteomic repertoire of a less differentiated stem cell. This reduction in the ability of mature specialized cells to produce the entire transcribable genome results in a proteomic deficit in aged subjects compared to younger subjects. The process of aging can be characterized by a deficit in the number of stem and progenitor cells available to renew and renovate the tissues of the organism in response to aging, injury or disease (or a combination thereof), resulting in a proteomic deficit due to matured, differentiated cells resident to the specialized tissues losing the ability to produce the entire proteomic repertoire available in the fully transcribed human genome.

The ability to restore the integrity of the regenerative engine and correct the proteomic deficit that occurs with advanced age resides in the ability to deliver viable, proliferative and synthetically active stem and progenitor cells in a therapeutic manner.

Thus, the present invention, in part, provides a method and mechanism to recover and produce populations of highly proliferative and proteomically intact stem and progenitor cells (e.g., derived from the placenta). In certain embodiments, the methods provided herein further comprise processing and/or manufacturing these cells in quantities and with the quality necessary to be stored under cryopreservation conditions. In certain embodiments, the cryopreserved cells can be serially administered at clinically prescribed intervals to restore the regenerative engine of the recipient and to correct the proteomic deficit which exists with advanced age.

In one aspect, provided herein is a method for maintaining or increasing the ratio of the number stem cells to the number of differentiated cells in a tissue of a subject in need thereof over time, comprising administering to the subject an effective amount of a population of stem cells, wherein the ratio is maintained or increased over time as compared to the ratio of the number stem cells to the number of differentiated cells in a tissue of a control subject over time. In one embodiment, the population of stem cells comprises a population of placental-derived stem cells (PDSC). In another embodiment, the population of stem cells consists essentially of a population of PDSC. In a specific embodiment, the population of stem cells consists of a population of PDSC.

In a second aspect, provided herein is a method of maintaining or increasing the number of stem cells in a tissue of a subject in need thereof over time, comprising administering to the subject an effective amount of a population of stem cells, wherein the number of stem cells in the tissue of the subject is maintained or increased over time as compared to the number of stem cells in the same tissue of a control subject. In one embodiment, the population of stem cells comprises a population of PDSC. In another embodiment, the population of stem cells consists essentially of a population of PDSC. In a specific embodiment, the population of stem cells consists of a population of PDSC.

In a third aspect, provided herein is a method of altering the phenotype of an aging stem cell resident in a tissue of a subject in need thereof, comprising administering to the subject an effective amount of a population of stem cells, wherein the amount is effective to alter the environmental niche of the aging stem cell such that the phenotype of the stem cell is altered as compared to the phenotype of the stem cell resident in the tissue of a control subject. In one embodiment, the population of stem cells comprises a population of PDSC. In another embodiment, the population of stem cells consists essentially of a population of PDSC. In a specific embodiment, the population of stem cells consists of a population of PDSC.

In one embodiment, provided herein is a method to alter an aging stem cell through modifying the environmental niche in which it resides. In some embodiments, the environmental niche is modified for the purpose of reconditioning the phenotype of the aging stem cell to one with a longer lifespan. In certain embodiments, the method comprises in vivo or in vitro co-cultivation of aging stem cells with progenitor cells from a younger source (e.g., PDSC). In some embodiments, the co-cultivation effectuates a molecular and/or genetic event, which can have the net effect of rejuvenating the aged cells. In certain embodiments, the various methods provided herein can be used to phenotypically modify an aging organism to express traits consistent with a younger phenotype.

In certain embodiments, the methods provided herein can encompass a range of in vivo and in vitro methods by which exposure of aging cells to younger progenitors results in the transfer or transition to a youthful phenotype. In some embodiments, the various methods provided herein can comprise co-cultivation in vitro. In other embodiments, the various methods provided herein include niche conditioning in vivo. In certain embodiments, the niche reconditioning is accomplished by delivering young progenitors (e.g., PDSC) to a physioanatomic niche (e.g., the bone marrow or organ system). In other embodiments, the conditioning of an aging niche is accomplished through the delivery of bioactive factors, such as paracrine factors, isolated from young progenitors.

In specific embodiments, the various methods provided herein will result in the modulation of the phenotype of aging cells by inducing the repertoire of genotypically expressed factors characteristic of a youthful phenotypic state by exposure. e.g., to placental cells and/or secreted factors thereof.

In a specific embodiment, the aging stem cell is in a tissue of a subject in need thereof. In other embodiments, the aging stem cell is derived from a subject in need thereof.

In some embodiments, the methods provided herein comprise controlled co-cultivation with placental cells (e.g., in situ or in vitro). In other embodiments, the methods provided herein include the therapeutic administration of placental cells (e.g., PDSC). In some embodiments, the administration is to a subject. In some embodiments of the various methods provided herein, the subject is a subject in need thereof. Such cells can be used to exploit the secretome of the transiently or permanently residing stem cells on the aging cells of the recipient. The placenta is the source of a range of highly proliferative stem and progenitor cells, which express a robust secretome of growth and regulatory factors as provided elsewhere herein. Such placental cells can induce immunologic tolerance. Such placental cells can also stimulate endogenous stem cell regeneration. Placental cells have also been successfully transplanted into humans for a variety of clinical indications including autoimmune disease, stroke and cancer.

Recent work by Conboy and others (Carlson et al., EMBO Mol Med 2009, 1(8-9):381-391) showed that molecular features of aging cells can be altered in the presence of younger cells. In addition, Hariri et al. showed almost 30 years ago that the age of the host influences the behavior of transplanted cells.

Various methods provided herein can be used, for example, to control the phenotype of cells (e.g., aging cells) by exposing them to cells of a youthful chronobiological age (e.g., PDSC). In some embodiments, the aging cells are exposed to PDSC using a placental bioreactor. In other embodiments, the aging cells are exposed to PDSC using a co-cultivation system. In yet other embodiments, the aging cells are exposed to PDSC following administration of placental cells to the subject, for example via intravenous infusion, direct injection, or other form of parenteral administration. In a specific embodiment, the aging cells are of a subject, such as a subject in need thereof. Although PDSC are exemplified herein, it is understood that other types of stem cells can be used.

In the certain embodiments of the various methods provided herein, stem cells, such as stem cells derived from newborn placenta (e.g., PDSC) are used in a co-culture environment as a “feeder” layer upon which cells from an older donor are cultivated. In certain embodiments, the cells from the older donor are stem cells, progenitor cells, or other cells which retain the ability to propagate when returned to the host. In some embodiments, the stem cells derived from the newborn placenta are expanded in vitro in culture. In other embodiments, the stem cells derived from the newborn placenta are unexpanded. In certain embodiments, after a period of co-cultivation, the donor cells will be isolated from the feeder layer. In a specific embodiment, the donor cells will then be reintroduced into the donor. In a specific embodiment, the host or donor is a subject in need thereof.

In another embodiment, the newborn cells (e.g., PDSC) are cultured in an extracorporeal device. In some embodiments, the extracorporeal device is placed in the circulatory circuit of a recipient subject such that the secreted factors of newborn cells are delivered to the subject. In a specific embodiment, the subject is a subject in need thereof.

In yet other embodiments of the various methods provided herein, the newborn cells (e.g., PDSC) are administered therapeutically (e.g., either systemically or locally). In some embodiments, the newborn cells are administered via injection. In other embodiments, the newborn cells are administered via infusion. In such methods, the cells can traffic through the recipient subject and take up short- or long-term residence in proximity to the recipients aging cells. The cells can then, in certain embodiments, effectively alter the aging phenotype of recipient cells to a more youthful phenotype. In some embodiments, the alteration is the result of direct or indirect contact of the aging cells with paracrine factors, endocrine factors and/or direct cell-to-cell interactions, e.g., with the newborn cells.

In another aspect, provided herein is a method of altering the proteome of an aging cell in a tissue of a subject in need thereof, comprising administering to the subject an effective amount of a population of stem cells, wherein the amount is effective to alter the proteome of the aging cell, wherein the altered proteome comprises one or more biomarkers found in a younger cell in the tissue of a control subject. In one embodiment, the population of stem cells comprises a population of PDSC. In another embodiment, the population of stem cells consists essentially of a population of PDSC. In a specific embodiment, the population of stem cells consists of a population of PDSC.

In some embodiments, the aging cell is a somatic cell. In some embodiments, the aging cell is a skeletal muscle cell. In some embodiments, the aging cell is a brain cell. In some embodiments, the aging cell is from the brain. In other embodiments, the aging cell is a cardiac cell. In some embodiments, the aging cell is from the heart. In some instances, the aging cell is a kidney cell. In some embodiments, the aging cell is from the kidney. In some embodiments, the aging cell is a liver cell. In some embodiments, the aging cell is from the liver. In other embodiments, the aging cell is a granulocyte, mast cell or macrophage. In some embodiments, the aging cell is from the bone marrow. In some instances, the aging cell is a skin cell. In some embodiments, the aging cell is from the skin.

In another aspect, provided herein is a method of altering the transcriptome of an aging cell in a tissue of a subject in need thereof, comprising administering to the subject an effective amount of a population of stem cells, wherein the amount is effective to alter the transcriptome of the aging cell, wherein the altered transcriptome comprises one or more transcripts found in a younger cell in the tissue of a control subject. In one embodiment, the population of stem cells comprises a population of PDSC. In another embodiment, the population of stem cells consists essentially of a population of PDSC. In a specific embodiment, the population of stem cells consists of a population of PDSC. In some embodiments, the one or more transcripts are identified using a transcript array analysis. In some embodiments, the one or more transcripts are identified using TaqMan® Low Density Arrays (TLDA) on 7900HT Real-Time PCR systems. In certain embodiments, the transcript is a transcript of a biomarker provided herein.

In some embodiments, the aging cell is a somatic cell. In some embodiments, the aging cell is a skeletal muscle cell. In some embodiments, the aging cell is a brain cell. In some embodiments, the aging cell is from the brain. In other embodiments, the aging cell is a cardiac cell. In some embodiments, the aging cell is from the heart. In some instances, the aging cell is a kidney cell. In some embodiments, the aging cell is from the kidney. In some embodiments, the aging cell is a liver cell. In some embodiments, the aging cell is from the liver. In other embodiments, the aging cell is a granulocyte, mast cell or macrophage. In some embodiments, the aging cell is from the bone marrow. In some instances, the aging cell is a skin cell. In some embodiments, the aging cell is from the skin.

In some embodiments of the various methods provided herein, the control subject is the same subject before administration of the population of stem cells (e.g., PDSC). In other embodiments, the control subject is a subject that has not received the population of stem cells (e.g., PDSC).

In certain embodiments of the various methods provided herein, the method further comprises (i) determining the number of stem cells and/or differentiated cells in the tissue before administration of the population of stem cells to the subject, and (ii) determining the number of stem cells and/or differentiated cells in the tissue after administration of the population of stem cells to the subject. In one embodiment, the population of stem cells comprises a population of PDSC. In another embodiment, the population of stem cells consists essentially of a population of PDSC. In a specific embodiment, the population of stem cells consists of a population of PDSC.

In some embodiments of the various methods provided herein, the method increases the number of stem cells in the tissue after administration as compared to before administration of the population of stem cells (e.g., PDSC). In one embodiment, the subject has an increased number of stem cells as compared to a subject that has not received an administration of population of stem cells (e.g., PDSC). In certain embodiments, the increase in the number of stem cells persists over time. In other embodiments, the increase in the number of stem cells is the result of an expansion of stem cells resident in the tissue. In one embodiment, the increase in the number of stem cells is the result of an expansion of the stem cells (e.g., PDSC) in the tissue. In another embodiment, the number of stem cells is assessed by stem cell colony forming units.

In some embodiments, the increase in the number of stem cells results in the remodeling, renewal, renovation, rejuvenation, repair and/or restoration of the tissue of the subject.

In some embodiments, the tissue is muscle. In one embodiment, the tissue is brain. In another embodiment, the tissue is skin. In some embodiments, the tissue is bone marrow. In one embodiment, the tissue is heart. In certain embodiments, the tissue is liver. In another embodiment, the tissue is kidney. In some embodiments, the tissue is pancreas. In other embodiments, the tissue is adipose tissue. In certain embodiments, the tissue is testis. In certain embodiments, the tissue is prostate. In some embodiments, the tissue is endometrium. In another embodiment, the tissue is ovary. In other embodiments, the tissue is lymphatic tissues. In certain embodiments, the tissue is testis. In certain embodiments, the tissue is lungs. In some embodiments, the tissue is adrenal glands. In another embodiment, the tissue is thyroid glands. In other embodiments, the tissue is spleen. In certain embodiments, the tissue is GI tract. In certain embodiments, the tissue is eye.

In certain embodiments of the various methods provided herein, the population of stem cells is administered systemically. In one embodiment, the population of stem cells is administered locally to the tissue. In some embodiments, the population of stem cells is administered by parenteral administration. In another embodiment, the population of stem cells is administered intravenously. In some embodiments, the population of stem cells is administered by continuous drip or as a bolus. In one embodiment, the population of stem cells is prepared to be administered in an injectable liquid suspension or other biocompatible medium. In other embodiments, the population of stem cells is administered using a catheter. In another embodiment, the population of stem cells is administered using a controlled-release system. In one embodiment, the population of stem cells is administered using an implantable substrate or matrix. In certain embodiments, the population of stem cells is administered intramuscularly. In some embodiments, the population of stem cells is administered subcutaneously. In one embodiment, the population of stem cells is administered subdermally. In another embodiment, the population of stem cells is administered intracompartmentally. In other embodiments, the method further comprises contacting the population of stem cells with young stem cells, young progenitor cells, or young precursor cells. In one embodiment, the population of stem cells comprises a population of PDSC. In another embodiment, the population of stem cells consists essentially of a population of PDSC. In a specific embodiment, the population of stem cells consists of a population of PDSC.

In one embodiment, the method further comprises contacting the population of stem cells (e.g., PDSC) with one or more additional factors isolated from young stem cells, young progenitor cells, or young precursor cells. In certain embodiments, the one or more additional factors are bioactive factors isolated from the secretome of a stem cell. In certain embodiments, the one or more additional factors are bioactive factors isolated from the secretome of a PDSC. In some embodiments, the one or more additional factors is selected from the group consisting of cytokines, hormones, promoters, repressors, proteins, nucleic acids, viruses, immunogens, angiogenic factors, growth factors, anti-apoptotic factors, and anti-oxidative factors, or any combination thereof. In another embodiment, the method further comprises culturing and/or expanding the population of stem cells (e.g., PDSC) prior to administration to the subject. In one embodiment, the culturing and/or expanding is in vitro. In certain embodiments, the culturing and/or expanding is in situ. In other embodiments, the population of stem cells (e.g., PDSC) is cultured and/or expanded in the presence of young stem cells, young progenitor cells, or young precursor cells. In one embodiment, the population of stem cells (e.g., PDSC) is cultured and/or expanded in the presence of additional factors isolated from young stem cells, young progenitor cells, or young precursor cells. In certain embodiments, the one or more additional factors is a bioactive factor isolated from the secretome of a stem cell. In certain embodiments, the one or more additional factors is a bioactive factor isolated from the secretome of a PDAC. In another embodiment, the one or more additional factors is selected from the group consisting of cytokines, hormones, promoters, repressors, proteins, nucleic acids, viruses, immunogens, angiogenic factors, growth factors, anti-apoptotic factors, and anti-oxidative factors, or any combination thereof. In some embodiments, the population of stem cells (e.g., PDSC) are cultured and/or expanded in an extracorporeal device. In some embodiments, the population of stem cells (e.g., PDSC) has been passaged at least three times. In one embodiment, the population of stem cells (e.g., PDSC) has been passaged no more than ten times.

In one embodiment, the population of stem cells has previously been cryopreserved. In another embodiment, the population of stem cells are cells from a placental stem cell bank. In one embodiment, the population of stem cells comprises cells obtained from a placenta that has been drained of cord blood. In one embodiment, the population of stem cells comprises cells obtained from a placenta that has been perfused to remove residual blood. In one embodiment, the stem cells comprise PDSC. In one embodiment, the stem cells consist essentially of PDSC. In one embodiment, the stem cells consist of PDSC. In one embodiment, the stem cells are PDSC.

In other embodiments, the PDSC are embryonic-like stem cells. In one embodiment, the stem cells are pluripotent or multipotent stem cells. In one embodiment, the PDSC are pluripotent or multipotent stem cells. In certain embodiments, the population of PDSC comprises cells that are CD34⁻, CD10⁺, SH2⁺, CD90⁺ placental multipotent cells. In another embodiment, the population of PDSC comprises cells that CD34⁻, CD38⁻, CD45⁻, CD10⁺, CD29⁺, CD44⁺, CD54⁺, CD90⁺, SH2⁺, SH3⁺, SH4⁺ and OCT-4⁺. In one embodiment, the population of PDSC comprises cells that are CD34, CD10⁺, CD105⁺, and CD200⁺. In one embodiment, the population of PDSC comprises cells that are CD73⁺. In one embodiment, the population of PDSC comprises cells that are CD73⁺ and CD105⁺. In some embodiments, the population of PDSC comprises cells that are CD200⁺. In one embodiment, the population of PDSC comprises cells that are CD34⁻, CD38⁻, CD45⁻, OCT-4⁺ and CD200⁺. In one embodiment, the population of PDSC comprises cells that are CD34⁻, CD38⁻, CD45⁻, CD73⁺, OCT-4⁺ and CD200⁺. In other embodiments, the population of PDSC comprises cells that are OCT-4⁺. In one embodiment, the population of PDSC comprises cells that are CD73⁺, CD105⁺, and OCT4⁺. In one embodiment, the population of PDSC comprises cells that are CD73⁺, CD105⁺, and CD200⁺. In another embodiment, the population of PDSC comprises cells that are CD73⁺ and CD105⁺. In some embodiments, the population of PDSC comprises cells that are CD200⁺ an OCT-4⁺. In one embodiment, the population of PDSC comprises cells that are CD73⁺, CD105⁺, and HLA-G⁺.

In certain embodiments, the population of PDSC comprises cells that are CD73⁺, CD105⁺, HLA-G⁺. In another embodiment, the population of PDSC comprises cells that are CD73⁺, CD105⁺, CD200⁺ and HLA-G⁺. In one embodiment, the population of PDSC comprises cells that are CD34⁻; CD38⁻; CD45⁻; CD34 and CD38⁻; CD34⁻ and CD45⁻; CD38⁻ and CD45⁻; or CD34⁻, CD38⁻ and CD45⁻. In other embodiments, the population of PDSC comprises cells that are CD34, CD38⁻, CD45 and HLA-G⁺.

In some embodiments, the population of PDSC comprises cells that are CD10⁺, CD38⁻, CD29⁺, CD34⁻, CD44⁺, CD45⁻, CD54⁺, CD90⁺, SH2⁺, SH3⁺, SH4⁺, SSEA3⁻, SSEA4⁻, OCT⁻4⁺, and ABC-p⁺. In other embodiments, the population of PDSC comprises cells that are CD34⁻, CD10⁺, SH2⁺, CD90⁺. In some embodiments, the population of PDSC comprises cells that are CD34, CD38⁻, CD45⁻, CD10⁺, CD29⁺, CD44⁺, CD54⁺, CD90⁺, SH2⁺, SH3⁺, SH4⁺ and OCT-4⁺. In one embodiment, the population of PDSC comprises cells that are CD29⁺, CD45⁻, CD90⁺, SH2⁺, SH3⁺, SH4⁺, or MHC Class II⁻. In some embodiments, the population of PDSC comprises cells that are CD34⁻, SH2⁺, SH3⁺ and SH4⁺. In some embodiments, the population of PDSC comprises cells that are CD34⁻ and MHC Class II⁻. In some embodiments, the population of PDSC comprises cells that are CD29⁺, CD34⁻, CD45⁻, CD90⁺, SH2⁺, SH3⁺, SH4⁺, and MHC Class II.

In some embodiments, the method further comprises characterizing the genome of the stem cells. In one embodiment, the genomic characterization is conducted prior to administration of the population of stem cells to the subject. In another embodiment, the genomic characterization is conducted after administration of the population of stem cells to the subject. In some embodiments, the genomic characterization is conducted prior to administration of the population of stem cells to the subject, and after administration of the population of stem cells to the subject. In one embodiment, the stem cells comprise PDSC. In one embodiment, the stem cells consist essentially of PDSC. In one embodiment, the stem cells consist of PDSC. In one embodiment, the stem cells are PDSC.

In some embodiments, the method further comprises characterizing the proteome of the stem cells. In other embodiments, the proteomic characterization is conducted prior to administration of the population of stem cells to the subject. In another embodiment, the proteomic characterization is conducted after administration of the population of stem cells to the subject. In one embodiment, the proteomic characterization is conducted prior to administration of the population of stem cells to the subject, and after administration of the population of stem cells to the subject. In one embodiment, the stem cells comprise PDSC. In one embodiment, the stem cells consist essentially of PDSC. In one embodiment, the stem cells consist of PDSC. In one embodiment, the stem cells are PDSC.

In certain embodiments, the population of stem cells is autologous to the subject. In some embodiments, the population of stem cells is allogeneic to the subject. In one embodiment, the population of stem cells is syngeneic to the subject. In another embodiment, the population of stem cells is a homogeneous cell population. In other embodiments, the population of stem cells is a mixed cell population. In one embodiment, the population of stem cells is an enriched stem cells population. In certain embodiments, the population of PDSC is autologous to the subject. In some embodiments, the population of stem cells is obtained from multiple donors, optionally without HLA typing. In some embodiments, the population of PDSC is allogeneic to the subject. In one embodiment, the population of PDSC is syngeneic to the subject. In another embodiment, the population of PDSC is a homogeneous cell population. In other embodiments, the population of PDSC is a mixed cell population. In one embodiment, the population of PDSC is an enriched PDSC population. In some embodiments, the population of PDSC comprises PSC-100 cells. In another embodiment, the population of PDSC comprises an enriched population of PSC-100 cells.

In one embodiment, the population of stem cells is administered at a dose of between 1×10⁵ cells and 1×10⁹ cells. In certain embodiments, the population of stem cells is administered at a dose of between 1×10⁵ cells and 1×10⁷ cells. In other embodiments, the population of stem cells is administered at a dose of between 1×10⁶ cells and 1×10⁷ cells. In one embodiment, the population of stem cells comprises a population of PDSC. In another embodiment, the population of stem cells consists essentially of a population of PDSC. In a specific embodiment, the population of stem cells consists of a population of PDSC. Other contemplated doses are provided elsewhere herein.

In one embodiment, the population of stem cells is administered as a single dose. In another embodiment, the population of stem cells is administered as multiple doses. In one embodiment, the population of stem cells is administered when the subject is 10-15 years of age, 15-20 years of age, 20-25 years of age, 25-30 years of age, 30-35 years of age, 35-40 years of age, 40-45 years of age, 45-50 years of age, 50-55 years of age, 55-60 years of age, 60-65 years of age, 65-70 years of age, 70-75 years of age, 75-80 years of age, 80-85 years of age, 85-90 years of age, 90-95 years of age, 95-100 years of age, or over 100 years of age. In some embodiments, it is the first administration of a population of stem cells. In some embodiments, populations of stem cells are serially administered over the lifetime of the subject. In one embodiment, the population of stem cells comprises a population of PDSC. In another embodiment, the population of stem cells consists essentially of a population of PDSC. In a specific embodiment, the population of stem cells consists of a population of PDSC.

In some embodiments, the method further comprises characterizing the genome of the stem cells and/or differentiated cells in the tissue. In another embodiment, the genomic characterization is conducted prior to administration of the population of stem cells (e.g., PDSC) to the subject. In one embodiment, the genomic characterization is conducted after administration of the population of stem cells (e.g., PDSC) to the subject. In some embodiments, the genomic characterization is conducted prior to administration of the population of stem cells (e.g., PDSC) to the subject, and after administration of the population of stem cells (e.g., PDSC) to the subject.

In certain embodiments, the method further comprises characterizing the proteome of the stem cells and/or differentiated cells in the tissue. In one embodiment, the proteomic characterization is conducted prior to administration of the population of stem cells (e.g., PDSC) to the subject. In another embodiment, the proteomic characterization is conducted after administration of the population of stem cells (e.g., PDSC) to the subject. In other embodiments, the proteomic characterization is conducted prior to administration of the population of stem cells (e.g., PDSC) to the subject, and after administration of the population of stem cells (e.g., PDSC) to the subject.

In another aspect, provided herein is a method to recover, isolate, characterize and/or expand cells derived from the remnants of birth (e.g., placenta) which retain the pluripotency, differentiation, and proteomic synthetic diversity of youthful tissue (e.g., PDSC). In one embodiment, the methods comprise recovering the cells. In another embodiment, the method comprises isolating the cells. In other embodiments, the method comprises characterizing the cells. In another embodiment, the method comprises expanding the cells. In particular embodiments, the methods provided herein are used for the purpose of cryopreserving the cells. In some embodiments, the cells are cryopreserved in a form that can be appropriated in the future, for example, to be administered to subjects. In some embodiments, the cells are autologous to the subject. In some embodiments, the population of stem cells is obtained from multiple donors, optionally without HLA typing. In other embodiments, the cells are allogeneic to the subject. In some embodiments, the methods provided herein restore, recharge and/or replenish the pool of stem and progenitor cells (e.g., those cells resident in a tissue). In some embodiments, the cells are restored. In other embodiments, the cells (e.g., those cells resident in a tissue) are recharged. In yet other embodiments, the cells (e.g., those cells resident in a tissue) are replenished. When the cells (e.g., those cells resident in a tissue) are restored, recharged and/or replenished, an improvement in the quality of the general physio-anatomic performance of the recipient can, in certain embodiments, occur. In certain embodiments, the cells (e.g., those cells resident in a tissue) are aged or injured cells.

In certain embodiments, additional methods can be employed for the characterization, expansion, qualification and clinical deployment of the cells for this purpose, and are described elsewhere herein.

In some embodiments, methods are provided herein for the characterization and qualification of expanded and unmanipulated cells. This characterization and qualification can be useful for the purpose of long term cryopreservation and subsequent clinical utilization. The clinical utilization can be any of the various methods provided herein. In certain embodiments, the method results in the restoration of the cellular regenerative potential of the recipient and/or the synthetic capacity of the recipient to combat, reverse, ameliorate the effects of aging; or any combination thereof.

Administration and delivery of cells, e.g., for the purpose of correcting the proteomic and other defects associated with aging, can include any method of parenteral administration, including intravenous infusion, direct intramuscular, subcutaneous, intracompartmental, intraperitoneal, and subdermal administration. The dose and formulation of said cells can also include any conventional means of suspending and injecting said product, including those provided elsewhere herein. In a specific embodiment, the cells are administered to a subject in need thereof.

### 3. BRIEF DESCRIPTION OF THE DRAWINGS

FIG. 1 depicts an exemplary method for isolation and expansion of PDSC.

FIGS. 2A-2C depict general characteristics of rats over life span (3-24 months), such as body mass (FIG. 2A), raw mass of a forelimb muscle (triceps, FIG. 2B), and raw mass of a hindlimb muscle (gastrocnemius, FIG. 2C).

FIGS. 3A-3D depict counts of NCAM (CD56)-positive skeletal muscle satellite cells (FIG. 3A) and relative triceps muscle mass (FIG. 3B) over rat lifespan (3-24 months), association between satellite cell counts and relative triceps muscle mass (FIG. 3C), and representative flow cytometry data (FIG. 3D).

FIGS. 4A-4B depict counts of Pax7-positive skeletal muscle satellite cells over rat lifespan (3-24 months) (FIG. 4A) and representative Pax7 immunofluorescence images of muscle satellite cells (FIG. 4B) at 3 months (top, left panel), 9 months (top, right panel), 18 months (bottom, left panel) and 24 months (bottom, right panel) of age.

FIGS. 5A-5B depict triceps collagen content over rat lifespan (3-24 months) (FIG. 5A) and representative Trichrome™ staining images of triceps (FIG. 5B) at 3 months (top, left panel), 9 months (top, right panel), 18 months (bottom, left panel) and 24 months (bottom, right panel) of age.

FIG. 6 depicts a muscle performance variable, muscle endurance (Rotarod time), over rat lifespan (3-24 months).

FIGS. 7A-7B depict counts of c-kit-positive cells in the heart over rat lifespan (3-24 months) (FIG. 7A) and representative flow cytometry data (FIG. 7B).

FIGS. 8A-8C depict cardiac functions over rat lifespan (3-24 months), including ejection fraction (FIG. 8A), fractional shortening (FIG. 8B), and posterior ventricle wall thickening during contraction (FIG. 8C).

FIGS. 9A-9B depict left ventricle collagen content over rat lifespan (3-24 months) (FIG. 9A) and representative Trichrome™ staining images of left ventricle (FIG. 9B) at 3 months (top, left panel), 9 months (top, right panel), 18 months (bottom, left panel) and 24 months (bottom, right panel) of age.

FIGS. 10A-10D depict counts of CD44-positive cells in the bone marrow (FIG. 10A) and relative femur mass (FIG. 10B) over rat lifespan (3-24 months), representative flow cytometry data (FIG. 10C), and association between bone stem cell counts and relative femur mass (FIG. 10D).

FIGS. 11A-11B depict counts of NCAM (CD56)-positive cells in the hippocampus over rat lifespan (3-24 months) (FIG. 11A) and representative flow cytometry data (FIG. 11B).

FIGS. 12A-12C depict counts of circulating CD31-positive cells (FIG. 12A), counts of Tbx3-positive cells in the liver (FIG. 12B), and counts of CD90-positive cells in the kidney (FIG. 12C) over rat lifespan (3-24 months).

FIGS. 13A-13B depict body mass after either subcutaneous or intravenous administration of PDSC to 11 month, 17 month, or 21 month old rats. FIG. 13A shows absolute body mass in grams, whereas FIG. 13B shows relative body mass normalized to sham (placebo) in each age group.

FIGS. 14A-14C depict absolute muscle performance variables, such as rotations (FIG. 14A), time (FIG. 14B), and distance (FIG. 14C), after either subcutaneous or intravenous administration of PDSC to 11 month, 17 month, or 21 month old rats.

FIGS. 15A-15C depict relative muscle performance variables, such as rotation (FIG. 15A), time (FIG. 15B), and distance (FIG. 15C), after either subcutaneous or intravenous administration of PDSC to 11 month, 17 month, or 21 month old rats. The relative muscle endurance parameters are normalized to sham (placebo) in each age group.

FIGS. 16A-16B depict absolute right gastrocnemius weight (FIG. 16A) and absolute left gastrocnemius weight (FIG. 16B) after either subcutaneous or intravenous administration of PDSC to 11 month, 17 month, or 21 month old rats.

FIGS. 17A-17B depict relative right gastrocnemius weight (FIG. 17A) and relative left gastrocnemius weight (FIG. 17B) after either subcutaneous or intravenous administration of PDSC to 11 month, 17 month, or 21 month old rats. The relative gastrocnemius weights are normalized to sham (placebo) in each age group.

FIGS. 18A-18B depicts the ratio of the left gastrocnemius weight per gram body mass (FIG. 18A) and right gastrocnemius weight per gram body mass (FIG. 18B) after either subcutaneous or intravenous administration of PDSC to 11 month, 17 month, or 21 month old rats.

FIG. 19 depicts flow cytometry data for CD45−CD44+CD73+CD90+CD105+CD271+ cells after either subcutaneous or intravenous administration of PDSC to 11 month, 17 month, or 21 month old rats.

FIGS. 20A-20F depict flow cytometry data for CD45-CD44+ cells (FIG. 20A), CD45−CD73+ cells (FIG. 20B), CD45−CD90+ cells (FIG. 20C), CD45−CD105+ cells (FIG. 20D), CD45−CD271+ cells (FIG. 20E), and CD45− cells (FIG. 20F), after either subcutaneous or intravenous administration of PDSC to 11 month, 17 month, or 21 month old rats.

FIG. 21 depicts flow cytometry data for CD1.1+CD34+CD45+CD47+ cells after either subcutaneous or intravenous administration of PDSC to 11 month, 17 month, or 21 month old rats.

FIGS. 22A-22D depict flow cytometry data for CD11+ cells (FIG. 22A), CD34+ cells (FIG. 22B), CD45+ cells (FIG. 22C), and CD47+ cells (FIG. 22D), after either subcutaneous or intravenous administration of PDSC to 11 month, 17 month, or 21 month old rats.

FIGS. 23A-23C are representative immunofluorescent images of Pax7 (FIG. 23A), Dapi (FIG. 23B), and Pax7/Dapi (FIG. 23C) staining of skeletal muscle cells.

FIGS. 24A-24B depict quantification of endogenous stem cells in plantaris (FIG. 24A) and soleus (FIG. 24B) by measuring Pax7+Dapi+ nuclei in field of view after either subcutaneous or intravenous administration of PDSC to 11 month, 17 month, or 21 month old rats.

FIGS. 25A-25C are representative immunofluorescent images of Ki-67 (FIG. 25A), Dapi (FIG. 25B), and Ki-67/Dapi (FIG. 25C) staining of the subventricular zone of the rat cortex.

FIG. 26 depicts quantification of endogenous stem cells in the subventricular zone of the rat cortex by measuring Ki-67+Dapi+ nuclei in field of view after either subcutaneous or intravenous administration of PDSC to 11 month, 17 month, or 21 month old rats.

FIGS. 27A-27B are representative immunofluorescent images of laminin (FIG. 27A) and an image analysis of the cross-sectional area of the fibers (FIG. 27B) of skeletal muscle cells.

FIGS. 28A-28B depict quantification of average muscle fiber cross-sectional area (CSA) in plantaris (FIG. 28A) and soleus (FIG. 28B) after either subcutaneous or intravenous administration of PDSC to 11 month, 17 month, or 21 month old rats.

FIGS. 29A-29B depict CSA frequency distribution in plantaris (FIG. 29A) or soleus (FIG. 29B) after either subcutaneous or intravenous administration of PDSC to 11 month old rats.

FIGS. 30A-30B depict CSA frequency distribution in plantaris (FIG. 30A) or soleus (FIG. 30B) after either subcutaneous or intravenous administration of PDSC to 17 month old rats.

FIGS. 31A-31B depict CSA frequency distribution in plantaris (FIG. 31A) or soleus (FIG. 31B) after either subcutaneous or intravenous administration of PDSC to 21 month old rats.

FIG. 32 depicts bone marrow CFU assay after either subcutaneous or intravenous administration of PDSC to 11, 17, or 21 month old rats.

FIGS. 33A-33K depict blood measurements for granulocytes (FIG. 33A), RBC (FIG. 33B), granulocytes % (FIG. 33C), HGB (FIG. 33D), HCT % (FIG. 33E), MCV (FIG. 33F), MCH (FIG. 33G), MCHC (FIG. 33H), PLT (FIG. 33I), PCT % (FIG. 33J), and MVP (FIG. 33K) after either subcutaneous or intravenous administration of PDSC to 11 month old rats.

FIGS. 34A-34M depict blood measurements for WBC (FIG. 34A), lymphocytes (FIG. 34B), monocytes (FIG. 34C), granulocytes (FIG. 34D), lymphocytes % (FIG. 34E), monocytes % (FIG. 34F), granulocytes % (FIG. 34G), RBC (FIG. 34H), HGB (FIG. 34I), HCT (FIG. 34J), MCV (FIG. 34K), MCH (FIG. 34L), and MCHC (FIG. 34M) after either subcutaneous or intravenous administration of PDSC to 17 month old rats.

FIGS. 35A-35M depict blood measurements for WBC (FIG. 35A), lymphocytes (FIG. 35B), monocytes (FIG. 35C), granulocytes (FIG. 35D), lymphocytes % (FIG. 35E), monocytes % (FIG. 35F), granulocytes % (FIG. 35G), RBC (FIG. 35H), HGB (FIG. 35I), HCT (FIG. 35J), MCV (FIG. 35K), MCH (FIG. 35L), and MCHC (FIG. 35M) after either subcutaneous or intravenous administration of PDSC to 21 month old rats.

FIG. 36 depicts exemplary pathways on which changes of gene expression in PDSC-treated rats may impact.

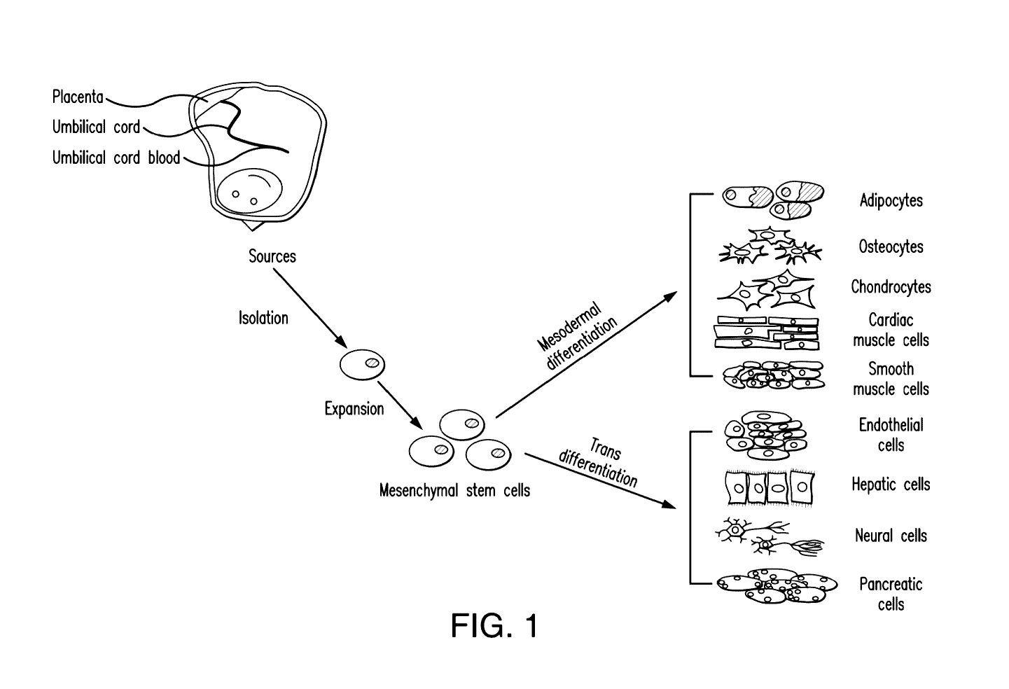

### 4. DETAILED DESCRIPTION

### 4.1 Definitions

All patents, applications, published applications and other publications are incorporated by reference in their entirety. Unless defined otherwise, all technical and scientific terms used herein have the same meaning as is commonly understood by one of ordinary skill in the art. All patents, applications, published applications and other publications are incorporated herein by reference in their entirety. In the event that there is a plurality of definitions for a term herein, those in this section prevail unless stated otherwise.

The term “about” or “approximately” means within 20%, within 10%, within 5%, within 1% or less of a given value or range.

As used herein, “administer” or “administration” refers to the act of injecting or otherwise physically delivering a substance as it exists outside the body (e.g., a PDSC or other stem cell provided herein) into a patient. Delivery, for example, can occur by any method including, but not limited to, intradermal, intravenous, intramuscular, subcutaneous delivery and/or any other method of physical delivery described herein or known in the art.

The term “autologous” as used herein refers to organs, tissues, cells, fluids or other bioactive molecules that are reimplanted in the same individual that they originated from.

As used herein, the term “composition” is intended to encompass a product containing the specified ingredients (e.g., PDSC or other stem cell provided herein) and, optionally, in the specified amounts, as well as any product which results, directly or indirectly, from combination of the specified ingredients in, optionally, the specified amounts.

The term “culturing” as used herein refers to propagating or cultivation of a cell, a population of cells, a tissue, or an organ, by incubating in an environment under conditions and for a period of time sufficient to support cell propagation or viability. Culturing can include expanding or proliferating a cell or population of cells, such as PDSC.

The term “effective amount” as used herein refers to the amount of a therapy (e.g., stem cells, such as PDSC, or a population of stem cells, such as PDSC, as provided herein) which is sufficient to achieve a desired result or specified outcome. In some embodiments, the effective amount is an amount sufficient to reduce and/or ameliorate the severity and/or duration of a given disease, disorder or condition (e.g., aging) and/or a symptom related thereto. This term also encompasses an amount necessary for the reduction or amelioration of the advancement or progression of a given disease, disorder or condition (e.g., aging), reduction or amelioration of the recurrence, development or onset of a given disease, disorder or condition (e.g., aging). In some embodiments, the effective amount of a population of PDSC provided herein is from about 1×10⁵ to about 1×10¹¹, e.g., about 3×10⁵, 5×10⁵, 1×10⁶, 3×10⁶, 5×10⁶, 1×10⁷, 3×10⁷, 5×10⁷, 1×10⁸, 2×10⁸, 3×10⁸, 4×10⁸, 5×10⁸, 6×10⁸, 7×10⁸, 8×10⁸, 8×10⁸, 1×10⁹, 2×10⁹, 3×10⁹, 4×10⁹, 5×10⁹, 1×10¹⁰, 5×10¹⁰, or 1×10¹¹ (or any range therein). In some embodiments, “effective amount” as used herein also refers to the amount of a population of PDSC provided herein to achieve a specified result.

As used herein, a population of cells is “expanded” when it is propagated in vitro or in vivo and gives rise by cell division to other cells. Expansion of cells can spontaneously occur as cells proliferate, e.g., in a culture, or can require certain growth conditions, such as confluence on the surface of a cell culture plate, a minimum cell density, or the addition of agents, such as growth, differentiation or signaling factors. In some embodiments, the cells are stem cells. In one embodiment, the stem cells comprise PDSC. In one embodiment, the stem cells consist essentially of PDSC. In one embodiment, the stem cells consist of PDSC. In certain embodiments, the cells are PDSC.

A placenta has the genotype of the fetus that develops within it, but is also in close physical contact with maternal tissues during gestation. As such, as used herein, the term “fetal genotype” means the genotype of the fetus, e.g., the genotype of the fetus associated with the placenta from which particular isolated placental cells, as described herein, are obtained, as opposed to the genotype of the mother that carried the fetus. As used herein, the term “maternal genotype” means the genotype of the mother that carried the fetus, e.g., the fetus associated with the placenta from which particular isolated placental cells, as described herein, are obtained.

The terms “generate,” “generation” and “generating” as used herein refer to the production of new cells in a subject and optionally the further differentiation into mature, functioning cells.

As used herein, “isolating” a cell (e.g., a PDSC) refers to a process of dissociating or otherwise removing a cell from a tissue sample (e.g., placental tissue), and separating the cells from other cells or non-cells in the tissue. Isolated cells will generally free from contamination by other cell types and will generally be able to be propagated and expanded.

As used herein, the term “isolated cell,” e.g., “isolated stem cell,” means a cell that is substantially separated from other, different cells of the tissue, e.g., placenta, from which the stem cell is derived. A stem cell is “isolated” if at least 50%, 60%, 70%, 80%, 90%, 95%, or at least 99% of the cells with which the population of cells, or cells from which the population of cells is derived, is naturally associated, i.e., stem cells displaying a different marker profile, are removed from the stem cell, e.g., during collection and/or culture of the stem cell. In some embodiments, an isolated cell exists in the presence of a small fraction of other cell types that do not interfere with the utilization of the cell for analysis, production or expansion of the cells. A population of isolated cells can be at least 30%, 40%, 50%, 60%, 70%, 80%, 85%, 90%, 95%, 98%, or 99% pure, or any interval thereof. In a specific embodiment, a population of isolated cells are at least 98% or at least 99% pure. As used herein, the term “population of isolated cells” means a population of cells that is substantially separated from other cells of a tissue, e.g., placenta, from which the population of cells is derived.

As used herein, “multipotent,” when referring to a cell, means that the cell has the ability to differentiate into some, but not necessarily all, types of cells of the body, or into cells having characteristics of some, but not all, types of cells of the body. In certain embodiments, for example, an isolated placental cell that has the capacity to differentiate into a cell having characteristics of neurogenic, chondrogenic and/or osteogenic cells is a multipotent cell.

The terms “optional” or “optionally” as used herein means that the subsequently described event or circumstance may or may not occur, and that the description includes, without limitation, instances where said event or circumstance occurs and instances in which it does not.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, the term “placental-derived stem cell” refers to a stem cell or progenitor cell that is derived from a mammalian placenta, or a portion thereof (e.g., amnion or chorion), regardless of morphology, cell surface markers, or the number of passages after a primary culture. The term “placental-derived stem cell” as used herein does not, however, refer to, and a placental stem cell is not, however, a trophoblast, angioblast, a hemangioblast, an embryonic germ cell, an embryonic stem cell, a cell obtained from an inner cell mass of a blastocyst, or a cell obtained from a gonadal ridge of a late embryo, e.g., an embryonic germ cell. A cell is considered a “stem cell” if the cell retains at least one attribute of a stem cell, e.g., a marker or gene expression profile associated with one or more types of stem cells; the ability to replicate at least 10-40 times in culture, the ability to differentiate into cells of all three germ layers; the lack of adult (i.e., differentiated) cell characteristics, or the like. The terms “placental stem cell,” “placenta-derived stem cell” and “PDSC” may be used interchangeably. Unless otherwise noted herein, the term “placental” includes the umbilical cord. The isolated placental cells disclosed herein, in certain embodiments, differentiate in vitro under differentiating conditions, differentiate in vivo, or both. Reference to a “PDSC” or a “population of PDSC” (that is, 2 or more PDSC) may also be used interchangeably herein for the sake of brevity. For example discussion, of administration of “PDSC” can also mean that a “population of PDSC” can be administered, and vice versa.

As used herein, a placental cell is “positive” for a particular marker when that marker is detectable above background. For example, a placental cell is positive for, e.g., CD73 because CD73 is detectable on placental cells in an amount detectably greater than background (in comparison to, e.g., an isotype control). A cell is also positive for a marker when that marker can be used to distinguish the cell from at least one other cell type, or can be used to select or isolate the cell when present or expressed by the cell. In the context of, e.g., antibody-mediated detection, “positive,” as an indication a particular cell surface marker is present, means that the marker is detectable using an antibody, e.g., a fluorescently-labeled antibody, specific for that marker; “positive” also refers to a cell exhibiting the marker in an amount that produces a signal, e.g., in a cytometer, that is detectably above background. For example, a cell is “CD200⁺” where the cell is detectably labeled with an antibody specific to CD200, and the signal from the antibody is detectably higher than that of a control (e.g., background or an isotype control). Conversely, “negative” in the same context means that the cell surface marker is not detectable using an antibody specific for that marker compared a control (e.g., background or an isotype control). For example, a cell is “CD34⁻” where the cell is not reproducibly detectably labeled with an antibody specific to CD34 to a greater degree than a control (e.g., background or an isotype control). Markers not detected, or not detectable, using antibodies are determined to be positive or negative in a similar manner, using an appropriate control. For example, a cell or population of cells can be determined to be OCT-4⁺ if the amount of OCT-4 RNA detected in RNA from the cell or population of cells is detectably greater than background as determined, e.g., by a method of detecting RNA such as RT-PCR, slot blots, etc. Unless otherwise noted herein, cluster of differentiation (“CD”) markers are detected using antibodies. In certain embodiments, OCT-4 is determined to be present, and a cell is “OCT-4⁺” if OCT-4 is detectable using RT-PCR.

As used herein, the terms “preserve,” “preservation of” and “preserving” in the context, e.g., of an aging or injured cell or tissue refers to protection and/or maintenance of the cell or tissue, or the functions thereof, such that the cell or tissue is not further aged, injured or otherwise compromised, or that the rate of further aging, injury or compromise is slowed relative to the rate in the absence of the intervention at issue. In certain embodiments, preserving cells or tissue comprises the prevention or reduction of the effects of aging. In certain embodiments, preserving cells or tissues comprises prevention or reduction of cell injury.

The terms “regenerate,” “regeneration” and “regenerating” as used herein in the context of aged or injured tissue refer to the process of growing and/or developing new tissue that is aged or has been injured, for example, injured due to disease. In certain embodiments, tissue regeneration comprises activation and/or enhancement of resident cell proliferation, including resident stem cell proliferation.

As used herein, the term “SH2” refers to an antibody that binds an epitope on the marker CD105. Thus, cells that are referred to as SH2⁺ are CD105⁺.

As used herein, the terms “SH3” and “SH4” refer to antibodies that bind epitopes present on the marker CD73. Thus, cells that are referred to as SH3⁺ and/or SH4⁺ are CD73⁺.

As used herein, the term “stem cells” refers to cells that have the capacity to self-renew and to generate differentiated progeny. The term “pluripotent stem cells” refers to stem cells that has complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type. The term “induced pluripotent stem cells” as used herein refers to differentiated mammalian somatic cells (e.g., adult somatic cells, such as skin) that have been reprogrammed to exhibit at least one characteristic of pluripotency. The term “multipotent stem cells” as used herein refers to a stem cell that has the capacity to grow into any subset of the fetal or adult mammalian body's approximately 260 cell types. For example, certain multipotent stem cells can differentiate into at least one cell type of ectoderm, mesoderm and endoderm germ layers. The term “embryonic stem cells” as used herein refers to stem cells derived from the inner cell mass of an early stage embryo, e.g., human, that can proliferate in vitro in an undifferentiated state and are pluripotent. The term “bone marrow stem cells” as used herein refers to stem cells obtained from or derived from bone marrow. The term “amniotic stem cells” as used herein refers to stem cells collected from amniotic fluid or amniotic membrane. The term “embryonic germ cells” as used herein refers to cells derived from primordial germ cells, which exhibit an embryonic pluripotent cell phenotype.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having or at risk of developing a disease, disorder or condition. In some embodiments, the subject is a subject in need thereof.

As used herein, “treat,” “treatment” and “treating” encompass the cure of, remediation of, improvement of, lessening of the severity of, or reduction in the time course of, a disease, disorder or condition, or any parameter or symptom thereof.

### 4.2 Methods of Using Stem Cells (e.g., PDSC)

In certain aspects, the present invention, in part, provides a method and mechanism to recover and produce populations of highly proliferative and proteomically intact stem and progenitor cells (e.g., derived from the placenta). In certain embodiments, the methods provided herein further comprise processing and/or manufacturing theses cells in quantities and with the quality necessary to be stored under cryopreservation conditions. In certain embodiments, the cryopreserved cells can be serially administered at clinically prescribed intervals to restore the regenerative engine of the recipient and to correct the proteomic deficit which exists with advanced age.

Although PDSC are exemplified herein, use of other stem cells is also contemplated.

For example, in some embodiments, the population of stem cells comprises embryonic stem cells. In other embodiments, the population of stem cells comprises adult stem cells. In one embodiment, the population of stem cells comprises mesenchymal stem cells. In another embodiment, the population of stem cells comprises tissue-specific stem cells. In other embodiments, the population of stem cells comprises blood stem cells. In some embodiments, the population of stem cells comprises skin stem cells. In one embodiment, the population of stem cells comprises cord blood stem cells. In other embodiments, the population of stem cells comprises limbal stem cells. In some embodiments, the population of stem cells comprises induced pluripotent stem cells. In another embodiment, the population of stem cells comprises hematopoietic stem cells. In one embodiment, the population of stem cells comprises neural stem cells. In other embodiments, the population of stem cells comprises heart-derived stem cells. In some embodiments, the population of stem cells comprises intestinal stem cells. In some embodiments, the population of stem cells comprises endothelial stem cells. In one embodiment, the population of stem cells comprises epithelial stem cells. In other embodiments, the population of stem cells comprises olfactory adult stem cells. In another embodiment, the population of stem cells comprises neural crest stem cells. In some embodiments, the population of stem cells comprises testicular stem cells. In one embodiment, the population of stem cells comprises placental derived stem cells. In other embodiments, the population of stem cells comprises amniotic fluid-derived stem cells. In specific embodiments, the population of stem cells comprises placental-derived stem cells. In some embodiments, the population of stem cells consists essentially of embryonic stem cells. In another embodiment, the population of stem cells consists essentially of adult stem cells. In other embodiments, the population of stem cells consists essentially of mesenchymal stem cells. In one embodiment, the population of stem cells consists essentially of tissue-specific stem cells. In some embodiments, the population of stem cells consists essentially of blood stem cells. In other embodiments, the population of stem cells consists essentially of skin stem cells. In some embodiments, the population of stem cells consists essentially of cord blood stem cells. In one embodiment, the population of stem cells consists essentially of limbal stem cells. In other embodiments, the population of stem cells consists essentially of induced pluripotent stem cells. In another embodiment, the population of stem cells consists essentially of hematopoietic stem cells. In some embodiments, the population of stem cells consists essentially of neural stem cells. In other embodiments, the population of stem cells consists essentially of heart-derived stem cells. In one embodiment, the population of stem cells consists essentially of intestinal stem cells. In some embodiments, the population of stem cells consists essentially of endothelial stem cells. In other embodiments, the population of stem cells consists essentially of epithelial stem cells. In another embodiment, the population of stem cells consists essentially of olfactory adult stem cells. In one embodiment, the population of stem cells consists essentially of neural crest stem cells. In other embodiments, the population of stem cells consists essentially of testicular stem cells. In some embodiments, the population of stem cells consists essentially of placental derived stem cells. In some embodiments, the population of stem cells consists essentially of amniotic fluid-derived stem cells. In specific embodiments, the population of stem cells consists essentially of placental-derived stem cells. In other embodiments, the population of stem cells consists of embryonic stem cells. In one embodiment, the population of stem cells consists of adult stem cells. In another embodiment, the population of stem cells consists of mesenchymal stem cells. In other embodiments, the population of stem cells consists of tissue-specific stem cells. In some embodiments, the population of stem cells consists of blood stem cells. In one embodiment, the population of stem cells consists of skin stem cells. In other embodiments, the population of stem cells consists of cord blood stem cells. In some embodiments, the population of stem cells consists of limbal stem cells. In another embodiment, the population of stem cells consists of induced pluripotent stem cells. In other embodiments, the population of stem cells consists of hematopoietic stem cells. In one embodiment, the population of stem cells consists of neural stem cells. In some embodiments, the population of stem cells consists of heart-derived stem cells. In other embodiments, the population of stem cells consists of intestinal stem cells. In some embodiments, the population of stem cells consists of endothelial stem cells. In one embodiment, the population of stem cells consists of epithelial stem cells. In other embodiments, the population of stem cells consists of olfactory adult stem cells. In another embodiment, the population of stem cells consists of neural crest stem cells. In some embodiments, the population of stem cells consists of testicular stem cells. In other embodiments, the population of stem cells consists of placental derived stem cells. In one embodiment, the population of stem cells consists of amniotic fluid-derived stem cells. In specific embodiments, the population of stem cells consists of placental-derived stem cells. In some embodiments, the population of stem cells comprises bone marrow mesenchymal stem cells. In other embodiments, the population of stem cells comprises amniotic membrane-derived mesenchymal stem cells. In another embodiment, the population of stem cells comprises adipose tissue-derived mesenchymal stem cells. In one embodiment, the population of stem cells comprises stem cells from human exfoliated deciduous teeth. In other embodiments, the population of stem cells comprises skeletal muscle-derived stem cells. In some embodiments, the population of stem cells does not comprise bone marrow mesenchymal stem cells. In some embodiments, the population of stem cells does not comprise amniotic membrane-derived mesenchymal stem cells. In other embodiments, the population of stem cells does not comprise adipose tissue-derived mesenchymal stem cells. In one embodiment, the population of stem cells does not comprise stem cells from human exfoliated deciduous teeth. In another embodiment, the population of stem cells does not comprise skeletal muscle-derived stem cells. In some embodiments, the population of stem cells consists essentially of bone marrow mesenchymal stem cells. In other embodiments, the population of stem cells consists essentially of amniotic membrane-derived mesenchymal stem cells. In one embodiment, the population of stem cells consists essentially of adipose tissue-derived mesenchymal stem cells. In some embodiments, the population of stem cells consists essentially of stem cells from human exfoliated deciduous teeth. In other embodiments, the population of stem cells consists essentially of skeletal muscle-derived stem cells. In another embodiment, the population of stem cells does not consist essentially of bone marrow mesenchymal stem cells. In one embodiment, the population of stem cells does not consist essentially of amniotic membrane-derived mesenchymal stem cells. In some embodiments, the population of stem cells does not consist essentially of adipose tissue-derived mesenchymal stem cells. In other embodiments, the population of stem cells does not consist essentially of stem cells from human exfoliated deciduous teeth. In some embodiments, the population of stem cells does not consist essentially of skeletal muscle-derived stem cells. In one embodiment, the population of stem cells consists of bone marrow mesenchymal stem cells. In other embodiments, the population of stem cells consists of amniotic membrane-derived mesenchymal stem cells. In another embodiment, the population of stem cells consists of adipose tissue-derived mesenchymal stem cells. In some embodiments, the population of stem cells consists of stem cells from human exfoliated deciduous teeth. In other embodiments, the population of stem cells consists of skeletal muscle-derived stem cells. In one embodiment, the population of stem cells does not consist of bone marrow mesenchymal stem cells. In some embodiments, the population of stem cells does not consist of amniotic membrane-derived mesenchymal stem cells. In other embodiments, the population of stem cells does not consist of adipose tissue-derived mesenchymal stem cells. In another embodiment, the population of stem cells does not consist of stem cells from human exfoliated deciduous teeth. In one embodiment, the population of stem cells does not consist of skeletal muscle-derived stem cells.

In one aspect, provided herein is a method for maintaining or increasing the ratio of the number stem cells to the number of differentiated cells in a tissue of a subject in need thereof over time. In one embodiment, the method comprises administering to the subject an effective amount of a population of stem cells, wherein the ratio is maintained or increased over time as compared to the ratio of the number stem cells to the number of differentiated cells in a tissue of a control subject over time. In one embodiment, the method comprises administering to the subject an effective amount of a population of PDSC, wherein the ratio is maintained or increased over time as compared to the ratio of the number stem cells to the number of differentiated cells in a tissue of a control subject over time.

In a second aspect, provided herein is a method of maintaining or increasing the number of stem cells in a tissue of a subject in need thereof over time. In certain embodiments, the method comprises administering to the subject an effective amount of a population of stem cells, wherein the number of stem cells in the tissue of the subject is maintained or increased over time as compared to the number of stem cells in the same tissue of a control subject. In certain embodiments, the method comprises administering to the subject an effective amount of a population of PDSC, wherein the number of stem cells in the tissue of the subject is maintained or increased over time as compared to the number of stem cells in the same tissue of a control subject. In one embodiment, the population of stem cells comprises a population of PDSC. In another embodiment, the population of stem cells consists essentially of a population of PDSC. In a specific embodiment, the population of stem cells consists of a population of PDSC.

In certain embodiments, the control subject is same subject before administration of the population of stem cells (e.g., PDSC). In other embodiments, the control subject is a subject that has not received the population of stem cells (e.g., PDSC).

In a third aspect, provided herein is a method of altering the phenotype of an aging stem cell resident in a tissue of a subject in need thereof, comprising administering to the subject an effective amount of a population of stem cells, wherein the amount is effective to alter the environmental niche of the aging stem cell such that the phenotype of the stem cell is altered as compared to the phenotype of the stem cell resident in the tissue of a control subject. Also provided herein is a method of altering the phenotype of an aging stem cell resident in a tissue of a subject in need thereof, comprising administering to the subject an effective amount of a population of PDSC, wherein the amount is effective to alter the environmental niche of the aging stem cell such that the phenotype of the stem cell is altered as compared to the phenotype of the stem cell resident in the tissue of a control subject.

In some embodiments of the various methods provided herein, such methods result in the preservation of the aging stem cell. In other embodiments of the various methods provided herein, such methods result in the preservation of differentiated cells, e.g., within the tissue of the subject. In yet other embodiments of the various methods provided herein, such methods result in the preservation of the tissue itself. In some embodiments of the various methods provided herein, such methods result in decreasing the effects of aging in a subject, such as a subject in need thereof. In other embodiments of the various methods provided herein, such methods result in increasing the lifespan of a subject, such as a subject in need thereof. In some embodiments, the methods result in the proliferation of previously quiescent cells. In some embodiments, the methods result in the restoration of the cellular regenerative potential of the cardiovascular system. For example, in some instances, the methods result in restoration of the cellular regenerative potential of the vascular endothelium. In other instances, the methods result in restoration of the cellular regenerative potential of the blood vessel wall. In some embodiments, the methods result in the maintenance or improvement of the structure of the vascular endothelium. In some instances, the methods result in the maintenance or improvement of the function of the vascular endothelium. In some embodiments, the methods result in the maintenance or improvement of the structure of the blood vessel wall. In some instances, the methods result in the maintenance or improvement of the function of the blood vessel wall. In other embodiments, the methods result in the restoration of the cellular regenerative potential of skeletal muscle. In some embodiments, the methods result in a decrease in the degree of fibrosis. In some instances, the methods result in the maintenance or improvement of the skeletal muscle structure. In other instances, the methods result in the maintenance or improvement of the skeletal muscle functionality. In some embodiments, the methods result in a reduction in calcium deposits. In some embodiments, the methods result in increased proliferation of skin cells. For example, in some instances, the methods result in increased proliferation of keratinocytes, melanocytes, Merkel cells, Langerhans cells, or a combination thereof. In other embodiments, the methods result in the maintenance or increase in the rate of epidermal cell replacement. In some embodiments, the methods result in the maintenance or improvement in the production of one or more proteins in a skin cells. For example, in some embodiments, the methods result in preservation or improvement of collagen production. In other embodiments, the methods result in preservation or improvement of elastin production. In some embodiments, the methods result in improved skin appearance. For example, in some embodiments, the methods result in smoother appearing skin. In other embodiments, the methods result in the maintenance or improvement in skin thickness. In some embodiments, the methods result in the maintenance or reduction of the skin's sensitivity to bruising or other types of injuries. In some embodiments, the methods result in a maintenance or increase in the quantity of fat cells, bone, and/or cartilage beneath the skin. In some embodiments, the methods result in the prevention of the loss of fat cells, bone, and/or cartilage beneath the skin. In some embodiments, the methods result in the restoration or improvement of the cellular regenerative potential of the liver. In some embodiments, the methods result in the maintenance or improvement of the functional anatomy of the liver. For example, in some instances, the methods result in the maintenance or improvement of the biliary anatomy, liver volume, hepatocyte morphology, blood supply, or any combination thereof. In some embodiments, the methods result in the maintenance or improvement of liver functionality. In some embodiments, the methods result in the restoration or improvement of the cellular regenerative potential of the kidney. In some embodiments, the methods result in the maintenance or improvement in functional renal anatomy. For example, in some embodiments, the methods result in the maintenance or improvement of parenchymal volume, glomerular unity density, kidney perfusion, or any combination thereof. In some embodiments, the methods result in the preservation or improvement of the renal parenchyma. In some embodiments, the methods result in the maintenance or improvement of kidney function. In some embodiments, the methods result in the restoration or improvement of the cellular regenerative potential of the brain. In some embodiments, the methods result in the maintenance or improvement of brain volume, cerebral perfusion, neurotransmitter synthesis, neurotransmitter metabolism, or a combination thereof. In some embodiments, the methods result in the modification of protein synthesis or degradation in brain cells. In some embodiments, the methods result in preservation or improvement of the cognitive functions of the subject. In some embodiments, the methods result in preservation or improvement of the motor functions of the subject. In some embodiments, the methods result in preservation or improvement of the cognitive and motor functions of the subject. In some embodiments, the methods result in the reduction in the rate of decline of the cognitive motor functions of the subject. In some embodiments, the methods result in the reduction in the rate of decline of the motor functions of the subject. In some embodiments, the methods result in the reduction in the rate of decline of the cognitive and motor functions of the subject. In some embodiments, the methods result in the restoration or improvement of the cellular regenerative potential of the bone marrow. In some embodiments, the methods result in the preservation or improvement of the colony forming unit potential of the bone marrow. In some embodiments, the methods result in the preservation or improvement of the cellularity of the bone marrow. In some embodiments, the methods result in increased hematopoiesis. In some embodiments, the methods result in the maintenance or improvement of stromal cell function. In some embodiments, the methods result in the preservation or improvement of the immune response. In specific embodiment, the maintenance or improvement is in a subject. In specific embodiments, the maintenance or improvement is in a subject in need thereof.

In some embodiments, the subject has a disease or disorder. In certain embodiments, the disease or disorder is sarcopenia. In other embodiments, the disease or disorder is a blood cancer. In other embodiments, the disease or disorder is a degenerative disorder. In some instances, the degenerative disorder is an age related degenerative disorder in a tissue or organ. In some embodiments, the disease or disorder is a metabolic disorder. In other embodiments, the disease or disorder is a cardiovascular disease. In some instances, the disease or disorder is a neurodegenerative disorder. In certain embodiments, the disease or disorder is osteoporosis. In other embodiments, the disease or disorder is normal aging of the skin. In some embodiments, the disease or disorder is a liver, kidney or immune disease.

In one embodiment, provided herein is a method to alter an aging stem cell through modifying the environmental niche in which it resides. In some embodiments, the environmental niche is modified for the purpose of reconditioning the phenotype of the aging stem cell to one with a longer lifespan. In certain embodiments, the method comprises in vivo or in vitro co-cultivation of aging stem cells with progenitor cells from a younger source (e.g., PDSC). In some embodiments, the co-cultivation effectuates a molecular and/or genetic event, which can have the net effect of rejuvenating the aged cells. In certain embodiments, the various methods provided herein can be used to phenotypically modify an aging organism to express traits consistent with a younger phenotype.

In certain embodiments, the methods provided herein can encompass a range of in vivo and in vitro methods by which exposure of aging cells to younger progenitors results in the transfer or transition to a youthful phenotype. In some embodiments, the various methods provided herein can comprise co-cultivation in vitro. In other embodiments, the various methods provided herein include niche conditioning in vivo. In certain embodiments, the niche reconditioning is accomplished by delivering young progenitors (e.g., PDSC) to a phyioanatomic niche (e.g., the bone marrow or organ system). In other embodiments, the conditioning of an aging niche is accomplished through the delivery of bioactive factors, such as paracrine factors, isolated from young progenitors.

In specific embodiments, the various methods provided herein will result in the modulation of the phenotype of aging cells by inducing the repertoire of genotypically expressed factors characteristic of a youthful phenotypic state by exposure. e.g., to placental cells and/or secreted factors thereof.

In a specific embodiment, the aging stem cell is in a tissue of a subject in need thereof. In other embodiments, the aging stem cell is derived from a subject in need thereof.

In some embodiments, the methods provided herein comprise controlled co-cultivation with stem cells (e.g., in situ or in vitro). In other embodiments, the methods provided herein include the therapeutic administration of stem cells.

In some embodiments, the methods provided herein comprise controlled co-cultivation with placental cells (e.g., in situ or in vitro). In other embodiments, the methods provided herein include the therapeutic administration of placental cells (e.g., PDSC). In some embodiments, the administration is to a subject, e.g., a subject in need thereof. Such cells can be used to exploit the secretome of the transiently or permanently residing stem cells on the aging cells of the recipient. The placenta is the source of a range of highly proliferative stem and progenitor cells, which express a robust secretome of growth and regulatory factors as provided elsewhere herein. Such placental cells can induce immunologic tolerance. Such placental cells can also stimulate endogenous stem cell regeneration. Placental cells have also been successfully transplanted into humans for a variety of clinical indications including autoimmune disease, stroke and cancer.

Various methods provided herein can be used, for example, to control the phenotype of cells (e.g., aging cells) by exposing them to cells of a youthful chronobiological age (e.g., PDSC). In some embodiments, the aging cells are exposed to PDSC using a placental bioreactor. In other embodiments, the aging cells are exposed to PDSC using a co-cultivation system. In yet other embodiments, the aging cells are exposed to PDSC following administration of placental cells to the subject, for example via intravenous infusion, direct injection, or other form of parenteral administration. In a specific embodiment, the aging cells are of a subject, such as a subject in need thereof.

In the certain embodiments of the various methods provided herein, stem cells, such as stem cells derived from newborn placenta (e.g., PDSC) are used in a co-culture environment as a “feeder” layer upon which cells from an older donor are cultivated. In certain embodiments, the cells from the older donor are stem cell, progenitor cells, or other cells which retain the ability to propagate when returned to the host. In some embodiments, the stem cells derived from the newborn placenta are expanded in vitro in culture. In other embodiments, the stem cells derived from the newborn placenta are unexpanded. In certain embodiments, after a period of co-cultivation, the donor cells will be isolated from the feeder layer. In a specific embodiment, the donor cells will then be reintroduced into the donor. In a specific embodiment, the host or donor is a subject in need thereof.

In another embodiment, the newborn cells (e.g., PDSC) are cultured in an extracorporeal device. In some embodiments, the extracorporeal device is placed in the circulatory circuit of a recipient subject such that the secreted factors of newborn cells are delivered to the subject. In a specific embodiment, the subject is a subject in need thereof.

In yet other embodiments of the various methods provided herein, the newborn cells (e.g., PDSC) are administered therapeutically (e.g., either systemically or locally). In some embodiments, the newborn cells are administered via injection. In other embodiments, the newborn cells are administered via infusion. In such methods, the cells can traffic through the recipient subject and take up short- or long-term residence in proximity to the recipients aging cells. The cells can then, in certain embodiments, effectively alter the aging phenotype of recipient cells to a more youthful phenotype. In some embodiments, the alteration is the result of direct or indirect contact of the aging cells with paracrine factors, endocrine factors and/or direct cell-to-cell interactions, e.g., with the newborn cells.

In another aspect, provided herein are methods of altering the proteome of an aging cell in a tissue of a subject in need thereof. In some embodiments, the method of altering the proteome of an aging cell in a tissue comprises administering to the subject an effective amount of a population of stem cells, wherein the amount is effective to alter the proteome of the aging cell, wherein the altered proteome comprises one or more biomarkers found in a younger cell in the tissue of a control subject. Also provided herein are methods of altering the proteome of an aging cell in a tissue of a subject in need thereof. In some embodiments, the method of altering the proteome of an aging cell in a tissue comprises administering to the subject an effective amount of a population of PDSC, wherein the amount is effective to alter the proteome of the aging cell, wherein the altered proteome comprises one or more biomarkers found in a younger cell in the tissue of a control subject. In some embodiments, the biomarker is increased relative to the same biomarker found in the younger cell. In other embodiments, the biomarker is decreased relative to the same biomarker found in the younger cell.

In some embodiments, one or more biomarkers are selected from the group consisting of myosin light chain 3 (MLCF3), myosin light polypeptide 2 (slow), myosin light chain 1 (MLC1F), myosin binding protein C (MYBPC1), myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), heat shock 27 kDa protein (Hsp27), disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, myosin heavy chain 2 (MYH2), troponin T type 1 (TNNT1), ryanodine receptor 1 (skeletal) (RYR1), calsequestrin 1 (fast-twitch, skeletal muscle) (CASQ1), junctophilin 1 (JPH1), adenosine monosphosphate deaminase (AMPD1), phosphorylase glycogen muscle (PYGM), and enolase 3 (beta, muscle) (ENO3). In some embodiments, two or more biomarkers are selected from the group consisting of MLCF3, myosin light polypeptide 2 (slow), MLC1F, MYBPC1, myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, MYH2, TNNT1, RYR1, CASQ1, JPH1, AMPD1, PYGM, and ENO3. In some embodiments, the biomarker is MLCF3. In some embodiments, three or more biomarkers are selected from the group consisting of MLCF3, myosin light polypeptide 2 (slow), MLC1F, MYBPC1, myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), Hsp20, disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, MYH2, TNNT1, RYR1, CASQ1, JPH1, AMPD1, PYGM, and ENO3. In some embodiments, the biomarker is MLCF3. In some embodiments, 4, 5, 6, 7, 8, 9, 10, 11, 12, 13, 14, 15, 16, 17, 18, 19, 20, 25, 30, 35, 40, or 45 or more biomarkers are selected from the group consisting of MLCF3, myosin light polypeptide 2 (slow), MLC1F, MYBPC1, myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), Hsp20, disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, MYH2, TNNT1, RYR1, CASQ1, JPH1, AMPD1, PYGM, and ENO3. In some embodiments, the biomarker is MLCF3. In some embodiments, the biomarker is myosin light polypeptide 2 (slow). In some embodiments, the biomarker is MLC1F. In some embodiments, the biomarker is myosin binding protein C (MYBPC1). In some embodiments, the biomarker is myosin binding protein H. In some embodiments, the biomarker is alpha actin (fragment). In some embodiments, the biomarker is actin (skeletal muscle). In some embodiments, the biomarker is actin alpha (cardiac). In some embodiments, the biomarker is troponin T class Ia alpha-1. In some embodiments, the biomarker is troponin T class IIa beta-1. In some embodiments, the biomarker is troponin T beta/alpha. In some embodiments, the biomarker is capZ beta. In some embodiments, the biomarker is desmin. In some embodiments, the biomarker is gelsolin (cytosolic). In some embodiments, the biomarker is beta-tubulin. In some embodiments, the biomarker is p23. In some embodiments, the biomarker is triosephosphate isomerase 1. In some embodiments, the biomarker is glycosylase I. In some embodiments, the biomarker is glyoxalase I. In some embodiments, the biomarker is enolase 3 (beta muscle). In some embodiments, the biomarker is glycerol 3-P dehydrogenase. In some embodiments, the biomarker is isocitrate dehydrogenase 3 (NAD+). In some embodiments, the biomarker is cytochrome c oxidase (polypeptide Va). In some embodiments, the biomarker is creatine kinase (muscle form). In some embodiments, the biomarker is Cu/Zn superoxide dismutase. In some embodiments, the biomarker is ferritin heavy chain (H-ferritin). In some embodiments, the biomarker is aldehyde dehydrogenase (mitochondrial). In some embodiments, the biomarker is glutathione transferase (omega 1). In some embodiments, the biomarker is heat shock 20 kDa protein (Hsp20). In some embodiments, the biomarker is Hsp20. In some embodiments, the biomarker is disulfide isomerase ER60 (ERp57). In some embodiments, the biomarker is 14-3-3 protein. In some embodiments, the biomarker is guanine deaminase (guanase). In some embodiments, the biomarker is Rho-GDI (alpha). In some embodiments, the biomarker is phosphohistidine phosphatase. In some embodiments, the biomarker is mRNA capping enzyme. In some embodiments, the biomarker is similar to apobec2 protein. In some embodiments, the biomarker is galectin 1. In some embodiments, the biomarker is albumin. In some embodiments, the biomarker is vitamin D binding protein prepeptide. In some embodiments, the biomarker is protein kinase C interacting protein-1. In some embodiments, the biomarker is RIKEN cDNA 1700012G19. In some embodiments, the biomarker is MYH2. In some embodiments, the biomarker is TNNT1. In some embodiments, the biomarker is RYR1. In some embodiments, the biomarker is CASQ1. In some embodiments, the biomarker is JPH1. In some embodiments, the biomarker is AMPD1. In some embodiments, the biomarker is PYGM. In some embodiments, the biomarker is ENO3. In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the one or more biomarkers is indicative of aging. In some embodiments, two or more biomarkers are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the two or more biomarkers is indicative of aging. In some embodiments, three or more biomarkers are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the three or more biomarkers is indicative of aging. In some embodiments, 4, 5, 6, 7, 8, 9, 10, 11, 12, 13, 14, 15, 16, 17, 18, 19, or 20 or more biomarkers are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the biomarkers is indicative of aging. In some embodiments, a decrease in the expression of MLCF3 is indicative of aging. In some embodiments, a decrease in the expression of myosin light polypeptide 2 (slow) is indicative of aging. In some embodiments, a decrease in the expression of MLC1F is indicative of aging. In some embodiments, a decrease in the expression of myosin binding protein C is indicative of aging. In some embodiments, a decrease in the expression of myosin binding protein H is indicative of aging. In some embodiments, a decrease in the expression of alpha actin (fragment) is indicative of aging. In some embodiments, a decrease in the expression of actin (skeletal muscle) is indicative of aging. In some embodiments, a decrease in the expression of actin alpha (cardiac) is indicative of aging. In some embodiments, a decrease in the expression of troponin T class IIa beta-1 is indicative of aging. In some embodiments, a decrease in the expression of troponin T beta/alpha is indicative of aging. In some embodiments, a decrease in the expression of capZ beta is indicative of aging. In some embodiments, a decrease in the expression of triosephosphate isomerase 1 is indicative of aging. In some embodiments, a decrease in the expression of glycosylase I is indicative of aging. In some embodiments, a decrease in the expression of glyoxalase I is indicative of aging. In some embodiments, a decrease in the expression of enolase 3 (beta muscle) is indicative of aging. In some embodiments, a decrease in the expression of glycerol 3-P dehydrogenase is indicative of aging. In some embodiments, a decrease in the expression of isocitrate dehydrogenase 3 (NAD+) is indicative of aging. In some embodiments, a decrease in the expression of cytochrome c oxidase (polypeptide Va) is indicative of aging. In some embodiments, a decrease in the expression of creatine kinase (muscle form) is indicative of aging. In some embodiments, a decrease in the expression of Cu/Zn superoxide dismutase is indicative of aging. In some embodiments, a decrease in the expression of phosphohistidine phosphatase is indicative of aging. In some embodiments, a decrease in the expression of protein kinase C interacting protein-1 is indicative of aging. In some embodiments, a decrease in the expression of RIKEN cDNA 1700012G19 is indicative of aging.

In some embodiments, the one or more biomarkers are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), heat shock 20 kDa protein (Hsp20), Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the one or more biomarkers is indicative of aging. In some embodiments, two or more biomarkers are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), heat shock 20 kDa protein (Hsp20), Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the biomarkers is indicative of aging. In some embodiments, three or more biomarkers are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), heat shock 20 kDa protein (Hsp20), Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the biomarkers is indicative of aging. In some embodiments, 4, 5, 6, 7, 8, 9, 10, 11, 12, 13, 14, 15, 16, 17, 18, 19, or 20 or more biomarkers are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), heat shock 20 kDa protein (Hsp20), Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the biomarkers is indicative of aging. In some embodiments, an increase in the expression of troponin T class Ia alpha-1 is indicative of aging. In some embodiments, an increase in the expression of troponin T class IIa beta-1 is indicative of aging. In some embodiments, an increase in the expression of desmin is indicative of aging. In some embodiments, an increase in the expression of gelsolin (cytosolic) is indicative of aging. In some embodiments, an increase in the expression of beta-tubulin is indicative of aging. In some embodiments, an increase in the expression of p23 is indicative of aging. In some embodiments, an increase in the expression of ferritin heavy chain (H-ferritin) is indicative of aging. In some embodiments, an increase in the expression of aldehyde dehydrogenase (mitochondrial) is indicative of aging. In some embodiments, an increase in the expression of glutathione transferase (omega 1) is indicative of aging. In some embodiments, an increase in the expression of heat shock 20 kDa protein (Hsp20) is indicative of aging. In some embodiments, an increase in the expression of Hsp20 is indicative of aging. In some embodiments, an increase in the expression of disulfide isomerase ER60 (ERp57) is indicative of aging. In some embodiments, an increase in the expression of 14-3-3 protein is indicative of aging. In some embodiments, an increase in the expression of guanine deaminase (guanase) is indicative of aging. In some embodiments, an increase in the expression of Rho-GDI (alpha) is indicative of aging. In some embodiments, an increase in the expression of mRNA capping enzyme is indicative of aging. In some embodiments, an increase in the expression of similar to apobec2 protein (Accession No. XP217334) is indicative of aging. In some embodiments, an increase in the expression of galectin 1 is indicative of aging. In some embodiments, an increase in the expression of albumin is indicative of aging. In some embodiments, an increase in the expression of vitamin D binding protein prepeptide is indicative of aging.

In some embodiments, one or more biomarkers are selected from the group consisting of myristoylated alanine-rich C-kinase substrate, alpha-internexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of myelin basic protein (MBP), and vimentin (VIM). In some embodiments, two or more biomarkers are selected from the group consisting of myristoylated alanine-rich C-kinase substrate, alpha-intemexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of MBP, and VIM. In some embodiments, three or more biomarkers are selected from the group consisting of myristoylated alanine-rich C-kinase substrate, alpha-intemexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of MBP, and VIM. In some embodiments, 4, 5, 6, 7, 8, 9, 10, 11, 12, 13, 14, 15, 16, 17, 18, 19, or 20 or more biomarkers are selected from the group consisting of myristoylated alanine-rich C-kinase substrate, alpha-intemexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of MBP, and VIM. In some embodiments, the biomarker is myristoylated alanine-rich C-kinase substrate. In some embodiments, the biomarker is alpha-internexin. In some embodiments, the biomarker is isoform B of methyl-CpG-binding protein 2. In some embodiments, the biomarker is histone H1.4. In some embodiments, the biomarker is isoform 1 of serum albumin. In some embodiments, the biomarker is guanine nucleotide-binding protein (G(1)/G(S)/G(T) subunit beta-1. In some embodiments, the biomarker is adenylate kinase 1. In some embodiments, the biomarker is fructose-biphosphate aldolase A. In some embodiments, the biomarker is tenascin-R. In some embodiments, the biomarker is isoform 2 of clusterin. In some embodiments, the biomarker is synaptic transmission. In some embodiments, the biomarker is cation transport. In some embodiments, the biomarker is isoform 1 of myeline proteolipid protein. In some embodiments, the biomarker is neuromodulin. In some embodiments, the biomarker is dihydropyrimidinase-related protein 2. In some embodiments, the biomarker is dihydropteridine reductase. In some embodiments, the biomarker is matrin-3. In some embodiments, the biomarker is alpha-enolase. In some embodiments, the biomarker is isoform 1 of gelsolin. In some embodiments, the biomarker is APP isoform of APP714 of amyloid beta A4 protein (fragment). In some embodiments, the biomarker is annexin A6. In some embodiments, the biomarker is isoform tau-E of microtubule-associated protein tau. In some embodiments, the biomarker is MAP1A 331 kDa protein. In some embodiments, the biomarker is neuroblast differentiation-associated protein AH NAK. In some embodiments, the biomarker is cell cycle exit and neuronal differentiation protein 1. In some embodiments, the biomarker is glyceraldehyde-3-phosphate dehydrogenase. In some embodiments, the biomarker is HIST1H1D. In some embodiments, the biomarker is isoform KGA of glutaminase kidney isoform. In some embodiments, the biomarker is superoxide dismutase (Mn) (SOD2). In some embodiments, the biomarker is isoform 1 of MBP. In some embodiments, the biomarker is VIM. In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of amyloid beta (A4) precursor protein (APP), myristoylated alanine-rich protein kinase C substrate (MARCKS), internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, two or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, three or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, 4, 5, 6, 7, 8, 9, 10, 11, 12, 13, 14, 15, 16, 17, 18, 19, 20, 25, 30, 35, 40, 45, or 50 or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, the biomarker is amyloid beta (A4) precursor protein (APP). In some embodiments, the biomarker is marcks. In some embodiments, the biomarker is internexin neuronal intermediate filament protein alpha (INA). In some embodiments, the biomarker is methyl CpG binding protein (MECP). In some embodiments, the biomarker is histone cluster 1 H1e (HIST1H1E). In some embodiments, the biomarker is albumin (ALB). In some embodiments, the biomarker is guanine nucleotide binding protein (G protein) beta polypeptide (GNB1). In some embodiments, the biomarker is adenylate kinase 1 (AK1). In some embodiments, the biomarker is aldose A fructose-biphosphate (ALDOA). In some embodiments, the biomarker is tenascin R (TNR). In some embodiments, the biomarker is clusterin (CLU). In some embodiments, the biomarker is synapsin 1 (SYN1). In some embodiments, the biomarker is ATP synthase. In some embodiments, the biomarker is H+ transporting. In some embodiments, the biomarker is mitochondrial F1 complex. In some embodiments, the biomarker is alpha subunit 1. In some embodiments, the biomarker is cardiac musle (ATP5A1). In some embodiments, the biomarker is proteolipid protein 1 (PLP1). In some embodiments, the biomarker is growth associated protein 43 (GAP43). In some embodiments, the biomarker is dihydropyrimidinase-like 2 (DPYSL2). In some embodiments, the biomarker is quinoid dihydropteridine reductase (QDPR). In some embodiments, the biomarker is matrin 3 (MATR3). In some embodiments, the biomarker is enolase 1 (alpha) (ENO1). In some embodiments, the biomarker is gelsolin (GSN). In some embodiments, the biomarker is annexin A6 (ANXA6). In some embodiments, the biomarker is microtubule associated protein tau (MAPT). In some embodiments, the biomarker is microtuble-associated protein 1A (MAP1A). In some embodiments, the biomarker is AHNAK nucleoprotein. In some embodiments, the biomarker is cell cycle exit and neuronal differentiation 1 (CEND1). In some embodiments, the biomarker is glyceraldehyde-3-phosphate dehydrogenase (GAPDH). In some embodiments, the biomarker is histone cluster 1. In some embodiments, the biomarker is Hid (HIST1H1D). In some embodiments, the biomarker is glutaminase (GLS). In some embodiments, the biomarker is superoxide dismutase (SOD2). In some embodiments, the biomarker is MBP. In some embodiments, the biomarker is VIM. In some embodiments, the biomarker is ELAV-like protein 3 (ELAVL3). In some embodiments, the biomarker is neurogranin (NRGN). In some embodiments, the biomarker is receptor expression enhancing protein 2 (REEP2). In some embodiments, the biomarker is glutamate decarboxylase 1 (GAD1). In some embodiments, the biomarker is protocadherin alpha-1 (PCDHA1). In some embodiments, the biomarker is glial fibrillary acidic protein (GFAP). In some embodiments, the biomarker is S100 calcium binding protein (S100B). In some embodiments, the biomarker is family with sequence similarity 19 (chemokine (C-C-motif)-like). In some embodiments, the biomarker is member A1 (FAM19A1). In some embodiments, the biomarker is aquaporin 4 (AQP4). In some embodiments, the biomarker is c-type lectin domain family 2. In some embodiments, the biomarker is member L (CLEC2L). In some embodiments, the biomarker is neurofilament triplet L protein (NF-L). In some embodiments, the biomarker is peroxiredoxin (EC 1.11.1.). In some embodiments, the biomarker is aconitate hydratase (EC 4.2.1.3). In some embodiments, the biomarker is enolase 2 (EC 4.2.1.11). In some embodiments, the biomarker is T-complex protein 1. In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU). In some embodiments, two or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU). In some embodiments, three or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU). In some embodiments, 4, 5, 6, 7, 8, 9 or 10 or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU). In some embodiments, the biomarker is amyloid beta (A4) precursor protein (APP). In some embodiments, the biomarker is marcks. In some embodiments, the biomarker is internexin neuronal intermediate filament protein alpha (INA). In some embodiments, the biomarker is methyl CpG binding protein (MECP). In some embodiments, the biomarker is histone cluster 1 H1e (HIST1H1E). In some embodiments, the biomarker is albumin (ALB). In some embodiments, the biomarker is guanine nucleotide binding protein (G protein) beta polypeptide (GNB1). In some embodiments, the biomarker is adenylate kinase 1 (AK1). In some embodiments, the biomarker is aldose A fructose-biphosphate (ALDOA). In some embodiments, the biomarker is tenascin R (TNR) and clusterin (CLU). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN). In some embodiments, two or more biomarkers are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN). In some embodiments, three or more biomarkers are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN). In some embodiments, four or more biomarkers are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN). In some embodiments, five or more biomarkers are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN). In some embodiments, the biomarker is proteolipid protein 1 (PLP1). In some embodiments, the biomarker is growth associated protein 43 (GAP43). In some embodiments, the biomarker is dihydropyrimidinase-like 2 (DPYSL2). In some embodiments, the biomarker is quinoid dihydropteridine reductase (QDPR). In some embodiments, the biomarker is matrin 3 (MATR3). In some embodiments, the biomarker is enolase 1 (alpha) (ENO1). In some embodiments, the biomarker is and gelsolin (GSN). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH). In some embodiments, two or more biomarkers are selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH). In some embodiments, three or more biomarkers are selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH). In some embodiments, four or more biomarkers is selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH). In some embodiments, the biomarker is microtubule associated protein tau (MAPT). In some embodiments, the biomarker is microtuble-associated protein 1A (MAP1A). In some embodiments, the biomarker is AHNAK nucleoprotein. In some embodiments, the biomarker is cell cycle exit and neuronal differentiation 1 (CEND1) and glyceraldehyde-3-phosphate dehydrogenase (GAPDH). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, two or more biomarkers are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, three or more biomarkers are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, four or more biomarkers are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, five or more biomarkers are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, the biomarker is neurofilament triplet L protein (NF-L). In some embodiments, the biomarker is peroxiredoxin (EC 1.11.1.). In some embodiments, the biomarker is aconitate hydratase (EC 4.2.1.3). In some embodiments, the biomarker is enolase 2 (EC 4.2.1.11). In some embodiments, the biomarker is and T-complex protein 1. In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, two or more biomarkers are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, three or more biomarkers are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, 4, 5, 6, 7, 8, 9, 10, 11, 12, 13, 14, 15, 16, 17, 18, 19, or 20 or more biomarkers are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, the biomarker is myosin, heavy chain 6, cardiac muscle, alpha (MYH6). In some embodiments, the biomarker is actin, alpha, cardiac muscle 1 (ACTC1). In some embodiments, the biomarker is troponin I type 3 (cardiac) (TNNI3). In some embodiments, the biomarker is natriuretic peptide A (NPPA). In some embodiments, the biomarker is A kinase (PRKA) anchor protein 6 (AKAP6). In some embodiments, the biomarker is nestin (NES). In some embodiments, the biomarker is ATPase, Na+, K+ transporting, alpha 3 polypeptide (ATP1A3). In some embodiments, the biomarker is cadherin 2, type 1, N-cadherin (neuronal) (CDH2). In some embodiments, the biomarker is plakophilin 2 (PKP2). In some embodiments, the biomarker is ATP synthase subunit d (Atp5h). In some embodiments, the biomarker is ATP synthase subunit o (Atp5o). In some embodiments, the biomarker is ATP synthase subunit delta (Atp5d). In some embodiments, the biomarker is ATP synthase subunit alpha (Atp5a1). In some embodiments, the biomarker is ATP synthase subunit beta (Atp5b). In some embodiments, the biomarker is cytochrome c (Cycs). In some embodiments, the biomarker is mito, pyruvate dehydgrenase E1 component subunit beta (Pdhb). In some embodiments, the biomarker is phosphoglycerate kinase 1 (Pgk1). In some embodiments, the biomarker is heat shock protein 70 (Hspa9). In some embodiments, the biomarker is 60 kDa heat shock protein (Hspd1). In some embodiments, the biomarker is desmin (Desm). In some embodiments, the biomarker is troponin T2 (Tnnt2). In some embodiments, the biomarker is tropomyosin alpha 1 (Tpm1). In some embodiments, the biomarker is voltage dependent anion channel-1 (Vdac1). In some embodiments, the biomarker is elongation factor 2 (Eef2). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, two or more biomarkers are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, the biomarker is ATP synthase subunit d (Atp5h). In some embodiments, three or more biomarkers are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, the biomarker is ATP synthase subunit d (Atp5h). In some embodiments, 4, 5, 6, 7, 8, 9, or 10 or more biomarkers are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, the biomarker is ATP synthase subunit d (Atp5h). In some embodiments, the biomarker is ATP synthase subunit o (Atp5o). In some embodiments, the biomarker is ATP synthase subunit delta (Atp5d). In some embodiments, the biomarker is ATP synthase subunit alpha (Atp5a1), ATP synthase subunit beta (Atp5b). In some embodiments, the biomarker is cytochrome c (Cycs). In some embodiments, the biomarker is mito, pyruvate dehydgrenase E1 component subunit beta (Pdhb). In some embodiments, the biomarker is phosphoglycerate kinase 1 (Pgk1). In some embodiments, the biomarker is heat shock protein 70 (Hspa9). In some embodiments, the biomarker is 60 kDa heat shock protein (Hspd1). In some embodiments, the biomarker is desmin (Desm). In some embodiments, the biomarker is troponin T2 (Tnnt2). In some embodiments, the biomarker is tropomyosin alpha 1 (Tpm1). In some embodiments, the biomarker is voltage dependent anion channel-1 (Vdac1). In some embodiments, the biomarker is elongation factor 2 (Eef2). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the one or more biomarkers is indicative of aging. In some embodiments, two or more biomarkers are selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the biomarkers is indicative of aging. In some embodiments, three or more biomarkers are selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the biomarkers is indicative of aging. In some embodiments, 4, 5, 6, 7, 8, 9, or 10 or more biomarkers is selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the biomarkers is indicative of aging. In some embodiments, a decrease in the expression of ATP synthase subunit alpha (Atp5a1) is indicative of aging. In some embodiments, a decrease in the expression of ATP synthase subunit beta (Atp5b) is indicative of aging. In some embodiments, a decrease in the expression of cytochrome c (Cycs) is indicative of aging. In some embodiments, a decrease in the expression of mito, pyruvate dehydgrenase E1 component subunit beta (Pdhb) is indicative of aging. In some embodiments, a decrease in the expression of phosphoglycerate kinase 1 (Pgk1) is indicative of aging. In some embodiments, a decrease in the expression of heat shock protein 70 (Hspa9) is indicative of aging. In some embodiments, a decrease in the expression of desmin (Desm) is indicative of aging. In some embodiments, a decrease in the expression of troponin T2 (Tnnt2), tropomyosin alpha 1 (Tpm1) is indicative of aging. In some embodiments, a decrease in the expression of voltage dependent anion channel-1 (Vdac1) is indicative of aging.

In some embodiments, the biomarker is elongation factor 2 (Eef2). In some embodiments, an increase in the expression of Eef2 is indicative of aging.

In some embodiments, one or more biomarkers are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH). In some embodiments, two or more biomarkers are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11 orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH). In some embodiments, three or more biomarkers are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11 orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH). In some embodiments, 4, 5, 6, 7, 8, 9, 10, 11, 12, 13, 14, 15, 16, 17, 18, 19, 20, 25, 30, 35, or 40 or more biomarkers are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11 orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH). In some embodiments, the biomarker is podocin (NPHS2). In some embodiments, the biomarker is nephrin (NPHS1). In some embodiments, the biomarker is kin of IRRE like (NEPH1 or KIRREL). In some embodiments, the biomarker is podocalyxin-like (PODXL). In some embodiments, the biomarker is fibroblast growth factor 1 (FGF1). In some embodiments, the biomarker is crumbs family member 2 (CRB2). In some embodiments, the biomarker is solute carrier family 22 (organic anion transporter), member 8 (SLC22A8). In some embodiments, the biomarker is solute carrier family 22 (organic anion transporter), member 13 (SLC22A13). In some embodiments, the biomarker is aminocarboxymuconate semialdehyde decarboxylase (ACMSD). In some embodiments, the biomarker is agmatine ureohydrolase (agmatinase) (AGMAT). In some embodiments, the biomarker is betaine-homocysteine S-methyltransferase (BHMT). In some embodiments, the biomarker is chromosome 11 open reading frame 54 (C11orf54). In some embodiments, the biomarker is cadherin 6, type 2, K-cadherin (fetal kidney) (CDH6). In some embodiments, the biomarker is dihycropyrimidinase (DPYS). In some embodiments, the biomarker is gamma-glutamyltransferase 1 (GGT1). In some embodiments, the biomarker is 4-hydroxyphenylpyruvate dioxygenase (HPD). In some embodiments, the biomarker is heat-responsive protein 12 (HRSP12). In some embodiments, the biomarker is low density lipoprotein receptor-related protein 2 (LRP2). In some embodiments, the biomarker is pyruvate kinase, liver and RBC (PKLR). In some embodiments, the biomarker is X-prolyl aminopeptidase (aminopeptidase P)2, membrane-bound (XPNPEP2). In some embodiments, the biomarker is uromodulin (UMOD). In some embodiments, the biomarker is calbindin (CALB1). In some embodiments, the biomarker is solute carrier family 12 (sodium/potassium/chloride transporter), member 1 (SLC12A1). In some embodiments, the biomarker is solute carrier family 12 (sodium/chloride transporter), member 3 (SLC12A3), calcium-sensing receptor (CASR). In some embodiments, the biomarker is aquaporin (AQP2). In some embodiments, the biomarker is ATPase, H+ transporting, lysosomal 38 kDa, V0 subunit d2 (ATP6V0D2). In some embodiments, the biomarker is parvalbumin (PVALB). In some embodiments, the biomarker is transmembrane protein 213 (TMEM213). In some embodiments, the biomarker is transferrin, isocitrate dehydrogenase 1 (IDH). In some embodiments, the biomarker is 3-hydroxyisobutyrate dehydrogenase. In some embodiments, the biomarker is afenopin. In some embodiments, the biomarker is heat shock protein (HSP) 9A. In some embodiments, the biomarker is ATP synthase. In some embodiments, the biomarker is ornithine aminotransferase. In some embodiments, the biomarker is glutamate dehydrogenase. In some embodiments, the biomarker is phosphoglycerate mutase. In some embodiments, the biomarker is catalase. In some embodiments, the biomarker is glutathione (GSH). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of transferrin, isocitrate dehydrogenase 1 (IDH), and 3-hydroxyisobutyrate dehydrogenase, wherein an increase in the expression of the one or more biomarkers is indicative of an aging. In some embodiments, two or more biomarkers are selected from the group consisting of transferrin, isocitrate dehydrogenase 1 (IDH), and 3-hydroxyisobutyrate dehydrogenase, wherein an increase in the expression of the biomarkers is indicative of an aging. In some embodiments, three biomarkers are selected from the group consisting of transferrin, isocitrate dehydrogenase 1 (IDH), and 3-hydroxyisobutyrate dehydrogenase, wherein an increase in the expression of the biomarkers is indicative of an aging. In some embodiments, increased expression of transferrin is indicative of aging. In some embodiments, increased expression of isocitrate dehydrogenase 1 (IDH) is indicative of aging. In some embodiments, increased expression of 3-hydroxyisobutyrate dehydrogenase is indicative of aging.

In some embodiments, one or more biomarkers are selected from the group consisting of afenopin, phosphoglycerate mutase, and glutathione (GSH), wherein a decrease in the expression of the one or more biomarkers is indicative of aging. In some embodiments, two or more biomarkers are selected from the group consisting of afenopin, phosphoglycerate mutase, and glutathione (GSH), wherein a decrease in the expression of the biomarkers is indicative of aging. In some embodiments, three biomarkers are selected from the group consisting of afenopin, phosphoglycerate mutase, and glutathione (GSH), wherein a decrease in the expression of the biomarkers is indicative of aging. In some embodiments, decreased expression of afenopin is indicative of aging. In some embodiments, decreased expression of phosphoglycerate mutase is indicative of aging. In some embodiments, decreased expression of glutathione (GSH) biomarkers is indicative of aging.

In some embodiments, the increase in expression of the one or more biomarkers is gender specific. For example, in some instances, the biomarker is ATP synthase and the expression of the ATP synthase in up-regulated in aging males. In some instances, the biomarker is catalase and the expression of the catalase is down-regulated in aging males. In other instances, the biomarker is ATP synthase and the expression of ATP synthase is down-regulated in aging females. In some embodiments, the biomarker is ornithine aminotransferase and the expression of the ornithine aminotransferase is up-regulated in aging females. In some embodiments, the biomarker is glutamate dehydrogenase and the expression of the glutamate dehydrogenase is down-regulated in aging females.

In some embodiments, one or more biomarkers are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase. In some embodiments, the biomarker is apolipoprotein B (APOB). In some embodiments, two or more biomarkers are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase. In some embodiments, three or more biomarkers are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase. In some embodiments, 4, 5, 6, 7, 8, 9, 10, 11, 12 or 13 or more biomarkers are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase. In some embodiments, the biomarker is apolipoprotein B (APOB). In some embodiments, the biomarker is apolipoprotein A-I (APOA1). In some embodiments, the biomarker is fibrinogen gamma chain (FGG). In some embodiments, the biomarker is complement component 2 (C2). In some embodiments, the biomarker is kininogen 1 (KNG1). In some embodiments, the biomarker is fibrinogen alpha chain (FGA). In some embodiments, the biomarker is hydroxyacid oxidase (glycolate oxidase) 1 (HAO1). In some embodiments, the biomarker is retinol dehydrogenase 16 (all-trans) (RDH16). In some embodiments, the biomarker is aldolase B. In some embodiments, the biomarker is fructose-bisphosphate (ALDOB). In some embodiments, the biomarker is bile acid CoA: amino acid N-acyltransferase (glycine N-choloyltransferase) (BAAT). In some embodiments, the biomarker is aldo-keto reductase family 1, member C4 (AKR1C4). In some embodiments, the biomarker is solute carrier family 27 (fatty acid transporter), member 5 (SLC27A5). In some embodiments, the biomarker is epoxide hydrolase. In some embodiments, the biomarker is 3-ketoacyl-CoA thiolase A. In some embodiments, the biomarker is sarcosine oxidase. In some embodiments, the biomarker is 2,4-dienoyl reductase. In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of epoxide hydroxylase, 3-ketoacyl-CoA thiolase A, sarcosine oxidase, and 2,4-dienoyl reductase, wherein an increase in expression of the one or more biomarkers is indicative of aging. In some embodiments, two or more biomarkers are selected from the group consisting of epoxide hydroxylase, 3-ketoacyl-CoA thiolase A, sarcosine oxidase, and 2,4-dienoyl reductase, wherein an increase in expression of the biomarkers is indicative of aging. In some embodiments, three or more biomarkers are selected from the group consisting of epoxide hydroxylase, 3-ketoacyl-CoA thiolase A, sarcosine oxidase, and 2,4-dienoyl reductase, wherein an increase in expression of the biomarkers is indicative of aging. In some embodiments, four biomarkers are selected from the group consisting of epoxide hydroxylase, 3-ketoacyl-CoA thiolase A, sarcosine oxidase, and 2,4-dienoyl reductase, wherein an increase in expression of the biomarkers is indicative of aging. In some embodiments, an increase in expression of epoxide hydroxylase is indicative of aging. In some embodiments, an increase in expression of 3-ketoacyl-CoA thiolase A is indicative of aging. In some embodiments, an increase in expression of sarcosine oxidase is indicative of aging. In some embodiments, an increase in expression of 2,4-dienoyl reductase is indicative of aging.

In some embodiments, one or more biomarkers are selected from the group consisting of defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, two or more biomarkers are selected from the group consisting of defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, threeor more biomarkers are selected from the group consisting of defensin, alpha 1 (DEFA1), defensin, alpha 1B (DEFAIB), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, 4, 5, 6, 7, 8, 9, 10, 11, 12, 13, 14, 15, 16, 17, 18, 19, 20, 25, 30, 35, or 40 or more biomarkers are selected from the group consisting of defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, the biomarker is defensin, alpha 1 (DEFA1). In some embodiments, the biomarker is defensin, alpha 1B (DEFA1B). In some embodiments, the biomarker is defensin, alpha 3 (DEFA3). In some embodiments, the biomarker is defensin, alpha 4 (DEFA4). In some embodiments, the biomarker is cathepsin G (CTSG). In some embodiments, the biomarker is myeloperoxidase (MPO). In some embodiments, the biomarker is hemoglobin, beta (HBB). In some embodiments, the biomarker is hemoglobin, alpha 1 (HBA1). In some embodiments, the biomarker is hemoglobin, alpha 2 (HBA2). In some embodiments, the biomarker is S100 calcium binding protein 12 (S100A12). In some embodiments, the biomarker is chromosome 19 open reading frame 59 (C19orf59). In some embodiments, the biomarker is pyruvate dehydrogenase (lipoamide) beta. In some embodiments, the biomarker is fatty acid-binding protein 5. In some embodiments, the biomarker is galectin-3. In some embodiments, the biomarker is c-synuclein. In some embodiments, the biomarker is heterogeneous nuclear ribonucleoprotein A1. In some embodiments, the biomarker is myosin light chain, regulatory B (Mrlcb). In some embodiments, the biomarker is transgelin. In some embodiments, the biomarker is similar to purine-nucleoside phosphorylase (punA). In some embodiments, the biomarker is heterogeneous nuclear ribonucleoprotein A2/B1 isoform A2 (Hnrpa2b1). In some embodiments, the biomarker is Huntingtin interacting protein K (HYPK). In some embodiments, the biomarker is beta-actin FE-3 (Actg1). In some embodiments, the biomarker is caldesmon 1 (Cald1, calponin-1 (Cnn1). In some embodiments, the biomarker is E-FABP (C-FABP) (Fabp5). In some embodiments, the biomarker is capping protein (actin filament), gelsolin-like (CAPG). In some embodiments, the biomarker is similar to coactosin-like 1 (Cotl1). In some embodiments, the biomarker is calponin-1 (calponin H1, smooth muscle; basic calponin) (Cnn1). In some embodiments, the biomarker is vinculin (VCL). In some embodiments, the biomarker is VIM. In some embodiments, the biomarker is beta-tropomyosin (TPM2). In some embodiments, the biomarker is transgelin 2 (Tagln2). In some embodiments, the biomarker is tropomyosin 1, alpha isoform c (TPM1). In some embodiments, the biomarker is calponin 3, acidic (CNN3). In some embodiments, the biomarker is calponin 2 isoform a (Calponin 2). In some embodiments, the biomarker is F-actin capping protein beta subunit (Capzb). In some embodiments, the biomarker is alpha-globulin (Hba1). In some embodiments, the biomarker is alpha-actin (aa 40-375) (Acta2). In some embodiments, the biomarker is smooth muscle protein SM22 homolog-bovine (fragments) (Tagln2). In some embodiments, the biomarker is thioredoxin 2 (Txn1). In some embodiments, the biomarker is peroxideroxin 2 (Prdx2). In some embodiments, the biomarker is peroxiderodoxin 5 precursor (Prdx5). In some embodiments, the biomarker is Cu—Zn superoxide dismutase A5 (GSTA5).

In some embodiments, one or more biomarkers are selected from the group consisting of fatty acid-binding protein 5, galectin-3, c-synuclein, heterogeneous nuclear ribonucleoprotein A1, myosin light chain, regulatory B, peroxiredoxin 5 precursor, and transgelin. In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterogeneous nuclear ribonucleoprotein A1 isoform a (HNRPA1), heterogeneous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, two or more biomarkers are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, three or more biomarkers are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, 4, 5, 6, 7, 8, 9, 10, 11, 12, 13, 14, 15, 16, 17, 18, 19, or 20 or more biomarkers are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, the biomarker is transgelin (Tagln). In some embodiments, the biomarker is capping protein (actin filament). In some embodiments, the biomarker is gelsolin-like (CAPG). In some embodiments, the biomarker is caldesmon 1 (Cald1). In some embodiments, the biomarker is beta-actin FE-3 (Actg1). In some embodiments, the biomarker is similar to coactosin-like 1 (Cotl1). In some embodiments, the biomarker is calphonin-1 (calphonin H1, smooth muscle; basic calponin) (Cnn1). In some embodiments, the biomarker is vinculin (VCL), VIM. In some embodiments, the biomarker is beta-tropomyosin (TPM2). In some embodiments, the biomarker is myosin light chain, regulatory B (Mrlcb). In some embodiments, the biomarker is transgelin 2 (Tagln2). In some embodiments, the biomarker is tropomyosin 1, alpha isoform c (TPM1). In some embodiments, the biomarker is calponin 3, acidid (CNN3). In some embodiments, the biomarker is calponin 2 isoform a (Calponin 2). In some embodiments, the biomarker is F-actin capping protein beta subunit (Capzb). In some embodiments, the biomarker is alpha-globulin (Hba1). In some embodiments, the biomarker is alpha-actin (aa 40-375) (Acta2). In some embodiments, the biomarker is smooth muscle protein SM22 homolog-bovine (fragments) (Tagln2). In some embodiments, the biomarker is thioredoxin 2 (Txn1). In some embodiments, the biomarker is peroxideroxin 2 (Prdx2). In some embodiments, the biomarker is peroxiderodoxin 5 precursor (Prdx5). In some embodiments, the biomarker is Cu—Zn superoxide dismutase A5 (GSTA5). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, two or more biomarkers are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, three or more biomarkers are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, 4, 5, 6, 7, 8, 9, 10, 11, 12, 13, 14, 15, 16, 17, 18, 19, 20, 25, 30, 35, 40, 45, or 50 or more biomarkers are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CDla molecule (CD A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, the biomarker is collagen, type XVII, alplha 1 (COL17A1). In some embodiments, the biomarker is tumor protein p73 (TP73). In some embodiments, the biomarker is keratin 10 (KRT10). In some embodiments, the biomarker is caspase 14, apoptosis-related cysteine peptidase (CASP14). In some embodiments, the biomarker is filaggrin (FLG). In some embodiments, the biomarker is keratinocyte proline-rich protein (KPRP). In some embodiments, the biomarker is corneodesmosin (CDSN). In some embodiments, the biomarker is kallikrein-related peptidase 5 (KLK5). In some embodiments, the biomarker is melan-A (MLANA). In some embodiments, the biomarker is dopachrome tautomerase (DCT). In some embodiments, the biomarker is tyrosinase (TYR). In some embodiments, the biomarker is CDla molecule (CD1A). In some embodiments, the biomarker is CD207 molecule, langerin, (CD207). In some embodiments, the biomarker is annexin A6 (ANXA6). In some embodiments, the biomarker is glutaminyl-tRNA synthetase (QARS). In some embodiments, the biomarker is cation-independent mannose-6-phosphate (IGF2R). In some embodiments, the biomarker is twinfilin-2 (TWF2). In some embodiments, the biomarker is 40S ribosomal protein S5 (RPS5). In some embodiments, the biomarker is putative pre-mRNA-splicing factor ATP-dependent RNA helicase DHX15 (DHX15). In some embodiments, the biomarker is 26S proteasome non-ATPase regulatory subunit 1 (PSMD1). In some embodiments, the biomarker is 40S ribosomal protein S29 (RPS29). In some embodiments, the biomarker is synaptopodin-2 (SYNPO2). In some embodiments, the biomarker is T-complex protein 1 subunit zeta (CCT6A). In some embodiments, the biomarker is annexin 5 (ANXA5). In some embodiments, the biomarker is tRNA-splicing ligase RtcB homolog (C22orf28). In some embodiments, the biomarker is serine/arginine-rich splicing factor 9 (SRSF9). In some embodiments, the biomarker is myosin light polypeptide 6 (MYL6). In some embodiments, the biomarker is protein phosphatase 1 regulatory subunit 7 (PPP1R7). In some embodiments, the biomarker is UPF0568 protein C14orfl66 (C14orfl66). In some embodiments, the biomarker is 26 proteasome non-ATPase regulatory subunit 14 (PSMD14). In some embodiments, the biomarker is serine hydroxymethyltransferase, mitochondrial (SHMT2). In some embodiments, the biomarker is heat shock 70 kDa protein 1A/1B (HSPA1A). In some embodiments, the biomarker is ATP-dependent RNA helicase DDX1 (DDX1). In some embodiments, the biomarker is calmodulin (CALM1). In some embodiments, the biomarker is AP-2 complex subunit alpha-2 (AP2A2). In some embodiments, the biomarker is Rho guanine nucleotide exchange factor 2 (ARHGEF2). In some embodiments, the biomarker is annexin A4 (ANXA4). In some embodiments, the biomarker is erythrocyte band 7 integral membrane protein (STOM). In some embodiments, the biomarker is ATP-dependent RNA helicase DDX3X (DDX3X). In some embodiments, the biomarker is calpain small subunit 1 (CAPNS1). In some embodiments, the biomarker is NAD(P)H dehydrogenase [quinone] 1 (NQO1). In some embodiments, the biomarker is Protein S100-A16 (S100A16). In some embodiments, the biomarker is clathrin light chain B (CLTB). In some embodiments, the biomarker is brain acid soluble protein 1 (BASP1). In some embodiments, the biomarker is DnaJ homolog subfamily C member 3 (DNAJC3). In some embodiments, the biomarker is AP-2 complex subunit alpha-1 (AP2A1). In some embodiments, the biomarker is 40S ribosomal protein (RPS6). In some embodiments, the biomarker is glycyl-tRNA synthetase (GARS). In some embodiments, the biomarker is EH domain-containing protein 2 (EHD2). In some embodiments, the biomarker is oligoribonuclease. In some embodiments, the biomarker is mitochondrial (REXO2). In some embodiments, the biomarker is thrombospondin-1 (THBS1). In some embodiments, the biomarker is glycylpeptide N-tetradecanoyltransferase 1 (NMT1). In some embodiments, the biomarker is adenylyl cyclase-associated protein 1 (CAP1). In some embodiments, the biomarker is heat shock-related 70 kDa protein 2 (HSPA2). In some embodiments, the biomarker is histone H2A type 1-A (HIST1H2AA). In some embodiments, the biomarker is T-complex protein 1 subunit alpha (TCP1). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the one or more biomarkers is indicative of aging. In some embodiments, two or more biomarkers are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the biomarkers is indicative of aging. In some embodiments, three or more biomarkers are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the biomarkers is indicative of aging. In some embodiments, four or more biomarkers are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the biomarkers is indicative of aging. In some embodiments, five biomarkers are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the biomarkers is indicative of aging. In some embodiments, an decrease in expression of cytochrome c oxidase II (MTCO2) is indicative of aging. In some embodiments, an decrease in expression of NADH dehydrogenase (ubiquinone) 1 alpha subcomplex, 5 (NDUFA5) is indicative of aging. In some embodiments, an decrease in expression of NADH dehydrogenase (ubiquinone) 1 alpha subcomplex, 9 (NDUFA9) is indicative of aging. In some embodiments, an decrease in expression of NADH dehydrogenase (ubiquinone) 1 alpha subcomplex, 10 (NDUFA10) is indicative of aging. In some embodiments, an decrease in expression of NADH dehydrogenase (ubiquinone) Fe—S protein 6, 13 kDa (NADH-coenzyme Q reductase) (NDUFS6) is indicative of aging.

In some embodiments, one or more biomarkers are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, two or more biomarkers are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, three or more biomarkers are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, 4, 5, 6, 7, 8, 9, 10, 11, 12, 13, 14, 15, 16, 17, 18, 19, 20, 25, 30, 35, or 40 or more biomarkers are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, the biomarker is annexin A6 (ANXA6). In some embodiments, the biomarker is glutaminyl-tRNA synthetase (QARS). In some embodiments, the biomarker is cation-independent mannose-6-phosphate (IGF2R). In some embodiments, the biomarker is twinfilin-2 (TWF2). In some embodiments, the biomarker is 40S ribosomal protein S5 (RPS5). In some embodiments, the biomarker is putative pre-mRNA-splicing factor ATP-dependent RNA helicase DHX15 (DHX15). In some embodiments, the biomarker is 26S proteasome non-ATPase regulatory subunit 1 (PSMD1). In some embodiments, the biomarker is 40S ribosomal protein S29 (RPS29). In some embodiments, the biomarker is synaptopodin-2 (SYNPO2). In some embodiments, the biomarker is T-complex protein 1 subunit zeta (CCT6A). In some embodiments, the biomarker is annexin 5 (ANXA5). In some embodiments, the biomarker is tRNA-splicing ligase RtcB homolog (C22orf28). In some embodiments, the biomarker is serine/arginine-rich splicing factor 9 (SRSF9). In some embodiments, the biomarker is myosin light polypeptide 6 (MYL6). In some embodiments, the biomarker is protein phosphatase 1 regulatory subunit 7 (PPP1R7). In some embodiments, the biomarker is UPF0568 protein C14orf166 (C14orf166). In some embodiments, the biomarker is 26 proteasome non-ATPase regulatory subunit 14 (PSMD14). In some embodiments, the biomarker is serine hydroxymethyltransferase, mitochondrial (SHMT2). In some embodiments, the biomarker is heat shock 70 kDa protein 1A/1B (HSPA1A). In some embodiments, the biomarker is ATP-dependent RNA helicase DDX1 (DDX1). In some embodiments, the biomarker is calmodulin (CALM1). In some embodiments, the biomarker is AP-2 complex subunit alpha-2 (AP2A2). In some embodiments, the biomarker is Rho guanine nucleotide exchange factor 2 (ARHGEF2). In some embodiments, the biomarker is annexin A4 (ANXA4). In some embodiments, the biomarker is erythrocyte band 7 integral membrane protein (STOM). In some embodiments, the biomarker is ATP-dependent RNA helicase DDX3X (DDX3X). In some embodiments, the biomarker is calpain small subunit 1 (CAPNS1). In some embodiments, the biomarker is NAD(P)H dehydrogenase [quinone] 1 (NQO1). In some embodiments, the biomarker is Protein S100-A16 (S100A16). In some embodiments, the biomarker is clathrin light chain B (CLTB). In some embodiments, the biomarker is brain acid soluble protein 1 (BASP1). In some embodiments, the biomarker is DnaJ homolog subfamily C member 3 (DNAJC3). In some embodiments, the biomarker is AP-2 complex subunit alpha-1 (AP2A1). In some embodiments, the biomarker is 40S ribosomal protein (RPS6). In some embodiments, the biomarker is glycyl-tRNA synthetase (GARS). In some embodiments, the biomarker is EH domain-containing protein 2 (EHD2). In some embodiments, the biomarker is oligoribonuclease, mitochondrial (REXO2). In some embodiments, the biomarker is thrombospondin-1 (THBS1). In some embodiments, the biomarker is glycylpeptide N-tetradecanoyltransferase 1 (NMT1). In some embodiments, the biomarker is adenylyl cyclase-associated protein 1 (CAP1). In some embodiments, the biomarker is heat shock-related 70 kDa protein 2 (HSPA2). In some embodiments, the biomarker is histone H2A type 1-A (HIST1H2AA). In some embodiments, the biomarker is T-complex protein 1 subunit alpha (TCP1). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, one or more biomarkers are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the one or more biomarkers is indicative of aging. In some embodiments, two or more biomarkers are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the biomarkers is indicative of aging. In some embodiments, three or more biomarkers are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the biomarkers is indicative of aging. In some embodiments, 4, 5, 6, 7, 8, 9, 10, 11, 12, 13, 14, 15, 16, 17, 18, 19, or 20 or more biomarkers are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the biomarkers is indicative of aging. In some embodiments, an increase in the expression of annexin A6 (ANXA6) is indicative of aging. In some embodiments, an increase in the expression of glutaminyl-tRNA synthetase (QARS) is indicative of aging. In some embodiments, an increase in the expression of cation-independent mannose-6-phosphate (IGF2R) is indicative of aging. In some embodiments, an increase in the expression of putative pre-mRNA-splicing factor ATP-dependent RNA helicase DHX15 (DHX15) is indicative of aging. In some embodiments, an increase in the expression of 40S ribosomal protein S29 (RPS29) is indicative of aging. In some embodiments, an increase in the expression of synaptopodin-2 (SYNPO2) is indicative of aging. In some embodiments, an increase in the expression of annexin 5 (ANXA5) is indicative of aging. In some embodiments, an increase in the expression of serine/arginine-rich splicing factor 9 (SRSF9) is indicative of aging. In some embodiments, an increase in the expression of myosin light polypeptide 6 (MYL6) is indicative of aging. In some embodiments, an increase in the expression of heat shock 70 kDa protein 1A/1B (HSPA1A) is indicative of aging. In some embodiments, an increase in the expression of calmodulin (CALM1) is indicative of aging. In some embodiments, an increase in the expression of annexin A4 (ANXA4) is indicative of aging. In some embodiments, an increase in the expression of erythrocyte band 7 integral membrane protein (STOM) is indicative of aging. In some embodiments, an increase in the expression of NAD(P)H dehydrogenase [quinone] 1 (NQO1) is indicative of aging. In some embodiments, an increase in the expression of clathrin light chain B (CLTB) is indicative of aging. In some embodiments, an increase in the expression of brain acid soluble protein 1 (BASP1) is indicative of aging. In some embodiments, an increase in the expression of 40S ribosomal protein (RPS6) is indicative of aging. In some embodiments, an increase in the expression of EH domain-containing protein 2 (EHD2) is indicative of aging. In some embodiments, an increase in the expression of thrombospondin-1 (THBS1) is indicative of aging. In some embodiments, an increase in the expression of heat shock-related 70 kDa protein 2 (HSPA2) is indicative of aging.

In some embodiments, one or more biomarkers are selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the one or more biomarkers is indicative of aging. In some embodiments, two or more biomarkers are selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the biomarkers is indicative of aging. In some embodiments, three or more biomarkers are selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the biomarkers is indicative of aging. In some embodiments, 4, 5, 6, 7, 8, 9, 10, 11, 12, 13, 14, 15, 16, 17, 18, 19, or 20 or more biomarkers are selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the biomarkers is indicative of aging. In some embodiments, an increase in the expression of twinfilin-2 (TWF2) is indicative of aging. In some embodiments, an increase in the expression of 40S ribosomal protein S5 (RPS5) is indicative of aging. In some embodiments, an increase in the expression of 26S proteasome non-ATPase regulatory subunit 1 (PSMD1) is indicative of aging. In some embodiments, an increase in the expression of T-complex protein 1 subunit zeta (CCT6A) is indicative of aging. In some embodiments, an increase in the expression of tRNA-splicing ligase RtcB homolog (C22orf28) is indicative of aging. In some embodiments, an increase in the expression of protein phosphatase 1 regulatory subunit 7 (PPP1R7) is indicative of aging. In some embodiments, an increase in the expression of UPF0568 protein C14orf166 (C14orf166) is indicative of aging. In some embodiments, an increase in the expression of 26 proteasome non-ATPase regulatory subunit 14 (PSMD14) is indicative of aging. In some embodiments, an increase in the expression of serine hydroxymethyltransferase is indicative of aging. In some embodiments, an increase in the expression of mitochondrial (SHMT2) is indicative of aging. In some embodiments, an increase in the expression of ATP-dependent RNA helicase DDX1 (DDX1) is indicative of aging. In some embodiments, an increase in the expression of AP-2 complex subunit alpha-2 (AP2A2) is indicative of aging. In some embodiments, an increase in the expression of Rho guanine nucleotide exchange factor 2 (ARHGEF2) is indicative of aging. In some embodiments, an increase in the expression of ATP-dependent RNA helicase DDX3X (DDX3X) is indicative of aging. In some embodiments, an increase in the expression of calpain small subunit 1 (CAPNS1) is indicative of aging. In some embodiments, an increase in the expression of Protein S100-A16 (S100A16) is indicative of aging. In some embodiments, an increase in the expression of DnaJ homolog subfamily C member 3 (DNAJC3) is indicative of aging. In some embodiments, an increase in the expression of AP-2 complex subunit alpha-1 (AP2A1) is indicative of aging. In some embodiments, an increase in the expression of glycyl-tRNA synthetase (GARS) is indicative of aging. In some embodiments, an increase in the expression of oligoribonuclease is indicative of aging. In some embodiments, an increase in the expression of mitochondrial (REXO2) is indicative of aging. In some embodiments, an increase in the expression of glycylpeptide N-tetradecanoyltransferase 1 (NMT1) is indicative of aging. In some embodiments, an increase in the expression of adenylyl cyclase-associated protein 1 (CAP1) is indicative of aging. In some embodiments, an increase in the expression of histone H2A type 1-A (HIST1H2AA) is indicative of aging. In some embodiments, an increase in the expression of T-complex protein 1 subunit alpha (TCP1) is indicative of aging.

In some embodiments, the aging cell is a somatic cell. In some embodiments, the aging cell is a skeletal muscle cell. In some embodiments, the aging cell is a brain cell. In some embodiments, the aging cell is from the brain. In other embodiments, the aging cell is a cardiac cell. In some embodiments, the aging cell is from the heart. In some instances, the aging cell is a kidney cell. In some embodiments, the aging cell is from the kidney. In some embodiments, the aging cell is a liver cell. In some embodiments, the aging cell is from the liver. In other embodiments, the aging cell is a granulocyte, mast cell or macrophage. In some embodiments, the aging cell is from the bone marrow. In some instances, the aging cell is a skin cell. In some embodiments, the aging cell is from the skin.

In some embodiments, the one or more biomarkers are a protein expressed in skeletal muscle. In some embodiments, the skeletal muscle comprises striated muscle cells. In some embodiments, the one or more biomarkers are a protein expressed in striated muscle cells. In some embodiments, the protein expressed in skeletal muscle is selected from the group consisting of MLCF3, myosin light polypeptide 2 (slow), MLC1F, MYBPC1, myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), heat shock 27 kDa protein (Hsp27), disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, MYH2, TNNT1, RYR1, CASQ1, JPH1, AMPD1, PYGM, and ENO3. In some embodiments, expression of the biomarker is increased in the skeletal muscle. In some embodiments, expression of the biomarker is increased in an aging cell of the skeletal muscle. In some embodiments, an increase in expression of the biomarker is indicative of aging of the skeletal muscle. In some embodiments, expression of the biomarker is decreased in the skeletal muscle. In some embodiments, expression of the biomarker is decreased in an aging cell of the skeletal muscle. In other embodiments, a decrease in expression of the biomarker is indicative of aging of the skeletal muscle. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, a decrease in expression of the protein expressed in skeletal muscle is indicative of aging. In some embodiments, the protein with decreased expression is selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19.

In other embodiments, an increase in expression of the protein expressed in skeletal muscle is indicative of aging. In some embodiments, the protein with increased expression is selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide.

In some embodiments, the one or more biomarkers are a protein expressed in the brain. In some embodiments, the brain comprises a neuron. In some embodiments, the one or more biomarkers are a protein expressed in the neuron. In some embodiments, the brain comprises a glial cell. In some embodiments, the one or more biomarkers are a protein expressed in the glial cell. In some embodiments, the brain comprises a hippocampus. In some embodiments, the one or more biomarkers are a protein expressed in the hippocampus. In some embodiments, the brain comprises a cortex. In some embodiments, the one or more biomarkers are a protein expressed in the parietal cortex. In some embodiments, the brain comprises a cerebellum. In some embodiments, the one or more biomarkers are a protein expressed in the cerebellum. In some embodiments, the protein expressed in the brain is selected from the group consisting of myristoylated alanine-rich C-kinase substrate, alpha-internexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of MBP, and VIM. In some embodiments, the protein expressed in the brain is selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, the protein expressed in the brain is selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU). In some embodiments, the protein expressed in the brain is selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN). In some embodiments, the protein expressed in the brain is selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH). In some embodiments, the protein expressed in the brain is selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1. In some embodiments, expression of the biomarker is increased in the brain. In some embodiments, expression of the biomarker is increased in an aging cell of the brain. In some embodiments, an increase in expression of the biomarker is indicative of aging of the brain. In some embodiments, expression of the biomarker is decreased in the brain. In some embodiments, expression of the biomarker is decreased in an aging cell of the brain. In other embodiments, a decrease in expression of the biomarker is indicative of aging of the brain. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, the one or more biomarkers are a protein expressed in the heart. In some embodiments, the heart comprises a cardiomyocyte. In some embodiments, the one or more biomarkers are a protein expressed in the cardiomyocyte. In some embodiments, the heart comprises an endothelial cell. In some embodiments, the one or more biomarkers are a protein expressed in the endothelial cell. In some embodiments, the protein expressed in the heart is selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, the protein expressed in the heart is selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2). In some embodiments, expression of the biomarker is increased in the heart. In some embodiments, expression of the biomarker is increased in an aging cell of the heart. In some embodiments, an increase in expression of the biomarker is indicative of aging of the heart. In some embodiments, expression of the biomarker is decreased in the heart. In some embodiments, expression of the biomarker is decreased in an aging cell of the heart. In other embodiments, a decrease in expression of the biomarker is indicative of aging of the heart. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, a decrease in expression of the protein expressed in the heart is indicative of aging. In some embodiments, the protein with decreased expression is selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1).

In some embodiments, an increase in expression of the protein expressed in the heart is indicative of aging. In some embodiments, the protein with increased expression is elongation factor 2 (Eef2).

In some embodiments, the one or more biomarkers are a protein expressed in the kidney. In some embodiments, the kidney comprises a glomerulus. In some embodiments, the one or more biomarkers are a protein expressed in the glomerulus. In some embodiments, the kidney comprises a proximal tube. In some embodiments, the one or more biomarkers are a protein expressed in the proximal tube. In some embodiments, the kidney comprises a distal tube. In some embodiments, the one or more biomarkers are a protein expressed in the distal tube. In some embodiments, the kidney comprises a collecting duct. In some embodiments, the one or more biomarkers are a protein expressed in the collecting duct. In some embodiments, the kidney comprises an intercalated cell. In some embodiments, the one or more biomarkers are a protein expressed in the intercalated cell. In some embodiments, the kidney comprises a podocyte. In some embodiments, the one or more biomarkers are a protein expressed in the podocyte. In some embodiments, the protein expressed in the kidney is selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR) and X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH). In some embodiments, expression of the biomarker is increased in the kidney. In some embodiments, expression of the biomarker is increased in an aging cell of the kidney. In some embodiments, an increase in expression of the biomarker is indicative of aging of the kidney. In some embodiments, expression of the biomarker is decreased in the kidney. In some embodiments, expression of the biomarker is decreased in an aging cell of the kidney. In other embodiments, a decrease in expression of the biomarker is indicative of aging of the kidney. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, an increase in expression of the protein expressed in skeletal muscle is indicative of aging. In some embodiments, the protein with increased expression is selected from the group consisting of transferrin, isocitrate dehydrogenase 1 (IDH), and 3-hydroxyisobutyrate dehydrogenase.

In some embodiments, a decrease in expression of the protein expressed in the kidney is indicative of aging. In some embodiments, the protein with decreased expression is selected from the group consisting of afenopin, phosphoglycerate mutase, and glutathione (GSH).

In some embodiments, the increase in expression of the protein expressed in the kidney is gender specific. For example, in some instances, the protein is ATP synthase and the expression of the ATP synthase in up-regulated in the kidney of aging males. In some instances, the protein is catalase and the expression of the catalase is down-regulated in the kidney of aging males. In other instances, the protein is ATP synthase and the expression of ATP synthase is down-regulated in the kidney aging females. In some embodiments, the protein is ornithine aminotransferase and the expression of the ornithine aminotransferase is up-regulated in the kidney of aging females. In some embodiments, the protein is glutamate dehydrogenase and the expression of the glutamate dehydrogenase is down-regulated in the kidney of aging females.

In some embodiments, the one or more biomarkers are a protein expressed in the liver. In some embodiments, the protein expressed in the liver is a plasma protein. In some embodiments, the protein expressed by the liver is a metabolic enzyme. In some embodiments, the protein expressed in the liver is a protein involved in bile acid synthesis. In some embodiments, the protein expressed in the liver is selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase. In some embodiments, expression of the biomarker is increased in the liver. In some embodiments, expression of the biomarker is increased in an aging cell of the liver. In some embodiments, an increase in expression of the biomarker is indicative of aging of the liver. In some embodiments, expression of the biomarker is decreased in the liver. In some embodiments, expression of the biomarker is decreased in an aging cell of the liver. In other embodiments, a decrease in expression of the biomarker is indicative of aging of the liver. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, an increase in the expression of the protein expressed in the liver is indicative of aging. In some embodiments, the protein with increased expression is selected from the group consisting of epoxide hydroxylase, 3-ketoacyl-CoA thiolase A, sarcosine oxidase, and 2,4-dienoyl reductase.

In some embodiments, the one or more biomarkers are a protein expressed in the bone marrow. In some embodiments, the bone marrow comprises red marrow. In some embodiments, the one or more biomarkers are a protein expressed in the red marrow. In some embodiments, the bone marrow comprises a hematopoietic cell. In some embodiments, the one or more biomarkers are a protein expressed in the hematopoietic cell. In some embodiments, the protein expressed in the bone marrow is selected from the group consisting of defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, the protein expressed in the bone marrow is selected from the group consisting of fatty acid-binding protein 5, galectin-3, c-synuclein, heterogeneous nuclear ribonucleoprotein A1, myosin light chain, regulatory B, peroxiredoxin 5 precursor, and transgelin. In some embodiments, the protein expressed in the bone marrow is selected from the group consisting of beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterogeneous nuclear ribonucleoprotein A1 isoform a (HNRPA1), heterogeneous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln). In some embodiments, the protein expressed in the bone marrow is selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5). In some embodiments, expression of the biomarker is increased in the bone marrow. In some embodiments, expression of the biomarker is increased in an aging cell of the bone marrow. In some embodiments, an increase in expression of the biomarker is indicative of aging of the bone marrow. In some embodiments, expression of the biomarker is decreased in the bone marrow. In some embodiments, expression of the biomarker is decreased in an aging cell of the bone marrow. In other embodiments, a decrease in expression of the biomarker is indicative of aging of the bone marrow. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, the one or more biomarkers are a protein expressed in the skin. In some embodiments, the skin comprises an epidermis. In some embodiments, the one or more biomarkers are a protein expressed in the epidermis. In some embodiments, the skin comprises a keratinocyte. In some embodiments, the one or more biomarkers are a protein expressed in the keratinocyte. In some embodiments, the skin comprises a melanocyte. In some embodiments, the one or more biomarkers are a protein expressed in the melanocyte. In some embodiments, the skin comprises a hair follicle. In some embodiments, the one or more biomarkers are a protein expressed in the hair follicle. In some embodiments, the skin comprises a dermal cell. In some embodiments, the one or more biomarkers are a protein expressed in the dermal cell. In some embodiments, the protein expressed in the skin is selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, the protein expressed in the skin is selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1). In some embodiments, expression of the biomarker is increased. In some embodiments, expression of the biomarker is increased in an aging cell. In some embodiments, an increase in expression of the biomarker is indicative of aging. In some embodiments, expression of the biomarker is decreased. In some embodiments, expression of the biomarker is decreased in an aging cell. In other embodiments, a decrease in expression of the biomarker is indicative of aging. In some embodiments, alterations in biomarker expression are gender specific. In some embodiment, expression of the biomarker is increased in aging males. In some embodiments, expression of the biomarker is decreased in aging males. In other embodiment, expression of the biomarker is increased in aging females. In other embodiments, expression of the biomarker is decreased in aging females.

In some embodiments, an increase in expression of the protein expressed in the skin is indicative of aging. In some embodiments, the protein with increased expression is selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6). In some embodiments, the protein with increased expression is selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2).

In some embodiments, a decrease in expression of the protein expressed in the skin is indicative of aging. In some embodiments, the protein with decreased expression is selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1).

In one embodiment, the biomarker is one or more biomarkers independently selected from the group consisting of Abcg1, Abra, Actn3, Alas2, Alox15, Angpt14, Apod, Apold1, Arc, Arhgap24, Arl4c, Amt1, Arrdc2, Asb5, Atf3, Bag2, Bcl11a, Bcl6, Bdh1, Bdnf, Best3, Bhlhe40, Calhm1, Calm13, Car12, Ccl5, Cd74, Cdc42se1, Chac1, Chst5, Ciart, Cidec, Cish, Cited4, Ckap4, Cldn2, Clic6, Cpt1a, Csrnp1, Cxcl13, Dbp, Dnajb5, Dynl11, Dyrk2, Edn1, Egr1, Egr3, Elfn1, Emb, Enah, Fam107b, Fam110a, Fam134b, Fam167a, Fam46a, Fasn, Fgfr3, Fhl2, Fos, Fosb, Frk, Fst, Gdf15, Gem, Gngt1, Gnl3, Hba1, Hba2, Hbb, Hbb-b1, Hbegf, Hmox1, Hpd1, Hspa1b, Id4, Il2rb, Irs1, Irs2, Junb, Jund, Kbtbd8, Kcnk5, Kctd7, Kirrel2, Ky, Lamc2, Lipg, LOC689064, Lonrf3, Lrrc38, Lrrc52, Lrrn2, Lsr, Maff, Mchr1, Mfrp, Mllt1, Mns1, Mogat1, Mphosph6, Mpz, Muc20, Mybpc2, Myf6, Myh1, Myh2, Myh4, Myocd, Nedd9, Nfil3, Nkg7, Nrld1, Nr4a2, Nr4a3, Ntf4, Nuak1, Parp16, Pdc, Pde7a, Pfkfb2, Pfkfb3, Pgam1, Phlda1, Pik3ip1, Plk3, Postn, Ppargc1a, Ppp1r14c, Pragmin, Prf1, Ptpn14, Pva1b, Rab23, Rab30, Rbm20, Rcan1, Rel11, Rfx1, RGD1307461, RGD1309676, RGD1359290, RGD1564428, Rhpn2, Rn45s, Rnd1, Rp1, Rrad, RT1-Ba, RT1-Bb, RT1-Da, RT1-Db1, Rtn4r11, Scd1, Sdc4, Sec1415, Siglec5, Sik1, Slc18a2, Slc2a5, Slc30a4, Slc4a1, Slc4a5, Slpi, Smad7, Snhg4, Spag8, Stc1, Sv2c, Terf2ip, Thrsp, Tmc8, Tmem171, Tmx4, Tnfrsfl2a, Tnni2, Ttc30b, Txnip, Ucp3, Unc5b, Zfp112, Zfp13, Zfp385b, Zfp474, Zfyve28, Zic1 or Zmynd10. In one embodiment, the one or more biomarkers is 2, 3, 4, 5, 6, 7, 8, 9, 10, 15, 20, 25, 30, 35, 40, 45, 50, 55, 60, 65, 70, 75 or more biomarkers, or any range or interval thereof. In one embodiment, the biomarker is Abcg1. In one embodiment, the biomarker is Abra. In one embodiment, the biomarker is Actn3. In one embodiment, the biomarker is Alas2. In one embodiment, the biomarker is Alox15. In one embodiment, the biomarker is Angpt14. In one embodiment, the biomarker is Apod. In one embodiment, the biomarker is Apold1. In one embodiment, the biomarker is Arc. In one embodiment, the biomarker is Arhgap24. In one embodiment, the biomarker is Arl4c. In one embodiment, the biomarker is Amt1. In one embodiment, the biomarker is Arrdc2. In one embodiment, the biomarker is Asb5. In one embodiment, the biomarker is Atf3. In one embodiment, the biomarker is Bag2. In one embodiment, the biomarker is Bcl11a. In one embodiment, the biomarker is Bcl6. In one embodiment, the biomarker is Bdh1. In one embodiment, the biomarker is Bdnf. In one embodiment, the biomarker is Best3. In one embodiment, the biomarker is Bhlhe40. In one embodiment, the biomarker is Calhm1. In one embodiment, the biomarker is Calm13. In one embodiment, the biomarker is Car12. In one embodiment, the biomarker is Ccl5. In one embodiment, the biomarker is Cd74. In one embodiment, the biomarker is Cdc42se1. In one embodiment, the biomarker is Chac1. In one embodiment, the biomarker is Chst5. In one embodiment, the biomarker is Ciart. In one embodiment, the biomarker is Cidec. In one embodiment, the biomarker is Cish. In one embodiment, the biomarker is Cited4. In one embodiment, the biomarker is Ckap4. In one embodiment, the biomarker is Cldn2. In one embodiment, the biomarker is Clic6. In one embodiment, the biomarker is Cpt1a. In one embodiment, the biomarker is Csrnp1. In one embodiment, the biomarker is Cxcl13. In one embodiment, the biomarker is Dbp. In one embodiment, the biomarker is Dnajb5. In one embodiment, the biomarker is Dynl11. In one embodiment, the biomarker is Dyrk2. In one embodiment, the biomarker is Edn1. In one embodiment, the biomarker is Egr1. In one embodiment, the biomarker is Egr3. In one embodiment, the biomarker is Elfn1. In one embodiment, the biomarker is Emb. In one embodiment, the biomarker is Enah. In one embodiment, the biomarker is Fam107b. In one embodiment, the biomarker is Fam110a. In one embodiment, the biomarker is Fam134b. In one embodiment, the biomarker is Fam167a. In one embodiment, the biomarker is Fam46a. In one embodiment, the biomarker is Fasn. In one embodiment, the biomarker is Fgfr3. In one embodiment, the biomarker is Fhl2. In one embodiment, the biomarker is Fos. In one embodiment, the biomarker is Fosb. In one embodiment, the biomarker is Frk. In one embodiment, the biomarker is Fst. In one embodiment, the biomarker is Gdf15. In one embodiment, the biomarker is Gem. In one embodiment, the biomarker is Gngt1. In one embodiment, the biomarker is Gnl3. In one embodiment, the biomarker is Hba1. In one embodiment, the biomarker is Hba2. In one embodiment, the biomarker is Hbb. In one embodiment, the biomarker is Hbb-b1. In one embodiment, the biomarker is Hbegf. In one embodiment, the biomarker is Hmox1. In one embodiment, the biomarker is Hpd1. In one embodiment, the biomarker is Hspa1b. In one embodiment, the biomarker is Id4. In one embodiment, the biomarker is Il2rb. In one embodiment, the biomarker is Irs1. In one embodiment, the biomarker is Irs2. In one embodiment, the biomarker is Junb. In one embodiment, the biomarker is Jund. In one embodiment, the biomarker is Kbtbd8. In one embodiment, the biomarker is Kcnk5. In one embodiment, the biomarker is Kctd7. In one embodiment, the biomarker is Kirrel2. In one embodiment, the biomarker is Ky. In one embodiment, the biomarker is Lamc2. In one embodiment, the biomarker is Lipg. In one embodiment, the biomarker is LOC689064. In one embodiment, the biomarker is Lonrf3. In one embodiment, the biomarker is Lrrc38. In one embodiment, the biomarker is Lrrc52. In one embodiment, the biomarker is Lrrn2. In one embodiment, the biomarker is Lsr. In one embodiment, the biomarker is Maff. In one embodiment, the biomarker is Mchr1. In one embodiment, the biomarker is Mfrp. In one embodiment, the biomarker is Mllt11. In one embodiment, the biomarker is Mns1. In one embodiment, the biomarker is Mogat1. In one embodiment, the biomarker is Mphosph6. In one embodiment, the biomarker is Mpz. In one embodiment, the biomarker is Muc20. In one embodiment, the biomarker is Mybpc2. In one embodiment, the biomarker is Myf6. In one embodiment, the biomarker is Myh1. In one embodiment, the biomarker is Myh2. In one embodiment, the biomarker is Myh4. In one embodiment, the biomarker is Myocd. In one embodiment, the biomarker is Nedd9. In one embodiment, the biomarker is Nfil3. In one embodiment, the biomarker is Nkg7. In one embodiment, the biomarker is Nrld1. In one embodiment, the biomarker is Nr4a2. In one embodiment, the biomarker is Nr4a3. In one embodiment, the biomarker is Ntf4. In one embodiment, the biomarker is Nuak1. In one embodiment, the biomarker is Parp16. In one embodiment, the biomarker is Pdc. In one embodiment, the biomarker is Pde7a. In one embodiment, the biomarker is Pfkfb2. In one embodiment, the biomarker is Pfkfb3. In one embodiment, the biomarker is Pgam1. In one embodiment, the biomarker is Pfkfb3. In one embodiment, the biomarker is Pgam1. In one embodiment, the biomarker is Phlda1. In one embodiment, the biomarker is Pik3ip1. In one embodiment, the biomarker is Plk3. In one embodiment, the biomarker is Postn. In one embodiment, the biomarker is Ppargc1a. In one embodiment, the biomarker is Ppp1r14c. In one embodiment, the biomarker is Pragmin. In one embodiment, the biomarker is Prf1. In one embodiment, the biomarker is Ptpn14. In one embodiment, the biomarker is Pva1b. In one embodiment, the biomarker is Rab23. In one embodiment, the biomarker is Rab30. In one embodiment, the biomarker is Rbm20. In one embodiment, the biomarker is Rcan1. In one embodiment, the biomarker is Rel11. In one embodiment, the biomarker is Rfx1. In one embodiment, the biomarker is RGD1307461. In one embodiment, the biomarker is RGD1309676. In one embodiment, the biomarker is RGD1359290. In one embodiment, the biomarker is RGD1564428. In one embodiment, the biomarker is Rhpn2. In one embodiment, the biomarker is Rn45s. In one embodiment, the biomarker is Rnd1. In one embodiment, the biomarker is Rp1. In one embodiment, the biomarker is Rrad. In one embodiment, the biomarker is RT1-Ba. In one embodiment, the biomarker is RT1-Bb. In one embodiment, the biomarker is RT1-Da. In one embodiment, the biomarker is RT1-Db1. In one embodiment, the biomarker is Rtn4rl1. In one embodiment, the biomarker is Scd1. In one embodiment, the biomarker is Sdc4. In one embodiment, the biomarker is Sec1415. In one embodiment, the biomarker is Siglec5. In one embodiment, the biomarker is Sik1. In one embodiment, the biomarker is Slc18a2. In one embodiment, the biomarker is Slc2a5. In one embodiment, the biomarker is Slc30a4. In one embodiment, the biomarker is Slc4a1. In one embodiment, the biomarker is Slc4a5. In one embodiment, the biomarker is Slpi. In one embodiment, the biomarker is Smad7. In one embodiment, the biomarker is Snhg4. In one embodiment, the biomarker is Spag8. In one embodiment, the biomarker is Stc1. In one embodiment, the biomarker is Sv2c. In one embodiment, the biomarker is Terf2ip. In one embodiment, the biomarker is Thrsp. In one embodiment, the biomarker is Tmc8. In one embodiment, the biomarker is Tmem171. In one embodiment, the biomarker is Tmx4. In one embodiment, the biomarker is Tnfrsfl2a. In one embodiment, the biomarker is Tnni2. In one embodiment, the biomarker is Ttc30b. In one embodiment, the biomarker is Txnip. In one embodiment, the biomarker is Ucp3. In one embodiment, the biomarker is Unc5b. In one embodiment, the biomarker is Zfp112. In one embodiment, the biomarker is Zfp13. In one embodiment, the biomarker is Zfp385b. In one embodiment, the biomarker is Zfp474. In one embodiment, the biomarker is Zfyve28. In one embodiment, the biomarker is Zic1. In one embodiment, the biomarker is Zmynd10.

In one embodiment, the biomarker is Abcg1, and the level of the biomarker is increased. In one embodiment, the biomarker is Abra, and the level of the biomarker is increased. In one embodiment, the biomarker is Actn3, and the level of the biomarker is decreased. In one embodiment, the biomarker is Actn3, and the level of the biomarker is increased. In one embodiment, the biomarker is Alas2, and the level of the biomarker is decreased. In one embodiment, the biomarker is Alox15, and the level of the biomarker is decreased. In one embodiment, the biomarker is Alox15, and the level of the biomarker is increased. In one embodiment, the biomarker is Angpt14, and the level of the biomarker is decreased. In one embodiment, the biomarker is Apod, and the level of the biomarker is decreased. In one embodiment, the biomarker is Apold1, and the level of the biomarker is decreased. In one embodiment, the biomarker is Arc, and the level of the biomarker is decreased. In one embodiment, the biomarker is Arhgap24, and the level of the biomarker is increased. In one embodiment, the biomarker is Arl4c, and the level of the biomarker is increased. In one embodiment, the biomarker is Amt1, and the level of the biomarker is increased. In one embodiment, the biomarker is Arrdc2, and the level of the biomarker is decreased. In one embodiment, the biomarker is Asb5, and the level of the biomarker is increased. In one embodiment, the biomarker is Atf3, and the level of the biomarker is increased. In one embodiment, the biomarker is Bag2, and the level of the biomarker is increased. In one embodiment, the biomarker is Bcl11a, and the level of the biomarker is increased. In one embodiment, the biomarker is Bcl6, and the level of the biomarker is increased. In one embodiment, the biomarker is Bdh1, and the level of the biomarker is increased. In one embodiment, the biomarker is Bdnf, and the level of the biomarker is increased. In one embodiment, the biomarker is Best3, and the level of the biomarker is increased. In one embodiment, the biomarker is Bhlhe40, and the level of the biomarker is decreased. In one embodiment, the biomarker is Calhm1, and the level of the biomarker is increased. In one embodiment, the biomarker is Calm13, and the level of the biomarker is increased. In one embodiment, the biomarker is Car12, and the level of the biomarker is increased. In one embodiment, the biomarker is Ccl5, and the level of the biomarker is decreased. In one embodiment, the biomarker is Cd74, and the level of the biomarker is increased. In one embodiment, the biomarker is Cdc42se1, and the level of the biomarker is increased. In one embodiment, the biomarker is Chac1, and the level of the biomarker is decreased as compared to a control subject (e.g., a subject that has not been administered a population of stem cells (e.g., PDAC)), and the level of the biomarker is decreased. In one embodiment, the biomarker is Chst5, and the level of the biomarker is increased. In one embodiment, the biomarker is Ciart, and the level of the biomarker is decreased. In one embodiment, the biomarker is Cidec, and the level of the biomarker is increased. In one embodiment, the biomarker is Cish, and the level of the biomarker is decreased. In one embodiment, the biomarker is Cited4, and the level of the biomarker is decreased. In one embodiment, the biomarker is Ckap4, and the level of the biomarker is increased. In one embodiment, the biomarker is Cldn2, and the level of the biomarker is increased. In one embodiment, the biomarker is Clic6, and the level of the biomarker is increased. In one embodiment, the biomarker is Cpt1a, and the level of the biomarker is decreased. In one embodiment, the biomarker is Csrnp1, and the level of the biomarker is increased. In one embodiment, the biomarker is Cxcl13, and the level of the biomarker is decreased. In one embodiment, the biomarker is Cxcl13, and the level of the biomarker is increased. In one embodiment, the biomarker is Dbp, and the level of the biomarker is decreased. In one embodiment, the biomarker is Dnajb5, and the level of the biomarker is increased. In one embodiment, the biomarker is Dynl11, and the level of the biomarker is increased. In one embodiment, the biomarker is Dyrk2, and the level of the biomarker is increased. In one embodiment, the biomarker is Edn1, and the level of the biomarker is increased. In one embodiment, the biomarker is Egr1, and the level of the biomarker is decreased. In one embodiment, the biomarker is Egr3, and the level of the biomarker is decreased. In one embodiment, the biomarker is Elfn1, and the level of the biomarker is increased. In one embodiment, the biomarker is Emb, and the level of the biomarker is increased. In one embodiment, the biomarker is Enah, and the level of the biomarker is increased. In one embodiment, the biomarker is Fam107b, and the level of the biomarker is increased. In one embodiment, the biomarker is Fam110a, and the level of the biomarker is increased. In one embodiment, the biomarker is Fam134b, and the level of the biomarker is increased. In one embodiment, the biomarker is Fam167a, and the level of the biomarker is increased. In one embodiment, the biomarker is Fam46a, and the level of the biomarker is increased. In one embodiment, the biomarker is Fasn, and the level of the biomarker is decreased. In one embodiment, the biomarker is Fgfr3, and the level of the biomarker is increased. In one embodiment, the biomarker is Fhl2, and the level of the biomarker is increased. In one embodiment, the biomarker is Fos, and the level of the biomarker is increased. In one embodiment, the biomarker is Fosb, and the level of the biomarker is decreased. In one embodiment, the biomarker is Fosb, and the level of the biomarker is increased. In one embodiment, the biomarker is Frk, and the level of the biomarker is increased. In one embodiment, the biomarker is Fst, and the level of the biomarker is increased. In one embodiment, the biomarker is Gdf15, and the level of the biomarker is increased. In one embodiment, the biomarker is Gem, and the level of the biomarker is increased. In one embodiment, the biomarker is Gngt1, and the level of the biomarker is increased. In one embodiment, the biomarker is Gnl3, and the level of the biomarker is increased. In one embodiment, the biomarker is Hba1, and the level of the biomarker is decreased. In one embodiment, the biomarker is Hba2, and the level of the biomarker is decreased. In one embodiment, the biomarker is Hbb, and the level of the biomarker is decreased. In one embodiment, the biomarker is Hbb-b1, and the level of the biomarker is decreased. In one embodiment, the biomarker is Hbegf, and the level of the biomarker is increased. In one embodiment, the biomarker is Hmox1, and the level of the biomarker is increased. In one embodiment, the biomarker is Hpd1, and the level of the biomarker is decreased. In one embodiment, the biomarker is Hspa1b, and the level of the biomarker is increased. In one embodiment, the biomarker is Id4, and the level of the biomarker is increased. In one embodiment, the biomarker is Il2rb, and the level of the biomarker is decreased. In one embodiment, the biomarker is Irs1, and the level of the biomarker is increased. In one embodiment, the biomarker is Irs2, and the level of the biomarker is increased. In one embodiment, the biomarker is Junb, and the level of the biomarker is decreased. In one embodiment, the biomarker is Jund, and the level of the biomarker is increased. In one embodiment, the biomarker is Kbtbd8, and the level of the biomarker is increased. In one embodiment, the biomarker is Kcnk5, and the level of the biomarker is increased. In one embodiment, the biomarker is Kctd7, and the level of the biomarker is decreased. In one embodiment, the biomarker is Kirrel2, and the level of the biomarker is increased. In one embodiment, the biomarker is Ky, and the level of the biomarker is decreased. In one embodiment, the biomarker is Lamc2, and the level of the biomarker is increased. In one embodiment, the biomarker is Lipg, and the level of the biomarker is increased. In one embodiment, the biomarker is LOC689064, and the level of the biomarker is decreased. In one embodiment, the biomarker is Lonrf3, and the level of the biomarker is increased. In one embodiment, the biomarker is Lrrc38, and the level of the biomarker is increased. In one embodiment, the biomarker is Lrrc52, and the level of the biomarker is increased. In one embodiment, the biomarker is Lrrn2, and the level of the biomarker is decreased. In one embodiment, the biomarker is Lsr, and the level of the biomarker is increased. In one embodiment, the biomarker is Maff, and the level of the biomarker is increased. In one embodiment, the biomarker is Mchr1, and the level of the biomarker is decreased. In one embodiment, the biomarker is Mfrp, and the level of the biomarker is increased. In one embodiment, the biomarker is Mllt11, and the level of the biomarker is increased. In one embodiment, the biomarker is Mns1, and the level of the biomarker is increased. In one embodiment, the biomarker is Mogat1, and the level of the biomarker is increased. In one embodiment, the biomarker is Mphosph6, and the level of the biomarker is increased. In one embodiment, the biomarker is Mpz, and the level of the biomarker is decreased. In one embodiment, the biomarker is Muc20, and the level of the biomarker is increased. In one embodiment, the biomarker is Mybpc2, and the level of the biomarker is decreased. In one embodiment, the biomarker is Myf6, and the level of the biomarker is increased. In one embodiment, the biomarker is Myh1, and the level of the biomarker is decreased. In one embodiment, the biomarker is Myh2, and the level of the biomarker is decreased. In one embodiment, the biomarker is Myh4, and the level of the biomarker is increased. In one embodiment, the biomarker is Myocd, and the level of the biomarker is increased. In one embodiment, the biomarker is Nedd9, and the level of the biomarker is increased. In one embodiment, the biomarker is Nfil3, and the level of the biomarker is increased. In one embodiment, the biomarker is Nkg7, and the level of the biomarker is decreased. In one embodiment, the biomarker is Nrld1, and the level of the biomarker is decreased. In one embodiment, the biomarker is Nr4a2, and the level of the biomarker is decreased. In one embodiment, the biomarker is Nr4a2, and the level of the biomarker is increased. In one embodiment, the biomarker is Nr4a3, and the level of the biomarker is increased. In one embodiment, the biomarker is Ntf4, and the level of the biomarker is decreased. In one embodiment, the biomarker is Nuak1, and the level of the biomarker is increased. In one embodiment, the biomarker is Parp16, and the level of the biomarker is decreased. In one embodiment, the biomarker is Pdc, and the level of the biomarker is increased. In one embodiment, the biomarker is Pde7a, and the level of the biomarker is increased. In one embodiment, the biomarker is Pfkfb2, and the level of the biomarker is increased. In one embodiment, the biomarker is Pfkfb3, and the level of the biomarker is decreased. In one embodiment, the biomarker is Pgam1, and the level of the biomarker is increased. In one embodiment, the biomarker is Phlda1, and the level of the biomarker is increased. In one embodiment, the biomarker is Pik3ip1, and the level of the biomarker is decreased. In one embodiment, the biomarker is Plk3, and the level of the biomarker is decreased. In one embodiment, the biomarker is Postn, and the level of the biomarker is increased. In one embodiment, the biomarker is Ppargc1a, and the level of the biomarker is increased. In one embodiment, the biomarker is Ppp1rl4c, and the level of the biomarker is increased. In one embodiment, the biomarker is Pragmin, and the level of the biomarker is increased. In one embodiment, the biomarker is Prf1, and the level of the biomarker is decreased. In one embodiment, the biomarker is Ptpn14, and the level of the biomarker is increased. In one embodiment, the biomarker is Pva1b, and the level of the biomarker is decreased. In one embodiment, the biomarker is Pva1b, and the level of the biomarker is increased. In one embodiment, the biomarker is Rab23, and the level of the biomarker is increased. In one embodiment, the biomarker is Rab30, and the level of the biomarker is increased. In one embodiment, the biomarker is Rbm20, and the level of the biomarker is increased. In one embodiment, the biomarker is Rcan1, and the level of the biomarker is increased. In one embodiment, the biomarker is Rel11, and the level of the biomarker is increased. In one embodiment, the biomarker is Rfx1, and the level of the biomarker is increased. In one embodiment, the biomarker is RGD1307461, and the level of the biomarker is decreased. In one embodiment, the biomarker is RGD1309676, and the level of the biomarker is increased. In one embodiment, the biomarker is RGD1359290, and the level of the biomarker is increased. In one embodiment, the biomarker is RGD1564428, and the level of the biomarker is increased. In one embodiment, the biomarker is Rhpn2, and the level of the biomarker is increased. In one embodiment, the biomarker is Rn45s, and the level of the biomarker is decreased. In one embodiment, the biomarker is Rnd1, and the level of the biomarker is increased. In one embodiment, the biomarker is Rp1, and the level of the biomarker is increased. In one embodiment, the biomarker is Rrad, and the level of the biomarker is increased. In one embodiment, the biomarker is RT1-Ba, and the level of the biomarker is increased. In one embodiment, the biomarker is RT1-Bb, and the level of the biomarker is increased as compared to a control subject (e.g., a subject that has not been administered a population of stem cells (e.g., PDAC)) In one embodiment, the biomarker is RT1-Da, and the level of the biomarker is increased. In one embodiment, the biomarker is RT1-Db1, and the level of the biomarker is increased. In one embodiment, the biomarker is Rtn4rl1, and the level of the biomarker is decreased. In one embodiment, the biomarker is Scd1, and the level of the biomarker is decreased. In one embodiment, the biomarker is Scd1, and the level of the biomarker is increased. In one embodiment, the biomarker is Sdc4, and the level of the biomarker is increased. In one embodiment, the biomarker is Sec1415, and the level of the biomarker is decreased. In one embodiment, the biomarker is Siglec5, and the level of the biomarker is decreased. In one embodiment, the biomarker is Sik1, and the level of the biomarker is increased. In one embodiment, the biomarker is Slc18a2, and the level of the biomarker is increased. In one embodiment, the biomarker is Slc2a5, and the level of the biomarker is decreased. In one embodiment, the biomarker is Slc30a4, and the level of the biomarker is increased. In one embodiment, the biomarker is Slc4a1, and the level of the biomarker is decreased. In one embodiment, the biomarker is Slc4a1, and the level of the biomarker is increased. In one embodiment, the biomarker is Slc4a5, and the level of the biomarker is increased. In one embodiment, the biomarker is Slpi, and the level of the biomarker is decreased. In one embodiment, the biomarker is Smad7, and the level of the biomarker is increased. In one embodiment, the biomarker is Snhg4, and the level of the biomarker is decreased. In one embodiment, the biomarker is Spag8, and the level of the biomarker is decreased. In one embodiment, the biomarker is Stc1, and the level of the biomarker is increased. In one embodiment, the biomarker is Sv2c, and the level of the biomarker is increased. In one embodiment, the biomarker is Terf2ip, and the level of the biomarker is increased. In one embodiment, the biomarker is Thrsp, and the level of the biomarker is decreased. In one embodiment, the biomarker is Tmc8, and the level of the biomarker is decreased. In one embodiment, the biomarker is Tmem171, and the level of the biomarker is increased. In one embodiment, the biomarker is Tmx4, and the level of the biomarker is increased. In one embodiment, the biomarker is Tnfrsfl2a, and the level of the biomarker is increased. In one embodiment, the biomarker is Tnni2, and the level of the biomarker is decreased. In one embodiment, the biomarker is Ttc30b, and the level of the biomarker is decreased. In one embodiment, the biomarker is Txnip, and the level of the biomarker is decreased. In one embodiment, the biomarker is Ucp3, and the level of the biomarker is decreased. In one embodiment, the biomarker is Unc5b, and the level of the biomarker is increased. In one embodiment, the biomarker is Zfp112, and the level of the biomarker is decreased. In one embodiment, the biomarker is Zfp13, and the level of the biomarker is decreased. In one embodiment, the biomarker is Zfp385b, and the level of the biomarker is increased. In one embodiment, the biomarker is Zfp474, and the level of the biomarker is increased. In one embodiment, the biomarker is Zfyve28, and the level of the biomarker is decreased. In one embodiment, the biomarker is Zic, and the level of the biomarker is increased. In one embodiment, the biomarker is Zmynd10, and the level of the biomarker is decreased. In certain embodiments, the increased level of the biomarker is as compared to a control subject. In certain embodiments, the decreased level of the biomarker is as compared to a control subject. In a specific embodiment, the control subject is a subject that has not been administered a population of stem cells (e.g., PDAC)).

In another aspect, provided herein is a method of altering the transcriptome of an aging cell in a tissue of a subject in need thereof, comprising administering to the subject an effective amount of a population of PDSC, wherein the amount is effective to alter the transcriptome of the aging cell, wherein the altered transcriptome comprises one or more transcripts found in a younger cell in the tissue of a control subject. In some embodiments, the one or more transcripts are identified using a transcript array analysis. In some embodiments, the one or more transcripts are identified using TaqMan® Low Density Arrays (TLDA) on 7900HT Real-Time PCR systems. In a specific embodiment, the transcript is a transcript of a biomarker provided herein. In some embodiments, the transcript is increased relative to the same transcript found in the younger cell. In other embodiments, the transcript is decreased relative to the same transcript found in the younger cell.

In some embodiments, the one or more transcripts are selected from the group consisting of MLCF3, myosin light polypeptide 2 (slow), MLC1F, MYBPC1, myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, MYH2, TNNT1, RYR1, CASQ1, JPH1, AMPD1, PYGM, and ENO3.

In some embodiments, the one or more transcripts are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the one or more transcripts is indicative of aging.

In some embodiments, the one or more transcripts are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the one or more transcripts is indicative of aging.

In some embodiments, the one or more transcripts are selected from the group consisting of myristoylated alanine-rich C-kinase substrate, alpha-internexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of MBP, and VIM.

In some embodiments, the one or more transcripts are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1.

In some embodiments, the one or more transcripts are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU).

In some embodiments, the one or more transcripts are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN).

In some embodiments, the one or more transcripts are selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH).

In some embodiments, the one or more transcripts are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1.

In some embodiments, the one or more transcripts are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2).

In some embodiments, the one or more transcripts are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2).

In some embodiments, the one or more transcripts are selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the one or more transcripts is indicative of aging.

In some embodiments, the transcript is elongation factor 2 (Eef2) and an increase in the expression of Eef2 is indicative of aging.

In some embodiments, the one or more transcripts are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH).

In some embodiments, the transcript is selected from the group consisting of transferrin, isocitrate dehydrogenase 1 (IDH), and 3-hydroxyisobutyrate dehydrogenase, wherein an increase in the expression of the one or more transcripts is indicative of an aging.

In some embodiments, the one or more transcripts are selected from the group consisting of afenopin, phosphoglycerate mutase, and glutathione (GSH), wherein a decrease in the expression of the one or more transcripts is indicative of aging.

In some embodiments, the increase in expression of the one or more transcripts is gender specific. For example, in some instances, the transcript is ATP synthase and the expression of the ATP synthase in up-regulated in aging males. In some instances, the transcript is catalase and the expression of the catalase is down-regulated in aging males. In other instances, the transcript is ATP synthase and the expression of ATP synthase is down-regulated in aging females. In some embodiments, the transcript is ornithine aminotransferase and the expression of the ornithine aminotransferase is up-regulated in aging females. In some embodiments, the transcript is glutamate dehydrogenase and the expression of the glutamate dehydrogenase is down-regulated in aging females.

In some embodiments, the one or more transcripts are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase.

In some embodiments, the one or more transcripts are selected from the group consisting of epoxide hydroxylase, 3-ketoacyl-CoA thiolase A, sarcosine oxidase, and 2,4-dienoyl reductase, wherein an increase in expression of the one or more transcripts is indicative of aging.

In some embodiments, the one or more transcripts are selected from the group consisting of defensin, alpha 1 (DEFA1), defensin, alpha 1B (DEFAIB), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5).

In some embodiments, the one or more transcripts are selected from the group consisting of fatty acid-binding protein 5, galectin-3, c-synuclein, heterogeneous nuclear ribonucleoprotein A1, myosin light chain, regulatory B, peroxiredoxin 5 precursor, and transgelin.

In some embodiments, the one or more transcripts are selected from the group consisting of beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterogeneous nuclear ribonucleoprotein A1 isoform a (HNRPA1), heterogeneous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln).

In some embodiments, the one or more transcripts are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5).

In some embodiments, the one or more transcripts are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1).

In some embodiments, the one or more transcripts are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the one or more transcripts is indicative of aging.

In some embodiments, the one or more transcripts are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1).

In some embodiments, the one or more transcripts are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the one or more transcripts is indicative of aging.

In some embodiments, the one or more transcripts are selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the one or more transcripts is indicative of aging.

In one embodiment, the transcript is one or more transcripts independently selected from the group consisting of Abcg1, Abra, Actn3, Alas2, Alox15, Angpt14, Apod, Apold1, Arc, Arhgap24, Arl4c, Amt1, Arrdc2, Asb5, Atf3, Bag2, Bcl11a, Bcl6, Bdh1, Bdnf, Best3, Bhlhe40, Calhm1, Calm13, Car12, Ccl5, Cd74, Cdc42se1, Chac1, Chst5, Ciart, Cidec, Cish, Cited4, Ckap4, Cldn2, Clic6, Cpt1a, Csrnp1, Cxcl13, Dbp, Dnajb5, Dynl11, Dyrk2, Edn1, Egr1, Egr3, Elfn1, Emb, Enah, Fam107b, Fam110a, Fam134b, Fam167a, Fam46a, Fasn, Fgfr3, Fhl2, Fos, Fosb, Frk, Fst, Gdf15, Gem, Gngt1, Gnl3, Hba1, Hba2, Hbb, Hbb-b1, Hbegf, Hmox1, Hpd1, Hspa1b, Id4, Il2rb, Irs1, Irs2, Junb, Jund, Kbtbd8, Kcnk5, Kctd7, Kirrel2, Ky, Lamc2, Lipg, LOC689064, Lonrf3, Lrrc38, Lrrc52, Lrrn2, Lsr, Maff, Mchr1, Mfrp, Mllt11, Mns1, Mogat1, Mphosph6, Mpz, Muc20, Mybpc2, Myf6, Myh1, Myh2, Myh4, Myocd, Nedd9, Nfil3, Nkg7, Nrld1, Nr4a2, Nr4a3, Ntf4, Nuak1, Parp16, Pdc, Pde7a, Pfkfb2, Pfkfb3, Pgam1, Phlda1, Pik3ip1, Plk3, Postn, Ppargc1a, Ppp1rl4c, Pragmin, Prf1, Ptpn14, Pva1b, Rab23, Rab30, Rbm20, Rcan1, Rel11, Rfx1, RGD1307461, RGD1309676, RGD1359290, RGD1564428, Rhpn2, Rn45s, Rnd1, Rp1, Rrad, RT1-Ba, RT1-Bb, RT1-Da, RT1-Db1, Rtn4rl1, Scd1, Sdc4, Sec1415, Siglec5, Sik1, Slc18a2, Slc2a5, Slc30a4, Slc4a1, Slc4a5, Slpi, Smad7, Snhg4, Spag8, Stc1, Sv2c, Terf2ip, Thrsp, Tmc8, Tmem171, Tmx4, Tnfrsfl2a, Tnni2, Ttc30b, Txnip, Ucp3, Unc5b, Zfp112, Zfp13, Zfp385b and Zfp474, Zfyve28, Zic1 or Zmynd10. In one embodiment, the one or more transcripts is 2, 3, 4, 5, 6, 7, 8, 9, 10, 15, 20, 25, 30, 35, 40, 45, 50, 55, 60, 65, 70, 75 or more transcripts, or any range or interval thereof. In one embodiment, the transcript is Abcg1. In one embodiment, the transcript is Abra. In one embodiment, the transcript is Actn3. In one embodiment, the transcript is Alas2. In one embodiment, the transcript is Alox15. In one embodiment, the transcript is Angpt14. In one embodiment, the transcript is Apod. In one embodiment, the transcript is Apold1. In one embodiment, the transcript is Arc. In one embodiment, the transcript is Arhgap24. In one embodiment, the transcript is Arl4c. In one embodiment, the transcript is Amt1. In one embodiment, the transcript is Arrdc2. In one embodiment, the transcript is Asb5. In one embodiment, the transcript is Atf3. In one embodiment, the transcript is Bag2. In one embodiment, the transcript is Bcl11a. In one embodiment, the transcript is Bcl6. In one embodiment, the transcript is Bdh1. In one embodiment, the transcript is Bdnf. In one embodiment, the transcript is Best3. In one embodiment, the transcript is Bhlhe40. In one embodiment, the transcript is Calhm1. In one embodiment, the transcript is Calm13. In one embodiment, the transcript is Car12. In one embodiment, the transcript is Ccl5. In one embodiment, the transcript is Cd74. In one embodiment, the transcript is Cdc42se1. In one embodiment, the transcript is Chac1. In one embodiment, the transcript is Chst5. In one embodiment, the transcript is Ciart. In one embodiment, the transcript is Cidec. In one embodiment, the transcript is Cish. In one embodiment, the transcript is Cited4. In one embodiment, the transcript is Ckap4. In one embodiment, the transcript is Cldn2. In one embodiment, the transcript is Clic6. In one embodiment, the transcript is Cpt1a. In one embodiment, the transcript is Csrnp1. In one embodiment, the transcript is Cxcl13. In one embodiment, the transcript is Dbp. In one embodiment, the transcript is Dnajb5. In one embodiment, the transcript is Dynl11. In one embodiment, the transcript is Dyrk2. In one embodiment, the transcript is Edn1. In one embodiment, the transcript is Egr1. In one embodiment, the transcript is Egr3. In one embodiment, the transcript is Elfn1. In one embodiment, the transcript is Emb. In one embodiment, the transcript is Enah. In one embodiment, the transcript is Fam107b. In one embodiment, the transcript is Fam110a. In one embodiment, the transcript is Fam134b. In one embodiment, the transcript is Fam167a. In one embodiment, the transcript is Fam46a. In one embodiment, the transcript is Fasn. In one embodiment, the transcript is Fgfr3. In one embodiment, the transcript is Fhl2. In one embodiment, the transcript is Fos. In one embodiment, the transcript is Fosb. In one embodiment, the transcript is Frk. In one embodiment, the transcript is Fst. In one embodiment, the transcript is Gdf15. In one embodiment, the transcript is Gem. In one embodiment, the transcript is Gngt1. In one embodiment, the transcript is Gnl3. In one embodiment, the transcript is Hba1. In one embodiment, the transcript is Hba2. In one embodiment, the transcript is Hbb. In one embodiment, the transcript is Hbb-b1. In one embodiment, the transcript is Hbegf. In one embodiment, the transcript is Hmox1. In one embodiment, the transcript is Hpd1. In one embodiment, the transcript is Hspa1b. In one embodiment, the transcript is Id4. In one embodiment, the transcript is Il2rb. In one embodiment, the transcript is Irs1. In one embodiment, the transcript is Irs2. In one embodiment, the transcript is Junb. In one embodiment, the transcript is Jund. In one embodiment, the transcript is Kbtbd8. In one embodiment, the transcript is Kcnk5. In one embodiment, the transcript is Kctd7. In one embodiment, the transcript is Kirrel2. In one embodiment, the transcript is Ky. In one embodiment, the transcript is Lamc2. In one embodiment, the transcript is Lipg. In one embodiment, the transcript is LOC689064. In one embodiment, the transcript is Lonrf3. In one embodiment, the transcript is Lrrc38. In one embodiment, the transcript is Lrrc52. In one embodiment, the transcript is Lrrn2. In one embodiment, the transcript is Lsr. In one embodiment, the transcript is Maff. In one embodiment, the transcript is Mchr1. In one embodiment, the transcript is Mfrp. In one embodiment, the transcript is Mllt11. In one embodiment, the transcript is Mns1. In one embodiment, the transcript is Mogat1. In one embodiment, the transcript is Mphosph6. In one embodiment, the transcript is Mpz. In one embodiment, the transcript is Muc20. In one embodiment, the transcript is Mybpc2. In one embodiment, the transcript is Myf6. In one embodiment, the transcript is Myh1. In one embodiment, the transcript is Myh2. In one embodiment, the transcript is Myh4. In one embodiment, the transcript is Myocd. In one embodiment, the transcript is Nedd9. In one embodiment, the transcript is Nfil3. In one embodiment, the transcript is Nkg7. In one embodiment, the transcript is Nrld1. In one embodiment, the transcript is Nr4a2. In one embodiment, the transcript is Nr4a3. In one embodiment, the transcript is Ntf4. In one embodiment, the transcript is Nuak1. In one embodiment, the transcript is Parp16. In one embodiment, the transcript is Pdc. In one embodiment, the transcript is Pde7a. In one embodiment, the transcript is Pfkfb2. In one embodiment, the transcript is Pfkfb3. In one embodiment, the transcript is Pgam1. In one embodiment, the transcript is Phlda1. In one embodiment, the transcript is Pik3ip1. In one embodiment, the transcript is Plk3. In one embodiment, the transcript is Postn. In one embodiment, the transcript is Ppargc1a. In one embodiment, the transcript is Ppp1r14c. In one embodiment, the transcript is Pragmin. In one embodiment, the transcript is Prf1. In one embodiment, the transcript is Ptpn14. In one embodiment, the transcript is Pva1b. In one embodiment, the transcript is Rab23. In one embodiment, the transcript is Rab30. In one embodiment, the transcript is Rbm20. In one embodiment, the transcript is Rcan1. In one embodiment, the transcript is Rel11. In one embodiment, the transcript is Rfx1. In one embodiment, the transcript is RGD1307461. In one embodiment, the transcript is RGD1309676. In one embodiment, the transcript is RGD1359290. In one embodiment, the transcript is RGD1564428. In one embodiment, the transcript is Rhpn2. In one embodiment, the transcript is Rn45s. In one embodiment, the transcript is Rnd1. In one embodiment, the transcript is Rp1. In one embodiment, the transcript is Rrad. In one embodiment, the transcript is RT1-Ba. In one embodiment, the transcript is RT1-Bb. In one embodiment, the transcript is RT1-Da. In one embodiment, the transcript is RT1-Db1. In one embodiment, the transcript is Rtn4rl1. In one embodiment, the transcript is Scd1. In one embodiment, the transcript is Sdc4. In one embodiment, the transcript is Sec1415. In one embodiment, the transcript is Siglec5. In one embodiment, the transcript is Sik1. In one embodiment, the transcript is Slc18a2. In one embodiment, the transcript is Slc2a5. In one embodiment, the transcript is Slc30a4. In one embodiment, the transcript is Slc4a1. In one embodiment, the transcript is Slc4a5. In one embodiment, the transcript is Slpi. In one embodiment, the transcript is Smad7. In one embodiment, the transcript is Snhg4. In one embodiment, the transcript is Spag8. In one embodiment, the transcript is Stc1. In one embodiment, the transcript is Sv2c. In one embodiment, the transcript is Terf2ip. In one embodiment, the transcript is Thrsp. In one embodiment, the transcript is Tmc8. In one embodiment, the transcript is Tmem171. In one embodiment, the transcript is Tmx4. In one embodiment, the transcript is Tnfrsfl2a. In one embodiment, the transcript is Tnni2. In one embodiment, the transcript is Ttc30b. In one embodiment, the transcript is Txnip. In one embodiment, the transcript is Ucp3. In one embodiment, the transcript is Unc5b. In one embodiment, the transcript is Zfp112. In one embodiment, the transcript is Zfp13. In one embodiment, the transcript is Zfp385b. In one embodiment, the transcript is Zfp474. In one embodiment, the transcript is Zfyve28. In one embodiment, the transcript is Zic1. In one embodiment, the transcript is Zmynd10.

In one embodiment, the transcript is Abcg1, and the transcript expression is increased. In one embodiment, the transcript is Abra, and the transcript expression is increased. In one embodiment, the transcript is Actn3, and the transcript expression is decreased. In one embodiment, the transcript is Actn3, and the transcript expression is increased. In one embodiment, the transcript is Alas2, and the transcript expression is decreased. In one embodiment, the transcript is Alox5, and the transcript expression is decreased. In one embodiment, the transcript is Alox5, and the transcript expression is increased. In one embodiment, the transcript is Angpt14, and the transcript expression is decreased. In one embodiment, the transcript is Apod, and the transcript expression is decreased. In one embodiment, the transcript is Apold1, and the transcript expression is decreased. In one embodiment, the transcript is Arc, and the transcript expression is decreased. In one embodiment, the transcript is Arhgap24, and the transcript expression is increased. In one embodiment, the transcript is Arl4c, and the transcript expression is increased. In one embodiment, the transcript is Amt1, and the transcript expression is increased. In one embodiment, the transcript is Arrdc2, and the transcript expression is decreased. In one embodiment, the transcript is Asb5, and the transcript expression is increased. In one embodiment, the transcript is Atf3, and the transcript expression is increased. In one embodiment, the transcript is Bag2, and the transcript expression is increased. In one embodiment, the transcript is Bcl1 1a, and the transcript expression is increased. In one embodiment, the transcript is Bcl6, and the transcript expression is increased. In one embodiment, the transcript is Bdh1, and the transcript expression is increased. In one embodiment, the transcript is Bdnf, and the transcript expression is increased. In one embodiment, the transcript is Best3, and the transcript expression is increased. In one embodiment, the transcript is Bhlhe40, and the transcript expression is decreased. In one embodiment, the transcript is Calhm1, and the transcript expression is increased. In one embodiment, the transcript is Calm13, and the transcript expression is increased. In one embodiment, the transcript is Car12, and the transcript expression is increased. In one embodiment, the transcript is Ccl5, and the transcript expression is decreased. In one embodiment, the transcript is Cd74, and the transcript expression is increased. In one embodiment, the transcript is Cdc42se1, and the transcript expression is increased. In one embodiment, the transcript is Chac1, and the transcript expression is decreased as compared to a control subject (e.g., a subject that has not been administered a population of stem cells (e.g., PDAC)), and the transcript expression is decreased. In one embodiment, the transcript is Chst5, and the transcript expression is increased. In one embodiment, the transcript is Ciart, and the transcript expression is decreased. In one embodiment, the transcript is Cidec, and the transcript expression is increased. In one embodiment, the transcript is Cish, and the transcript expression is decreased. In one embodiment, the transcript is Cited4, and the transcript expression is decreased. In one embodiment, the transcript is Ckap4, and the transcript expression is increased. In one embodiment, the transcript is Cldn2, and the transcript expression is increased. In one embodiment, the transcript is Clic6, and the transcript expression is increased. In one embodiment, the transcript is Cpt1a, and the transcript expression is decreased. In one embodiment, the transcript is Csrnp1, and the transcript expression is increased. In one embodiment, the transcript is Cxcl13, and the transcript expression is decreased. In one embodiment, the transcript is Cxcl13, and the transcript expression is increased. In one embodiment, the transcript is Dbp, and the transcript expression is decreased. In one embodiment, the transcript is Dnajb5, and the transcript expression is increased. In one embodiment, the transcript is Dynl11, and the transcript expression is increased. In one embodiment, the transcript is Dyrk2, and the transcript expression is increased. In one embodiment, the transcript is Edn1, and the transcript expression is increased. In one embodiment, the transcript is Egr1, and the transcript expression is decreased. In one embodiment, the transcript is Egr3, and the transcript expression is decreased. In one embodiment, the transcript is Elfn1, and the transcript expression is increased. In one embodiment, the transcript is Emb, and the transcript expression is increased. In one embodiment, the transcript is Enah, and the transcript expression is increased. In one embodiment, the transcript is Fam107b, and the transcript expression is increased. In one embodiment, the transcript is Fam110a, and the transcript expression is increased. In one embodiment, the transcript is Fam134b, and the transcript expression is increased. In one embodiment, the transcript is Fam167a, and the transcript expression is increased. In one embodiment, the transcript is Fam46a, and the transcript expression is increased. In one embodiment, the transcript is Fasn, and the transcript expression is decreased. In one embodiment, the transcript is Fgfr3, and the transcript expression is increased. In one embodiment, the transcript is Fhl2, and the transcript expression is increased. In one embodiment, the transcript is Fos, and the transcript expression is increased. In one embodiment, the transcript is Fosb, and the transcript expression is decreased. In one embodiment, the transcript is Fosb, and the transcript expression is increased. In one embodiment, the transcript is Frk, and the transcript expression is increased. In one embodiment, the transcript is Fst, and the transcript expression is increased. In one embodiment, the transcript is Gdf15, and the transcript expression is increased. In one embodiment, the transcript is Gem, and the transcript expression is increased. In one embodiment, the transcript is Gngt1, and the transcript expression is increased. In one embodiment, the transcript is Gnl3, and the transcript expression is increased. In one embodiment, the transcript is Hba1, and the transcript expression is decreased. In one embodiment, the transcript is Hba2, and the transcript expression is decreased. In one embodiment, the transcript is Hbb, and the transcript expression is decreased. In one embodiment, the transcript is Hbb-b1, and the transcript expression is decreased. In one embodiment, the transcript is Hbegf, and the transcript expression is increased. In one embodiment, the transcript is Hmox1, and the transcript expression is increased. In one embodiment, the transcript is Hpd1, and the transcript expression is decreased. In one embodiment, the transcript is Hspa1b, and the transcript expression is increased. In one embodiment, the transcript is Id4, and the transcript expression is increased. In one embodiment, the transcript is Il2rb, and the transcript expression is decreased. In one embodiment, the transcript is Irs1, and the transcript expression is increased. In one embodiment, the transcript is Irs2, and the transcript expression is increased. In one embodiment, the transcript is Junb, and the transcript expression is decreased. In one embodiment, the transcript is Jund, and the transcript expression is increased. In one embodiment, the transcript is Kbtbd8, and the transcript expression is increased. In one embodiment, the transcript is Kcnk5, and the transcript expression is increased. In one embodiment, the transcript is Kctd7, and the transcript expression is decreased. In one embodiment, the transcript is Kirrel2, and the transcript expression is increased. In one embodiment, the transcript is Ky, and the transcript expression is decreased. In one embodiment, the transcript is Lamc2, and the transcript expression is increased. In one embodiment, the transcript is Lipg, and the transcript expression is increased. In one embodiment, the transcript is LOC689064, and the transcript expression is decreased. In one embodiment, the transcript is Lonrf3, and the transcript expression is increased. In one embodiment, the transcript is Lrrc38, and the transcript expression is increased. In one embodiment, the transcript is Lrrc52, and the transcript expression is increased. In one embodiment, the transcript is Lrrn2, and the transcript expression is decreased. In one embodiment, the transcript is Lsr, and the transcript expression is increased. In one embodiment, the transcript is Maff, and the transcript expression is increased. In one embodiment, the transcript is Mchr1, and the transcript expression is decreased. In one embodiment, the transcript is Mfrp, and the transcript expression is increased. In one embodiment, the transcript is Mllt11, and the transcript expression is increased. In one embodiment, the transcript is Mns1, and the transcript expression is increased. In one embodiment, the transcript is Mogat1, and the transcript expression is increased. In one embodiment, the transcript is Mphosph6, and the transcript expression is increased. In one embodiment, the transcript is Mpz, and the transcript expression is decreased. In one embodiment, the transcript is Muc20, and the transcript expression is increased. In one embodiment, the transcript is Mybpc2, and the transcript expression is decreased. In one embodiment, the transcript is Myf6, and the transcript expression is increased. In one embodiment, the transcript is Myh1, and the transcript expression is decreased. In one embodiment, the transcript is Myh2, and the transcript expression is decreased. In one embodiment, the transcript is Myh4, and the transcript expression is increased. In one embodiment, the transcript is Myocd, and the transcript expression is increased. In one embodiment, the transcript is Nedd9, and the transcript expression is increased. In one embodiment, the transcript is Nfil3, and the transcript expression is increased. In one embodiment, the transcript is Nkg7, and the transcript expression is decreased. In one embodiment, the transcript is Nrld1, and the transcript expression is decreased. In one embodiment, the transcript is Nr4a2, and the transcript expression is decreased. In one embodiment, the transcript is Nr4a2, and the transcript expression is increased. In one embodiment, the transcript is Nr4a3, and the transcript expression is increased. In one embodiment, the transcript is Ntf4, and the transcript expression is decreased. In one embodiment, the transcript is Nuak1, and the transcript expression is increased. In one embodiment, the transcript is Parp16, and the transcript expression is decreased. In one embodiment, the transcript is Pdc, and the transcript expression is increased. In one embodiment, the transcript is Pde7a, and the transcript expression is increased. In one embodiment, the transcript is Pfkfb2, and the transcript expression is increased. In one embodiment, the transcript is Pfkfb3, and the transcript expression is decreased. In one embodiment, the transcript is Pgam1, and the transcript expression is increased. In one embodiment, the transcript is Phlda1, and the transcript expression is increased. In one embodiment, the transcript is Pik3ip1, and the transcript expression is decreased. In one embodiment, the transcript is Plk3, and the transcript expression is decreased. In one embodiment, the transcript is Postn, and the transcript expression is increased. In one embodiment, the transcript is Ppargc1a, and the transcript expression is increased. In one embodiment, the transcript is Ppp1r14c, and the transcript expression is increased. In one embodiment, the transcript is Pragmin, and the transcript expression is increased. In one embodiment, the transcript is Prf1, and the transcript expression is decreased. In one embodiment, the transcript is Ptpn14, and the transcript expression is increased. In one embodiment, the transcript is Pva1b, and the transcript expression is decreased. In one embodiment, the transcript is Pva1b, and the transcript expression is increased. In one embodiment, the transcript is Rab23, and the transcript expression is increased. In one embodiment, the transcript is Rab30, and the transcript expression is increased. In one embodiment, the transcript is Rbm20, and the transcript expression is increased. In one embodiment, the transcript is Rcan1, and the transcript expression is increased. In one embodiment, the transcript is Rel11, and the transcript expression is increased. In one embodiment, the transcript is Rfx1, and the transcript expression is increased. In one embodiment, the transcript is RGD1307461, and the transcript expression is decreased. In one embodiment, the transcript is RGD1309676, and the transcript expression is increased. In one embodiment, the transcript is RGD1359290, and the transcript expression is increased. In one embodiment, the transcript is RGD1564428, and the transcript expression is increased. In one embodiment, the transcript is Rhpn2, and the transcript expression is increased. In one embodiment, the transcript is Rn45s, and the transcript expression is decreased. In one embodiment, the transcript is Rnd1, and the transcript expression is increased. In one embodiment, the transcript is Rp1, and the transcript expression is increased. In one embodiment, the transcript is Rrad, and the transcript expression is increased. In one embodiment, the transcript is RT1-Ba, and the transcript expression is increased. In one embodiment, the transcript is RT1-Bb, and the transcript expression is increased as compared to a control subject (e.g., a subject that has not been administered a population of stem cells (e.g., PDAC)) In one embodiment, the transcript is RT1-Da, and the transcript expression is increased. In one embodiment, the transcript is RT1-Db1, and the transcript expression is increased. In one embodiment, the transcript is Rtn4rl11, and the transcript expression is decreased. In one embodiment, the transcript is Scd1, and the transcript expression is decreased. In one embodiment, the transcript is Scd1, and the transcript expression is increased. In one embodiment, the transcript is Sdc4, and the transcript expression is increased. In one embodiment, the transcript is Sec1415, and the transcript expression is decreased. In one embodiment, the transcript is Siglec5, and the transcript expression is decreased. In one embodiment, the transcript is Sik1, and the transcript expression is increased. In one embodiment, the transcript is Slc18a2, and the transcript expression is increased. In one embodiment, the transcript is Slc2a5, and the transcript expression is decreased. In one embodiment, the transcript is Slc30a4, and the transcript expression is increased. In one embodiment, the transcript is Slc4a1, and the transcript expression is decreased. In one embodiment, the transcript is Slc4a1, and the transcript expression is increased. In one embodiment, the transcript is Slc4a5, and the transcript expression is increased. In one embodiment, the transcript is Slpi, and the transcript expression is decreased. In one embodiment, the transcript is Smad7, and the transcript expression is increased. In one embodiment, the transcript is Snhg4, and the transcript expression is decreased. In one embodiment, the transcript is Spag8, and the transcript expression is decreased. In one embodiment, the transcript is Stc1, and the transcript expression is increased. In one embodiment, the transcript is Sv2c, and the transcript expression is increased. In one embodiment, the transcript is Terf2ip, and the transcript expression is increased. In one embodiment, the transcript is Thrsp, and the transcript expression is decreased. In one embodiment, the transcript is Tmc8, and the transcript expression is decreased. In one embodiment, the transcript is Tmem171, and the transcript expression is increased. In one embodiment, the transcript is Tmx4, and the transcript expression is increased. In one embodiment, the transcript is Tnfrsfl2a, and the transcript expression is increased. In one embodiment, the transcript is Tnni2, and the transcript expression is decreased. In one embodiment, the transcript is Ttc30b, and the transcript expression is decreased. In one embodiment, the transcript is Txnip, and the transcript expression is decreased. In one embodiment, the transcript is Ucp3, and the transcript expression is decreased. In one embodiment, the transcript is Unc5b, and the transcript expression is increased. In one embodiment, the transcript is Zfp112, and the transcript expression is decreased. In one embodiment, the transcript is Zfp13, and the transcript expression is decreased. In one embodiment, the transcript is Zfp385b, and the transcript expression is increased. In one embodiment, the transcript is Zfp474, and the transcript expression is increased. In one embodiment, the transcript is Zfyve28, and the transcript expression is decreased. In one embodiment, the transcript is Zic, and the transcript expression is increased. In one embodiment, the transcript is Zmynd10, and the transcript expression is decreased. In certain embodiments, the increased transcript expression is as compared to a control subject. In certain embodiments, the decreased transcript expression is as compared to a control subject. In a specific embodiment, the control subject is a subject that has not been administered a population of stem cells (e.g., PDAC)).

In some embodiments, a gene expression is modulated in a subject following administration of the stem cells (e.g., PDSC). In one embodiment, the gene is a gene provided in any one of Table 5-9. In one embodiment, the gene is selected from a gene provided in Table 5. In one embodiment, the gene is selected from a gene provided in Table 6. In one embodiment, the gene is selected from a gene provided in Table 7. In one embodiment, the gene is selected from a gene provided in Table 8. In one embodiment, the gene is selected from a gene provided in Table 9. In one embodiment, the gene is one or more genes independently selected from the group consisting of Abcg1, Abra, Actn3, Alas2, Alox15, Angpt14, Apod, Apold1, Arc, Arhgap24, Arl4c, Amt1, Arrdc2, Asb5, Atf3, Bag2, Bcl11a, Bcl6, Bdh1, Bdnf, Best3, Bhlhe40, Calhm1, Calm13, Car12, Ccl5, Cd74, Cdc42se1, Chac1, Chst5, Ciart, Cidec, Cish, Cited4, Ckap4, Cldn2, Clic6, Cpt1a, Csrnp1, Cxcl13, Dbp, Dnajb5, Dynl11, Dyrk2, Edn1, Egr1, Egr3, Elfn1, Emb, Enah, Fam107b, Fam110a, Fam134b, Fam167a, Fam46a, Fasn, Fgfr3, Fhl2, Fos, Fosb, Frk, Fst, Gdf15, Gem, Gngt1, Gnl3, Hba1, Hba2, Hbb, Hbb-b1, Hbegf, Hmox1, Hpd1, Hspa1b, Id4, Il12rb, Irs1, Irs2, Junb, Jund, Kbtbd8, Kcnk5, Kctd7, Kirrel2, Ky, Lamc2, Lipg, LOC689064, Lonrf3, Lrrc38, Lrrc52, Lrrn2, Lsr, Maff, Mchr1, Mfrp, Mllt11, Mns1, Mogat1, Mphosph6, Mpz, Muc20, Mybpc2, Myf6, Myh1, Myh2, Myh4, Myocd, Nedd9, Nfil3, Nkg7, Nrld1, Nr4a2, Nr4a3, Ntf4, Nuak1, Parp16, Pdc, Pde7a, Pfkfb2, Pfkfb3, Pgam1, Phlda1, Pik3ip1, Plk3, Postn, Ppargc1a, Ppp1rl4c, Pragmin, Prf1, Ptpn14, Pva1b, Rab23, Rab30, Rbm20, Rcan1, Rel11, Rfx1, RGD1307461, RGD1309676, RGD1359290, RGD1564428, Rhpn2, Rn45s, Rnd1, Rp1, Rrad, RT1-Ba, RT1-Bb, RT1-Da, RT1-Db1, Rtn4rl1, Scd1, Sdc4, Sec1415, Siglec5, Sik1, Slc18a2, Slc2a5, Slc30a4, Slc4a1, Slc4a5, Slpi, Smad7, Snhg4, Spag8, Stc1, Sv2c, Terf2ip, Thrsp, Tmc8, Tmem171, Tmx4, Tnfrsfl2a, Tnni2, Ttc30b, Txnip, Ucp3, Unc5b, Zfp112, Zfp13, Zfp385b and Zfp474, Zfyve28, Zic1 or Zmynd10. In one embodiment, the one or more genes is 2, 3, 4, 5, 6, 7, 8, 9, 10, 15, 20, 25, 30, 35, 40, 45, 50, 55, 60, 65, 70, 75 or more genes, or any range or interval thereof. In one embodiment, the gene is Abcg1. In one embodiment, the gene is Abra. In one embodiment, the gene is Actn3. In one embodiment, the gene is Alas2. In one embodiment, the gene is Alox15. In one embodiment, the gene is Angpt14. In one embodiment, the gene is Apod. In one embodiment, the gene is Apold1. In one embodiment, the gene is Arc. In one embodiment, the gene is Arhgap24. In one embodiment, the gene is Arl4c. In one embodiment, the gene is Arnt1. In one embodiment, the gene is Arrdc2. In one embodiment, the gene is Asb5. In one embodiment, the gene is Atf3. In one embodiment, the gene is Bag2. In one embodiment, the gene is Bcl11a. In one embodiment, the gene is Bcl6. In one embodiment, the gene is Bdh1. In one embodiment, the gene is Bdnf. In one embodiment, the gene is Best3. In one embodiment, the gene is Bhlhe40. In one embodiment, the gene is Calhm1. In one embodiment, the gene is Calm13. In one embodiment, the gene is Car12. In one embodiment, the gene is Ccl5. In one embodiment, the gene is Cd74. In one embodiment, the gene is Cdc42se1. In one embodiment, the gene is Chac1. In one embodiment, the gene is Chst5. In one embodiment, the gene is Ciart. In one embodiment, the gene is Cidec. In one embodiment, the gene is Cish. In one embodiment, the gene is Cited4. In one embodiment, the gene is Ckap4. In one embodiment, the gene is Cldn2. In one embodiment, the gene is Clic6. In one embodiment, the gene is Cpt1a. In one embodiment, the gene is Csrnp1. In one embodiment, the gene is Cxcl13. In one embodiment, the gene is Dbp. In one embodiment, the gene is Dnajb5. In one embodiment, the gene is Dynl11. In one embodiment, the gene is Dyrk2. In one embodiment, the gene is Edn1. In one embodiment, the gene is Egr1. In one embodiment, the gene is Egr3. In one embodiment, the gene is Elfn1. In one embodiment, the gene is Emb. In one embodiment, the gene is Enah. In one embodiment, the gene is Fam107b. In one embodiment, the gene is Fam110a. In one embodiment, the gene is Fam134b. In one embodiment, the gene is Fam167a. In one embodiment, the gene is Fam46a. In one embodiment, the gene is Fasn. In one embodiment, the gene is Fgfr3. In one embodiment, the gene is Fhl2. In one embodiment, the gene is Fos. In one embodiment, the gene is Fosb. In one embodiment, the gene is Frk. In one embodiment, the gene is Fst. In one embodiment, the gene is Gdf15. In one embodiment, the gene is Gem. In one embodiment, the gene is Gngt1. In one embodiment, the gene is Gnl3. In one embodiment, the gene is Hba1. In one embodiment, the gene is Hba2. In one embodiment, the gene is Hbb. In one embodiment, the gene is Hbb-b1. In one embodiment, the gene is Hbegf. In one embodiment, the gene is Hmox1. In one embodiment, the gene is Hpd1. In one embodiment, the gene is Hspa1b. In one embodiment, the gene is Id4. In one embodiment, the gene is Il2rb. In one embodiment, the gene is Irs1. In one embodiment, the gene is Irs2. In one embodiment, the gene is Junb. In one embodiment, the gene is Jund. In one embodiment, the gene is Kbtbd8. In one embodiment, the gene is Kcnk5. In one embodiment, the gene is Kctd7. In one embodiment, the gene is Kirrel2. In one embodiment, the gene is Ky. In one embodiment, the gene is Lamc2. In one embodiment, the gene is Lipg. In one embodiment, the gene is LOC689064. In one embodiment, the gene is Lonrf3. In one embodiment, the gene is Lrrc38. In one embodiment, the gene is Lrrc52. In one embodiment, the gene is Lrrn2. In one embodiment, the gene is Lsr. In one embodiment, the gene is Maff. In one embodiment, the gene is Mchr1. In one embodiment, the gene is Mfrp. In one embodiment, the gene is Mllt1. In one embodiment, the gene is Mns1. In one embodiment, the gene is Mogat1. In one embodiment, the gene is Mphosph6. In one embodiment, the gene is Mpz. In one embodiment, the gene is Muc20. In one embodiment, the gene is Mybpc2. In one embodiment, the gene is Myf6. In one embodiment, the gene is Myh1. In one embodiment, the gene is Myh2. In one embodiment, the gene is Myh4. In one embodiment, the gene is Myocd. In one embodiment, the gene is Nedd9. In one embodiment, the gene is Nfil3. In one embodiment, the gene is Nkg7. In one embodiment, the gene is Nrld1. In one embodiment, the gene is Nr4a2. In one embodiment, the gene is Nr4a3. In one embodiment, the gene is Ntf4. In one embodiment, the gene is Nuak1. In one embodiment, the gene is Parp16. In one embodiment, the gene is Pdc. In one embodiment, the gene is Pde7a. In one embodiment, the gene is Pfkfb2. In one embodiment, the gene is Pfkfb3. In one embodiment, the gene is Pgam1. In one embodiment, the gene is Phlda1. In one embodiment, the gene is Pik3ip1. In one embodiment, the gene is Plk3. In one embodiment, the gene is Postn. In one embodiment, the gene is Ppargc1a. In one embodiment, the gene is Ppp1r14c. In one embodiment, the gene is Pragmin. In one embodiment, the gene is Prf1. In one embodiment, the gene is Ptpn14. In one embodiment, the gene is Pva1b. In one embodiment, the gene is Rab23. In one embodiment, the gene is Rab30. In one embodiment, the gene is Rbm20. In one embodiment, the gene is Rcan1. In one embodiment, the gene is Rel11. In one embodiment, the gene is Rfx1. In one embodiment, the gene is RGD1307461. In one embodiment, the gene is RGD1309676. In one embodiment, the gene is RGD1359290. In one embodiment, the gene is RGD1564428. In one embodiment, the gene is Rhpn2. In one embodiment, the gene is Rn45s. In one embodiment, the gene is Rnd1. In one embodiment, the gene is Rp1. In one embodiment, the gene is Rrad. In one embodiment, the gene is RT1-Ba. In one embodiment, the gene is RT1-Bb. In one embodiment, the gene is RT1-Da. In one embodiment, the gene is RT1-Db1. In one embodiment, the gene is Rtn4rl1. In one embodiment, the gene is Scd1. In one embodiment, the gene is Sdc4. In one embodiment, the gene is Sec1415. In one embodiment, the gene is Siglec5. In one embodiment, the gene is Sik1. In one embodiment, the gene is Slc18a2. In one embodiment, the gene is Slc2a5. In one embodiment, the gene is Slc30a4. In one embodiment, the gene is Slc4a1. In one embodiment, the gene is Slc4a5. In one embodiment, the gene is Slpi. In one embodiment, the gene is Smad7. In one embodiment, the gene is Snhg4. In one embodiment, the gene is Spag8. In one embodiment, the gene is Stc1. In one embodiment, the gene is Sv2c. In one embodiment, the gene is Terf2ip. In one embodiment, the gene is Thrsp. In one embodiment, the gene is Tmc8. In one embodiment, the gene is Tmem171. In one embodiment, the gene is Tmx4. In one embodiment, the gene is Tnfrsfl2a. In one embodiment, the gene is Tnni2. In one embodiment, the gene is Ttc30b. In one embodiment, the gene is Txnip. In one embodiment, the gene is Ucp3. In one embodiment, the gene is Unc5b. In one embodiment, the gene is Zfp112. In one embodiment, the gene is Zfp13. In one embodiment, the gene is Zfp385b. In one embodiment, the gene is Zfp474. In one embodiment, the gene is Zfyve28. In one embodiment, the gene is Zic1. In one embodiment, the gene is Zmynd10. In some embodiments, the gene that is modulated is upregulated. In other embodiments, the gene that is modulated is downregulated. In certain embodiments, the modulation of the gene is as compared to the same subject prior to administration of a population of stem cells (e.g., PDSC). In certain embodiments, the modulation of the gene is as compared to a control subject that has not been administered a population of stem cells (e.g., PDSC). In certain embodiments, the modulation of the gene is as compared to a younger subject. In certain embodiments, the modulation of the gene is as compared to a older subject.

In one embodiment, the gene is Abcg1, and the gene expression is increased. In one embodiment, the gene is Abra, and the gene expression is increased. In one embodiment, the gene is Actn3, and the gene expression is decreased. In one embodiment, the gene is Actn3, and the gene expression is increased. In one embodiment, the gene is Alas2, and the gene expression is decreased. In one embodiment, the gene is Alox5, and the gene expression is decreased. In one embodiment, the gene is Alox5, and the gene expression is increased. In one embodiment, the gene is Angpt14, and the gene expression is decreased. In one embodiment, the gene is Apod, and the gene expression is decreased. In one embodiment, the gene is Apold1, and the gene expression is decreased. In one embodiment, the gene is Arc, and the gene expression is decreased. In one embodiment, the gene is Arhgap24, and the gene expression is increased. In one embodiment, the gene is Arl4c, and the gene expression is increased. In one embodiment, the gene is Amt1, and the gene expression is increased. In one embodiment, the gene is Arrdc2, and the gene expression is decreased. In one embodiment, the gene is Asb5, and the gene expression is increased. In one embodiment, the gene is Atf3, and the gene expression is increased. In one embodiment, the gene is Bag2, and the gene expression is increased. In one embodiment, the gene is Bcl11a, and the gene expression is increased. In one embodiment, the gene is Bcl6, and the gene expression is increased. In one embodiment, the gene is Bdh1, and the gene expression is increased. In one embodiment, the gene is Bdnf, and the gene expression is increased. In one embodiment, the gene is Best3, and the gene expression is increased. In one embodiment, the gene is Bhlhe40, and the gene expression is decreased. In one embodiment, the gene is Calhm1, and the gene expression is increased. In one embodiment, the gene is Calm13, and the gene expression is increased. In one embodiment, the gene is Car12, and the gene expression is increased. In one embodiment, the gene is Ccl5, and the gene expression is decreased. In one embodiment, the gene is Cd74, and the gene expression is increased. In one embodiment, the gene is Cdc42se1, and the gene expression is increased. In one embodiment, the gene is Chac1, and the gene expression is decreased as compared to a control subject (e.g., a subject that has not been administered a population of stem cells (e.g., PDAC)), and the gene expression is decreased. In one embodiment, the gene is Chst5, and the gene expression is increased. In one embodiment, the gene is Ciart, and the gene expression is decreased. In one embodiment, the gene is Cidec, and the gene expression is increased. In one embodiment, the gene is Cish, and the gene expression is decreased. In one embodiment, the gene is Cited4, and the gene expression is decreased. In one embodiment, the gene is Ckap4, and the gene expression is increased. In one embodiment, the gene is Cldn2, and the gene expression is increased. In one embodiment, the gene is Clic6, and the gene expression is increased. In one embodiment, the gene is Cpt1a, and the gene expression is decreased. In one embodiment, the gene is Csrnp1, and the gene expression is increased. In one embodiment, the gene is Cxcl13, and the gene expression is decreased. In one embodiment, the gene is Cxcl13, and the gene expression is increased. In one embodiment, the gene is Dbp, and the gene expression is decreased. In one embodiment, the gene is Dnajb5, and the gene expression is increased. In one embodiment, the gene is Dynl11, and the gene expression is increased. In one embodiment, the gene is Dyrk2, and the gene expression is increased. In one embodiment, the gene is Edn1, and the gene expression is increased. In one embodiment, the gene is Egr1, and the gene expression is decreased. In one embodiment, the gene is Egr3, and the gene expression is decreased. In one embodiment, the gene is Elfn1, and the gene expression is increased. In one embodiment, the gene is Emb, and the gene expression is increased. In one embodiment, the gene is Enah, and the gene expression is increased. In one embodiment, the gene is Fam107b, and the gene expression is increased. In one embodiment, the gene is Fam110a, and the gene expression is increased. In one embodiment, the gene is Fam134b, and the gene expression is increased. In one embodiment, the gene is Fam167a, and the gene expression is increased. In one embodiment, the gene is Fam46a, and the gene expression is increased. In one embodiment, the gene is Fasn, and the gene expression is decreased. In one embodiment, the gene is Fgfr3, and the gene expression is increased. In one embodiment, the gene is Fhl2, and the gene expression is increased. In one embodiment, the gene is Fos, and the gene expression is increased. In one embodiment, the gene is Fosb, and the gene expression is decreased. In one embodiment, the gene is Fosb, and the gene expression is increased. In one embodiment, the gene is Frk, and the gene expression is increased. In one embodiment, the gene is Fst, and the gene expression is increased. In one embodiment, the gene is Gdf15, and the gene expression is increased. In one embodiment, the gene is Gem, and the gene expression is increased. In one embodiment, the gene is Gngt1, and the gene expression is increased. In one embodiment, the gene is Gnl3, and the gene expression is increased. In one embodiment, the gene is Hba1, and the gene expression is decreased. In one embodiment, the gene is Hba2, and the gene expression is decreased. In one embodiment, the gene is Hbb, and the gene expression is decreased. In one embodiment, the gene is Hbb-b1, and the gene expression is decreased. In one embodiment, the gene is Hbegf, and the gene expression is increased. In one embodiment, the gene is Hmox1, and the gene expression is increased. In one embodiment, the gene is Hpd1, and the gene expression is decreased. In one embodiment, the gene is Hspa1b, and the gene expression is increased. In one embodiment, the gene is Id4, and the gene expression is increased. In one embodiment, the gene is Il2rb, and the gene expression is decreased. In one embodiment, the gene is Irs1, and the gene expression is increased. In one embodiment, the gene is Irs2, and the gene expression is increased. In one embodiment, the gene is Junb, and the gene expression is decreased. In one embodiment, the gene is Jund, and the gene expression is increased. In one embodiment, the gene is Kbtbd8, and the gene expression is increased. In one embodiment, the gene is Kcnk5, and the gene expression is increased. In one embodiment, the gene is Kctd7, and the gene expression is decreased. In one embodiment, the gene is Kirrel2, and the gene expression is increased. In one embodiment, the gene is Ky, and the gene expression is decreased. In one embodiment, the gene is Lamc2, and the gene expression is increased. In one embodiment, the gene is Lipg, and the gene expression is increased. In one embodiment, the gene is LOC689064, and the gene expression is decreased. In one embodiment, the gene is Lonrf3, and the gene expression is increased. In one embodiment, the gene is Lrrc38, and the gene expression is increased. In one embodiment, the gene is Lrrc52, and the gene expression is increased. In one embodiment, the gene is Lrrn2, and the gene expression is decreased. In one embodiment, the gene is Lsr, and the gene expression is increased. In one embodiment, the gene is Maff, and the gene expression is increased. In one embodiment, the gene is Mchr1, and the gene expression is decreased. In one embodiment, the gene is Mfrp, and the gene expression is increased. In one embodiment, the gene is Mllt11, and the gene expression is increased. In one embodiment, the gene is Mns1, and the gene expression is increased. In one embodiment, the gene is Mogat1, and the gene expression is increased. In one embodiment, the gene is Mphosph6, and the gene expression is increased. In one embodiment, the gene is Mpz, and the gene expression is decreased. In one embodiment, the gene is Muc20, and the gene expression is increased. In one embodiment, the gene is Mybpc2, and the gene expression is decreased. In one embodiment, the gene is Myf6, and the gene expression is increased. In one embodiment, the gene is Myh1, and the gene expression is decreased. In one embodiment, the gene is Myh2, and the gene expression is decreased. In one embodiment, the gene is Myh4, and the gene expression is increased. In one embodiment, the gene is Myocd, and the gene expression is increased. In one embodiment, the gene is Nedd9, and the gene expression is increased. In one embodiment, the gene is Nfil3, and the gene expression is increased. In one embodiment, the gene is Nkg7, and the gene expression is decreased. In one embodiment, the gene is Nrld1, and the gene expression is decreased. In one embodiment, the gene is Nr4a2, and the gene expression is decreased. In one embodiment, the gene is Nr4a2, and the gene expression is increased. In one embodiment, the gene is Nr4a3, and the gene expression is increased. In one embodiment, the gene is Ntf4, and the gene expression is decreased. In one embodiment, the gene is Nuak1, and the gene expression is increased. In one embodiment, the gene is Parp16, and the gene expression is decreased. In one embodiment, the gene is Pdc, and the gene expression is increased. In one embodiment, the gene is Pde7a, and the gene expression is increased. In one embodiment, the gene is Pfkfb2, and the gene expression is increased. In one embodiment, the gene is Pfkfb3, and the gene expression is decreased. In one embodiment, the gene is Pgam1, and the gene expression is increased. In one embodiment, the gene is Phlda1, and the gene expression is increased. In one embodiment, the gene is Pik3ip1, and the gene expression is decreased. In one embodiment, the gene is Plk3, and the gene expression is decreased. In one embodiment, the gene is Postn, and the gene expression is increased. In one embodiment, the gene is Ppargc1a, and the gene expression is increased. In one embodiment, the gene is Ppp1r14c, and the gene expression is increased. In one embodiment, the gene is Pragmin, and the gene expression is increased. In one embodiment, the gene is Prf1, and the gene expression is decreased. In one embodiment, the gene is Ptpn14, and the gene expression is increased. In one embodiment, the gene is Pva1b, and the gene expression is decreased. In one embodiment, the gene is Pva1b, and the gene expression is increased. In one embodiment, the gene is Rab23, and the gene expression is increased. In one embodiment, the gene is Rab30, and the gene expression is increased. In one embodiment, the gene is Rbm20, and the gene expression is increased. In one embodiment, the gene is Rcan1, and the gene expression is increased. In one embodiment, the gene is Rel11, and the gene expression is increased. In one embodiment, the gene is Rfx1, and the gene expression is increased. In one embodiment, the gene is RGD1307461, and the gene expression is decreased. In one embodiment, the gene is RGD1309676, and the gene expression is increased. In one embodiment, the gene is RGD1359290, and the gene expression is increased. In one embodiment, the gene is RGD1564428, and the gene expression is increased. In one embodiment, the gene is Rhpn2, and the gene expression is increased. In one embodiment, the gene is Rn45s, and the gene expression is decreased. In one embodiment, the gene is Rnd1, and the gene expression is increased. In one embodiment, the gene is Rp1, and the gene expression is increased. In one embodiment, the gene is Rrad, and the gene expression is increased. In one embodiment, the gene is RT1-Ba, and the gene expression is increased. In one embodiment, the gene is RT1-Bb, and the gene expression is increased as compared to a control subject (e.g., a subject that has not been administered a population of stem cells (e.g., PDAC)) In one embodiment, the gene is RT1-Da, and the gene expression is increased. In one embodiment, the gene is RT1-Db1, and the gene expression is increased. In one embodiment, the gene is Rtn4rl1, and the gene expression is decreased. In one embodiment, the gene is Scd1, and the gene expression is decreased. In one embodiment, the gene is Scd1, and the gene expression is increased. In one embodiment, the gene is Sdc4, and the gene expression is increased. In one embodiment, the gene is Sec1415, and the gene expression is decreased. In one embodiment, the gene is Siglec5, and the gene expression is decreased. In one embodiment, the gene is Sik1, and the gene expression is increased. In one embodiment, the gene is Slc18a2, and the gene expression is increased. In one embodiment, the gene is Slc2a5, and the gene expression is decreased. In one embodiment, the gene is Slc30a4, and the gene expression is increased. In one embodiment, the gene is Slc4a1, and the gene expression is decreased. In one embodiment, the gene is Slc4a1, and the gene expression is increased. In one embodiment, the gene is Slc4a5, and the gene expression is increased. In one embodiment, the gene is Slpi, and the gene expression is decreased. In one embodiment, the gene is Smad7, and the gene expression is increased. In one embodiment, the gene is Snhg4, and the gene expression is decreased. In one embodiment, the gene is Spag8, and the gene expression is decreased. In one embodiment, the gene is Stc1, and the gene expression is increased. In one embodiment, the gene is Sv2c, and the gene expression is increased. In one embodiment, the gene is Terf2ip, and the gene expression is increased. In one embodiment, the gene is Thrsp, and the gene expression is decreased. In one embodiment, the gene is Tmc8, and the gene expression is decreased. In one embodiment, the gene is Tmem171, and the gene expression is increased. In one embodiment, the gene is Tmx4, and the gene expression is increased. In one embodiment, the gene is Tnfrsfl2a, and the gene expression is increased. In one embodiment, the gene is Tnni2, and the gene expression is decreased. In one embodiment, the gene is Ttc30b, and the gene expression is decreased. In one embodiment, the gene is Txnip, and the gene expression is decreased. In one embodiment, the gene is Ucp3, and the gene expression is decreased. In one embodiment, the gene is Unc5b, and the gene expression is increased. In one embodiment, the gene is Zfp112, and the gene expression is decreased. In one embodiment, the gene is Zfp13, and the gene expression is decreased. In one embodiment, the gene is Zfp385b, and the gene expression is increased. In one embodiment, the gene is Zfp474, and the gene expression is increased. In one embodiment, the gene is Zfyve28, and the gene expression is decreased. In one embodiment, the gene is Zic, and the gene expression is increased. In one embodiment, the gene is Zmynd10, and the gene expression is decreased. In certain embodiments, the increased gene expression is as compared to a control subject. In certain embodiments, the decreased gene expression is as compared to a control subject. In a specific embodiment, the control subject is a subject that has not been administered a population of stem cells (e.g., PDAC)).

In some embodiments, a protein expression is modulated in a subject following administration of the stem cells (e.g., PDSC). In one embodiment, the protein is encoded by a gene provided in any one of Table 5-9. In one embodiment, the protein is encoded by a gene provided in Table 5. In one embodiment, the protein is encoded by a gene provided in Table 6. In one embodiment, the protein is encoded by a gene provided in Table 7. In one embodiment, t the protein is encoded by a gene provided in Table 8. In one embodiment, t the protein is encoded by a gene provided in Table 9. In one embodiment, the protein is one or more proteins independently selected from the group consisting of Abcg1, Abra, Actn3, Alas2, Alox15, Angpt14, Apod, Apold1, Arc, Arhgap24, Arl4c, Amt1, Arrdc2, Asb5, Atf3, Bag2, Bcl11a, Bcl6, Bdh1, Bdnf, Best3, Bhlhe40, Calhm1, Calm13, Car12, Ccl5, Cd74, Cdc42se1, Chac1, Chst5, Ciart, Cidec, Cish, Cited4, Ckap4, Cldn2, Clic6, Cpt1a, Csrnp1, Cxcl13, Dbp, Dnajb5, Dynl11, Dyrk2, Edn1, Egr1, Egr3, Elfn1, Emb, Enah, Fam107b, Fam110a, Fam134b, Fam167a, Fam46a, Fasn, Fgfr3, Fhl2, Fos, Fosb, Frk, Fst, Gdf15, Gem, Gngt1, Gnl3, Hba1, Hba2, Hbb, Hbb-b1, Hbegf, Hmox1, Hpd1, Hspa1b, Id4, Il12rb, Irs1, Irs2, Junb, Jund, Kbtbd8, Kcnk5, Kctd7, Kirrel2, Ky, Lamc2, Lipg, LOC689064, Lonrf3, Lrrc38, Lrrc52, Lrrn2, Lsr, Maff, Mchr1, Mfrp, Mllt11, Mns1, Mogat1, Mphosph6, Mpz, Muc20, Mybpc2, Myf6, Myh1, Myh2, Myh4, Myocd, Nedd9, Nfil3, Nkg7, Nrld1, Nr4a2, Nr4a3, Ntf4, Nuak1, Parp16, Pdc, Pde7a, Pfkfb2, Pfkfb3, Pgam1, Phlda1, Pik3ip1, Plk3, Postn, Ppargc1a, Ppp1r14c, Pragmin, Prf1, Ptpn14, Pva1b, Rab23, Rab30, Rbm20, Rcan1, Rel11, Rfx1, RGD1307461, RGD1309676, RGD1359290, RGD1564428, Rhpn2, Rn45s, Rnd1, Rp1, Rrad, RT1-Ba, RT1-Bb, RT1-Da, RT1-Db1, Rtn4rl1, Scd1, Sdc4, Sec1415, Siglec5, Sik1, Slc18a2, Slc2a5, Slc30a4, Slc4a1, Slc4a5, Slpi, Smad7, Snhg4, Spag8, Stc1, Sv2c, Terf2ip, Thrsp, Tmc8, Tmem171, Tmx4, Tnfrsfl2a, Tnni2, Ttc30b, Txnip, Ucp3, Unc5b, Zfp112, Zfp13, Zfp385b and Zfp474, Zfyve28, Zic1 or Zmynd10. In one embodiment, the one or more proteins is 2, 3, 4, 5, 6, 7, 8, 9, 10, 15, 20, 25, 30, 35, 40, 45, 50, 55, 60, 65, 70, 75 or more proteins, or any range or interval thereof. In one embodiment, the protein is Abcg1. In one embodiment, the protein is Abra. In one embodiment, the protein is Actn3. In one embodiment, the protein is Alas2. In one embodiment, the protein is Alox15. In one embodiment, the protein is Angpt14. In one embodiment, the protein is Apod. In one embodiment, the protein is Apold1. In one embodiment, the protein is Arc. In one embodiment, the protein is Arhgap24. In one embodiment, the protein is Arl4c. In one embodiment, the protein is Amt1. In one embodiment, the protein is Arrdc2. In one embodiment, the protein is Asb5. In one embodiment, the protein is Atf3. In one embodiment, the protein is Bag2. In one embodiment, the protein is Bcl11a. In one embodiment, the protein is Bcl6. In one embodiment, the protein is Bdh1. In one embodiment, the protein is Bdnf. In one embodiment, the protein is Best3. In one embodiment, the protein is Bhlhe40. In one embodiment, the protein is Calhm1. In one embodiment, the protein is Calm13. In one embodiment, the protein is Car12. In one embodiment, the protein is Ccl5. In one embodiment, the protein is Cd74. In one embodiment, the protein is Cdc42se1. In one embodiment, the protein is Chac1. In one embodiment, the protein is Chst5. In one embodiment, the protein is Ciart. In one embodiment, the protein is Cidec. In one embodiment, the protein is Cish. In one embodiment, the protein is Cited4. In one embodiment, the protein is Ckap4. In one embodiment, the protein is Cldn2. In one embodiment, the protein is Clic6. In one embodiment, the protein is Cpt1a. In one embodiment, the protein is Csrnp1. In one embodiment, the protein is Cxcl13. In one embodiment, the protein is Dbp. In one embodiment, the protein is Dnajb5. In one embodiment, the protein is Dynl11. In one embodiment, the protein is Dyrk2. In one embodiment, the protein is Edn1. In one embodiment, the protein is Egr1. In one embodiment, the protein is Egr3. In one embodiment, the protein is Elfn1. In one embodiment, the protein is Emb. In one embodiment, the protein is Enah. In one embodiment, the protein is Fam107b. In one embodiment, the protein is Fam110a. In one embodiment, the protein is Fam134b. In one embodiment, the protein is Fam167a. In one embodiment, the protein is Fam46a. In one embodiment, the protein is Fasn. In one embodiment, the protein is Fgfr3. In one embodiment, the protein is Fhl2. In one embodiment, the protein is Fos. In one embodiment, the protein is Fosb. In one embodiment, the protein is Frk. In one embodiment, the protein is Fst. In one embodiment, the protein is Gdf15. In one embodiment, the protein is Gem. In one embodiment, the protein is Gngt1. In one embodiment, the protein is Gnl3. In one embodiment, the protein is Hba1. In one embodiment, the protein is Hba2. In one embodiment, the protein is Hbb. In one embodiment, the protein is Hbb-b1. In one embodiment, the protein is Hbegf. In one embodiment, the protein is Hmox1. In one embodiment, the protein is Hpd1. In one embodiment, the protein is Hspa1b. In one embodiment, the protein is Id4. In one embodiment, the protein is Il2rb. In one embodiment, the protein is Irs1. In one embodiment, the protein is Irs2. In one embodiment, the protein is Junb. In one embodiment, the protein is Jund. In one embodiment, the protein is Kbtbd8. In one embodiment, the protein is Kcnk5. In one embodiment, the protein is Kctd7. In one embodiment, the protein is Kirrel2. In one embodiment, the protein is Ky. In one embodiment, the protein is Lamc2. In one embodiment, the protein is Lipg. In one embodiment, the protein is LOC689064. In one embodiment, the protein is Lonrf3. In one embodiment, the protein is Lrrc38. In one embodiment, the protein is Lrrc52. In one embodiment, the protein is Lrrn2. In one embodiment, the protein is Lsr. In one embodiment, the protein is Maff. In one embodiment, the protein is Mchr1. In one embodiment, the protein is Mfrp. In one embodiment, the protein is Mllt11. In one embodiment, the protein is Mns1. In one embodiment, the protein is Mogat1. In one embodiment, the protein is Mphosph6. In one embodiment, the protein is Mpz. In one embodiment, the protein is Muc20. In one embodiment, the protein is Mybpc2. In one embodiment, the protein is Myf6. In one embodiment, the protein is Myh1. In one embodiment, the protein is Myh2. In one embodiment, the protein is Myh4. In one embodiment, the protein is Myocd. In one embodiment, the protein is Nedd9. In one embodiment, the protein is Nfil3. In one embodiment, the protein is Nkg7. In one embodiment, the protein is Nrld1. In one embodiment, the protein is Nr4a2. In one embodiment, the protein is Nr4a3. In one embodiment, the protein is Ntf4. In one embodiment, the protein is Nuak1. In one embodiment, the protein is Parp16. In one embodiment, the protein is Pdc. In one embodiment, the protein is Pde7a. In one embodiment, the protein is Pfkfb2. In one embodiment, the protein is Pfkfb3. In one embodiment, the protein is Pgam1. In one embodiment, the protein is Phlda1. In one embodiment, the protein is Pik3ip1. In one embodiment, the protein is Plk3. In one embodiment, the protein is Postn. In one embodiment, the protein is Ppargc1a. In one embodiment, the protein is Ppp1r14c. In one embodiment, the protein is Pragmin. In one embodiment, the protein is Prf1. In one embodiment, the protein is Ptpn14. In one embodiment, the protein is Pva1b. In one embodiment, the protein is Rab23. In one embodiment, the protein is Rab30. In one embodiment, the protein is Rbm20. In one embodiment, the protein is Rcan1. In one embodiment, the protein is Rel11. In one embodiment, the protein is Rfx1. In one embodiment, the protein is RGD1307461. In one embodiment, the protein is RGD1309676. In one embodiment, the protein is RGD1359290. In one embodiment, the protein is RGD1564428. In one embodiment, the protein is Rhpn2. In one embodiment, the protein is Rn45s. In one embodiment, the protein is Rnd1. In one embodiment, the protein is Rp1. In one embodiment, the protein is Rrad. In one embodiment, the protein is RT1-Ba. In one embodiment, the protein is RT1-Bb. In one embodiment, the protein is RT1-Da. In one embodiment, the protein is RT1-Db1. In one embodiment, the protein is Rtn4rl1. In one embodiment, the protein is Scd1. In one embodiment, the protein is Sdc4. In one embodiment, the protein is Sec1415. In one embodiment, the protein is Siglec5. In one embodiment, the protein is Sik1. In one embodiment, the protein is Slc18a2. In one embodiment, the protein is Slc2a5. In one embodiment, the protein is Slc30a4. In one embodiment, the protein is Slc4a1. In one embodiment, the protein is Slc4a5. In one embodiment, the protein is Slpi. In one embodiment, the protein is Smad7. In one embodiment, the protein is Snhg4. In one embodiment, the protein is Spag8. In one embodiment, the protein is Stc1. In one embodiment, the protein is Sv2c. In one embodiment, the protein is Terf2ip. In one embodiment, the protein is Thrsp. In one embodiment, the protein is Tmc8. In one embodiment, the protein is Tmem171. In one embodiment, the protein is Tmx4. In one embodiment, the protein is Tnfrsfl2a. In one embodiment, the protein is Tnni2. In one embodiment, the protein is Ttc30b. In one embodiment, the protein is Txnip. In one embodiment, the protein is Ucp3. In one embodiment, the protein is Unc5b. In one embodiment, the protein is Zfp112. In one embodiment, the protein is Zfp13. In one embodiment, the protein is Zfp385b. In one embodiment, the protein is Zfp474. In one embodiment, the protein is Zfyve28. In one embodiment, the protein is Zic1. In one embodiment, the protein is Zmynd10. In some embodiments, the protein that is modulated is upregulated. In other embodiments, the protein that is modulated is downregulated. In certain embodiments, the modulation of the protein is as compared to the same subject prior to administration of a population of stem cells (e.g., PDSC). In certain embodiments, the modulation of the protein is as compared to a control subject that has not been administered a population of stem cells (e.g., PDSC). In certain embodiments, the modulation of the protein is as compared to a younger subject. In certain embodiments, the modulation of the protein is as compared to a older subject.

In one embodiment, the protein is Abcg1, and the protein expression is increased. In one embodiment, the protein is Abra, and the protein expression is increased. In one embodiment, the protein is Actn3, and the protein expression is decreased. In one embodiment, the protein is Actn3, and the protein expression is increased. In one embodiment, the protein is Alas2, and the protein expression is decreased. In one embodiment, the protein is Alox5, and the protein expression is decreased. In one embodiment, the protein is Alox5, and the protein expression is increased. In one embodiment, the protein is Angpt14, and the protein expression is decreased. In one embodiment, the protein is Apod, and the protein expression is decreased. In one embodiment, the protein is Apold1, and the protein expression is decreased. In one embodiment, the protein is Arc, and the protein expression is decreased. In one embodiment, the protein is Arhgap24, and the protein expression is increased. In one embodiment, the protein is Arl4c, and the protein expression is increased. In one embodiment, the protein is Amt1, and the protein expression is increased. In one embodiment, the protein is Arrdc2, and the protein expression is decreased. In one embodiment, the protein is Asb5, and the protein expression is increased. In one embodiment, the protein is Atf3, and the protein expression is increased. In one embodiment, the protein is Bag2, and the protein expression is increased. In one embodiment, the protein is Bcl11a, and the protein expression is increased. In one embodiment, the protein is Bcl6, and the protein expression is increased. In one embodiment, the protein is Bdh1, and the protein expression is increased. In one embodiment, the protein is Bdnf, and the protein expression is increased. In one embodiment, the protein is Best3, and the protein expression is increased. In one embodiment, the protein is Bhlhe40, and the protein expression is decreased. In one embodiment, the protein is Calhm1, and the protein expression is increased. In one embodiment, the protein is Calm13, and the protein expression is increased. In one embodiment, the protein is Car12, and the protein expression is increased. In one embodiment, the protein is Ccl5, and the protein expression is decreased. In one embodiment, the protein is Cd74, and the protein expression is increased. In one embodiment, the protein is Cdc42se1, and the protein expression is increased. In one embodiment, the protein is Chac1, and the protein expression is decreased as compared to a control subject (e.g., a subject that has not been administered a population of stem cells (e.g., PDAC)), and the protein expression is decreased. In one embodiment, the protein is Chst5, and the protein expression is increased. In one embodiment, the protein is Ciart, and the protein expression is decreased. In one embodiment, the protein is Cidec, and the protein expression is increased. In one embodiment, the protein is Cish, and the protein expression is decreased. In one embodiment, the protein is Cited4, and the protein expression is decreased. In one embodiment, the protein is Ckap4, and the protein expression is increased. In one embodiment, the protein is Cldn2, and the protein expression is increased. In one embodiment, the protein is Clic6, and the protein expression is increased. In one embodiment, the protein is Cpt1a, and the protein expression is decreased. In one embodiment, the protein is Csrnp1, and the protein expression is increased. In one embodiment, the protein is Cxcl13, and the protein expression is decreased. In one embodiment, the protein is Cxcl13, and the protein expression is increased. In one embodiment, the protein is Dbp, and the protein expression is decreased. In one embodiment, the protein is Dnajb5, and the protein expression is increased. In one embodiment, the protein is Dynl11, and the protein expression is increased. In one embodiment, the protein is Dyrk2, and the protein expression is increased. In one embodiment, the protein is Edn1, and the protein expression is increased. In one embodiment, the protein is Egr1, and the protein expression is decreased. In one embodiment, the protein is Egr3, and the protein expression is decreased. In one embodiment, the protein is Elfn1, and the protein expression is increased. In one embodiment, the protein is Emb, and the protein expression is increased. In one embodiment, the protein is Enah, and the protein expression is increased. In one embodiment, the protein is Fam107b, and the protein expression is increased. In one embodiment, the protein is Fam110a, and the protein expression is increased. In one embodiment, the protein is Fam134b, and the protein expression is increased. In one embodiment, the protein is Fam167a, and the protein expression is increased. In one embodiment, the protein is Fam46a, and the protein expression is increased. In one embodiment, the protein is Fasn, and the protein expression is decreased. In one embodiment, the protein is Fgfr3, and the protein expression is increased. In one embodiment, the protein is Fhl2, and the protein expression is increased. In one embodiment, the protein is Fos, and the protein expression is increased. In one embodiment, the protein is Fosb, and the protein expression is decreased. In one embodiment, the protein is Fosb, and the protein expression is increased. In one embodiment, the protein is Frk, and the protein expression is increased. In one embodiment, the protein is Fst, and the protein expression is increased. In one embodiment, the protein is Gdf15, and the protein expression is increased. In one embodiment, the protein is Gem, and the protein expression is increased. In one embodiment, the protein is Gngt1, and the protein expression is increased. In one embodiment, the protein is Gnl3, and the protein expression is increased. In one embodiment, the protein is Hba1, and the protein expression is decreased. In one embodiment, the protein is Hba2, and the protein expression is decreased. In one embodiment, the protein is Hbb, and the protein expression is decreased. In one embodiment, the protein is Hbb-b1, and the protein expression is decreased. In one embodiment, the protein is Hbegf, and the protein expression is increased. In one embodiment, the protein is Hmox1, and the protein expression is increased. In one embodiment, the protein is Hpd1, and the protein expression is decreased. In one embodiment, the protein is Hspa1b, and the protein expression is increased. In one embodiment, the protein is Id4, and the protein expression is increased. In one embodiment, the protein is Il2rb, and the protein expression is decreased. In one embodiment, the protein is Irs1, and the protein expression is increased. In one embodiment, the protein is Irs2, and the protein expression is increased. In one embodiment, the protein is Junb, and the protein expression is decreased. In one embodiment, the protein is Jund, and the protein expression is increased. In one embodiment, the protein is Kbtbd8, and the protein expression is increased. In one embodiment, the protein is Kcnk5, and the protein expression is increased. In one embodiment, the protein is Kctd7, and the protein expression is decreased. In one embodiment, the protein is Kirrel2, and the protein expression is increased. In one embodiment, the protein is Ky, and the protein expression is decreased. In one embodiment, the protein is Lamc2, and the protein expression is increased. In one embodiment, the protein is Lipg, and the protein expression is increased. In one embodiment, the protein is LOC689064, and the protein expression is decreased. In one embodiment, the protein is Lonrf3, and the protein expression is increased. In one embodiment, the protein is Lrrc38, and the protein expression is increased. In one embodiment, the protein is Lrrc52, and the protein expression is increased. In one embodiment, the protein is Lrrn2, and the protein expression is decreased. In one embodiment, the protein is Lsr, and the protein expression is increased. In one embodiment, the protein is Maff, and the protein expression is increased. In one embodiment, the protein is Mchr1, and the protein expression is decreased. In one embodiment, the protein is Mfrp, and the protein expression is increased. In one embodiment, the protein is Mllt11, and the protein expression is increased. In one embodiment, the protein is Mns1, and the protein expression is increased. In one embodiment, the protein is Mogat1, and the protein expression is increased. In one embodiment, the protein is Mphosph6, and the protein expression is increased. In one embodiment, the protein is Mpz, and the protein expression is decreased. In one embodiment, the protein is Muc20, and the protein expression is increased. In one embodiment, the protein is Mybpc2, and the protein expression is decreased. In one embodiment, the protein is Myf6, and the protein expression is increased. In one embodiment, the protein is Myh1, and the protein expression is decreased. In one embodiment, the protein is Myh2, and the protein expression is decreased. In one embodiment, the protein is Myh4, and the protein expression is increased. In one embodiment, the protein is Myocd, and the protein expression is increased. In one embodiment, the protein is Nedd9, and the protein expression is increased. In one embodiment, the protein is Nfil3, and the protein expression is increased. In one embodiment, the protein is Nkg7, and the protein expression is decreased. In one embodiment, the protein is Nrld1, and the protein expression is decreased. In one embodiment, the protein is Nr4a2, and the protein expression is decreased. In one embodiment, the protein is Nr4a2, and the protein expression is increased. In one embodiment, the protein is Nr4a3, and the protein expression is increased. In one embodiment, the protein is Ntf4, and the protein expression is decreased. In one embodiment, the protein is Nuak1, and the protein expression is increased. In one embodiment, the protein is Parp16, and the protein expression is decreased. In one embodiment, the protein is Pdc, and the protein expression is increased. In one embodiment, the protein is Pde7a, and the protein expression is increased. In one embodiment, the protein is Pfkfb2, and the protein expression is increased. In one embodiment, the protein is Pfkfb3, and the protein expression is decreased. In one embodiment, the protein is Pgam1, and the protein expression is increased. In one embodiment, the protein is Phlda1, and the protein expression is increased. In one embodiment, the protein is Pik3ip1, and the protein expression is decreased. In one embodiment, the protein is Plk3, and the protein expression is decreased. In one embodiment, the protein is Postn, and the protein expression is increased. In one embodiment, the protein is Ppargc1a, and the protein expression is increased. In one embodiment, the protein is Ppp1r14c, and the protein expression is increased. In one embodiment, the protein is Pragmin, and the protein expression is increased. In one embodiment, the protein is Prf1, and the protein expression is decreased. In one embodiment, the protein is Ptpn14, and the protein expression is increased. In one embodiment, the protein is Pva1b, and the protein expression is decreased. In one embodiment, the protein is Pva1b, and the protein expression is increased. In one embodiment, the protein is Rab23, and the protein expression is increased. In one embodiment, the protein is Rab30, and the protein expression is increased. In one embodiment, the protein is Rbm20, and the protein expression is increased. In one embodiment, the protein is Rcan1, and the protein expression is increased. In one embodiment, the protein is Rel11, and the protein expression is increased. In one embodiment, the protein is Rfx1, and the protein expression is increased. In one embodiment, the protein is RGD1307461, and the protein expression is decreased. In one embodiment, the protein is RGD1309676, and the protein expression is increased. In one embodiment, the protein is RGD1359290, and the protein expression is increased. In one embodiment, the protein is RGD1564428, and the protein expression is increased. In one embodiment, the protein is Rhpn2, and the protein expression is increased. In one embodiment, the protein is Rn45s, and the protein expression is decreased. In one embodiment, the protein is Rnd1, and the protein expression is increased. In one embodiment, the protein is Rp1, and the protein expression is increased. In one embodiment, the protein is Rrad, and the protein expression is increased. In one embodiment, the protein is RT1-Ba, and the protein expression is increased. In one embodiment, the protein is RT1-Bb, and the protein expression is increased as compared to a control subject (e.g., a subject that has not been administered a population of stem cells (e.g., PDAC)) In one embodiment, the protein is RT1-Da, and the protein expression is increased. In one embodiment, the protein is RT1-Db1, and the protein expression is increased. In one embodiment, the protein is Rtn4rl1, and the protein expression is decreased. In one embodiment, the protein is Scd1, and the protein expression is decreased. In one embodiment, the protein is Scd1, and the protein expression is increased. In one embodiment, the protein is Sdc4, and the protein expression is increased. In one embodiment, the protein is Sec1415, and the protein expression is decreased. In one embodiment, the protein is Siglec5, and the protein expression is decreased. In one embodiment, the protein is Sik1, and the protein expression is increased. In one embodiment, the protein is Slc18a2, and the protein expression is increased. In one embodiment, the protein is Slc2a5, and the protein expression is decreased. In one embodiment, the protein is Slc30a4, and the protein expression is increased. In one embodiment, the protein is Slc4a1, and the protein expression is decreased. In one embodiment, the protein is Slc4a1, and the protein expression is increased. In one embodiment, the protein is Slc4a5, and the protein expression is increased. In one embodiment, the protein is Slpi, and the protein expression is decreased. In one embodiment, the protein is Smad7, and the protein expression is increased. In one embodiment, the protein is Snhg4, and the protein expression is decreased. In one embodiment, the protein is Spag8, and the protein expression is decreased. In one embodiment, the protein is Stc1, and the protein expression is increased. In one embodiment, the protein is Sv2c, and the protein expression is increased. In one embodiment, the protein is Terf2ip, and the protein expression is increased. In one embodiment, the protein is Thrsp, and the protein expression is decreased. In one embodiment, the protein is Tmc8, and the protein expression is decreased. In one embodiment, the protein is Tmem171, and the protein expression is increased. In one embodiment, the protein is Tmx4, and the protein expression is increased. In one embodiment, the protein is Tnfrsfl2a, and the protein expression is increased. In one embodiment, the protein is Tnni2, and the protein expression is decreased. In one embodiment, the protein is Ttc30b, and the protein expression is decreased. In one embodiment, the protein is Txnip, and the protein expression is decreased. In one embodiment, the protein is Ucp3, and the protein expression is decreased. In one embodiment, the protein is Unc5b, and the protein expression is increased. In one embodiment, the protein is Zfp112, and the protein expression is decreased. In one embodiment, the protein is Zfp13, and the protein expression is decreased. In one embodiment, the protein is Zfp385b, and the protein expression is increased. In one embodiment, the protein is Zfp474, and the protein expression is increased. In one embodiment, the protein is Zfyve28, and the protein expression is decreased. In one embodiment, the protein is Zic, and the protein expression is increased. In one embodiment, the protein is Zmynd10, and the protein expression is decreased. In certain embodiments, the increased protein expression is as compared to a control subject. In certain embodiments, the decreased protein expression is as compared to a control subject. In a specific embodiment, the control subject is a subject that has not been administered a population of stem cells (e.g., PDAC)).

In other embodiments, the aging cell is a somatic cell. In some embodiments, the aging cell is a skeletal muscle cell. In some embodiments, the aging cell is a brain cell. In some embodiments, the aging cell is from the brain. In other embodiments, the aging cell is a cardiac cell. In some embodiments, the aging cell is from the heart. In some instances, the aging cell is a kidney cell. In some embodiments, the aging cell is from the kidney. In some embodiments, the aging cell is a liver cell. In some embodiments, the aging cell is from the liver. In other embodiments, the aging cell is a granulocyte, mast cell or macrophage. In some embodiments, the aging cell is from the bone marrow. In some instances, the aging cell is a skin cell. In some embodiments, the aging cell is from the skin.

In some embodiments, the methods disclosed herein reference a subject. In some embodiments, the subject is 10-15 years of age. In some embodiments, the subject is 15-20 years of age. In some embodiments, the subject is 20-25 years of age. In some embodiments, the subject is 25-30 years of age. In some embodiments, the subject is 30-35 years of age. In some embodiments, the subject is 35-40 years of age. In some embodiments, the subject is 40-45 years of age. In some embodiments, the subject is 45-50 years of age. In some embodiments, the subject is 50-55 years of age. In some embodiments, the subject is 55-60 years of age. In some embodiments, the subject is 60-65 years of age. In some embodiments, the subject is 65-70 years of age. In some embodiments, the subject is 70-75 years of age. In some embodiments, the subject is 75-80 years of age. In some embodiments, the subject is 80-85 years of age. In some embodiments, the subject is 85-90 years of age. In some embodiments, the subject is 90-95 years of age. In some embodiments, the subject is 95-100 years of age. In some embodiments, the subject is or over 100 years of age.

In some embodiments, the methods disclosed herein reference a control subject. In some embodiments, the control subject is the same subject before administration of the population of stem cells (e.g., PDSC). In other embodiments, the control subject is a subject that has not received the population of stem cells (e.g., PDSC).

In certain embodiments, of the various methods provided herein, the method further comprises (i) determining the number of stem cells and/or differentiated cells in the tissue before administration of the population of stem cells (e.g., PDSC) to the subject, and (ii) determining the number of stem cells and/or differentiated cells in the tissue after administration of the population of stem cells (e.g., PDSC) to the subject.

In some embodiments, the method increases the number of stem cells in the tissue after administration as compared to before administration of the population of stem cells (e.g., PDSC). In one embodiment, the subject has an increased number of stem cells as compared to a subject that has not received an administration of population of stem cells (e.g., PDSC). In certain embodiments, the increase in the number of stem cells persists over time. In other embodiments, the increase in the number of stem cells is the result of an expansion of stem cells resident in the tissue. In one embodiment, the increase in the number of stem cells is the result of an expansion of the stem cells (e.g., PDSC) in the tissue.

In another embodiment, the number of stem cells is assessed by stem cell colony forming units.

In some embodiments, the number of stem cells is increased from about 10% to about 100%. In one embodiment, the number of stem cells in increased about 10%. In one embodiment, the number of stem cells in increased about 15%. In one embodiment, the number of stem cells in increased about 20%. In one embodiment, the number of stem cells in increased about 25%. In one embodiment, the number of stem cells in increased about 30%. In one embodiment, the number of stem cells in increased about 35%. In one embodiment, the number of stem cells in increased about 40%. In one embodiment, the number of stem cells in increased about 45%. In one embodiment, the number of stem cells in increased about 50%. In one embodiment, the number of stem cells in increased about 55%. In one embodiment, the number of stem cells in increased about 60%. In one embodiment, the number of stem cells in increased about 65%. In one embodiment, the number of stem cells in increased about 70%. In one embodiment, the number of stem cells in increased about 75%. In one embodiment, the number of stem cells in increased about 80%. In one embodiment, the number of stem cells in increased about 85%. In one embodiment, the number of stem cells in increased about 90%. In one embodiment, the number of stem cells in increased about 95%. In one embodiment, the number of stem cells in increased about 100%. Any range or interval thereof is also contemplated. In some embodiments, the number of stem cells is increased by from about 10% to about 10-fold. In some embodiments, the number of stem cells is increased by from about 2-fold to about 10-fold. In certain embodiments, number of stem cells is increased about 2-fold, about 3-fold, about 4 fold, about 5-fold, about 6-fold, about 7-fold, about 8-fold, about 9-fold or about 10-fold, or any range thereof. In one embodiment, the number of stem cells in increased about 2-fold. In one embodiment, the number of stem cells in increased about 3-fold. In one embodiment, the number of stem cells in increased about 4-fold. In one embodiment, the number of stem cells in increased about 5-fold. In one embodiment, the number of stem cells in increased about 6-fold. In one embodiment, the number of stem cells in increased about 7-fold. In one embodiment, the number of stem cells in increased about 8-fold. In one embodiment, the number of stem cells in increased about 9-fold. In one embodiment, the number of stem cells in increased about 10-fold. Any range or interval thereof is also contemplated. In some embodiments, number of stem cells is increased from about 10% to about 10-fold, or any range thereof. In some embodiments, the number of stem cells is increased to an amount within about 20%, about 10%, or about 5% of the number of number of stem cells present in a control (e.g., the subject before receiving administration of the stem cells; a subject that has not received administration of the stem cells; or the general population, such as determined by an average or median).

Certain methods provided herein reference an increase in the ratio stem cells to differentiated cells. In some embodiments, the ratio of stem cells to differentiated cells is increased from about 10% to about 100%, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

In some embodiments, the ratio of stem cells to differentiated cells is increased about 10%. In some embodiments, the ratio of stem cells to differentiated cells is increased about 15%. In some embodiments, the ratio of stem cells to differentiated cells is increased about 20%. In some embodiments, the ratio of stem cells to differentiated cells is increased about 25%. In some embodiments, the ratio of stem cells to differentiated cells is increased about 30%. In some embodiments, the ratio of stem cells to differentiated cells is increased about 35%. In some embodiments, the ratio of stem cells to differentiated cells is increased about 40%. In some embodiments, the ratio of stem cells to differentiated cells is increased about 45%. In some embodiments, the ratio of stem cells to differentiated cells is increased about 50%. In some embodiments, the ratio of stem cells to differentiated cells is increased about 55%. In some embodiments, the ratio of stem cells to differentiated cells is increased about 60%. In some embodiments, the ratio of stem cells to differentiated cells is increased about 65%. In some embodiments, the ratio of stem cells to differentiated cells is increased about 70%. In some embodiments, the ratio of stem cells to differentiated cells is increased about 75%. In some embodiments, the ratio of stem cells to differentiated cells is increased about 80%. In some embodiments, the ratio of stem cells to differentiated cells is increased about 85%. In some embodiments, the ratio of stem cells to differentiated cells is increased about 90%. In some embodiments, the ratio of stem cells to differentiated cells is increased about 95%. In some embodiments, the ratio of stem cells to differentiated cells is increased about 100%. Any range or interval thereof is also contemplated. In some embodiments, the ratio of stem cells to differentiated cells is increased from about 10% to about 10-fold. In some embodiments, the ratio of stem cells to differentiated cells is increased from about 2-fold to about 10-fold. In some embodiments, the ratio of stem cells to differentiated cells is increased about 2-fold. In some embodiments, the ratio of stem cells to differentiated cells is increased about 3-fold. In some embodiments, the ratio of stem cells to differentiated cells is increased about 4-fold. In some embodiments, the ratio of stem cells to differentiated cells is increased about 5-fold. In some embodiments, the ratio of stem cells to differentiated cells is increased about 6-fold. In some embodiments, the ratio of stem cells to differentiated cells is increased about 7-fold. In some embodiments, the ratio of stem cells to differentiated cells is increased about 8-fold. In some embodiments, the ratio of stem cells to differentiated cells is increased about 9-fold. In some embodiments, the ratio of stem cells to differentiated cells is increased about 10-fold. Any range or interval thereof is also contemplated. In some embodiments, ratio of stem cells to differentiated cells is increased from about 10% to about 10-fold, or any range thereof. In some embodiments, the ratio of stem cells to differentiated cells is increased to an amount within about 20%, about 10%, or about 5% of the number of ratio of stem cells to differentiated cells present in a control (e.g., the subject before receiving administration of the stem cells; a subject that has not received administration of the stem cells; or the general population, such as determined by an average or median).

In some embodiments, the increase in the number of stem cells (or ratio of stem cells to differentiated cells) results in the remodeling, renewal, renovation, rejuvenation, repair and/or restoration of the tissue of the subject. In other embodiments, the increase in the number of stem cells results in the remodeling of the tissue of the subject. In some embodiments, the increase in the number of stem cells results in the renewal of the tissue of the subject. In other embodiments, the increase in the number of stem cells results in the renovation of the tissue of the subject. In some embodiments, the increase in the number of stem cells results in the rejuvenation of the tissue of the subject. In yet other embodiments, the increase in the number of stem cells results in the repair of the tissue of the subject. In some embodiments, the increase in the number of stem cells results in the restoration of the tissue of the subject.

In one embodiment, the method further comprises contacting the population of stem cells (e.g., PDSC) with one or more additional factors isolated from young stem cells, young progenitor cells, or young precursor cells. In certain embodiments, the one or more additional factors are bioactive factors isolated from the secretome of a stem cell. In certain embodiments, the one or more additional factors are bioactive factors isolated from the secretome of a PDSC. In some embodiments, the one or more additional factors is selected from the group consisting of cytokines, hormones, promoters, repressors, proteins, nucleic acids, viruses, immunogens, angiogenic factors, growth factors, anti-apoptotic factors, and anti-oxidative factors, or any combination thereof. In another embodiment, the method further comprises culturing and/or expanding the population of stem cells (e.g., PDSC) prior to administration to the subject. In one embodiment, the culturing and/or expanding is in vitro. In certain embodiments, the culturing and/or expanding is in situ. In other embodiments, the population of stem cells (e.g., PDSC) is cultured and/or expanded in the presence of young stem cells, young progenitor cells, or young precursor cells. In one embodiment, the population of stem cells (e.g., PDSC) is cultured and/or expanded in the presence of additional factors isolated from young stem cells, young progenitor cells, or young precursor cells. In certain embodiments, the one or more additional factors is a bioactive factor isolated from the secretome of a stem cell. In certain embodiments, the one or more additional factors is a bioactive factor isolated from the secretome of a PDAC. In another embodiment, the one or more additional factors is selected from the group consisting of cytokines, hormones, promoters, repressors, proteins, nucleic acids, viruses, immunogens, angiogenic factors, growth factors, anti-apoptotic factors, and anti-oxidative factors, or any combination thereof. In some embodiments, the population of stem cells (e.g., PDSC) are cultured and/or expanded in an extracorporeal device. In some embodiments, the population of stem cells (e.g., PDSC) has been passaged at least three times. In one embodiment, the population of stem cells (e.g., PDSC) has been passaged no more than ten times.

In some embodiments, the method further comprises characterizing the genome of the stem cells. In one embodiment, the genomic characterization is conducted prior to administration of the population of stem cells to the subject. In another embodiment, the genomic characterization is conducted after administration of the population of stem cells to the subject. In some embodiments, the genomic characterization is conducted prior to administration of the population of stem cells to the subject, and after administration of the population of stem cells to the subject. In some embodiments, the method further comprises characterizing the genome of the PDSC. In one embodiment, the genomic characterization is conducted prior to administration of the population of PDSC to the subject. In another embodiment, the genomic characterization is conducted after administration of the population of PDSC to the subject. In some embodiments, the genomic characterization is conducted prior to administration of the population of PDSC to the subject, and after administration of the population of PDSC to the subject. In situations where stem cells derived from multiple donors are administered, genome characterization can facilitate the selection of the different stem cells that are used to make the composition to be administered. This allows one to include in the composition a mixture of stem cell preparations, where each preparation of stem cells used in the mixture has a particular desired genotype. In some embodiments, the preparations of stem cells from different donors are selected without use of HLA typing to determine compatibility with the recipient.

In some embodiments, the method further comprises characterizing the proteome of the stem cells. In other embodiments, the proteomic characterization is conducted prior to administration of the population of stem cells to the subject. In another embodiment, the proteomic characterization is conducted after administration of the population of stem cells to the subject. In one embodiment, the proteomic characterization is conducted prior to administration of the population of stem cells to the subject, and after administration of the population of stem cells to the subject. In some embodiments, the method further comprises characterizing the proteome of the PDSC. In other embodiments, the proteomic characterization is conducted prior to administration of the population of PDSC to the subject. In another embodiment, the proteomic characterization is conducted after administration of the population of PDSC to the subject. In one embodiment, the proteomic characterization is conducted prior to administration of the population of PDSC to the subject, and after administration of the population of PDSC to the subject.

In some embodiments, the method further comprises characterizing the genome of the stem cells and/or differentiated cells in the tissue. In another embodiment, the genomic characterization is conducted prior to administration of the population of stem cells (e.g., PDSC) to the subject. In one embodiment, the genomic characterization is conducted after administration of the population of stem cells (e.g., PDSC) to the subject. In some embodiments, the genomic characterization is conducted prior to administration of the population of stem cells (e.g., PDSC) to the subject, and after administration of the population of stem cells (e.g., PDSC) to the subject.

In certain embodiments, the method further comprises characterizing the proteome of the stem cells and/or differentiated cells in the tissue. In one embodiment, the proteomic characterization is conducted prior to administration of the population of stem cells (e.g., PDSC) to the subject. In another embodiment, the proteomic characterization is conducted after administration of the population of stem cells (e.g., PDSC) to the subject. In other embodiments, the proteomic characterization is conducted prior to administration of the population of stem cells (e.g., PDSC) to the subject, and after administration of the population of stem cells (e.g., PDSC) to the subject.

The level of expression of the biomarkers (e.g., nucleic acids or proteins) provided herein can be used in the methods provided herein. For example, in certain embodiments, the expression of biomarkers can be used to confirm the identity of a population of isolated stem cells (e.g., PDSC), to identify a population of cells as comprising at least a plurality of isolated stem cells (e.g., PDSC), or the like. Populations of isolated stem cells (e.g., PDSC), the identity of which is confirmed, can be clonal, e.g., populations of isolated stem cells (e.g., PDSC) expanded from a single isolated stem cells (e.g., PDSC), or a mixed population of stem cells (e.g., PDSC), e.g., a population of cells comprising isolated stem cells (e.g., PDSC) that are expanded from multiple isolated stem cells (e.g., PDSC), or a population of cells comprising isolated stem cells (e.g., PDSC), as described herein, and at least one other type of cell.

The level of expression of these genes can also be used to select populations of isolated stem cells (e.g., PDSC). For example, a population of cells, e.g., clonally-expanded stem cells (e.g., PDSC), may be selected if the expression of one or more of the genes provided herein is significantly higher in a sample from the population of cells than in an equivalent population of bone marrow-derived mesenchymal stem cells. Such selecting can be of a population from a plurality of isolated placental stem cell populations, from a plurality of cell populations, the identity of which is not known, etc.

Isolated stem cells (e.g., PDSC) can be selected on the basis of the level of expression of one or more such genes as compared to the level of expression in said one or more genes in a control cell (e.g., stem cell, such as an irrelevant stem cell).

For example, isolated PDSC can be selected on the basis of the level of expression of one or more such genes as compared to the level of expression in said one or more genes in e.g., a bone marrow-derived mesenchymal stem cell control. In one embodiment, the level of expression of said one or more genes in a sample comprising an equivalent number of bone marrow-derived mesenchymal stem cells is used as a control. In another embodiment, the control, for isolated PDSC tested under certain conditions, is a numeric value representing the level of expression of said one or more genes in bone marrow-derived mesenchymal stem cells under said conditions. For example, in some embodiments, a method for selecting isolated PDSC or populations of isolated PDSC on the basis of gene expression of one or more genes comprises selecting cells that express one or more genes at a detectably higher level than a bone marrow-derived mesenchymal stem cell, wherein said one or more genes are selected from the group consisting of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A, and wherein said bone marrow derived stem cell has undergone a number of passages in culture equivalent to the number of passages said PDSC has undergone. In a more specific embodiment, said selecting comprises selecting cells that express ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN and ZC3H12A at a detectably higher level than a bone marrow-derived mesenchymal stem cell.

In some embodiments, the tissue is muscle. In one embodiment, the tissue is brain. In another embodiment, the tissue is skin. In some embodiments, the tissue is bone marrow. In one embodiment, the tissue is heart. In certain embodiments, the tissue is liver. In another embodiment, the tissue is kidney.

In some embodiments, the methods disclosed herein comprise administration of a population of stem cells to a subject. In one embodiment, the population of stem cells comprises a population of stem cells. In another embodiment, the population of stem cells consists essentially of a population of stem cells. In a specific embodiment, the population of stem cells consists of a population of stem cells.

In certain embodiments, about 1×10⁵ to about 1×10⁸ stem cells per recipient kilogram, such as about 1×10⁶ to about 1×10⁷ stem cells per recipient kilogram. In various embodiments, stem cells administered to an individual or subject comprise at least 1×10⁵, 3×10⁵, 5×10⁵, 1×10⁶, 3×10⁶, 5×10⁶, 1×10⁷, 3×10⁷, 5×10⁷, 1×10⁸, 2×10⁸, 3×10⁸, 4×10⁸, 5×10⁸, 6×10⁸, 7×10⁸, 8×10⁸, 8×10⁸, 1×10⁹, 2×10⁹, 3×10⁹, 4×10⁹, 5×10⁹, 1×10¹⁰, 5×10¹⁰, or 1×10¹¹ or more stem cells. In one embodiment, a population of stem cells administered to a subject comprises at least 1×10⁵, 3×10⁵, 5×10⁵, 1×10⁶, 3×10⁶, 5×10⁶, 1×10⁷, 3×10⁷, 5×10⁷, 1×10⁸, 2×10⁸, 3×10⁸, 4×10⁸, 5×10⁸, 6×10⁸, 7×10⁸, 8×10⁸, 8×10⁸, 1×10⁹, 2×10⁹, 3×10⁹, 4×10⁹, 5×10⁹, 1×10¹⁰, 5×10¹⁰, or 1×10¹¹ or more stem cells. In one embodiment, a population of stem cells administered to a subject comprises at least 1×10⁵ stem cells. In one embodiment, a population of stem cells administered to a subject comprises at least 3×10⁵ stem cells. In one embodiment, a population of stem cells administered to a subject comprises at least 5×10⁵ stem cells. In one embodiment, a population of stem cells administered to a subject comprises at least 1×10⁶ Stem cells. In one embodiment, a population of stem cells administered to a subject comprises at least 3×10⁶ Stem cells. In one embodiment, a population of stem cells administered to a subject comprises at least 5×10⁶ Stem cells. In one embodiment, a population of stem cells administered to a subject comprises at least 1×10⁷ stem cells. In one embodiment, a population of stem cells administered to a subject comprises at least 3×10⁷ stem cells. In one embodiment, a population of stem cells administered to a subject comprises at least 5×10⁷ stem cells. In one embodiment, a population of stem cells administered to a subject comprises at least 1×10⁸ stem cells. In one embodiment, a population of stem cells administered to a subject comprises at least 2×10⁸ stem cells. In one embodiment, a population of stem cells administered to a subject comprises at least 3×10⁸ stem cells. In one embodiment, a population of stem cells administered to a subject comprises at least 4×10⁸ stem cells. In one embodiment, a population of stem cells administered to a subject comprises at least 5×10⁸ stem cells. In one embodiment, a population of stem cells administered to a subject comprises at least 6×10⁸ stem cells. In one embodiment, a population of stem cells administered to a subject comprises at least 7×10⁸ stem cells. In one embodiment, a population of stem cells administered to a subject comprises at least 8×10⁸ stem cells. In one embodiment, a population of stem cells administered to a subject comprises at least 8×10⁸ stem cells. In one embodiment, a population of stem cells administered to a subject comprises at least 1×10⁹ stem cells. In one embodiment, a population of stem cells administered to a subject comprises at least 2×10⁹ stem cells. In one embodiment, a population of stem cells administered to a subject comprises at least 3×10⁹ stem cells. In one embodiment, a population of stem cells administered to a subject comprises at least 4×10⁹ stem cells. In one embodiment, a population of stem cells administered to a subject comprises at least 5×10⁹ stem cells. In one embodiment, a population of stem cells administered to a subject comprises at least 1×10¹⁰ stem cells. In one embodiment, a population of stem cells administered to a subject comprises at least 5×10¹⁰ stem cells. In one embodiment, a population of stem cells administered to a subject comprises at least or 1×10¹¹ stem cells.

In one embodiment, a population of stem cells administered to a subject comprises 1×10⁵ stem cells. In one embodiment, a population of stem cells administered to a subject comprises 3×10⁵ stem cells. In one embodiment, a population of stem cells administered to a subject comprises 5×10⁵ stem cells. In one embodiment, a population of stem cells administered to a subject comprises 1×10⁶ stem cells. In one embodiment, a population of stem cells administered to a subject comprises 3×10⁶ stem cells. In one embodiment, a population of stem cells administered to a subject comprises 5×10⁶ Stem cells. In one embodiment, a population of stem cells administered to a subject comprises 1×10⁷ stem cells. In one embodiment, a population of stem cells administered to a subject comprises 3×10⁷ stem cells. In one embodiment, a population of stem cells administered to a subject comprises 5×10⁷ stem cells. In one embodiment, a population of stem cells administered to a subject comprises 1×10⁸ stem cells. In one embodiment, a population of stem cells administered to a subject comprises 2×10⁸ stem cells. In one embodiment, a population of stem cells administered to a subject comprises 3×10⁸ stem cells. In one embodiment, a population of stem cells administered to a subject comprises 4×10⁸ stem cells. In one embodiment, a population of stem cells administered to a subject comprises 5×10⁸ stem cells. In one embodiment, a population of stem cells administered to a subject comprises 6×10⁸ stem cells. In one embodiment, a population of stem cells administered to a subject comprises 7×10⁸ stem cells. In one embodiment, a population of stem cells administered to a subject comprises 8×10⁸ stem cells. In one embodiment, a population of stem cells administered to a subject comprises 8×10⁸ stem cells. In one embodiment, a population of stem cells administered to a subject comprises 1×10⁹ stem cells. In one embodiment, a population of stem cells administered to a subject comprises 2×10⁹ stem cells. In one embodiment, a population of stem cells administered to a subject comprises 3×10⁹ stem cells. In one embodiment, a population of stem cells administered to a subject comprises 4×10⁹ stem cells. In one embodiment, a population of stem cells administered to a subject comprises 5×10⁹ stem cells. In one embodiment, a population of stem cells administered to a subject comprises 1×10¹⁰ stem cells. In one embodiment, a population of stem cells administered to a subject comprises 5×10¹⁰ stem cells. In one embodiment, a population of stem cells administered to a subject comprises or 1×10¹¹ stem cells.

In some embodiments, the methods disclosed herein comprise administration of a population of PDSC to a subject. In one embodiment, the population of stem cells comprises a population of PDSC. In another embodiment, the population of stem cells consists essentially of a population of PDSC. In a specific embodiment, the population of stem cells consists of a population of PDSC.

In certain embodiments, about 1×10⁵ to about 1×10⁸ PDSC per recipient kilogram, such as about 1×10⁶ to about 1×10⁷ PDSC per recipient kilogram. In various embodiments, PDSC administered to an individual or subject comprise at least 1×10⁵, 3×10⁵, 5×10⁵, 1×10⁶, 3×10⁶, 5×10⁶, 1×10⁷, 3×10⁷, 5×10⁷, 1×10⁸, 2×10⁸, 3×10⁸, 4×10⁸, 5×10⁸, 6×10⁸, 7×10⁸, 8×10⁸, 8×10⁸, 1×10⁹, 2×10⁹, 3×10⁹, 4×10⁹, 5×10⁹, 1×10¹⁰, 5×10¹⁰, or 1×10¹¹ or more PDSC. In one embodiment, a population of PDSC administered to a subject comprises at least 1×10⁵, 3×10⁵, 5×10⁵, 1×10⁶, 3×10⁶, 5×10⁶, 1×10⁷, 3×10⁷, 5×10⁷, 1×10⁸, 2×10⁸, 3×10⁸, 4×10⁸, 5×10⁸, 6×10⁸, 7×10⁸, 8×10⁸, 8×10⁸, 1×10⁹, 2×10⁹, 3×10⁹, 4×10⁹, 5×10⁹, 1×10¹⁰, 5×10¹⁰, or 1×10¹¹ or more PDSC. In one embodiment, a population of PDSC administered to a subject comprises at least 1×10⁵ PDSC. In one embodiment, a population of PDSC administered to a subject comprises at least 3×10⁵ PDSC. In one embodiment, a population of PDSC administered to a subject comprises at least 5×1 0⁵ PDSC. In one embodiment, a population of PDSC administered to a subject comprises at least 1×10⁶ PDSC. In one embodiment, a population of PDSC administered to a subject comprises at least 3×10⁶ PDSC. In one embodiment, a population of PDSC administered to a subject comprises at least 5×10⁶ PDSC. In one embodiment, a population of PDSC administered to a subject comprises at least 1×10⁷ PDSC. In one embodiment, a population of PDSC administered to a subject comprises at least 3×10⁷ PDSC. In one embodiment, a population of PDSC administered to a subject comprises at least 5×10⁷ PDSC. In one embodiment, a population of PDSC administered to a subject comprises at least 1×10⁸ PDSC. In one embodiment, a population of PDSC administered to a subject comprises at least 2×10⁸ PDSC. In one embodiment, a population of PDSC administered to a subject comprises at least 3×10⁸ PDSC. In one embodiment, a population of PDSC administered to a subject comprises at least 4×10⁸ PDSC. In one embodiment, a population of PDSC administered to a subject comprises at least 5×10⁸ PDSC. In one embodiment, a population of PDSC administered to a subject comprises at least 6×10⁸ PDSC. In one embodiment, a population of PDSC administered to a subject comprises at least 7×10⁸ PDSC. In one embodiment, a population of PDSC administered to a subject comprises at least 8×10⁸ PDSC. In one embodiment, a population of PDSC administered to a subject comprises at least 8×10⁸ PDSC. In one embodiment, a population of PDSC administered to a subject comprises at least 1×10⁹ PDSC. In one embodiment, a population of PDSC administered to a subject comprises at least 2×10⁹ PDSC. In one embodiment, a population of PDSC administered to a subject comprises at least 3×10⁹ PDSC. In one embodiment, a population of PDSC administered to a subject comprises at least 4×10⁹ PDSC. In one embodiment, a population of PDSC administered to a subject comprises at least 5×10⁹ PDSC. In one embodiment, a population of PDSC administered to a subject comprises at least 1×10¹o PDSC. In one embodiment, a population of PDSC administered to a subject comprises at least 5×10¹⁰ PDSC. In one embodiment, a population of PDSC administered to a subject comprises at least or 1×10¹¹ PDSC.

In one embodiment, a population of PDSC administered to a subject comprises 1×10⁵ PDSC. In one embodiment, a population of PDSC administered to a subject comprises 3×10⁵ PDSC. In one embodiment, a population of PDSC administered to a subject comprises 5×10⁵ PDSC. In one embodiment, a population of PDSC administered to a subject comprises 1×10⁶ PDSC. In one embodiment, a population of PDSC administered to a subject comprises 3×10⁶ PDSC. In one embodiment, a population of PDSC administered to a subject comprises 5×10⁶ PDSC. In one embodiment, a population of PDSC administered to a subject comprises 1×10⁷ PDSC. In one embodiment, a population of PDSC administered to a subject comprises 3×10⁷ PDSC. In one embodiment, a population of PDSC administered to a subject comprises 5×10⁷ PDSC. In one embodiment, a population of PDSC administered to a subject comprises 1×10⁸ PDSC. In one embodiment, a population of PDSC administered to a subject comprises 2×10⁸ PDSC. In one embodiment, a population of PDSC administered to a subject comprises 3×10⁸ PDSC. In one embodiment, a population of PDSC administered to a subject comprises 4×10⁸ PDSC. In one embodiment, a population of PDSC administered to a subject comprises 5×10⁸ PDSC. In one embodiment, a population of PDSC administered to a subject comprises 6×10⁸ PDSC. In one embodiment, a population of PDSC administered to a subject comprises 7×10⁸ PDSC. In one embodiment, a population of PDSC administered to a subject comprises 8×10⁸ PDSC. In one embodiment, a population of PDSC administered to a subject comprises 8×10⁸ PDSC. In one embodiment, a population of PDSC administered to a subject comprises 1×10⁹ PDSC. In one embodiment, a population of PDSC administered to a subject comprises 2×10⁹ PDSC. In one embodiment, a population of PDSC administered to a subject comprises 3×10⁹ PDSC. In one embodiment, a population of PDSC administered to a subject comprises 4×10⁹ PDSC. In one embodiment, a population of PDSC administered to a subject comprises 5×10⁹ PDSC. In one embodiment, a population of PDSC administered to a subject comprises 1×10¹⁰ PDSC. In one embodiment, a population of PDSC administered to a subject comprises 5×10¹⁰ PDSC. In one embodiment, a population of PDSC administered to a subject comprises or 1×10¹¹ PDSC.

The PDSC can also be administered with one or more second types of stem cells, e.g., mesenchymal stem cells from bone marrow, neural stem cells from brain or spinal cord, or stem cells from fat tissue. Such second stem cells can be administered to an individual with said PDSC in a ratio of, e.g., between about 1:10 to about 10:1.

To facilitate contacting, or proximity, of PDSC and immune cells in vivo, the PDSC can be administered to an individual by any route sufficient to bring the PDSC and immune cells into contact with or proximity to each other. For example, the PDSC can be administered to the individual, e.g., intravenously, intramuscularly, intraperitoneally, intraocularly, parenterally, intrathecally, intraarterially, subcutaneously, or directly into an organ. The PDSC can be formulated as a pharmaceutical composition as described elsewhere herein. In a specific embodiment, the PDSC are administered subcutaneously.

In some embodiments, the population of stem cells (e.g., PDSC) is administered systemically. In one embodiment, the population of stem cells (e.g., PDSC) is administered locally to the tissue. In other embodiments, the population of stem cells (e.g., PDSC) is administered by parenteral administration. In another embodiment, the population of stem cells (e.g., PDSC) is administered intravenously. In some embodiments, the population of stem cells (e.g., PDSC) is administered by continuous drip or as a bolus. In one embodiment, the population of stem cells (e.g., PDSC) is prepared to be administered in an injectable liquid suspension or other biocompatible medium. In other embodiments, the population of stem cells (e.g., PDSC) is administered using a catheter. In another embodiment, the population of stem cells (e.g., PDSC) is administered using a controlled-release system. In one embodiment, the population of stem cells (e.g., PDSC) is administered using an implantable substrate or matrix. In certain embodiments, the population of stem cells (e.g., PDSC) is administered intramuscularly. In some embodiments, the population of stem cells (e.g., PDSC) is administered subcutaneously. In one embodiment, the population of stem cells (e.g., PDSC) is administered subdermally. In another embodiment, the population of stem cells (e.g., PDSC) is administered intracompartmentally. In some embodiments, the population of stem cells (e.g., PDSC) is administered by intraperitoneal administration. In other embodiments, the method further comprises contacting the population of stem cells (e.g., PDSC) with young stem cells, young progenitor cells, or young precursor cells.

In certain embodiments, the stem cells (e.g., PDSC) are administered in a biocompatible medium which is, or becomes a semi-solid or solid matrix in situ. In some embodiments, the matrix is an injectable liquid which polymerizes to a semi-solid gel, such as collagen and its derivatives, polylactic acid or polyglycolic acid. In other embodiments, the matrix is one or more layers of a flexible, solid matrix that is implanted in its final form, such as impregnated fibrous matrices. The matrix can be, for example, Gelfoam® (Upjohn, Kalamazoo, Mich.) or a biologic matrix. In certain embodiments, the matrix is permanent. In other embodiments, the matrix is degradable or biodegradable. In some embodiments, the stem cells are embedded into a tissue-engineered patch containing, for example, a collagen matrix. Such a patch can then be attached or otherwise delivered to a tissue, for example, with a sealant (e.g., fibrin).

In certain embodiments, the stem cells (e.g., PDSC) are administered as a cell suspension in a pharmaceutically acceptable liquid medium (e.g., saline or buffer), for example, for systemic administration or local administration directly into a tissue of the subject.

An effective amount or dose of stem cells for use in the methods provided herein will vary depending on the stem cell type used and/or the delivery site (e.g., intravenously or locally), and such doses can be readily determined by a physician.

In one embodiment, the population of stem cells (e.g., PDSC) is administered as a single dose. In another embodiment, the population of stem cells (e.g., PDSC) is administered as multiple doses.

In one embodiment, the stem cells (e.g., PDSC) (or population of stem cells (e.g., PDSC)) are administered at a dose of between 1×10⁵ cells and 1×10¹¹ In one embodiment, the stem cells (e.g., PDSC) are administered at a dose of between 1×10⁵ cells and 1×10⁹ cells. In certain embodiments, the population of stem cells (e.g., PDSC) is administered at a dose of between 1×10⁵ cells and 1×10⁷ cells. In other embodiments, the population of stem cells (e.g., PDSC) is administered at a dose of between 1×10⁶ cells and 1×10⁷ cells. In other embodiments, the population of stem cells (e.g., PDSC) is administered at a dose of between 1×10⁸ cells and 1×10⁹ cells. In some embodiments, the population of stem cells (e.g., PDSC) is administered at a dose of about 1×10⁶ cells. In some embodiments, the population of stem cells (e.g., PDSC) is administered at a dose of about 1×10⁷ cells. In some embodiments, the population of stem cells (e.g., PDSC) is administered at a dose of about 1×10⁸ cells. In some embodiments, the population of stem cells (e.g., PDSC) is administered at a dose of about 1×10⁹ cells.

For example, stem cells (e.g., PDSC) can be administered in a dose between about 1×10⁶ and 1×10⁸, such as between 1×10⁷ and 5×10⁷. Depending on the amount of aging or injury, more or less cells can be used. More damage may require a larger dose of cells (e.g., in an older subject), and a less damage may require a smaller dose of cells (e.g., in a younger subject). On the basis of body weight of the recipient, an effective dose may be between 1×10⁵ and 1×10⁷ per kg of body weight, such as between 1×10⁶ and 5×10⁶ cells per kg of body weight. Patient age, general condition, and immunological status may be used as factors in determining the dose administered, and will be readily determined by the physician.

In one embodiment, the population of stem cells (e.g., PDSC) administered when the subject is 10-15 years of age, 15-20 years of age, 20-25 years of age, 25-30 years of age, 30-35 years of age, 35-40 years of age, 40-45 years of age, 45-50 years of age, 50-55 years of age, 55-60 years of age, 60-65 years of age, 65-70 years of age, 70-75 years of age, 75-80 years of age, 80-85 years of age, 85-90 years of age, 90-95 years of age, 95-100 years of age, or over 100 years of age. In some embodiments, the administration is the first administration. In one embodiment, the stem cells (e.g., PDSC) are administered between 10 to 15 years of age. In another embodiment, the stem cells (e.g., PDSC) are administered between 15 and 20 years of age. In another embodiment, the stem cells (e.g., PDSC) are administered between 20 to 25 years of age. In one embodiment, the stem cells (e.g., PDSC) are administered between 25 to 30 years of age. In a specific embodiment, the stem cells (e.g., PDSC) are administered between 30 to 35 years of age. In another embodiment, the stem cells (e.g., PDSC) are administered between 35 to 40 years of age. In one embodiment, the stem cells (e.g., PDSC) are administered between 40 to 45 years of age. In a specific embodiment, the stem cells (e.g., PDSC) are administered between 45 to 50 years of age. In a specific embodiment, the stem cells (e.g., PDSC) are administered between 50 to 55 years of age. In one embodiment, the stem cells (e.g., PDSC) are administered between 60 to 65 years of age. In another embodiment, the stem cells (e.g., PDSC) are administered between 65 to 70 years of age. In a specific embodiment, the stem cells (e.g., PDSC) are administered between 70 to 75 years of age. In one embodiment, the stem cells (e.g., PDSC) are administered between 75 to 80 years of age. In another embodiment, the stem cells (e.g., PDSC) are administered between 80 to 85 years of age. In a specific embodiment, the stem cells (e.g., PDSC) are administered between 85 to 90 years of age. In one embodiment, the stem cells (e.g., PDSC) are administered between 90 to 95 years of age. In another embodiment, the stem cells (e.g., PDSC) are administered between 95 to 100 years of age. In a specific embodiment, the stem cells (e.g., PDSC) are administered after 100 years of age. In some embodiments, the populations of stem cells (e.g., PDSC) are serially administered over the lifetime of the subject.

In certain embodiments, the stem cells (e.g., PDSC) are administered to a subject once. In other embodiments, stem cells (e.g., PDSC) are administered to a subject more than one time. In a specific embodiment, the stem cells (e.g., PDSC) are administered serially over the course of a lifetime of the subject. In some embodiments, the population of stem cells (e.g., PDSC) is administered.

In one embodiment, the stem cells (e.g., PDSC) are administered once per month, every other month, every third month, every fourth month, twice per year, once per year. In some embodiment, the stem cells (e.g., PDSC) are administered once every other year, once every third year, once every four years, once every 5 years, once every ten years, once every 15 years, once every 20 years, or once every 25 years.

In certain embodiments, the stem cells (e.g., PDSC) are administered for 1, 2, 3, 4, 5, 6, 7, 8, 9, 10, 15, 20, 25, 30, 35, 40, 45, 50, 55, 60, 65, 70, 75, 80, 85, 90 years or longer.

In some embodiments, the stem cells (e.g., PDSC) are administered as one done. In certain embodiments, the stem cells (e.g., PDSC) are administered in two or more doses, such as 2, 3, 4, 5, 6, 7, 8, 9, 10 or more doses, or any interval thereof.

In some embodiments, the stem cells (e.g., PDSC) are delivered yearly for two or more years, such as about 2 years, about 3 years, about 4 years, about 5 years, about 6 years, about 7 years, about 8 years, about 9 years, about 10 years, about 15 years, about 20 years, about 25 years or longer, about 30 years, about 35 years, about 40 years, about 45 years, about 50 years, about 55 years, about 60 years, about 65 years, about 70 years, about 75 years, about 80 years, about 85 years, about 90 years, about 95 years, about 100 years, or any interval thereof, or for the lifetime of the subject.

In other embodiments, the stem cells (e.g., PDSC) are delivered every two years for four or more years, such as about 4 years, about 6 years, about 8 years, about 10 years, about 20 years, about 30 years, about 40 years, about 50 years, about 60 years, about 70 years, about 80 years, about 90 years, about 100 years, or any interval thereof, or for the lifetime of the subject.

In other embodiments, the stem cells (e.g., PDSC) are delivered every five years for ten or more years, such as about 10 years, about 15 years, about 20 years, about 25 years or longer, about 30 years, about 35 years, about 40 years, about 45 years, about 50 years, about 55 years, about 60 years, about 65 years, about 70 years, about 75 years, about 80 years, about 85 years, about 90 years, about 95 years, about 100 years, or any interval thereof, or for the lifetime of the subject.

In other embodiments, the stem cells (e.g., PDSC) are delivered every ten years for twenty or more years, such as about 20 years, about 30 years, about 40 years, about 50 years, about 60 years, about 70 years, about 80 years, about 90 years, about 100 years, or any interval thereof, or for the lifetime of the subject.

In some embodiments, the subject is administered stem cells (e.g., PDSC) two, three, four, five, six, seven, eight, nine, ten, eleven, twelve times, thirteen, fourteen, fifteen, sixteen, seventeen, eighteen, nineteen, twenty, twenty-one, twenty-two, twenty-three, twenty-four, twenty-five, or more times at various intervals over the course of their lifetime. Dosages of the stem cells (e.g., PDSC) serially administered to a subject at different intervals may or may not be identical as prior dosages.

In certain embodiments, the route of administration for a dose of stem cells (e.g., PDSC) to a subject is intravenous, intramuscular, intracompartmental, or a combination thereof, but other routes described herein are also acceptable. Each dose may or may not be administered by an identical route of administration. In some embodiments, an antibody can be administered via multiple routes of administration simultaneously or subsequently to other doses of the same or a different population of stem cells (e.g., PDSC) provided herein.

Various combinations of ages, dosing and frequency of administration provided here are also contemplated.

One or more of the methods of delivery or compositions provided herein can be used to administer the stem cells (e.g., PDSC) provided herein. For example, in certain embodiments, stem cells (e.g., PDSC) can be administered to the subject by a first method of delivery and/or in a first formulation (e.g., direct needle injection of liquid formulation), and stem cells (e.g., PDSC) can be concurrently or sequentially administered to the subject using a second method of delivery and/or in a second formulation (e.g., matrix).

In another aspect, provided herein is a method to recover, isolate, characterize and/or expand cells derived from the remnants of birth (e.g., placenta) which retain the pluripotency, differentiation, and proteomic synthetic diversity of youthful tissue (e.g., PDSC). In one embodiment, the methods comprise recovering the cells. In another embodiment, the method comprises isolating the cells. In other embodiments, the method comprises characterizing the cells. In another embodiment, the method comprises expanding the cells. Exemplary methods of recovering, isolating, characterizing and expanding cells, such as PDSC, are provided elsewhere herein.

In particular embodiments, the methods provided herein are used for the purpose of cryopreserving the cells. In some embodiments, the cells are cryopreserved in a form that can be appropriated in the future, for example, to administered to subjects. In some embodiments, the cells are autologous to the subject. In some embodiments, the cells are allogeneic to the subject. Exemplary methods of cryopreservation and placental stem cell baking are provided elsewhere herein.

In some embodiments, the methods provided herein restore, recharge and/or replenish the pool of stem and progenitor cells (e.g., those cells resident in a tissue). In some embodiments, the cells are restored. In other embodiments, the cells (e.g., those cells resident in a tissue) are recharged. In yet other embodiments, the cells (e.g., those cells resident in a tissue) are replenished. When the cells (e.g., those cells resident in a tissue) are restored, recharged and/or replenished, an improvement in the quality of the general physio-anatomic performance of the recipient can, in certain embodiments, occur. In certain embodiments, the cells (e.g., those cells resident in a tissue) are aged or injured cells.

In certain embodiments, additional methods can be employed for the characterization, expansion, qualification and clinical deployment of the cells for this purpose, and are described elsewhere herein.

In some embodiments, methods are provided herein for the characterization and qualification of expanded and unmanipulated cells. This characterization and qualification can be useful for the purpose of long term cryopreservation and subsequent clinical utilization. The clinical utilization can be any of the various methods provided herein. In certain embodiments, the method results in the restoration of the cellular regenerative potential of the recipient and/or the synthetic capacity of the recipient to combat, reverse, ameliorate the effects of aging; or any combination thereof.

Administration and delivery of cells, e.g., for the purpose of correcting the proteomic and other defects associated with aging, can include any method of parenteral administration, including intravenous infusion, direct intramuscular, subcutaneous, intracompartmental, intraperitoneal, and subdermal administration. Other exemplary delivery methods are provided elsewhere herein. The dose and formulation of said cells can also include any conventional means of suspending and injecting said product, including those provided elsewhere herein. In a specific embodiment, the cells are administered to a subject in need thereof.

### 4.3 Placental-Derived Stem Cells (PDSC)

Placental-derived stem cells are stem cells, obtainable from a placenta or part thereof, that have the capacity to differentiate into non-placental cell types. In a specific embodiment, the PDSC adhere to a tissue culture substrate. The PDSC can be either fetal or maternal in origin (that is, can have the genotype of either the fetus or mother, respectively). In certain embodiments, the PDSC populations provided herein are fetal in origin. Populations of PDSC, or populations of cells comprising PDSC, can comprise PDSC that are solely fetal or maternal in origin, or can comprise a mixed population of PDSC of both fetal and maternal origin. The PDSC, and populations of cells comprising the PDSC, can be identified and selected by the morphological, marker, and culture characteristic discussed below.

In one embodiment, the population of PDSC has previously been cryopreserved. In another embodiment, the population of PDSC are cells from a placental stem cell bank. In one embodiment, the population of PDSC comprises cells obtained from a placenta that has been drained of cord blood. In one embodiment, the population of PDSC comprises cells obtained from a placenta that has been perfused to remove residual blood.

4.3.1 PDSC Cell Surface, Molecular and Genetic Markers

Placental-derived stem cells of disclosed herein, and populations of placental-derived stem cells, express a plurality of markers that can be used to identify and/or isolate the stem cells, or populations of cells that comprise the stem cells. The PDSC, and PDSC populations provided herein (that is, two or more PDSC) include stem cells and stem cell-containing cell populations obtained directly from the placenta, or any part thereof (e.g., amnion, chorion, placental cotyledons, and the like). Placental stem cell populations also includes populations of (that is, two or more) PDSC in culture, and a population in a container, e.g., a bag. PDSC are not, however, trophoblasts.

In other embodiments, the PDSC are embryonic-like stem cells. In one embodiment, the PDSC are pluripotent or multipotent stem cells. In certain embodiments, the population of PDSC comprises cells that are CD34⁻, CD10⁺, SH2⁺, CD90⁺ placental multipotent cells. In another embodiment, the population of PDSC comprises cells that CD34⁻, CD38⁻, CD45⁻, CD10⁺, CD29⁺, CD44⁺, CD54⁺, CD90⁺, SH2⁺, SH3⁺, SH4⁺ and OCT-4⁺. In one embodiment, the population of PDSC comprises cells that are CD34⁻, CD10⁺, CD105⁺, and CD200⁺. In one embodiment, the population of PDSC comprises cells that are CD73⁺. In one embodiment, the population of PDSC comprises cells that are CD73⁺ and CD105⁺. In some embodiments, the population of PDSC comprises cells that are CD200⁺. In one embodiment, the population of PDSC comprises cells that are CD34⁻, CD38⁻, CD45⁻, OCT-4⁺ and CD200⁺. In one embodiment, the population of PDSC comprises cells that are CD34⁻, CD38⁻, CD45⁻, CD73⁺, OCT-4⁺ and CD200⁺. In other embodiments, the population of PDSC comprises cells that are OCT-4⁺. In one embodiment, the population of PDSC comprises cells that are CD73⁺, CD105⁺, and OCT4⁺. In one embodiment, the population of PDSC comprises cells that are CD73⁺, CD105⁺, and CD200⁺. In another embodiment, the population of PDSC comprises cells that are CD73⁺ and CD105⁺. In some embodiments, the population of PDSC comprises cells that are CD200⁺ an OCT-4⁻. In one embodiment, the population of PDSC comprises cells that are CD73⁺, CD105⁺, and HLA-G⁺.

In certain embodiments, the population of PDSC comprises cells that are CD73⁺, CD105⁺, HLA-G⁺. In another embodiment, the population of PDSC comprises cells that are CD73⁺, CD105⁺, CD200⁺ and HLA-G⁺. In one embodiment, the population of PDSC comprises cells that are CD34⁻; CD38⁻: CD45⁻; CD34⁻ and CD38⁻; CD34⁻ and CD45⁻; CD38⁻ and CD45⁻; or CD34, CD38⁻ and CD45⁻. In other embodiments, the population of PDSC comprises cells that are CD34⁻, CD38⁻, CD45 and HLA-G⁺.

In certain embodiments, the population of PDSC comprises cells that are CD73, CD105, CD200, HLA-G, and/or OCT-4, and do not express CD34, CD38, or CD45. In some embodiments, the population of PDSC comprises cells that also express HLA-ABC (MHC-1) and HLA-DR. These markers can be used to identify PDSC, and to distinguish PDSC from other stem cell types. Because the PDSC can express CD73 and CD105, they can have mesenchymal stem cell-like characteristics. However, because the PDSC can express CD200 and HLA-G, a fetal-specific marker, they can be distinguished from mesenchymal stem cells, e.g., bone marrow-derived mesenchymal stem cells, which express neither CD200 nor HLA-G. In the same manner, the lack of expression of CD34, CD38 and/or CD45 identifies the PDSC as non-hematopoietic stem cells.

In certain embodiments, the population of PDSC comprises cells that are CD200⁺ or HLA-G⁺. In a specific embodiment, the population of PDSC comprises cells that are CD200⁺ and HLA-G⁺. In a specific embodiment, the population of PDSC comprises cells that are CD73⁺ and CD105⁺. In another specific embodiment, the population of PDSC comprises cells that are CD34⁻, CD38⁻ or CD45⁻. In another specific embodiment, the population of PDSC comprises cells that are CD34⁻, CD38⁻ and CD45⁻. In another specific embodiment, the population of PDSC comprises cells that are CD34⁻, CD38⁻, CD45⁻, CD73⁺ and CD105⁺. In another specific embodiment, said CD200⁺ or HLA-G⁺ stem cell facilitates the formation of embryoid-like bodies in a population of placental cells comprising the stem cells, under conditions that allow the formation of embryoid-like bodies. In another specific embodiment, said PDSC is isolated away from placental cells that are not stem cells (e.g., said PDSC). In another specific embodiment, said PDSC is isolated away from PDSC that do not display these markers.

In some embodiments, a PDSC is selected from a plurality of placental cells by a method comprising selecting a CD200⁺ or HLA-G⁺ placental cell, whereby said cell is a PDSC. In a specific embodiment, said population is a population of placental cells. In various embodiments, at least about 10%, at least about 20%, at least about 30%, at least about 40%, at least about 50%, or at least about 60% of said cells are CD200⁺, HLA-G⁺ stem cells. In some embodiments, at least about 70% of said cells are CD200⁺, HLA-G⁺ stem cells. In other embodiments, at least about 90%, 95%, or 99% of said cells are CD200⁺, HLA-G⁺ stem cells. In a specific embodiment of the isolated populations, said stem cells are also CD73⁺ and CD105⁺. In another specific embodiment, said stem cells are also CD34⁻, CD38⁺ or CD45⁻. In a more specific embodiment, said stem cells are also CD34⁻, CD38⁻, CD45⁻, CD73⁺ and CD105⁺. In another embodiment, said isolated population produces one or more embryoid-like bodies when cultured under conditions that allow the formation of embryoid-like bodies. In another specific embodiment, said population of PDSC is isolated away from placental cells that are not stem cells. In another specific embodiment, said population of PDSC is isolated away from PDSC that do not display these markers.

In some embodiments, a PDSC is selected from a plurality of placental cells by a method comprising selecting a population of placental cells wherein at least about 10%, at least about 20%, at least about 30%, at least about 40%, at least about 50% at least about 60%, at least about 70%, at least about 80%, at least about 90%, or at least about 95% of said cells are CD200⁺, HLA-G⁺ stem cells. In a specific embodiment, said selecting comprises selecting stem cells that are also CD73⁺ and CD105⁺. In another specific embodiment, said selecting comprises selecting stem cells that are also CD34⁻, CD38⁻ or CD45⁻. In another specific embodiment, said selecting comprises selecting stem cells that are also CD34⁻, CD38⁻, CD45⁻, CD73⁺ and CD105⁺. In another specific embodiment, said selecting also comprises selecting a population of PDSC that forms one or more embryoid-like bodies when cultured under conditions that allow the formation of embryoid-like bodies.

In another embodiment, the population of PDSC comprises cells that are CD73⁺, CD105⁺, and CD200⁺. In another specific embodiment, the population of PDSC comprises cells that are HLA-G⁺. In another specific embodiment, the population of PDSC comprises cells that are CD34⁻, CD38⁻ or CD45⁻. In another specific embodiment, the population of PDSC comprises cells that are CD34⁻, CD38 and CD45⁻. In a more specific embodiment, the population of PDSC comprises cells that are CD34⁻, CD38⁻, CD45⁻, and HLA-G⁺. In another specific embodiment, the isolated CD73⁺, CD105⁺, and CD200⁺ stem cell facilitates the formation of one or more embryoid-like bodies in a population of placental cells comprising the stem cell, when the population is cultured under conditions that allow the formation of embryoid-like bodies. In another specific embodiment, said PDSC is isolated away from placental cells that are not stem cells. In another specific embodiment, said PDSC is isolated away from PDSC that do not display these markers.

In some embodiments, the PDSC is selected from a plurality of placental cells by a method comprising selecting a CD73⁺, CD105⁺, and CD200⁺ placental cell, whereby said placental cell is a PDSC. In various embodiments, at least about 10%, at least about 20%, at least about 30%, at least about 40%, at least about 50%, or at least about 60% of said cells are CD73⁺, CD105⁺, CD200⁺ stem cells. In another embodiment, at least about 70% of said cells in said population of cells are CD73⁺, CD105⁺, CD200⁺ stem cells. In another embodiment, at least about 90%, 95% or 99% of said cells in said population of PDSC are CD73⁺, CD105⁺, CD200⁺ stem cells. In a specific embodiment, the population of PDSC comprises cells that are HLA-G⁺. In another specific embodiment, the population of PDSC comprises cells that are CD34⁻, CD38⁻ or CD45⁻. In another specific embodiment, the population of PDSC comprises cells that are CD34⁻, CD38⁻ and CD45⁻. In a more specific embodiment, the population of PDSC comprises cells that are CD34⁻, CD38⁻, CD45⁻, and HLA-G⁺. In another specific embodiment, said population of cells produces one or more embryoid-like bodies when cultured under conditions that allow the formation of embryoid-like bodies. In another specific embodiment, said population of PDSC is isolated away from placental cells that are not stem cells. In another specific embodiment, said population of PDSC is isolated away from PDSC that do not display these characteristics.

In another embodiment, a PDSC population is selected from a plurality of placental cells by a method comprising selecting a population of placental cells wherein at least about 10%, at least about 20%, at least about 30%, at least about 40%, at least about 50%, at least about 60%, at least about 70%, at least about 80%, at least about 90%, or at least about 95% of said cells are CD73⁺, CD105⁺, CD200⁺ stem cells. In a specific embodiment, said selecting comprises selecting stem cells that are also HLA-G⁺. In another specific embodiment, said selecting comprises selecting stem cells that are also CD34⁻, CD38⁻ or CD45⁻. In another specific embodiment, said selecting comprises selecting stem cells that are also CD34⁻, CD38⁻ and CD45⁻. In another specific embodiment, said selecting comprises selecting stem cells that are also CD34⁻, CD38⁻, CD45⁻, and HLA-G⁺. In another specific embodiment, said selecting additionally comprises selecting a population of placental cells that produces one or more embryoid-like bodies when the population is cultured under conditions that allow the formation of embryoid-like bodies.

In some embodiments, the population of PDSC comprises cells that are CD200⁺ and OCT-4⁺. In a specific embodiment, the population of PDSC comprises cells that are CD73⁺ and CD105⁺. In another specific embodiment, the population of PDSC comprises cells that are HLA-G⁺. In another specific embodiment, the population of PDSC comprises cells that are CD34⁻, CD38⁻ or CD45⁻. In another specific embodiment, the population of PDSC comprises cells that are CD34⁻, CD38⁻ and CD45⁻. In a more specific embodiment, the population of PDSC comprises cells that are CD34⁻, CD38⁻, CD45⁻, CD73⁺, CD105⁺ and HLA-G⁺. In another specific embodiment, the PDSC facilitates the production of one or more embryoid-like bodies by a population of placental cells that comprises the stem cell, when the population is cultured under conditions that allow the formation of embryoid-like bodies. In another specific embodiment, said PDSC is isolated away from placental cells that are not stem cells. In another specific embodiment, said PDSC is isolated away from PDSC that do not display these markers.

In another embodiment, a PDSC is selected from a plurality of placental cells by a method comprising selecting a CD200⁺ and OCT-4⁺ placental cell, whereby said cell is a PDSC. In a specific embodiment, said selecting comprises selecting a placental cell that is also HLA-G⁺. In another specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also CD34⁻, CD38⁻, CD45⁻, CD73⁺, CD105⁺ and HLA-G⁺. In another specific embodiment, said selecting comprises selecting a PDSC that also facilitates the production of one or more embryoid-like bodies by a population of placental cells that comprises the stem cell, when the population is cultured under conditions that allow the formation of embryoid-like bodies.

In some embodiments, the population of PDSC comprises, e.g., that is enriched for, CD200⁺, OCT-4⁺ stem cells. In various embodiments, at least about 10%, at least about 20%, at least about 30%, at least about 40%, at least about 50%, or at least about 60% of said cells are CD200⁺, OCT-4⁺ stem cells. In another embodiment, at least about 70% of said cells are said CD200⁺, OCT-4⁺ stem cells. In another embodiment, at least about 90%, 95%, or 99% of said cells are said CD200⁺, OCT-4⁺ stem cells. In a specific embodiment, the population of PDSC comprises cells that are CD73⁺ and CD105⁺. In another specific embodiment, the population of PDSC comprises cells that are HLA-G⁺. In another specific embodiment, the population of PDSC comprises cells that are CD34⁻, CD38⁻ and CD45⁻. In a more specific embodiment, the population of PDSC comprises cells that are CD34⁻, CD38⁻, CD45⁻, CD73⁺, CD105⁺ and HLA-G⁺. In another specific embodiment, the population produces one or more embryoid-like bodies when cultured under conditions that allow the formation of embryoid-like bodies. In another specific embodiment, said population of PDSC is isolated away from placental cells that are not stem cells. In another specific embodiment, said population of PDSC is isolated away from PDSC that do not display these characteristics.

In another embodiment, a PDSC population is selected from a plurality of placental cells by a method comprising selecting a population of placental cells wherein at least about 10%, at least about 20%, at least about 30%, at least about 40%, at least about 50% at least about 60%, at least about 70%, at least about 80%, at least about 90%, or at least about 95% of said cells are CD200⁺, OCT-4⁺ stem cells. In a specific embodiment, said selecting comprises selecting stem cells that are also CD73⁺ and CD105⁺. In another specific embodiment, said selecting comprises selecting stem cells that are also HLA-G⁺. In another specific embodiment, said selecting comprises selecting stem cells that are also CD34⁻, CD38⁻ and CD45⁻. In another specific embodiment, said stem cells are also CD34⁻, CD38⁻, CD45⁻, CD73⁺, CD105⁺ and HLA-G⁺.

In some embodiments, the population of PDSC comprises cells that are CD73⁺, CD105⁺ and HLA-G⁺. In another specific embodiment, the population of PDSC comprises cells that are CD34⁻, CD38⁻ or CD45⁻. In another specific embodiment, the population of PDSC comprises cells that are CD34⁻, CD38⁻ and CD45⁻. In another specific embodiment, the population of PDSC comprises cells that are OCT-4⁺. In another specific embodiment, the population of PDSC comprises cells that are CD200⁺. In a more specific embodiment, the population of PDSC comprises cells that are CD34⁻, CD38⁻, CD45⁻, OCT-4⁺ and CD200⁺. In another specific embodiment, said PDSC facilitates the formation of embryoid-like bodies in a population of placental cells comprising said stem cell, when the population is cultured under conditions that allow the formation of embryoid-like bodies. In another specific embodiment, said PDSC is isolated away from placental cells that are not stem cells. In another specific embodiment, said PDSC is isolated away from PDSC that do not display these characteristics.

In another embodiment, a PDSC is selected from a plurality of placental cells, comprising selecting a CD73⁺, CD105⁺ and HLA-G⁺ placental cell, whereby said cell is a PDSC. In various embodiments, at least about 10%, at least about 20%, at least about 30%, at least about 40%, at least about 50%, or at least about 60% of said cells are CD73⁺, CD105⁺ and HLA-G⁺ stem cells. In another embodiment, at least about 70% of said cells are CD73⁺, CD105⁺ and HLA-G⁺. In another embodiment, at least about 90%, 95% or 99% of said cells are CD73⁺, CD105⁺ and HLA-G⁺ stem cells. In a specific embodiment of the above populations, said stem cells are CD34⁻, CD38⁻ or CD45⁻. In another specific embodiment, said stem cells are CD34⁻, CD38⁻ and CD45⁻. In another specific embodiment, said stem cells are OCT-4⁺. In another specific embodiment, said stem cells are CD200⁺. In a more specific embodiment, said stem cells are CD34⁻, CD38⁻, CD45⁻, OCT-4⁺ and CD200⁺. In another specific embodiment, said population of PDSC is isolated away from placental cells that are not stem cells. In another specific embodiment, said population of PDSC is isolated away from PDSC that do not display these characteristics.

In another embodiment, a PDSC population is selected from a plurality of placental cells by a method comprising selecting a population of placental cells wherein a majority of said cells are CD73⁺, CD105⁺ and HLA-G⁺. In a specific embodiment, said majority of cells are also CD34⁻, CD38⁻ and/or CD45⁻. In another specific embodiment, said majority of cells are also CD200⁺. In another specific embodiment, said majority of cells are also CD34⁻, CD38⁻, CD45⁻, OCT-4⁺ and CD200⁺.

In another embodiment, the population of PDSC comprises cells that are CD73⁺ and CD105⁺ and which facilitate the formation of one or more embryoid-like bodies in a population of isolated placental cells comprising said stem cell when said population is cultured under conditions that allow formation of embryoid-like bodies. In various embodiments, at least about 10%, at least about 20%, at least about 30%, at least about 40%, at least about 50% at least about 60%, at least about 70%, at least about 80%, at least about 90%, or at least about 95% of said isolated placental cells are CD73⁺, CD105⁺ stem cells. In a specific embodiment, the population of PDSC comprises cells that are CD34⁻, CD38⁻ or CD45⁻. In another specific embodiment, the population of PDSC comprises cells that are CD34⁻, CD38⁻ and CD45⁻. In another specific embodiment, the population of PDSC comprises cells that are OCT-4⁺. In a more specific embodiment, the population of PDSC comprises cells that are OCT-4⁺, CD34⁻, CD38⁻ and CD45⁻. In other specific embodiments, said population has been expanded, for example, has been passaged at least once, at least three times, at least five times, at least 10 times, at least 15 times, or at least 20 times. In another specific embodiment, said population of PDSC is isolated away from placental cells that are not stem cells. In another specific embodiment, said population of PDSC is isolated away from PDSC that do not display these characteristics.

In some embodiments, the population of PDSC comprises cells that are OCT-4⁺ and which facilitate formation of one or more embryoid-like bodies in a population of isolated placental cells comprising said stem cell when cultured under conditions that allow formation of embryoid-like bodies. In various embodiments, at least 10%, at least about 20%, at least about 30%, at least about 40%, at least about 50% at least about 60%, at least about 70%, at least about 80%, at least about 90%, or at least about 95% of said isolated placental cells are OCT4⁺ stem cells. In a specific embodiment, the population of PDSC comprises cells that are CD73⁻ and CD105⁺. In another specific embodiment, the population of PDSC comprises cells that are CD34⁻, CD38⁻, or CD45⁻. In another specific embodiment, the population of PDSC comprises cells that are CD200⁺. In a more specific embodiment, the population of PDSC comprises cells that are CD73⁺, CD105⁺, CD200⁺, CD34⁻, CD38⁻, and CD45⁻. In another specific embodiment, said population has been expanded, for example, passaged at least once, at least three times, at least five times, at least 10 times, at least 15 times, or at least 20 times. In another specific embodiment, said population of PDSC is isolated away from placental cells that are not stem cells. In another specific embodiment, said population of PDSC is isolated away from PDSC that do not display these characteristics.

In another embodiment, the population of PDSC comprises cells that are CD10⁺, CD34⁻, CD105⁺, and CD200⁺. In some embodiments, at least about 70%, at least about 80%, at least about 90%, at least about 95% or at least about 99% of said PDSC in the population of PDSC are CD10⁺, CD34⁻, CD105⁺, CD200⁺. In a specific embodiment, the population of PDSC comprises cells that are additionally CD90⁺ and CD45⁻. In a specific embodiment, said stem cell or population of PDSC is isolated away from placental cells that are not stem cells. In another specific embodiment, said stem cell or population of PDSC is isolated away from PDSC that do not display these characteristics. In another specific embodiment, said isolated PDSC is non-maternal in origin. In another specific embodiment, at least about 90%, at least about 95%, or at least about 99% of said cells in said isolated population of PDSC, are non-maternal in origin.

In another embodiment, the population of PDSC comprises cells that are HLA-A,B,C⁻, CD45⁻, CD133⁻ and CD34⁻. In some embodiments, at least about 70%, at least about 80%, at least about 90%, at least about 95% or at least about 99% of said PDSC in the population of PDSC are HLA-A,B,C⁻, CD45⁻, CD133⁻ and CD34⁻. In a specific embodiment, said stem cell or population of PDSC is isolated away from placental cells that are not stem cells. In another specific embodiment, said population of PDSC is isolated away from PDSC that do not display these characteristics. In another specific embodiment, said isolated PDSC is non-maternal in origin. In another specific embodiment, at least about 90%, at least about 95%, or at least about 99% of said cells in said isolated population of PDSC, are non-maternal in origin. In another embodiment, the a method of obtaining a PDSC that is HLA-A,B,C⁻, CD45⁻, CD133⁻ and CD34 comprises isolating said cell from placental perfusate.

In another embodiment, the population of PDSC comprises cells that are CD10⁺, CD13⁺, CD33⁺, CD45⁻, CD117⁻ and CD133⁻. In some embodiments, at least about 70%, at least about 80%, at least about 90%, at least about 95% or at least about 99% of said PDSC in the population of PDSC are CD10⁺, CD13⁺, CD33⁺, CD45⁻, CD117⁻ and CD133⁻. In a specific embodiment, said stem cell or population of PDSC is isolated away from placental cells that are not stem cells. In another specific embodiment, said isolated PDSC is non-maternal in origin. In another specific embodiment, at least about 90%, at least about 95%, or at least about 99% of said cells in said isolated population of PDSC, are non-maternal in origin. In another specific embodiment, said stem cell or population of PDSC is isolated away from PDSC that do not display these characteristics. In another embodiment, the method of obtaining a PDSC that is CD10⁺, CD13⁺, CD33⁺, CD45⁻, CD117⁻ and CD133⁻ comprises isolating said cell from placental perfusate.

In another embodiment, provided herein are isolated PDSC that are CD10⁻, CD33⁻, CD44⁺, CD45⁻, and CD117⁻. Also provided is an isolated population of PDSC, wherein at least about 70%, at least about 80%, at least about 90%, at least about 95% or at least about 99% of said PDSC are CD10⁻, CD33⁻, CD44⁺, CD45⁻, and CD117⁻. In a specific embodiment, said stem cell or population of PDSC is isolated away from placental cells that are not stem cells. In another specific embodiment, said isolated PDSC is non-maternal in origin. In another specific embodiment, at least about 90%, at least about 95%, or at least 99% of said cells in said isolated population of PDSC, are non-maternal in origin. In another specific embodiment, said stem cell or population of PDSC is isolated away from PDSC that do not display these characteristics. In another embodiment, provided is a method of obtaining a PDSC that is CD10⁻, CD33⁻, CD44⁺, CD45⁻, CD117⁻ comprising isolating said cell from placental perfusate.

In another embodiment, the population of PDSC comprises cells that are CD10⁻, CD13⁻, CD33⁻, CD45⁻, and CD117⁻. In some embodiments, at least about 70%, at least about 80%, at least about 90%, at least about 95% or at least about 99% of said PDSC of the population of PDSC are CD10⁻, CD13⁻, CD33⁻, CD45⁻, and CD117⁻. In a specific embodiment, said stem cell or population of PDSC is isolated away from placental cells that are not stem cells. In another specific embodiment, said isolated PDSC is non-maternal in origin. In another specific embodiment, at least about 90%, at least about 95%, or at least 99% of said cells in said isolated population of PDSC, are non-maternal in origin. In another specific embodiment, said stem cell or population of PDSC is isolated away from PDSC that do not display these characteristics. In another embodiment, the method of obtaining a PDSC that is CD10⁻, CD13⁻, CD33⁻, CD45⁻, and CD117⁺comprises isolating said cell from placental perfusate.

In another embodiment, the population of PDSC comprises cells that are HLA A,B,C⁻, CD45⁻, CD34⁻, CD133⁻, positive for CD10, CD13, CD38, CD44, CD90, CD105, CD200 and/or HLA-G, and/or negative for CD117. In some embodiments, the population of PDSC comprises cells that are HLA A,B,C⁻, CD45⁻, CD34⁻, CD133⁻, and at least about 20%, 25%, 30%, 35%, 40%, 45%, 50%, 55%, 60%, 65%, 70%, 75%, 80%, 85%, 90%, 95%, 98% or about 99% of the stem cells in the population are positive for CD10, CD13, CD38, CD44, CD90, CD105, CD200 and/or HLA-G, and/or negative for CD117. In a specific embodiment, said stem cell or population of PDSC is isolated away from placental cells that are not stem cells. In another specific embodiment, said isolated PDSC is non-maternal in origin. In another specific embodiment, at least about 90%, at least about 95%, or at least about 99%, of said cells in said isolated population of PDSC, are non-maternal in origin. In another specific embodiment, said stem cell or population of PDSC is isolated away from PDSC that do not display these characteristics. In another embodiment, the method of obtaining a PDSC that is HLA-A,B,C⁻, CD45⁻, CD34⁻, CD133⁻ and positive for CD10, CD13, CD38, CD44, CD90, CD105, CD200 and/or HLA-G, and/or negative for CD117 comprises isolating said cell from placental perfusate.

In another embodiment, the population of PDSC comprises cells that are CD200⁺ and CD10⁺, as determined by antibody binding, and CD117⁻, as determined by both antibody binding and RT-PCR. In another embodiment, the population of PDSC comprises cells that are CD10⁺, CD29⁻, CD54⁺, CD200⁺, HLA-G⁺, HLA class I⁻ and β-2-microglobulin⁻. In another embodiment, the population of PDSC comprises cells that express at least one marker that is at least two-fold higher than for a mesenchymal stem cell (e.g., a bone marrow-derived mesenchymal stem cell). In another specific embodiment, said isolated PDSC is non-maternal in origin. In another specific embodiment, at least about 90%, at least about 95%, or at least 99%, of said cells in said isolated population of PDSC, are non-maternal in origin.

In another embodiment, the population of PDSC is an isolated population of PDSC, wherein a plurality of said PDSC are positive for aldehyde dehydrogenase (ALDH), as assessed by an aldehyde dehydrogenase activity assay. Such assays are known in the art (see, e.g., Bostian and Betts, Biochem. J. 1978, 173:787-798). In a specific embodiment, said ALDH assay uses ALDEFLUOR™ (Aldagen, Inc., Ashland, Oreg.) as a marker of aldehyde dehydrogenase activity. In a specific embodiment, said plurality is between about 3% and about 25% of cells in said population of cells. In another embodiment, provided is a population of umbilical cord stem cells, wherein a plurality of said umbilical cord stem cells are positive for aldehyde dehydrogenase, as assessed by an aldehyde dehydrogenase activity assay that uses ALDEFLUOR™ as an indicator of aldehyde dehydrogenase activity. In a specific embodiment, said plurality is between about 3% and about 25% of cells in said population of cells. In another embodiment, said population of PDSC or umbilical cord stem cells shows at least three-fold, or at least five-fold, higher ALDH activity than a population of bone marrow-derived mesenchymal stem cells having the same number of cells and cultured under the same conditions.

In certain embodiments, the PDSC, or population of PDSC, has been passaged at least 1, 2, 3, 4, 5, 6, 7, 8, 9, 10, 12, 14, 16, 18, or 20 times, or more, or proliferated (expanded) at least, about or nor more than, 1, 2, 3, 4, 5, 6, 7, 8, 9, 10, 12, 14, 16, 18, 20, 22, 24, 26, 28, 30, 32, 34, 36, 38 or 40 population doublings.

In a specific embodiment of the isolated PDSC or populations of cells comprising the isolated PDSC, said cells or population have been passaged at least, about, or no more than 3 times, 4 times, 5 times, or 6 times. In a specific embodiment of the isolated PDSC or populations of cells comprising the isolated PDSC, said cells or population have been passaged at least, about, or no more than 3-10 times, 4-8 times, or 5-7 times. In a specific embodiment of the isolated PDSC or populations of cells comprising the isolated PDSC, said cells or population have been proliferated for at least, about, or no more than, 2, 3, 4, 5, or 6 population doublings. In a specific embodiment of the isolated PDSC or populations of cells comprising the isolated PDSC, said cells or population have been proliferated for at least, about, or no more than, 3-10, 4-8, or 5-7 population doublings. In a specific embodiment of the isolated PDSC or populations of cells comprising the isolated PDSC, said cells or population have been proliferated for at least, about, or no more than, 6-10, 11-14, 15-30, 30-45, or 18-26, or 24-38 population doublings. In another specific embodiment of said isolated PDSC or populations of cells comprising the isolated PDSC, said cells or population are primary isolates. In another specific embodiment of the isolated PDSC, or populations of cells comprising isolated PDSC, that are disclosed herein, said isolated PDSC are fetal in origin (that is, have the fetal genotype).

In certain embodiments, said isolated PDSC do not differentiate during culturing in growth medium, i.e., medium formulated to promote proliferation, e.g., during proliferation in growth medium. In another specific embodiment, said isolated PDSC do not require a feeder layer in order to proliferate. In another specific embodiment, said isolated PDSC do not differentiate in culture in the absence of a feeder layer, solely because of the lack of a feeder cell layer.

In certain embodiments of any of the populations of cells comprising the isolated PDSC described herein, the PDSC in said populations of cells are substantially free of cells having a maternal genotype; e.g., at least 40%, 45%, 50%, 55%, 60%, 65%, 70%, 75%, 80%, 85%, 90%, 95%, 98%, or 99% of the PDSC in said population have a fetal genotype. In certain other embodiments of any of the populations of cells comprising the isolated PDSC described herein, the populations of cells comprising said PDSC are substantially free of cells having a maternal genotype; e.g., at least 40%, 45%, 50%, 55%, 60%, 65%, 70%, 75%, 80%, 85%, 90%, 95%, 98%, or 99% of the cells in said population have a fetal genotype.

In a specific embodiment of any of the above PDSC or cell populations, the karyotype of the cells, or at least about 95% or about 99% of the cells in said population, is normal. In another specific embodiment of any of the above PDSC or cell populations, the cells, or cells in the population of cells, are non-maternal in origin.

In a specific embodiment of any of the embodiments of PDSC disclosed herein, the PDSC are genetically stable, displaying a normal diploid chromosome count and a normal karyotype.

Isolated PDSC, or isolated populations of PDSC, bearing any of the above combinations of markers, can be combined in any ratio. In certain embodiments, it is contemplated that any two or more of the above PDSC populations can be isolated or enriched to form a PDSC population. For example, provided herein is an isolated population of PDSC comprising a first population of PDSC defined by one of the marker combinations described above and a second population of PDSC defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In a similar manner, any three, four, five or more of the above-described PDSC or PDSC populations can be combined.

In other embodiments, also provided herein are PDSC that are obtained by disruption of placental tissue, with or without enzymatic digestion, followed by culture or perfusion, as provided elsewhere herein. For example, in certain embodiments, provided is an isolated population of PDSC that is produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental cells that comprises PDSC; and isolating a plurality of said PDSC from said population of cells. In a specific embodiment, the perfusion solution is passed through both the umbilical vein and umbilical arteries and collected after it exudes from the placenta. Populations of PDSC produced by this method typically comprise a mixture of fetal and maternal cells. In another specific embodiment, the perfusion solution is passed through the umbilical vein and collected from the umbilical arteries, or passed through the umbilical arteries and collected from the umbilical vein. Populations of PDSC produced by this method typically are substantially exclusively fetal in origin; that is, e.g., greater than 90%, 95%, 99%, or 99.5% of the PDSC in the population are fetal in origin.

In various embodiments, the PDSC, contained within a population of cells obtained from perfusion of a placenta, are at least 50%, 60%, 70%, 80%, 90%, 95%, 99% or at least 99.5% of said population of placental cells. In another specific embodiment, the PDSC collected by perfusion comprise fetal and maternal cells. In another specific embodiment, the PDSC collected by perfusion are at least 50%, 60%, 70%, 80%, 90%, 95%, 99% or at least 99.5% fetal cells.

In another specific embodiment, provided herein is a composition comprising a population of the isolated PDSC, as described herein, collected (isolated) by perfusion, wherein said composition comprises at least a portion of the perfusion solution used to isolate the PDSC.

In some embodiments, an isolated population of the PDSC described herein that is produced according to a method comprising digesting placental tissue with a tissue-disrupting enzyme to obtain a population of placental cells comprising PDSC, and isolating a plurality of PDSC from the remainder of said placental cells. The whole, or any part of, the placenta can be digested to obtain PDSC. In specific embodiments, for example, said placental tissue is a whole placenta, an amniotic membrane, chorion, a combination of amnion and chorion, or a combination of any of the foregoing. In other specific embodiment, the tissue-disrupting enzyme is trypsin or collagenase. In various embodiments, the PDSC, contained within a population of cells obtained from digesting a placenta, are at least 50%, 60%, 70%, 80%, 90%, 95%, 99% or at least 99.5% of said population of placental cells.

Gene profiling confirms that isolated PDSC, and populations of isolated PDSC, are distinguishable from other cells, e.g., mesenchymal stem cells or bone marrow-derived stem cells. The PDSC described herein, can be distinguished from mesenchymal stem cells on the basis of the expression of one or more genes, the expression of which is specific to PDSC or umbilical cord stem cells in comparison to bone marrow-derived mesenchymal stem cells. In particular, As provided in more detail elsewhere herein, PDSC can be distinguished from mesenchymal stem cells on the basis of the expression of one or more gene, the expression of which is significantly higher (that is, at least twofold higher) in PDSC than in mesenchymal stem cells, wherein the one or more gene is (are) ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, ZC3H12A, or a combination of any of the foregoing, wherein the expression of these genes is higher in PDSC or umbilical cord stem cells than in bone marrow-derived stem cells, when the stem cells are grown under equivalent conditions. In a specific embodiment, the PDSC-specific or umbilical cord stem cell-specific gene is CD200.

The level of expression of these genes can be used to confirm the identity of a population of placental cells, to identify a population of cells as comprising at least a plurality of PDSC, or the like. The population of PDSC, the identity of which is confirmed, can be clonal, e.g., a population of PDSC expanded form a single PDSC, or a mixed population of stem cells, e.g., a population of cells comprising solely PDSC that are expanded from multiple PDSC, or a population of cells comprising PDSC and at least one other type of cell.

The level of expression of these genes can be used to select populations of PDSC. For example, a population of cells, e.g., clonally-expanded cells, is selected if the expression of one or more of these genes is significantly higher in a sample from the population of cells than in an equivalent population of mesenchymal stem cells. Such selecting can be of a population from a plurality of PDSC populations, from a plurality of cell populations, the identity of which is not known, etc.

PDSC can be selected on the basis of the level of expression of one or more such genes as compared to the level of expression in said one or more genes in a mesenchymal stem cell control. In one embodiment, the level of expression of said one or more genes in a sample comprising an equivalent number of mesenchymal stem cells is used as a control. In another embodiment, the control, for PDSC tested under certain conditions, is a numeric value representing the level of expression of said one or more genes in mesenchymal stem cells under said conditions.

The PDSC of the population of PDSC display the above characteristics (e.g., combinations of cell surface markers and/or gene expression profiles) in primary culture, or during proliferation in medium comprising 60% DMEM-LG (Gibco), 40% MCDB-201(Sigma), 2% fetal calf serum (FCS) (Hyclone Laboratories), 1× insulin-transferrin-selenium (ITS), 1× lenolenic-acid-bovine-serum-albumin (LA-BSA), 10⁻⁹ M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin.

In another specific embodiment, provided herein is a composition comprising a population of the isolated PDSC, as described herein, collected (isolated) by perfusion, wherein said composition comprises at least a portion of the perfusion solution used to isolate the PDSC.

Populations of the isolated PDSC described herein can be produced by digesting placental tissue with a tissue-disrupting enzyme to obtain a population of placental cells comprising the PDSC, and isolating, or substantially isolating, a plurality of the PDSC from the remainder of said placental cells. The whole, or any part of, the placenta can be digested to obtain the isolated PDSC described herein. In specific embodiments, for example, said placental tissue can be a whole placenta (e.g., including an umbilical cord), an amniotic membrane, chorion, a combination of amnion and chorion, or a combination of any of the foregoing. In other specific embodiments, the tissue-disrupting enzyme is trypsin or collagenase. In various embodiments, the isolated PDSC, contained within a population of cells obtained from digesting a placenta, are at least 50%, 60%, 70%, 80%, 90%, 95%, 99%, or at least 99.5% of said population of placental cells.

The populations of isolated PDSC described above, and populations of isolated PDSC generally, can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more of the isolated PDSC. Populations of isolated PDSC useful in the methods described herein comprise at least 50%, 55%, 60%, 65%, 70%, 75%, 80%, 85%, 90%, 95%, 98%, or 99% viable isolated PDSC, e.g., as determined by, e.g., trypan blue exclusion.

For any of the above PDSC, or populations of PDSC, the cells or population of PDSC are, or can comprise, cells that have been passaged at least 1, 2, 3, 4, 5, 6, 7, 8, 9, 10, 12, 14, 16, 18, or 20 times, or more, or expanded for 1, 2, 3, 4, 5, 6, 7, 8, 9, 10, 12, 14, 16, 18, 20, 22, 24, 26, 28, 30, 32, 34, 36, 38, or 40 population doublings, or more.

In a specific embodiment of any of the above PDSC populations, the karyotype of the cells, or at least about 95% or about 99% of the cells in said population, is normal. In another specific embodiment of any of the above PDSC populations, the cells, or cells in the population of cells, are non-maternal in origin.

Isolated PDSC, or populations of isolated PDSC, bearing any of the above combinations of markers, can be combined in any ratio. Any two or more of the above PDSC populations can be isolated, or enriched, to form a PDSC population. For example, a population of isolated PDSC comprising a first population of PDSC defined by one of the marker combinations described above can be combined with a second population of PDSC defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described PDSC or PDSC populations can be combined.

In a specific embodiment of the above-mentioned PDSC, the PDSC constitutively secrete IL-6, IL-8 and monocyte chemoattractant protein (MCP-1).

The isolated populations of PDSC described above, and populations of PDSC generally, can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more PDSC.

In certain embodiments, the PDSC useful in the methods provided herein, do not express CD34, as detected by immunolocalization, after exposure to 1 to 100 ng/mL VEGF for 4 to 21 days. In a specific embodiment, said PDSC are adherent to tissue culture plastic. In another specific embodiment, said PDSC induce endothelial cells to form sprouts or tube-like structures, e.g., when cultured in the presence of an angiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™.

In another aspect, the PDSC provided herein, or a population of cells, e.g., a population of PDSC, or a population of cells wherein at least about 50%, 60%, 70%, 80%, 90%, 95%, or 98% of cells in said population of cells are PDSC, secrete one or more, or all, of VEGF, HGF, IL-8, MCP-3, FGF2, follistatin, G-CSF, EGF, ENA-78, GRO, IL-6, MCP-1, PDGF-BB, TIMP-2, uPAR, or galectin-1, e.g., into culture medium in which the cell, or cells, are grown. In another embodiment, the PDSC express increased levels of CD202b, IL-8 and/or VEGF under hypoxic conditions (e.g., less than about 5% O₂) compared to normoxic conditions (e.g., about 20% or about 21% O₂).

In another embodiment, any of the PDSC or populations of cells comprising PDSC described herein can cause the formation of sprouts or tube-like structures in a population of endothelial cells in contact with or proximity to said PDSC. In a specific embodiment, the PDSC are co-cultured with human endothelial cells, which form sprouts or tube-like structures, or support the formation of endothelial cell sprouts, e.g., when cultured in the presence of extracellular matrix proteins such as collagen type I and IV, and/or angiogenic factors such as VEGF, EGF, PDGF, or bFGF, e.g., in or on a substrate such as placental collagen or MATRIGEL™ for at least 4 days. In another embodiment, any of the populations of cells comprising PDSC, described herein, secrete angiogenic factors such as VEGF, hepatocyte growth factor (HGF), PDGF, bFGF, or Interleukin-8 (IL-8) and thereby can induce human endothelial cells to form sprouts or tube-like structures when cultured in the presence of extracellular matrix proteins such as collagen type I and IV, e.g., in or on a substrate such as placental collagen or MATRIGEL™.

In another embodiment, any of the above populations of cells comprising PDSC secretes angiogenic factors. In specific embodiments, the population of cells secretes VEGF, HGF, PDGF, bFGF, or IL-8. In other specific embodiments, the population of cells comprising PDSC secretes one or more angiogenic factors and thereby induces human endothelial cells to migrate in an in vitro wound healing assay. In other specific embodiments, the population of cells comprising PDSC induces maturation, differentiation or proliferation of human endothelial cells, endothelial progenitors, myocytes or myoblasts.

4.3.2 Growth in Culture

The growth of the PDSC described herein, as for any mammalian cell, depends in part upon the particular medium selected for growth. Under optimum conditions, PDSC typically double in number in 3-5 days. During culture, the PDSC provided herein can adhere to a substrate in culture, e.g., the surface of a tissue culture container (e.g., tissue culture dish plastic, fibronectin-coated plastic, and the like) and form a monolayer.

In some embodiments, populations of isolated placental cells that comprise PDSC, when cultured under appropriate conditions, can form embryoid-like bodies, that is, three-dimensional clusters of cells grow atop the adherent stem cell layer. Cells within the embryoid-like bodies express markers associated with very early stem cells, e.g., OCT-4, Nanog, SSEA3 and SSEA4. Cells within the embryoid-like bodies are typically not adherent to the culture substrate, as are the PDSC described herein, but remain attached to the adherent cells during culture. Embryoid-like body cells are dependent upon the adherent PDSC for viability, as embryoid-like bodies do not form in the absence of the adherent stem cells. The adherent PDSC thus facilitate the growth of one or more embryoid-like bodies in a population of placental cells that comprise the adherent PDSC. Without wishing to be bound by theory, the cells of the embryoid-like bodies are thought to grow on the adherent PDSC much as embryonic stem cells grow on a feeder layer of cells. Mesenchymal stem cells, e.g., bone marrow-derived mesenchymal stem cells, do not develop embryoid-like bodies in culture.

In certain embodiments, the population of PDSC are autologous to the subject. In some embodiments, the population of PDSC are allogeneic to the subject. In one embodiment, the population of PDSC are syngeneic to the subject.

The stem cells can be a homogeneous composition or a mixed cell population, for example, enriched with a particular type of stem cell. Homogeneous cell compositions can be obtained, for example, by cell surface markers characteristic of stem cells, or particular types of stem cells, in conjunction with monoclonal antibodies directed to the specific cell surface markers. In some embodiments, the population of PDSC is a homogeneous cell population. In other embodiments, the population of PDSC is a mixed cell population. In one embodiment, the population of PDSC is an enriched PDSC population. In some embodiments, the population of PDSC comprises PSC-100 cells. In another embodiment, the population of PDSC comprises an enriched population of PSC-100 cells. In some embodiments, the population of PDSC comprises PDA-001 cells. In another embodiment, the population of PDSC comprises an enriched population of PDA-001 cells.

4.3.3 Differentiation

The placental cells, useful in the methods provided herein, in certain embodiments are differentiable into different committed cell lineages. For example, in certain embodiments, the placental cells can be differentiated into cells of an adipogenic, chondrogenic, neurogenic, or osteogenic lineage. Such differentiation can be accomplished, e.g., by any method known in the art for differentiating, e.g., bone marrow-derived mesenchymal stem cells into similar cell lineages, or by methods described elsewhere herein. Specific methods of differentiating placental cells into particular cell lineages are disclosed in, e.g., U.S. Pat. Nos. 7,311,905 and 8,057,788, the disclosures of which are hereby incorporated by reference in their entireties.

The PDSC provided herein can exhibit the capacity to differentiate into a particular cell lineage in vitro, in vivo, or in vitro and in vivo. In a specific embodiment, the PDSC provided herein can be differentiated in vitro when placed in conditions that cause or promote differentiation into a particular cell lineage, but do not detectably differentiate in vivo, e.g., in a NOD-SCID mouse model.

### 4.4 Methods of Obtaining PDSC

4.4.1 Stem Cell Collection Composition

Generally, stem cells are obtained from a mammalian placenta using a physiologically-acceptable solution, e.g., a stem cell collection composition. A stem cell collection composition is described in detail in U.S. Provisional Application No. 60/754,969, entitled “Improved Medium for Collecting Placental Cells and Preserving Organs,” filed on Dec. 29, 2005.

The stem cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl, etc.), a culture medium (e.g., DMEM, HDMEM, etc.), and the like.

The stem cell collection composition can comprise one or more components that tend to preserve PDSC, that is, prevent the PDSC from dying, or delay the death of the PDSC, reduce the number of PDSC in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-indol-3-yl)-3-pentylamino-maleimide, pyrrolidine dithiocarbamate, or clonazepam); a TNF-alpha inhibitor; and/or an oxygen-carrying perfluorocarbon (e.g., perfluorooctyl bromide, perfluorodecyl bromide, etc.).

The stem cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE™, hyaluronidase, and the like.

The stem cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The stem cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/L to about 100 g/L, or about 40 g/L to about 60 g/L); an antioxidant (e.g., butylated hydroxyanisole, butylated hydroxytoluene, glutathione, vitamin C or vitamin E present at about 25 M to about 100 M); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/L to about 100,000 units/L); or an amiloride containing compound (e.g., amiloride, ethyl isopropyl amiloride, hexamethylene amiloride, dimethyl amiloride or isobutyl amiloride present at about 1.0 M to about 5 M).

4.4.2 Collection and Handling of Placenta

Generally, a human placenta is recovered shortly after its expulsion after birth. In one embodiment, the placenta is recovered from a patient after informed consent and after a complete medical history of the patient is taken and is associated with the placenta. In some embodiments, the medical history continues after delivery. Such a medical history can be used to coordinate subsequent use of the placenta or the stem cells harvested therefrom. For example, human PDSC can be used, in light of the medical history, for personalized medicine for the infant associated with the placenta, or for parents, siblings or other relatives of the infant.

Prior to recovery of PDSC, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. In some embodiments, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta can be transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. Pat. No. 7,147,626. The placenta can be delivered to the laboratory four to twenty-four hours following delivery. In certain embodiments, the proximal umbilical cord is clamped, such as within 4-5 cm (centimeter) of the insertion into the placental disc prior to cord blood recovery. In other embodiments, the proximal umbilical cord is clamped after cord blood recovery but prior to further processing of the placenta.

The placenta, prior to stem cell collection, can be stored under sterile conditions and at either room temperature or at a temperature of 5 to 25° C. (centigrade). The placenta may be stored for a period of for a period of four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta can be stored in an anticoagulant solution at a temperature of 5 to 25° C. (centigrade). Suitable anticoagulant solutions are well known in the art. For example, a solution of heparin or warfarin sodium can be used. In one embodiment, the anticoagulant solution comprises a solution of heparin (e.g., 1% w/w in 1:1000 solution). The exsanguinated placenta is can be stored for no more than 36 hours before PDSC are collected.

The mammalian placenta or a part thereof, once collected and prepared generally as above, can be treated in any art-known manner, e.g., can be perfused or disrupted, e.g., digested with one or more tissue-disrupting enzymes, to obtain stem cells.

4.4.3 Physical Disruption and Enzymatic Digestion of Placental Tissue

In one embodiment, stem cells are collected from a mammalian placenta by physical disruption of part of all of the organ. For example, the placenta, or a portion thereof, may be, e.g., crushed, sheared, minced, diced, chopped, macerated or the like. The tissue can then be cultured to obtain a population of stem cells. Typically, the placental tissue is disrupted using, e.g., in a stem cell collection composition, as provided elsewhere herein.

The placenta can be dissected into components prior to physical disruption and/or enzymatic digestion and stem cell recovery. PDSC can be obtained from all or a portion of the amniotic membrane, chorion, umbilical cord, placental cotyledons, or any combination thereof, including from a whole placenta. PDSC can be obtained from placental tissue comprising amnion and chorion. Typically, PDSC can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume. Any method of physical disruption can be used, provided that the method of disruption leaves a plurality, or even a majority, such as at least 60%, 70%, 80%, 90%, 95%, 98%, or 99% of the cells in said organ viable, as determined by, e.g., trypan blue exclusion.

Stem cells can generally be collected from a placenta, or portion thereof, at any time within about the first three days post-expulsion, such as between about 8 hours and about 18 hours post-expulsion.

In a specific embodiment, the disrupted tissue is cultured in tissue culture medium suitable for the proliferation of PDSC, e.g., as described elsewhere herein.

In another specific embodiment, stem cells are collected by physical disruption of placental tissue, wherein the physical disruption includes enzymatic digestion, which can be accomplished by use of one or more tissue-digesting enzymes. The placenta, or a portion thereof, may also be physically disrupted and digested with one or more enzymes, and the resulting material then immersed in, or mixed into, a stem cell collection composition.

An exemplary stem cell collection composition comprises one or more tissue-disruptive enzyme(s). Enzymatic digestion can use a combination of enzymes, e.g., a combination of a matrix metalloprotease and a neutral protease, for example, a combination of collagenase and dispase. In one embodiment, enzymatic digestion of placental tissue uses a combination of a matrix metalloprotease, a neutral protease, and a mucolytic enzyme for digestion of hyaluronic acid, such as a combination of collagenase, dispase, and hyaluronidase or a combination of LIBERASE™ (Boehringer Mannheim Corp., Indianapolis, Ind.) and hyaluronidase. Other enzymes that can be used to disrupt placenta tissue include papain, deoxyribonucleases, serine proteases, such as trypsin, chymotrypsin, or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate can be diluted so as to avoid trapping stem cells within the viscous digest.

Any combination of tissue digestion enzymes can be used. Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to liberate PDSC. For example, in one embodiment, a placenta, or part thereof, is digested first with an appropriate amount of collagenase I at about 1 to about 2 mg/ml for, e.g., 30 minutes, followed by digestion with trypsin, at a concentration of about 0.25%, for, e.g., 10 minutes, at 37° C. Serine proteases can be used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

In one embodiment, a digestion can proceed as follows. Approximately a gram of placental tissue is obtained and minced. The tissue is digested in 10 mL of a solution comprising about 1 mg/mL collagenase 1A and about 0.25% trypsin at 37° C. in a shaker at about 100 RPM. The digestate is washed three times with culture medium, and the washed cells are seeded into 2 T-75 flasks. The cells are then isolated by differential adherence, and characterized for, e.g., viability, cell surface markers, differentiation, and the like.

It will be appreciated that where an entire placenta, or portion of a placenta comprising both fetal and maternal cells (for example, where the portion of the placenta comprises the chorion or cotyledons), the PDSC collected will comprise a mix of PDSC derived from both fetal and maternal sources. Where a portion of the placenta that comprises no, or a negligible number of, maternal cells (for example, amnion), the PDSC collected will comprise almost exclusively fetal PDSC.

Stem cells can be isolated from disrupted tissue by differential trypsinization followed by culture in one or more new culture containers in fresh proliferation medium, optionally followed by a second differential trypsinization step.

4.4.4. Placental Perfusion

PDSC can also be obtained by perfusion of the mammalian placenta. Methods of perfusing mammalian placenta to obtain stem cells are disclosed, e.g., in Hariri, U.S. Application Publication No. 2002/0123141, and in related U.S. Provisional Application No. 60/754,969, entitled “Improved Medium for Collecting PDSC and Preserving Organs,” filed on Dec. 29, 2005.

PDSC can be collected by perfusion, e.g., through the placental vasculature, using, e.g., a stem cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. The perfusion solution can be forced through the placenta using a pump, e.g., a peristaltic pump. The umbilical vein can be, e.g., cannulated with a cannula, e.g., a TEFLON™ or plastic cannula, that is connected to a sterile connection apparatus, such as sterile tubing. The sterile connection apparatus is connected to a perfusion manifold.

In preparation for perfusion, the placenta can be oriented (e.g., suspended) in such a manner that the umbilical artery and umbilical vein are located at the highest point of the placenta. The placenta can be perfused by passage of a perfusion fluid through the placental vasculature and surrounding tissue. The placenta can also be perfused by passage of a perfusion fluid into the umbilical vein and collection from the umbilical arteries, or passage of a perfusion fluid into the umbilical arteries and collection from the umbilical vein.

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

It will be appreciated that perfusion using the pan method, that is, whereby perfusate is collected after it has exuded from the maternal side of the placenta, results in a mix of fetal and maternal cells. As a result, the cells collected by this method comprise a mixed population of PDSC of both fetal and maternal origin. In contrast, perfusion solely through the placental vasculature in the closed circuit method, whereby perfusion fluid is passed through one or two placental vessels and is collected solely through the remaining vessel(s), results in the collection of a population of PDSC almost exclusively of fetal origin.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 mL of perfusion solution. Cells in the perfusate are then collected, e.g., by centrifugation.

In one embodiment, the proximal umbilical cord is clamped during perfusion, such as, within 4-5 cm of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 mL (milliliter) of perfusion fluid is adequate to initially exsanguinate the placenta, but more or less perfusion fluid may be used depending on the observed results.

The volume of perfusion liquid used to collect PDSC may vary depending upon the number of stem cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the stem cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., P3-mercaptoethanol (0.1 mM); antibiotics such as streptomycin (e.g., at 40-100 μg/mL), penicillin (e.g., at 40 U/mL), amphotericin B (e.g., at 0.5 μg/mL). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In one embodiment, perfusion of the placenta and collection of perfusion solution, e.g., stem cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled. In a specific embodiment, stem cells are collected at a time or times between about 8 hours and about 18 hours post-expulsion.

Without wishing to be bound by any theory, after exsanguination and a sufficient time of perfusion of the placenta, PDSC are believed to migrate into the exsanguinated and perfused microcirculation of the placenta where, according to methods provided herein, they are collected, such as by washing into a collecting vessel by perfusion. Perfusing the isolated placenta not only serves to remove residual cord blood but also provide the placenta with the appropriate nutrients, including oxygen. The placenta may be cultivated and perfused with a similar solution which was used to remove the residual cord blood cells, e.g., without the addition of anticoagulant agents.

Perfusion according to the methods provided herein can result in the collection of significantly more PDSC than the number obtainable from a mammalian placenta not perfused with said solution, and not otherwise treated to obtain stem cells (e.g., by tissue disruption, e.g., enzymatic digestion). In this context, “significantly more” means at least 10% more. Perfusion according to the methods provided herein can yield significantly more PDSC than, e.g., the number of PDSC obtainable from culture medium in which a placenta, or portion thereof, has been cultured.

Stem cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The stem cells are washed after several minutes with a cold (e.g., 4° C.) stem cell collection composition. 4.4.5 Isolation, Sorting, and Characterization of PDSC

Stem cells from mammalian placenta, whether obtained by perfusion or enzymatic digestion, can initially be purified from (i.e., be isolated from) other cells by Ficoll® gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for stem cell maintenance, e.g., IMDM serum-free medium containing 2 U/mL heparin and 2 mM EDTA (GibcoBRL, NY). The total mononuclear cell fraction can be isolated, e.g., using Lymphoprep™ (Nycomed Pharma, Oslo, Norway) according to the manufacturer's recommended procedure.

As used herein, “isolating” PDSC means to remove at least 20%, 30%, 40%, 50%, 60%, 70%, 80%, 90%, 95% or 99% of the cells with which the stem cells are normally associated in the intact mammalian placenta. A stem cell from an organ is “isolated” when it is present in a population of cells that comprises fewer than 50% of the cells with which the stem cell is normally associated in the intact organ.

Placental cells obtained by perfusion or digestion can, for example, be further, or initially, isolated by differential trypsinization using, e.g., a solution of 0.05% trypsin with 0.2% EDTA (Sigma, St. Louis, Mo.). Differential trypsinization is possible because PDSC typically detach from plastic surfaces within about five minutes whereas other adherent populations typically require more than 20-30 minutes incubation. The detached PDSC can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, such as fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks can be examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. In specific embodiments, the technique is flow cytometry. In other specific embodiments, the technique is FACS. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺ Antibodies to cell surface markers (e.g., CD markers such as CD34) and the sequence of stem cell-specific genes, such as OCT-4, are well-known in the art.

Placental cells, particularly cells that have been isolated by Ficoll® separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, Methods Enzymol 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, stem cells from placenta are sorted on the basis of expression of the markers CD34, CD38, CD44, CD45, CD73, CD105, OCT-4 and/or HLA-G. This can be accomplished in connection with procedures to select stem cells on the basis of their adherence properties in culture. For example, an adherence selection stem can be accomplished before or after sorting on the basis of marker expression. In one embodiment, for example, cells are sorted first on the basis of their expression of CD34; CD34⁻ cells are retained, and cells that are CD200⁺HLA-G⁺, are separated from all other CD34⁻ cells. In another embodiment, cells from placenta are based on their expression of markers CD200 and/or HLA-G; for example, cells displaying either of these markers are isolated for further use. Cells that express, e.g., CD200 and/or HLA-G can, in a specific embodiment, be further sorted based on their expression of CD73 and/or CD105, or epitopes recognized by antibodies SH2, SH3 or SH4, or lack of expression of CD34, CD38 or CD45. For example, in one embodiment, placental cells are sorted by expression, or lack thereof, of CD200, HLA-G, CD73, CD105, CD34, CD38 and CD45, and placental cells that are CD200⁺, HLA-G⁺, CD73⁺, CD105⁺, CD34⁻, CD38⁻ and CD45⁻ are isolated from other placental cells for further use.

With respect to antibody-mediated detection and sorting of PDSC, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against HLA-G (available from Serotec, Raleigh, N.C.), CD10 (available from BD Immunocytometry Systems, San Jose, Calif.), CD44 (available from BD Biosciences Pharmingen, San Jose, Calif.), and CD105 (available from R&D Systems Inc., Minneapolis, Minn.); phycoerythrin (PE) conjugated monoclonal antibodies against CD44, CD200, CD117, and CD13 (BD Biosciences Pharmingen); phycoerythrin-Cy7 (PE Cy7) conjugated monoclonal antibodies against CD33 and CD10 (BD Biosciences Pharmingen); allophycocyanin (APC) conjugated streptavidin and monoclonal antibodies against CD38 (BD Biosciences Pharmingen); and Biotinylated CD90 (BD Biosciences Pharmingen). Other antibodies that can be used include, but are not limited to, CD133-APC (Miltenyi), KDR-Biotin (CD309, Abcam), CytokeratinK-Fitc (Sigma or Dako), HLA ABC-Fitc (BD), HLA DRDQDP-PE (BD), beta-2-microglobulin-PE (BD), CD80-PE (BD) and CD86-APC (BD).

Other antibody/label combinations that can be used include, but are not limited to, CD45-PerCP (peridin chlorophyll protein); CD44-PE; CD19-PE; CD10-F (fluorescein); HLA-G-F and 7-amino-actinomycin-D (7-AAD); HLA-ABC-F; and the like.

PDSC can be assayed for CD117 or CD133 using, for example, phycoerythrin-Cy5 (PE Cy5) conjugated streptavidin and biotin conjugated monoclonal antibodies against CD117 or CD133; however, using this system, the cells can appear to be positive for CD117 or CD133, respectively, because of a relatively high background.

PDSC can be labeled with an antibody to a single marker and detected and/sorted. PDSC can also be simultaneously labeled with multiple antibodies to different markers.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

PDSC can also be characterized and/or sorted based on cell morphology and growth characteristics. For example, PDSC can be characterized as having, and/or selected on the basis of, e.g., a fibroblastoid appearance in culture. PDSC can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4⁺ placental cells that produce one or more embryoid-like bodies in culture are isolated from other placental cells.

In another embodiment, PDSC can be identified and characterized by a colony forming unit assay. Colony forming unit assays are commonly known in the art, such as MESEN CULT™ medium (Stem Cell Technologies, Inc., Vancouver, British Columbia).

PDSC can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

PDSC can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 4.6 Culture of PDSC

4.6.1 Culture Media

Isolated PDSC, or PDSC population, or cells or placental tissue from which PDSC grow out, can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornmithine, vitronectin, and extracellular membrane protein (e.g., MATRIGEL™ (BD Discovery Labware, Bedford, Mass.)).

PDSC can be cultured in any medium, and under any conditions, recognized in the art as acceptable for the culture of stem cells. The culture medium can comprise serum. PDSC can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 10% FBS, EGF, and heparin; alpha-MEM (minimal essential medium) comprising 10% FBS, GLUTAMAX™ and gentamicin; DMEM comprising 10% FBS, GLUTAMAX™ and gentamicin, etc. An exemplary medium is DMEM-LG/MCDB-201 comprising 2% FBS, ITS, LA+BSA, dextrose, L-ascorbic acid, PDGF, EGF, and penicillin/streptomycin.

Other media in that can be used to culture PDSC include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMEM/F12, RPMI 1640, advanced DMEM (Gibco), DMEM/MCDB201 (Sigma), and CELL-GRO FREE.

The culture medium can be supplemented with one or more components including, for example, serum (e.g., fetal bovine serum (FBS), such as about 2-15% (v/v); equine (horse) serum (ES); human serum (HS)); beta-mercaptoethanol (BME), such as about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

PDSC can be cultured in standard tissue culture conditions, e.g., in tissue culture dishes or multiwell plates. PDSC can also be cultured using a hanging drop method. In this method, PDSC are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the stem cells are cultured.

In one embodiment, the PDSC are cultured in the presence of a compound that acts to maintain an undifferentiated phenotype in the PDSC. In a specific embodiment, the compound is a substituted 3,4-dihydropyridimol[4,5-d]pyrimidine. The compound can be contacted with a PDSC, or population of PDSC, at a concentration of, for example, between about 1 M to about 10 M.

4.6.2 Expansion and Proliferation of PDSC

Once an isolated PDSC, or isolated population of stem cells (e.g., a stem cell or population of stem cells separated from at least 50% of the placental cells with which the stem cell or population of stem cells is normally associated in vivo), the stem cell or population of stem cells can be proliferated and expanded in vitro. For example, a population of PDSC can be cultured in tissue culture containers, e.g., dishes, flasks, multiwell plates, or the like, for a sufficient time for the stem cells to proliferate to 70-90% confluence, that is, until the stem cells and their progeny occupy 70-90% of the culturing surface area of the tissue culture container.

PDSC can be seeded in culture vessels at a density that allows cell growth. For example, the cells may be seeded at low density (e.g., about 1,000 to about 5,000 cells/cm²) to high density (e.g., about 50,000 or more cells/cm²). In one embodiment, the cells are cultured at about 0 to about 5 percent by volume CO₂ in air. In some embodiments, the cells are cultured at about 2 to about 25 percent O₂ in air, such as about 5 to about 20 percent O₂ in air. The cells can be cultured at about 25° C. to about 40° C., such as 37° C. The cells can be cultured in an incubator. The culture medium can be static or agitated, for example, using a bioreactor. PDSC can be grown under low oxidative stress (e.g., with addition of glutathione, ascorbic acid, catalase, tocopherol, N-acetylcysteine, or the like).

Once 70%-90% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 stem cells, such as about 50,000 stem cells, are passaged to a new culture container containing fresh culture medium. Typically, the new medium is the same type of medium from which the stem cells were removed. Populations of PDSC that have been passaged at least 1, 2, 3, 4, 5, 6, 7, 8, 9, 10, 12, 14, 16, 18, or 20 times, or more, can also be used in the methods provided herein.

In a specific embodiment, PDSC provided herein have been passaged at least one time in culture. In another specific embodiment, PDSC provided herein has a different phenotype (e.g., has one or more different cell surface markers) as compared to the phenotype of the PDSC when resident in the placenta. In yet another specific embodiment, PDSC provided herein has a different phenotype (e.g., has one or more different cell surface markers) after passaging as compared to the phenotype of the PDSC before passaging.

### 4.7 Production of a Placental Stem Cell Bank

Stem cells from postpartum placentas can be cultured in a number of different ways to produce a set of lots, e.g., a set of individually-administrable doses, of PDSC. Such lots can, for example, be obtained from stem cells from placental perfusate or from enzyme-digested placental tissue. Sets of lots of PDSC, obtained from a plurality of placentas, can be arranged in a bank of PDSC for, e.g., long-term storage. Generally, adherent stem cells are obtained from an initial culture of placental material to form a seed culture, which is expanded under controlled conditions to form populations of cells from approximately equivalent numbers of doublings. Lots can be derived from the tissue of a single placenta, but can be derived from the tissue of a plurality of placentas.

In one embodiment, stem cell lots are obtained as follows. Placental tissue is first disrupted, e.g., by mincing, digested with a suitable enzyme, e.g., collagenase, as provided elsewhere herein. The placental tissue can comprise, e.g., the entire amnion, entire chorion, or both, from a single placenta, but can comprise only a part of either the amnion or chorion. The digested tissue is cultured, e.g., for about 1-3 weeks, such as about 2 weeks. After removal of non-adherent cells, high-density colonies that form are collected, e.g., by trypsinization. These cells are collected and resuspended in a convenient volume of culture medium, and defined as Passage 0 cells.

Passage 0 cells are then used to seed expansion cultures. Expansion cultures can be any arrangement of separate cell culture apparatuses, e.g., a Cell Factory by NUNC™. Cells in the Passage 0 culture can be subdivided to any degree so as to seed expansion cultures with, e.g., 1×10³, 2×10³, 3×10³, 4×10³, 5×10³, 6×10³, 7×10³, 8×10³, 9×10³, 1×10⁴, 1×10⁴, 2×10⁴, 3×10⁴, 4×10⁴, 5×10⁴, 6×10⁴, 7×10⁴, 8×10⁴, 9×10⁴, or 10×10⁴ stem cells. In some embodiments, from about 2×10⁴ to about 3×10⁴ Passage 0 cells are used to seed each expansion culture. The number of expansion cultures can depend upon the number of Passage 0 cells, and may be greater or fewer in number depending upon the particular placenta(s) from which the stem cells are obtained.

Expansion cultures are grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings can be maintained during expansion culture(s). The cells from a Passage 0 culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. In certain embodiments, however, the number of population doublings, prior to dividing the population of cells into individual doses, is between about 15 and about 30, such as about 20 doublings. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 100 million cells per ml, and can comprise between about 10⁶ and about 10⁹ cells in total.

In a specific embodiment, of the method, Passage 0 cells are cultured for a first number of doublings, e.g., approximately 4 doublings, then frozen in a first cell bank. Cells from the first cell bank are frozen and used to seed a second cell bank, the cells of which are expanded for a second number of doublings, e.g., about another eight doublings. Cells at this stage are collected and frozen and used to seed new expansion cultures that are allowed to proceed for a third number of doublings, e.g., about eight additional doublings, bringing the cumulative number of cell doublings to about 20. Cells at the intermediate points in passaging can be frozen in units of about 100,000 to about 10 million cells per mL, or about 1 million cells per mL for use in subsequent expansion culture. Cells at about 20 doublings can be frozen in individual doses of between about 1 million to about 100 million cells per mL for administration or use in making a stem cell-containing composition.

In one embodiment, therefore, provided is a method of making a placental stem cell bank, comprising: expanding primary culture PDSC from a human post-partum placenta for a first plurality of population doublings; cryopreserving said PDSC to form a Master Cell Bank; expanding a plurality of PDSC from the Master Cell Bank for a second plurality of population doublings; cryopreserving said PDSC to form a Working Cell Bank; expanding a plurality of PDSC from the Working Cell Bank for a third plurality of population doublings; and cryopreserving said PDSC in individual doses, wherein said individual doses collectively compose a placental stem cell bank. In a specific embodiment, the total number of population doublings is about 20. In another specific embodiment, said first plurality of population doublings is about four population doublings; said second plurality of population doublings is about eight population doublings; and said third plurality of population doublings is about eight population doublings. In another specific embodiment, said primary culture PDSC comprise PDSC from placental perfusate. In another specific embodiment, said primary culture PDSC comprise PDSC from digested placental tissue. In another specific embodiment, said primary culture PDSC comprise PDSC from placental perfusate and from digested placental tissue. In another specific embodiment, all of said PDSC in said placental stem cell primary culture are from the same placenta. In another specific embodiment, the method further comprises the step of selecting CD200⁺ or HLA-G⁺PDSC from said plurality of said PDSC from said Working Cell Bank to form individual doses. In another specific embodiment, said individual doses comprise from about 10⁴ to about 10⁵ PDSC. In another specific embodiment, said individual doses comprise from about 10⁵ to about 10⁶ PDSC. In another specific embodiment, said individual doses comprise from about 10⁶ to about 10⁷ PDSC. In another specific embodiment, said individual doses comprise from about 10⁷ to about 10⁸ PDSC.

In one embodiment, the donor from which the placenta is obtained (e.g., the mother) is tested for at least one pathogen. If the mother tests positive for a tested pathogen, the entire lot from the placenta is discarded. Such testing can be performed at any time during production of placental stem cell lots, including before or after establishment of Passage 0 cells, or during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

### 4.8 Preservation of PDSC

PDSC can be preserved, that is, placed under conditions that allow for long-term storage, or conditions that inhibit cell death by, e.g., apoptosis or necrosis.

PDSC can be preserved using, e.g., a composition comprising an apoptosis inhibitor, necrosis inhibitor and/or an oxygen-carrying perfluorocarbon, as described in related U.S. Provisional Application No. 60/754,969, entitled “Improved Medium for Collecting PDSC and Preserving Organs,” filed on Dec. 25, 2005. In one embodiment, provided is a method of preserving a population of stem cells comprising contacting said population of stem cells with a stem cell collection composition comprising an inhibitor of apoptosis and an oxygen-carrying perfluorocarbon, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of said stem cells. In another embodiment, said stem cell collection composition comprises said inhibitor of apoptosis and said oxygen-carrying perfluorocarbon in separate phases. In another embodiment, said stem cell collection composition comprises said inhibitor of apoptosis and said oxygen-carrying perfluorocarbon in an emulsion. In another embodiment, the stem cell collection composition additionally comprises an emulsifier, e.g., lecithin. In another embodiment, said apoptosis inhibitor and said perfluorocarbon are between about 0° C. and about 25° C. at the time of contacting the stem cells. In another more specific embodiment, said apoptosis inhibitor and said perfluorocarbon are between about 2° C. and 10° C., or between about 2° C. and about 5° C., at the time of contacting the stem cells. In another more specific embodiment, said contacting is performed during transport of said population of stem cells. In another more specific embodiment, said contacting is performed during freezing and thawing of said population of stem cells.

In another embodiment, provided is a method of preserving a population of PDSC comprising contacting said population of stem cells with an inhibitor of apoptosis and an organ-preserving compound, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, the organ-preserving compound is UW solution (described in U.S. Pat. No. 4,798,824; also known as ViaSpan®; see also Southard et al., Transplantation 1990, 49(2):251-257) or a solution described in Stern et al., U.S. Pat. No. 5,552,267. In another embodiment, said organ-preserving compound is hydroxyethyl starch, lactobionic acid, raffinose, or a combination thereof. In another embodiment, the stem cell collection composition additionally comprises an oxygen-carrying perfluorocarbon, either in two phases or as an emulsion.

In another embodiment of the method, PDSC are contacted with a stem cell collection composition comprising an apoptosis inhibitor and oxygen-carrying perfluorocarbon, organ-preserving compound, or combination thereof, during perfusion. In another embodiment, said stem cells are contacted during a process of tissue disruption, e.g., enzymatic digestion. In another embodiment, PDSC are contacted with said stem cell collection compound after collection by perfusion, or after collection by tissue disruption, e.g., enzymatic digestion.

Typically, during placental cell collection, enrichment and isolation, cell stress due to hypoxia and mechanical stress is minimized or eliminated. In another embodiment of the method, therefore, a stem cell, or population of stem cells, is exposed to a hypoxic condition during collection, enrichment or isolation for less than six hours during said preservation, wherein a hypoxic condition is a concentration of oxygen that is less than normal blood oxygen concentration. In a more specific embodiment, said population of stem cells is exposed to said hypoxic condition for less than two hours during said preservation. In another more specific embodiment, said population of stem cells is exposed to said hypoxic condition for less than one hour, or less than thirty minutes, or is not exposed to a hypoxic condition, during collection, enrichment or isolation. In another specific embodiment, said population of stem cells is not exposed to shear stress during collection, enrichment or isolation.

The PDSC provided herein can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium can comprise DMSO (dimethylsulfoxide), at a concentration of, e.g., about 10% (v/v). Cryopreservation medium may comprise additional agents, for example, methylcellulose and/or glycerol. PDSC can be cooled at about 1° C./min during cryopreservation. An exemplary cryopreservation temperature is about −80° C. to about −180° C., such as about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreservation can also be done using a controlled-rate freezer. Cryopreserved cells can be thawed at a temperature of about 25° C. to about 40° C., such as to a temperature of about 37° C.

### 4.9 Pharmaceutical Compositions

Populations of isolated placental cells or populations of cells comprising the isolated placental cells, can be formulated into pharmaceutical compositions for use in vivo, e.g., in the methods provided herein. Such pharmaceutical compositions comprise a population of isolated placental cells, or a population of cells comprising isolated placental cells, in a pharmaceutically-acceptable carrier, e.g., a saline solution or other accepted physiologically-acceptable solution for in vivo administration. Pharmaceutical compositions comprising the isolated placental cells described herein can comprise any, or any combination, of the isolated placental cell populations, or isolated placental cells, described elsewhere herein. The pharmaceutical compositions can comprise fetal, maternal, or both fetal and maternal isolated placental cells. The pharmaceutical compositions provided herein can further comprise isolated placental cells obtained from a single individual or placenta, or from a plurality of individuals or placentae.

The pharmaceutical compositions provided herein can comprise any number of isolated placental cells. For example, a single unit dose of isolated placental cells can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×100, 1×10¹¹ or more isolated placental cells.

The pharmaceutical compositions provided herein comprise populations of cells that comprise 50% viable cells or more (that is, at least 50% of the cells in the population are functional or living). In certain embodiments, at least 60% of the cells in the population are viable. In a specific embodiment, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, plasmalyte, and the like.

When formulated as an injectable solution, in one embodiment, the pharmaceutical composition comprises about 1% to 1.5% HSA and about 2.5% dextran. In one embodiment, the pharmaceutical composition comprises from about 5×10⁶ cells per milliliter to about 2×10⁷ cells per milliliter in a solution comprising 5% HSA and 10% dextran, optionally comprising an immunosuppressant, e.g., cyclosporine A at, e.g., 10 mg/kg.

In other embodiments, the pharmaceutical composition, e.g., a solution, comprises a plurality of cells, e.g., isolated placental cells, for example, PDSC, wherein said pharmaceutical composition comprises between about 1.0±0.3×10⁶ cells per milliliter to about 5.0±1.5×10⁶ cells per milliliter. In other embodiments, the pharmaceutical composition comprises between about 1.5×10⁶ cells per milliliter to about 3.75×10⁶ cells per milliliter. In other embodiments, the pharmaceutical composition comprises between about 1×10⁶ cells/mL to about 50×10⁶ cells/mL, about 1×10⁶ cells/mL to about 40×10⁶ cells/mL, about 1×10⁶ cells/mL to about 30×10⁶ cells/mL, about 1×10⁶ cells/mL to about 20×10⁶ cells/mL, about 1×10⁶ cells/mL to about 15×10⁶ cells/mL, or about 1×10⁶ cells/mL to about 10×10⁶ cells/mL. In certain embodiments, the pharmaceutical composition comprises no visible cell clumps (i.e., no macro cell clumps), or substantially no such visible clumps. As used herein, “macro cell clumps” means an aggregation of cells visible without magnification, e.g., visible to the naked eye, and generally refers to a cell aggregation larger than about 150 microns In some embodiments, the pharmaceutical composition comprises about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran, e.g., dextran-40. In a specific embodiment, said composition comprises about 7.5% to about 9% dextran-40. In a specific embodiment, said composition comprises about 5.5% dextran-40. In certain embodiments, the pharmaceutical composition comprises from about 1% to about 15% human serum albumin (HSA). In specific embodiments, the pharmaceutical composition comprises about 1%, 2%, 3%, 4%, 5%, 65, 75, 8%, 9%, 10%, 11%, 12%, 13%, 14% or 15% HSA. In a specific embodiment, said cells have been cryopreserved and thawed. In another specific embodiment, said cells have been filtered through a 70 μM to 100 μM filter. In another specific embodiment, said composition comprises no visible cell clumps. In another specific embodiment, said composition comprises fewer than about 200 cell clumps per 10⁶ cells, wherein said cell clumps are visible only under a microscope, e.g., a light microscope. In another specific embodiment, said composition comprises fewer than about 150 cell clumps per 10⁶ cells, wherein said cell clumps are visible only under a microscope, e.g., a light microscope. In another specific embodiment, said composition comprises fewer than about 100 cell clumps per 10⁶ cells, wherein said cell clumps are visible only under a microscope, e.g., a light microscope.

In a specific embodiment, the pharmaceutical composition comprises about 1.0±0.3×10⁶ cells per milliliter, about 5.5% dextran-40 (w/v), about 10% HSA (w/v), and about 5% DMSO (v/v).

In other embodiments, the pharmaceutical composition comprises a plurality of cells, e.g., a plurality of isolated placental cells in a solution comprising 10% dextran-40, wherein the pharmaceutical composition comprises between about 1.0±0.3×10⁶ cells per milliliter to about 5.0±1.5×10⁶ cells per milliliter, and wherein said composition comprises no cell clumps visible with the unaided eye (i.e., comprises no macro cell clumps). In some embodiments, the pharmaceutical composition comprises between about 1.5×10⁶ cells per milliliter to about 3.75×10⁶ cells per milliliter. In a specific embodiment, said cells have been cryopreserved and thawed. In another specific embodiment, said cells have been filtered through a 70 μM to 100 μM filter. In another specific embodiment, said composition comprises fewer than about 200 micro cell clumps (that is, cell clumps visible only with magnification) per 10⁶ cells. In another specific embodiment, the pharmaceutical composition comprises fewer than about 150 micro cell clumps per 10⁶ cells. In another specific embodiment, the pharmaceutical composition comprises fewer than about 100 micro cell clumps per 10⁶ cells. In another specific embodiment, the pharmaceutical composition comprises less than 15%, 14%, 13%, 12%, 11%, 10%, 9%, 8%, 7%, 6%, 5%, 4%, 3%, or 2% DMSO, or less than 1%, 0.9%, 0.8%, 0.7%, 0.6%, 0.5%, 0.4%, 0.3%, 0.2%, or 0.1% DMSO.

Further provided herein are compositions comprising cells, wherein said compositions are produced by one of the methods disclosed herein. For example, in one embodiment, the pharmaceutical composition comprises cells, wherein the pharmaceutical composition is produced by a method comprising filtering a solution comprising PDSC, to form a filtered cell-containing solution; diluting the filtered cell-containing solution with a first solution to about 1 to 50×10⁶, 1 to 40×10⁶, 1 to 30×10⁶, 1 to 20×10⁶, 1 to 15×10⁶, or 1 to 10×10⁶ cells per milliliter, e.g., prior to cryopreservation; and diluting the resulting filtered cell-containing solution with a second solution comprising dextran, but not comprising human serum albumin (HSA) to produce said composition. In certain embodiments, said diluting is to no more than about 15×10⁶ cells per milliliter. In certain embodiments, said diluting is to no more than about 10±3×10⁶ cells per milliliter. In certain embodiments, said diluting is to no more than about 7.5×10⁶ cells per milliliter. In other certain embodiments, if the filtered cell-containing solution, prior to the dilution, comprises less than about 15×10⁶ cells per milliliter, filtration is optional. In other certain embodiments, if the filtered cell-containing solution, prior to the dilution, comprises less than about 10±3×10⁶ cells per milliliter, filtration is optional. In other certain embodiments, if the filtered cell-containing solution, prior to the dilution, comprises less than about 7.5×10⁶ cells per milliliter, filtration is optional.

In a specific embodiment, the cells are cryopreserved between said diluting with a first dilution solution and said diluting with said second dilution solution. In another specific embodiment, the first dilution solution comprises dextran and HSA. The dextran in the first dilution solution or second dilution solution can be dextran of any molecular weight, e.g., dextran having a molecular weight of from about 10 kDa to about 150 kDa. In some embodiments, said dextran in said first dilution solution or said second solution is about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran. In another specific embodiment, the dextran in said first dilution solution or said second dilution solution is dextran-40. In another specific embodiment, the dextran in said first dilution solution and said second dilution solution is dextran-40. In another specific embodiment, said dextran-40 in said first dilution solution is 5.0% dextran-40. In another specific embodiment, said dextran-40 in said first dilution solution is 5.5% dextran-40. In another specific embodiment, said dextran-40 in said second dilution solution is 10% dextran-40. In another specific embodiment, said HSA in said solution comprising HSA is 1 to 15% HSA. In another specific embodiment, said HSA in said solution comprising HSA is about 1%, 2%, 3%, 4%, 5%, 6%, 7%, 8%, 9%, 10%, 11%, 12%, 13%, 14% or 15% HSA. In another specific embodiment, said HSA in said solution comprising HSA is 10% HSA. In another specific embodiment, said first dilution solution comprises HSA. In another specific embodiment, said HSA in said first dilution solution is 10% HSA. In another specific embodiment, said first dilution solution comprises a cryoprotectant. In another specific embodiment, said cryoprotectant is DMSO. In another specific embodiment, said dextran-40 in said second dilution solution is about 10% dextran-40. In another specific embodiment, said composition comprising cells comprises about 7.5% to about 9% dextran. In another specific embodiment, the pharmaceutical composition comprises from about 1.0±0.3×10⁶ cells per milliliter to about 5.0±1.5×10⁶ cells per milliliter. In another specific embodiment, the pharmaceutical composition comprises from about 1.5×10⁶ cells per milliliter to about 3.75×10⁶ cells per milliliter.

In another embodiment, the pharmaceutical composition is made by a method comprising (a) filtering a cell-containing solution comprising PDSC prior to cryopreservation to produce a filtered cell-containing solution; (b) cryopreserving the cells in the filtered cell-containing solution at about 1 to 50×10⁶, 1 to 40×10⁶, 1 to 30×10⁶, 1 to 20×10⁶, 1 to 15×10⁶, or 1 to 10×10⁶ cells per milliliter; (c) thawing the cells; and (d) diluting the filtered cell-containing solution about 1:1 to about 1:11 (v/v) with a dextran-40 solution. In certain embodiments, if the number of cells is less than about 10±3×10⁶ cells per milliliter prior to step (a), filtration is optional. In another specific embodiment, the cells in step (b) are cryopreserved at about 10±3×10⁶ cells per milliliter. In another specific embodiment, the cells in step (b) are cryopreserved in a solution comprising about 5% to about 10% dextran-40 and HSA. In certain embodiments, said diluting in step (b) is to no more than about 15×10⁶ cells per milliliter.

In another embodiment, the pharmaceutical composition is made by a method comprising: (a) suspending PDSC in a 5.5% dextran-40 solution that comprises 10% HSA to form a cell-containing solution; (b) filtering the cell-containing solution through a 70 μM filter; (c) diluting the cell-containing solution with a solution comprising 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (d) cryopreserving the cells; (e) thawing the cells; and (f) diluting the cell-containing solution 1:1 to 1:11 (v/v) with 10% dextran-40. In certain embodiments, said diluting in step (c) is to no more than about 15×10⁶ cells per milliliter. In certain embodiments, said diluting in step (c) is to no more than about 10±3×10⁶ cells/mL. In certain embodiments, said diluting in step (c) is to no more than about 7.5×10⁶ cells/mL.

In another embodiment, the composition comprising cells is made by a method comprising: (a) centrifuging a plurality of cells to collect the cells; (b) resuspending the cells in 5.5% dextran-40; (c) centrifuging the cells to collect the cells; (d) resuspending the cells in a 5.5% dextran-40 solution that comprises 10% HSA; (e) filtering the cells through a 70 μM filter; (f) diluting the cells in 5.5% dextran-40, 10% HSA, and 5% DMSO to about 1 to 50×10⁶, 1 to 40×10⁶, 1 to 30×10⁶, 1 to 20×10⁶, 1 to 15×10⁶, or 1 to 10×10⁶ cells per milliliter; (g) cryopreserving the cells; (h) thawing the cells; and (i) diluting the cells 1:1 to 1:11 (v/v) with 10% dextran-40. In certain embodiments, said diluting in step (f) is to no more than about 15×10⁶ cells per milliliter. In certain embodiments, said diluting in step (f) is to no more than about 10±3×10⁶ cells per milliliter. In certain embodiments, said diluting in step (f) is to no more than about 7.5×10⁶ cells per milliliter. In other certain embodiments, if the number of cells is less than about 10±3×10⁶ cells per milliliter, filtration is optional.

The compositions, e.g., pharmaceutical compositions comprising the isolated placental cells, described herein can comprise any of the isolated placental cells described herein.

Other injectable formulations, suitable for the administration of cellular products, may be used.

In one embodiment, the pharmaceutical composition comprises isolated placental cells that are substantially, or completely, non-maternal in origin, that is, have the fetal genotype; e.g., at least about 90%, 95%, 98%, 99% or about 100% are non-maternal in origin. For example, in one embodiment a pharmaceutical composition comprises a population of isolated placental cells that are CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺ and facilitate the formation of one or more embryoid-like bodies in a population of placental cells comprising said population of isolated placental cell when said population of placental cells is cultured under conditions that allow the formation of an embryoid-like body; or OCT-4⁺ and facilitate the formation of one or more embryoid-like bodies in a population of placental cells comprising said population of isolated placental cell when said population of placental cells is cultured under conditions that allow the formation of an embryoid-like body; or a combination of the foregoing, wherein at least 70%, 80%, 90%, 95% or 99% of said isolated placental cells are non-maternal in origin. In another embodiment, a pharmaceutical composition comprises a population of isolated placental cells that are CD10⁺, CD105⁺ and CD34⁻; CD10⁺, CD105⁺, CD200⁺ and CD34⁻; CD10⁺, CD105⁺, CD200⁺, CD34⁻ and at least one of CD90⁺ or CD45⁻; CD10⁺, CD90⁺, CD105⁺, CD200⁺, CD34⁻ and CD45⁻; CD10⁺, CD90⁺, CD105⁺, CD200⁺, CD34⁻ and CD45⁻; CD200⁺ and HLA-G⁻; CD73⁺, CD105⁺, and CD200⁺; CD200⁺ and OCT-4⁺; CD73⁺, CD105⁺ and HLA-G⁻; CD73⁺ and CD105⁺ and facilitate the formation of one or more embryoid-like bodies in a population of placental cells comprising said isolated placental cells when said population of placental cells is cultured under conditions that allow the formation of an embryoid-like body; OCT-4⁺ and facilitate the formation of one or more embryoid-like bodies in a population of placental cells comprising said isolated placental cells when said population of placental cells is cultured under conditions that allow the formation of an embryoid-like body; or one or more of CD117⁻, CD133⁻, KDR⁻, CD80⁻, CD86⁻, HLA-A,B,C⁺, HLA-DP, DQ, DR⁻, and/or PDL1⁺; or a combination of the foregoing, wherein at least 70%, 80%, 90%, 95% or 99% of said isolated placental cells are non-maternal in origin. In a specific embodiment, the pharmaceutical composition additionally comprises a stem cell that is not obtained from a placenta.

Isolated placental cells in the compositions, e.g., pharmaceutical compositions, provided herein, can comprise placental cells derived from a single donor, or from multiple donors. The isolated placental cells can be completely HLA-matched to an intended recipient, or partially or completely HLA-mismatched.

### 4.10 Matrices Comprising Isolated Placental Cells

Further provided herein are compositions comprising matrices, hydrogels, scaffolds, and the like that comprise a placental cell, or a population of isolated placental cells. Such compositions can be used in the place of, or in addition to, cells in liquid suspension.

The isolated placental cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Exemplary placental biomaterials on which isolated placental cells can be seeded are described in Hariri, U.S. Application Publication No. 2004/0048796, the disclosure of which is incorporated herein by reference in its entirety.

The isolated placental cells described herein can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. Isolated placental cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see, e.g., U.S. Patent Application Publication 2002/0022676, the disclosure of which is incorporated herein by reference in its entirety; Anseth et al., J. Control Release 2002, 78(1-3): 199-209; Wang et al., Biomaterials 2003, 24(22):3969-3980.

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (e.g., fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In other embodiments, the cells are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL®, Ethicon, Inc., Somerville, N.J.). Foams, composed of, e.g., poly(E-caprolactone)/poly(glycolic acid) (PCL/PGA) copolymer, formed by processes such as freeze-drying, or lyophilization (see, e.g., U.S. Pat. No. 6,355,699), can also be used as scaffolds.

The isolated placental cells described herein or co-cultures thereof can be seeded onto a three-dimensional framework or scaffold and implanted in vivo. Such a framework can be implanted in combination with any one or more growth factors, cells, drugs or other components that, e.g., stimulate tissue formation.

Examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL®, Ethicon, Inc., Somerville, N.J.). Foams, composed of, e.g., poly(E-caprolactone)/poly(glycolic acid) (PCL/PGA) copolymer, formed by processes such as freeze-drying, or lyophilization (see, e.g., U.S. Pat. No. 6,355,699), can also be used as scaffolds.

In another embodiment, isolated placental cells can be seeded onto, or contacted with, a felt, which can be, e.g., composed of a multifilament yarn made from a bioabsorbable material such as PGA, PLA, PCL copolymers or blends, or hyaluronic acid.

The isolated placental cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1 M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN® (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with isolated placental cells.

The placental cells (e.g., PDSC) provided herein can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE®(CanMedica Corp., Canada), ENDOBON® (Merck Biomaterial France, France), CEROS®(Mathys, AG, Bettlach, Switzerland), and mineralized collagen bone grafting products such as HEALOS™ (DePuy, Inc., Raynham, Mass.) and VITOSS®, RHAKOSS™, and CORTOSS®(Orthovita, Malvern, Pa.). The framework can be a mixture, blend or composite of natural and/or synthetic materials.

In one embodiment, the isolated placental cells are seeded onto, or contacted with, a suitable scaffold at about 0.5×10⁶ to about 8×10⁶ cells/mL.

### 4.11 Immortalized Placental Cell Lines

PDSC can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In one embodiment the growth-promoting gene is an oncogene such as, but not limited to, v-myc, N-myc, c-myc, p53, SV40 large T antigen, polyoma large T antigen, E1a adenovirus or E7 protein of human papillomavirus.

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. In one embodiment, a tetracycline (tet)-controlled gene expression system can be employed (see Gossen et al., Proc. Natl. Acad. Sci. USA 1992, 89:5547-5551; Hoshimaru et al., Proc. Natl. Acad. Sci. USA 1996, 93:1518-1523). In the absence of tet, a tet-controlled transactivator (tTA) within this vector strongly activates transcription from phCMV*-1, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. In some embodiments, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized placental cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized human placental cell line prepared as described above. In general, such clonal cell lines may be isolated using standard techniques, such as by limit dilution or using cloning rings, and expanded. Clonal cell lines may generally be fed and passaged as described above.

Conditionally-immortalized human placental cell lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 g/mL tetracycline for 4-5 days is sufficient to initiate differentiation. To promote further differentiation, additional agents may be included in the growth medium.

### 4.12 PDSC Conditioned Media

The PDSC provided herein can be used to produce conditioned medium, that is, medium comprising one or more biomolecules secreted or excreted by the stem cells. Such conditioned medium can be used in the various methods provided herein. In certain embodiments, the conditioned medium comprises medium in which PDSC have grown for at least 1, 2, 3, 4, 5, 6, 7, 8, 9, 10, 11, 12, 13, 14 or more days. In other embodiments, the conditioned medium comprises medium in which PDSC have grown to at least 30%, 40%, 50%, 60%, 70%, 80%, 90% confluence, or up to 100% confluence. Such conditioned medium can be used to support the culture of a separate population of PDSC, or stem cells of another kind. In another embodiment, the conditioned medium comprises medium in which PDSC have been differentiated into an adult cell type. In another embodiment, the conditioned medium comprises medium in which PDSC and non-PDSC have been cultured.

### 4.13 Assays

The PDSC (or other cells) provided herein can be used in assays to determine the influence of culture conditions, environmental factors, molecules (e.g., biomolecules, small inorganic molecules, etc.) and the like on stem cell proliferation, expansion, and/or differentiation, compared to PDSC not exposed to such conditions.

In one embodiment, PDSC (or other cells) provided herein are assayed for changes in proliferation, expansion or differentiation upon contact with a molecule. In one embodiment, for example, provided is a method of identifying a compound that modulates the proliferation of a plurality of PDSC, comprising contacting said plurality of PDSC with said compound under conditions that allow proliferation, wherein if said compound causes a detectable change in proliferation of said plurality of PDSC compared to a plurality of PDSC not contacted with said compound, said compound is identified as a compound that modulates proliferation of PDSC. In a specific embodiment, said compound is identified as an inhibitor of proliferation. In another specific embodiment, said compound is identified as an enhancer of proliferation.

In another embodiment, provided is a method of identifying a compound that modulates the expansion of a plurality of PDSC, comprising contacting said PDSC with said compound under conditions that allow expansion, wherein if said compound causes a detectable change in expansion of said PDSC compared to a plurality of PDSC not contacted with said compound, said compound is identified as a compound that modulates expansion of PDSC. In a specific embodiment, said compound is identified as an inhibitor of expansion. In another specific embodiment, said compound is identified as an enhancer of expansion.

In another embodiment, provided is a method of identifying a compound that modulates the differentiation of a PDSC, comprising contacting said stem cells with said compound under conditions that allow differentiation, wherein if said compound causes a detectable change in differentiation of said PDSC compared to a PDSC not contacted with said compound, said compound is identified as a compound that modulates proliferation of PDSC. In a specific embodiment, said compound is identified as an inhibitor of differentiation. In another specific embodiment, said compound is identified as an enhancer of differentiation.

### 4.14 Kits

In another aspect, provided herein are kits, suitable for the methods provided herein, comprising PDSC in a container separate from remaining kit contents, and instructions for use. In certain embodiments, the placental cells are provided in a pharmaceutically-acceptable solution, e.g., a solution suitable for administration, such as intravenous administration. In certain embodiments, the PDSC are any of the CD10⁺, CD34⁻, CD105⁺ placental cells described herein, e.g., CD10⁺, CD34⁻, CD105⁺, CD200⁺ placental cells or CD10⁺, CD34⁻, CD45⁻, CD90⁺, CD105⁺, CD200⁺ placental cells.

In certain embodiments, the kits comprise one or more components that facilitate delivery of the placental cells to the individual. For example, in certain embodiments, the kit comprises components that facilitate intravenous or other parenteral delivery of the placental cells to the individual. In such embodiments, the kit can comprise, e.g., syringes and needles suitable for delivery of cells to the individual, and the like. In such embodiments, the placental cells may be contained in the kit in a bag, or in one or more vials. In certain other embodiments, the kit comprises components that facilitate intravenous or intra-arterial delivery of the placental cells to the individual. In such embodiments, the placental cells may be contained, e.g., within a bottle or bag (for example, a blood bag or similar bag able to contain up to about 1.5 L solution comprising the cells), and the kit additionally comprises tubing and needles suitable for the delivery of cells to the individual.

Additionally, the kit may comprise one or more compounds that reduce pain or inflammation in the individual (e.g., an analgesic, steroidal or non-steroidal anti-inflammatory compound, or the like. The kit may also comprise an antibacterial or antiviral compound (e.g., one or more antibiotics), a compound to reduce anxiety in the individual (e.g., alaprazolam), a compound that reduces an immune response in the individual (e.g., cyclosporine A), an antihistamine (diphenhydramine, loratadine, desloratadine, quetiapine, fexofenadine, cetirizine, promethazine, chlorepheniramine, levocetirizine, cimetidine, famotidine, ranitidine, nizatidine, roxatidine, lafutidine, or the like).

Additionally, the kit can comprise disposables, e.g., sterile wipes, disposable paper goods, gloves, or the like, which facilitate preparation of the individual for delivery, or which reduce the likelihood of infection in the individual as a result of the administration of the placental cells.

### 4.15 Additional Embodiments

Other embodiments of the various methods described herein are provided below:

In one aspect provided herein is a method for maintaining or increasing the ratio of the number of stem cells to the number of differentiated cells in a tissue of a subject over time, comprising administering to the subject an effective amount of a population of stem cells, wherein the ratio is maintained or increased over time as compared to the ratio of the number of stem cells to the number of differentiated cells in a tissue of a control subject over time. In one embodiment, the population of stem cells comprises a population of PDSC. In another embodiment, the population of stem cells consists essentially of a population of PDSC. In a specific embodiment, the population of stem cells consists of a population of PDSC.

In another aspect, provided herein is a method of maintaining or increasing the number of stem cells in a tissue of a subject over time, comprising administering to the subject an effective amount of a population of stem cells, wherein the number of stem cells in the tissue of the subject is maintained or increased over time as compared to the number of stem cells in the same tissue of a control subject. In one embodiment, the population of stem cells comprises a population of PDSC. In another embodiment, the population of stem cells consists essentially of a population of PDSC. In a specific embodiment, the population of stem cells consists of a population of PDSC.

In another aspect, provided herein is a method of altering the phenotype of an aging stem cell resident in a tissue of a subject, comprising administering to the subject an effective amount of a population of stem cells, wherein the amount is effective to alter the environmental niche of the aging stem cell such that the phenotype of the stem cell is altered as compared to the phenotype of the stem cell resident in the tissue of a control subject. In one embodiment, the population of stem cells comprises a population of PDSC. In another embodiment, the population of stem cells consists essentially of a population of PDSC. In a specific embodiment, the population of stem cells consists of a population of PDSC.

In another aspect, provided herein is a method of altering the proteome of an aging cell in a tissue of a subject, comprising administering to the subject an effective amount of a population of stem cells, wherein the amount is effective to alter the proteome of the aging cell, wherein the altered proteome comprises one or more biomarkers found in a younger cell in the tissue of a control subject. In one embodiment, the population of stem cells comprises a population of PDSC. In another embodiment, the population of stem cells consists essentially of a population of PDSC. In a specific embodiment, the population of stem cells consists of a population of PDSC. In other embodiments, the one or more biomarkers are a protein expressed in a skeletal muscle, brain, heart, liver, kidney, bone marrow, or skin. In some embodiments, the one or more biomarkers are a protein expressed in a skeletal muscle. In some embodiments, the one or more biomarkers are a protein expressed in the brain. In other embodiments, the one or more biomarkers are a protein expressed in the heart. In certain embodiments, the one or more biomarkers are a protein expressed in the liver. In some embodiments, the one or more biomarkers are a protein expressed in the kidney. In other embodiments, the one or more biomarkers are a protein expressed in the bone marrow. In some embodiments, the one or more biomarkers are a protein expressed in the skin.

In another aspect, provided herein is a method of altering the transcriptome of an aging cell in a tissue of a subject, comprising administering to the subject an effective amount of a population of stem cells, wherein the amount is effective to alter the transcriptome of the aging cell, wherein the altered transcriptome comprises one or more transcripts found in a younger cell in the tissue of a control subject. In one embodiment, the population of stem cells comprises a population of PDSC. In another embodiment, the population of stem cells consists essentially of a population of PDSC. In a specific embodiment, the population of stem cells consists of a population of PDSC. In certain embodiments, the one or more transcripts are identified using a transcript array analysis.

In certain embodiments of the various methods provided herein, the aging cell is a somatic cell.

In other embodiments of the various methods provided herein, the control subject is the same subject before administration of the population of stem cells. In some embodiments of the various methods provided herein, the control subject is the same subject before administration of the population of stem cells. In some embodiments of the various methods provided herein, the control subject is a subject that has not received the population of stem cells. In one embodiment, the population of stem cells comprises a population of PDSC. In another embodiment, the population of stem cells consists essentially of a population of PDSC. In a specific embodiment, the population of stem cells consists of a population of PDSC.

In other embodiments of the various methods provided herein, the method further comprises (i) determining the number of stem cells and/or differentiated cells in the tissue before administration of the population of stem cells to the subject, and (ii) determining the number of stem cells and/or differentiated cells in the tissue after administration of the population of stem cells to the subject. In certain embodiments of the various methods provided herein, the control subject is a subject that has not received the population of stem cells.

In some embodiments of the various methods provided herein, the method increases the number of stem cells in the tissue after administration as compared to before administration of the population of stem cells. In other embodiments of the various methods provided herein, the subject has an increased number of stem cells as compared to a subject that has not received an administration of population of stem cells. In some embodiments of the various methods provided herein, the increase in the number of stem cells persists over time. In certain embodiments of the various methods provided herein, the number of stem cells is the result of an expansion of stem cells resident in the tissue. In other embodiments of the various methods provided herein, the increase in the number of stem cells is the result of an expansion of the stem cells in the tissue. In some embodiments of the various methods provided herein, the increase in the number of stem cells is the result of an expansion of the stem cells (e.g., the population of stem cells, the population of PDSC) in the tissue.

In some embodiments of the various methods provided herein, the number of stem cells is assessed by stem cell colony forming units.

In other embodiments of the various methods provided herein, the increase in the number of stem cells results in the remodeling, renewal, renovation, rejuvenation, repair and/or restoration of the tissue of the subject.

In certain embodiments of the various methods provided herein, the tissue is muscle. In some embodiments of the various methods provided herein, the tissue is brain. In other embodiments of the various methods provided herein, the tissue is skin. In some embodiments of the various methods provided herein, the tissue is bone marrow. In certain embodiments of the various methods provided herein, the tissue is heart. In other embodiments of the various methods provided herein, the tissue is liver. In some embodiments of the various methods provided herein, the tissue is kidney.

In some embodiments of the various methods provided herein, the population of stem cells is administered systemically. In other embodiments of the various methods provided herein, the population of stem cells is administered locally to the tissue. In certain embodiments of the various methods provided herein, the population of stem cells is administered by parenteral administration. In some embodiments of the various methods provided herein, the population of stem cells is administered intravenously. In other embodiments of the various methods provided herein, the population of stem cells is administered by continuous drip or as a bolus. In some embodiments of the various methods provided herein, the population of stem cells is prepared to be administered in an injectable liquid suspension or other biocompatible medium. In certain embodiments of the various methods provided herein, the population of stem cells is administered using a catheter. In other embodiments of the various methods provided herein, the population of stem cells is administered using a controlled-release system. In some embodiments of the various methods provided herein, the population of stem cells is administered using an implantable substrate or matrix. In some embodiments of the various methods provided herein, the population of stem cells is administered intramuscularly. In other embodiments of the various methods provided herein, the In certain embodiments of the various methods provided herein, the population of stem cells is administered subdermally. In some embodiments of the various methods provided herein, the population of stem cells is administered intracompartmentally. In a specific embodiment, the population of stem cells is a population of PDSC.

In other embodiments of the various methods provided herein, the method further comprises contacting the population of stem cells with young stem cells, young progenitor cells, or young precursor cells. In one embodiment, the population of stem cells comprises a population of PDSC. In another embodiment, the population of stem cells consists essentially of a population of PDSC. In a specific embodiment, the population of stem cells consists of a population of PDSC.

In some embodiments of the various methods provided herein, the method further comprises contacting the population of stem cells with one or more additional factors isolated from young stem cells, young progenitor cells, or young precursor cells. In certain embodiments of the various methods provided herein, the one or more additional factors is selected from the group consisting of cytokines, hormones, promoters, repressors, proteins, nucleic acids, viruses, immunogens, angiogenic factors, growth factors, anti-apoptotic factors, and anti-oxidative factors. In one embodiment, the population of stem cells comprises a population of PDSC. In another embodiment, the population of stem cells consists essentially of a population of PDSC. In a specific embodiment, the population of stem cells consists of a population of PDSC.

In other embodiments of the various methods provided herein, the method further comprises culturing and/or expanding the population of stem cells prior to administration to the subject. In some embodiments of the various methods provided herein, the culturing and/or expanding is in vitro. In some embodiments of the various methods provided herein, the culturing and/or expanding is in situ. In other embodiments of the various methods provided herein, the population of stem cells is cultured and/or expanded in the presence of young stem cells, young progenitor cells, or young precursor cells. In certain embodiments of the various methods provided herein, the population of stem cells is cultured and/or expanded in the presence of additional factors isolated from young stem cells, young progenitor cells, or young precursor cells. In some embodiments of the various methods provided herein, the one or more additional factors is selected from the group consisting of cytokines, hormones, promoters, repressors, proteins, nucleic acids, viruses, immunogens, angiogenic factors, growth factors, anti-apoptotic factors, and anti-oxidative factors. In other embodiments of the various methods provided herein, the population of stem cells are cultured and/or expanded in an extracorporeal device. In one embodiment, the population of stem cells comprises a population of PDSC. In another embodiment, the population of stem cells consists essentially of a population of PDSC. In a specific embodiment, the population of stem cells consists of a population of PDSC.

In some embodiments of the various methods provided herein, the population of stem cells has previously been cryopreserved. In certain embodiments of the various methods provided herein, the population of stem cells has been passaged at least three times. In other embodiments of the various methods provided herein, the population of stem cells has been passaged no more than ten times. In some embodiments of the various methods provided herein, the population of stem cells are cells from a placental stem cell bank. In some embodiments of the various methods provided herein, the stem cells are embryonic-like stem cells. In other embodiments of the various methods provided herein, the stem cells are pluripotent or multipotent stem cells. In certain embodiments of the various methods provided herein, the population of stem cells comprises cells obtained from a placenta that has been drained of cord blood. In some embodiments of the various methods provided herein, the population of stem cells comprises cells obtained from a placenta that has been perfused to remove residual blood. In one embodiment, the population of stem cells comprises a population of PDSC. In another embodiment, the population of stem cells consists essentially of a population of PDSC. In a specific embodiment, the population of stem cells consists of a population of PDSC. In one embodiment, the stem cells comprise PDSC. In one embodiment, the stem cells consist essentially of PDSC. In one embodiment, the stem cells consist of PDSC. In one embodiment, the stem cells are PDSC. In a specific embodiment, the stem cells are PDSC. In other embodiments of the various methods provided herein, the population of PDSC has previously been cryopreserved. In some embodiments of the various methods provided herein, the population of PDSC has been passaged at least three times. In certain embodiments of the various methods provided herein, the population of PDSC has been passaged no more than ten times. In other embodiments of the various methods provided herein, the population of PDSC are cells from a placental stem cell bank. In some embodiments of the various methods provided herein, the PDSC are embryonic-like stem cells. In some embodiments of the various methods provided herein, the PDSC are pluripotent or multipotent stem cells. In other embodiments of the various methods provided herein, the population of PDSC comprises cells obtained from a placenta that has been drained of cord blood. In certain embodiments of the various methods provided herein, the population of PDSC comprises cells obtained from a placenta that has been perfused to remove residual blood. In some embodiments of the various methods provided herein, the population of PDSC consists of cells obtained from a placenta that has been perfused to remove residual blood. In other embodiments of the various methods provided herein, the population of PDSC consists essentially of cells obtained from a placenta that has been perfused to remove residual blood.

In some embodiments of the various methods provided herein, population of PDSC comprises cells that are CD34⁻, CD10⁺, SH2⁺, CD90⁺ placental multipotent cells. In certain embodiments of the various methods provided herein, the population of PDSC comprises cells that CD34⁻, CD38⁻, CD45⁻, CD10⁺, CD29⁺, CD44⁺, CD54⁺, CD90⁺, SH2⁺, SH3⁺, SH4⁺ and OCT-4⁺. In other embodiments of the various methods provided herein, the population of PDSC comprises cells that are CD34⁻, CD10⁺, CD105⁺, and CD200⁺. In some embodiments of the various methods provided herein, the population of PDSC comprises cells that are CD73⁺. In some embodiments of the various methods provided herein, the population of PDSC comprises cells that are CD73⁺ and CD105⁺. In other embodiments of the various methods provided herein, the population of PDSC comprises cells that are CD200⁺. In certain embodiments of the various methods provided herein, the population of PDSC comprises cells that are CD34⁻, CD38⁻, CD45⁻, OCT-4⁺ and CD200⁺. In some embodiments of the various methods provided herein, the population of PDSC comprises cells that are CD34⁻, CD38⁻, CD45⁻, CD73⁺, OCT-4⁺ and CD200⁺. In other embodiments of the various methods provided herein, the population of PDSC comprises cells that are OCT-4⁺. In some embodiments of the various methods provided herein, the population of PDSC comprises cells that are CD73⁺, CD105⁺, and OCT4⁺. In certain embodiments of the various methods provided herein, the population of PDSC comprises cells that are CD73⁺, CD105⁺, and CD200⁺. In other embodiments of the various methods provided herein, the population of PDSC comprises cells that are CD73⁺ and CD105⁺. In some embodiments of the various methods provided herein, the population of PDSC comprises cells that are CD200⁺ an OCT-4⁺. In some embodiments of the various methods provided herein, the population of PDSC comprises cells that are CD73⁺, CD105⁺, and HLA-G⁺. In other embodiments of the various methods provided herein, the population of PDSC comprises cells that are CD73⁺, CD105⁺, HLA-G⁺. In certain embodiments of the various methods provided herein, the population of PDSC comprises cells that are CD73⁺, CD105⁺, CD200⁺ and HLA-G⁺. In some embodiments of the various methods provided herein, the population of PDSC comprises cells that are CD34⁻, CD38⁻, CD45⁻ and HLA-G⁺. In other embodiments of the various methods provided herein, the population of PDSC comprises cells that are CD34⁻; CD38⁻; CD45⁻; CD34⁻ and CD38⁻; CD34⁻ and CD45⁻; CD38⁻ and CD45⁻; or CD34⁻, CD38⁻ and CD45⁻.

In some embodiments of the various methods provided herein, the method further comprises characterizing the genome of the stem cells. In certain embodiments of the various methods provided herein, the genomic characterization is conducted prior to administration of the population of stem cells to the subject. In other embodiments of the various methods provided herein, the genomic characterization is conducted after administration of the population of stem cells to the subject. In some embodiments of the various methods provided herein, the genomic characterization is conducted prior to administration of the population of stem cells to the subject, and after administration of the population of stem cells to the subject. In one embodiment, the stem cells comprise PDSC. In one embodiment, the stem cells consist essentially of PDSC. In one embodiment, the stem cells consist of PDSC. In one embodiment, the stem cells are PDSC. In some embodiments of the various methods provided herein, the method further comprises characterizing the genome of the PDSC. In other embodiments of the various methods provided herein, the genomic characterization is conducted prior to administration of the population of PDSC to the subject. In certain embodiments of the various methods provided herein, the genomic characterization is conducted after administration of the population of PDSC to the subject. In some embodiments of the various methods provided herein, the genomic characterization is conducted prior to administration of the population of PDSC to the subject, and after administration of the population of PDSC to the subject.

In other embodiments of the various methods provided herein, the method further comprises characterizing the proteome of the stem cells. In some embodiments of the various methods provided herein, the proteomic characterization is conducted prior to administration of the population of stem cells to the subject. In certain embodiments of the various methods provided herein, the proteomic characterization is conducted after administration of the population of stem cells to the subject. In other embodiments of the various methods provided herein, the proteomic characterization is conducted prior to administration of the population of stem cells to the subject, and after administration of the population of stem cells to the subject. In one embodiment, the stem cells comprise PDSC. In one embodiment, the stem cells consist essentially of PDSC. In one embodiment, the stem cells consist of PDSC. In one embodiment, the stem cells are PDSC. In some embodiments of the various methods provided herein, the method further comprises characterizing the proteome of the PDSC. In some embodiments of the various methods provided herein, the proteomic characterization is conducted prior to administration of the population of PDSC to the subject. In other embodiments of the various methods provided herein, the proteomic characterization is conducted after administration of the population of PDSC to the subject. In certain embodiments of the various methods provided herein, the proteomic characterization is conducted prior to administration of the population of PDSC to the subject, and after administration of the population of PDSC to the subject.

In some embodiments of the various methods provided herein, the population of stem cells are autologous to the subject. In other embodiments of the various methods provided herein, the population of stem cells are allogeneic to the subject. In some embodiments of the various methods provided herein, the population of stem cells are syngeneic to the subject. In certain embodiments of the various methods provided herein, the population of stem cells is a homogeneous cell population. In other embodiments of the various methods provided herein, the population of stem cells is a mixed cell population. In some embodiments of the various methods provided herein, the population of stem cells is an enriched stem cell population. In one embodiment, the stem cells comprise PDSC. In one embodiment, the stem cells consist essentially of PDSC. In one embodiment, the stem cells consist of PDSC. In one embodiment, the stem cells are PDSC. In some embodiments of the various methods provided herein, the population of PDSC are autologous to the subject. In other embodiments of the various methods provided herein, the population of PDSC are allogeneic to the subject. In certain embodiments of the various methods provided herein, the population of PDSC are syngeneic to the subject. In some embodiments of the various methods provided herein, the population of PDSC is a homogeneous cell population. In other embodiments of the various methods provided herein, the population of PDSC is a mixed cell population. In some embodiments of the various methods provided herein, the population of PDSC is an enriched PDSC population. In certain embodiments of the various methods provided herein, the population of stem cells comprises PSC-100 cells. In other embodiments of the various methods provided herein, the population of PDSC comprises PSC-100 cells. In some embodiments of the various methods provided herein, the population of PDSC is an enriched population of PSC-100 cells.

In some embodiments of the various methods provided herein, the population of stem cells is administered at a dose of between 1×10⁵ cells and 1×10⁹ cells. In other embodiments of the various methods provided herein, the population of stem cells is administered at a dose of between 1×10⁵ cells and 1×10⁷ cells. In certain embodiments of the various methods provided herein, the population of stem cells is administered at a dose of between 1×10⁶ cells and 1×10⁷ cells. In some embodiments of the various methods provided herein, the population of stem cells is administered as a single dose. In other embodiments of the various methods provided herein, the population of stem cells is administered as multiple doses. In some embodiments of the various methods provided herein, the population of stem cells is the first administration to the subject. In certain embodiments of the various methods provided herein, the population of stem cells is administered when the subject is 10-15 years of age, 15-20 years of age, 20-25 years of age, 25-30 years of age, 30-35 years of age, 35-40 years of age, 40-45 years of age, 45-50 years of age, 50-55 years of age, 55-60 years of age, 60-65 years of age, 65-70 years of age, 70-75 years of age, 75-80 years of age, 80-85 years of age, 85-90 years of age, 90-95 years of age, 95-100 years of age, or over 100 years of age. In other embodiments of the various methods provided herein, populations of stem cells are serially administered over the lifetime of the subject. In one embodiment, the population of stem cells comprises a population of PDSC. In another embodiment, the population of stem cells consists essentially of a population of PDSC. In a specific embodiment, the population of stem cells consists of a population of PDSC.

In some embodiments of the various methods provided herein, the method further comprises characterizing the genome of the stem cells and/or differentiated cells in the tissue. In some embodiments of the various methods provided herein, wherein the genomic characterization is conducted prior to administration of the population of stem cells to the subject. In other embodiments of the various methods provided herein, the genomic characterization is conducted after administration of the population of stem cells to the subject. In certain embodiments of the various methods provided herein, the genomic characterization is conducted prior to administration of the population of stem cells to the subject, and after administration of the population of stem cells to the subject. In one embodiment, the population of stem cells comprises a population of PDSC. In another embodiment, the population of stem cells consists essentially of a population of PDSC. In a specific embodiment, the population of stem cells consists of a population of PDSC.

In some embodiments of the various methods provided herein, the method further comprises characterizing the proteome of the stem cells and/or differentiated cells in the tissue. In other embodiments of the various methods provided herein, the proteomic characterization is conducted prior to administration of the population of stem cells to the subject. In some embodiments of the various methods provided herein, the proteomic characterization is conducted after administration of the population of stem cells to the subject. In certain embodiments of the various methods provided herein, the proteomic characterization is conducted prior to administration of the population of stem cells to the subject, and after administration of the population of stem cells to the subject. In one embodiment, the population of stem cells comprises a population of PDSC. In another embodiment, the population of stem cells consists essentially of a population of PDSC. In a specific embodiment, the population of stem cells consists of a population of PDSC.

In other embodiments of the various methods provided herein, the one or more biomarkers are a protein expressed in a skeletal muscle cell and/or a striated muscle cell.

In some embodiments of the various methods provided herein, the one or more biomarkers are selected from the group consisting of myosin light chain 3 (MLCF3), myosin light polypeptide 2 (slow), myosin light chain 1 (MLC1F), myosin binding protein C (MYBPC1), myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), heat shock 27 kDa protein (Hsp27), disulfide iotherrase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, myosin heavy chain 2 (MYH2), troponin T type 1 (TNNT1), ryanodine receptor 1 (skeletal) (RYR1), calsequestrin 1 (fast-twitch, skeletal muscle) (CASQ1), junctophilin 1 (JPH1), adenosine monosphosphate deaminase (AMPD1), phosphorylase glycogen muscle (PYGM), and enolase 3 (beta, muscle) (ENO3).

In certain embodiments of the various methods provided herein, the one or more biomarkers are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the one or more biomarkers is indicative of aging. In other embodiments of the various methods provided herein, the one or more biomarkers are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the one or more biomarkers is indicative of aging.

In certain embodiments of the various methods provided herein, the one or more biomarkers are a protein expressed in the brain.

In other embodiments of the various methods provided herein, the one or more biomarkers are selected from the group consisting of myristoylated alanine-rich C-kinase substrate, alpha-internexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of myelin basic protein (MBP), and vimentin (VIM).

In some embodiments of the various methods provided herein, the one or more biomarkers are selected from the group consisting of amyloid beta (A4) precursor protein (APP), myristoylated alanine-rich protein kinase C substrate (MARCKS), internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1.

In some embodiments of the various methods provided herein, the one or more biomarkers are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU).

In other embodiments of the various methods provided herein, the one or more biomarkers are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN).

In certain embodiments of the various methods provided herein, the one or more biomarkers are selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH).

In some embodiments of the various methods provided herein, the one or more biomarkers are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1.

In other embodiments of the various methods provided herein, wherein the one or more biomarkers are a protein expressed in the heart.

In some embodiments of the various methods provided herein, the one or more biomarkers are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2).

In certain embodiments of the various methods provided herein, the one or more biomarkers are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2).

In other embodiments of the various methods provided herein, the one or more biomarkers are selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the one or more biomarkers is indicative of aging.

In some embodiments of the various methods provided herein, the biomarker is elongation factor 2 (Eef2) and an increase in the expression of Eef2 is indicative of aging.

In some embodiments of the various methods provided herein, the one or more biomarkers are a protein expressed in the kidney.

In other embodiments of the various methods provided herein, the one or more biomarkers are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromocertain 11 open reading frame 54 (C11 orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH).

In some embodiments of the various methods provided herein, the biomarker is selected from the group consisting of transferrin, isocitrate dehydrogenase 1 (IDH), and 3-hydroxyisobutyrate dehydrogenase, wherein an increase in the expression of the one or more biomarkers is indicative of an aging.

In other embodiments of the various methods provided herein, the one or more biomarkers are selected from the group consisting of afenopin, phosphoglycerate mutase, and glutathione (GSH), wherein a decrease in the expression of the one or more biomarkers is indicative of aging.

In some embodiments of the various methods provided herein, the increase in expression of the one or more biomarkers is gender specific. In certain embodiments of the various methods provided herein, the biomarker is ATP synthase and the expression of the ATP synthase in up-regulated in aging males. In other embodiments of the various methods provided herein, the biomarker is catalase and the expression of the catalase is down-regulated in aging males. In some embodiments of the various methods provided herein, the biomarker is ATP synthase and the expression of ATP synthase is down-regulated in aging females. In some embodiments of the various methods provided herein, the biomarker is ornithine aminotransferase and the expression of the ornithine aminotransferase is up-regulated in aging females. In other embodiments of the various methods provided herein, the biomarker is glutamate dehydrogenase and the expression of the glutamate dehydrogenase is down-regulated in aging females.

In certain embodiments of the various methods provided herein, the one or more biomarkers are a protein expressed in the liver.

In some embodiments of the various methods provided herein, the one or more biomarkers are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase.

In other embodiments of the various methods provided herein, the one or more biomarkers are selected from the group consisting of epoxide hydroxylase, 3-ketoacyl-CoA thiolase A, sarcosine oxidase, and 2,4-dienoyl reductase, wherein an increase in expression of the one or more biomarkers is indicative of aging.

In some embodiments of the various methods provided herein, the one or more biomarkers are a protein expressed in bone marrow.

In certain embodiments of the various methods provided herein, the one or more biomarkers are selected from the group consisting of defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromoother 19 open reading frame 59 (C19orf59), pyruvate dehydrogenase (lipoamide) beta, fatty acid-binding protein 5, galectin-3, c-synuclein, heterobiomarkerous nuclear ribonucleoprotein A1, myosin light chain, regulatory B (Mrlcb), transgelin, similar to purine-nucleoside phosphorylase (punA), heterobiomarkerous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5).

In some embodiments of the various methods provided herein, the one or more biomarkers are selected from the group consisting of fatty acid-binding protein 5, galectin-3, c-synuclein, heterobiomarkerous nuclear ribonucleoprotein A1, myosin light chain, regulatory B, peroxiredoxin 5 precursor, and transgelin.

In some embodiments of the various methods provided herein, the one or more biomarkers are selected from the group consisting of beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterobiomarkerous nuclear ribonucleoprotein A1 isoform a (HNRPA1), heterobiomarkerous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln).

In other embodiments of the various methods provided herein, the one or more biomarkers are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5).

In certain embodiments of the various methods provided herein, the one or more biomarkers are a protein expressed in the skin.

In some embodiments of the various methods provided herein, the one or more biomarkers are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CDla molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteaother non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1).

In certain embodiments of the various methods provided herein, the one or more biomarkers are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the one or more biomarkers is indicative of aging.

In other embodiments of the various methods provided herein, the one or more biomarkers are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1).

In other embodiments of the various methods provided herein, the one or more biomarkers are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the one or more biomarkers is indicative of aging.

In certain embodiments of the various methods provided herein, the one or more biomarkers are selected from the group consisting of twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteaother non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the one or more biomarkers is indicative of aging.

In some embodiments of the various methods provided herein, the one or more transcripts are a transcript expressed in a skeletal muscle.

In certain embodiments of the various methods provided herein, the one or more transcripts are selected from the group consisting of MLCF3, myosin light polypeptide 2 (slow), MLC1F, MYBPC1, myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate iotherrase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, MYH2, TNNT1, RYR1, CASQ1, JPH1, AMPD1, PYGM, and ENO3.

In some embodiments of the various methods provided herein, the one or more transcripts are selected from the group consisting of MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate iotherrase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the one or more transcripts is indicative of aging.

In certain embodiments of the various methods provided herein, the one or more transcripts are selected from the group consisting of troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the one or more transcripts is indicative of aging.

In other embodiments of the various methods provided herein, the one or more transcripts are a transcript expressed in a brain.

In some embodiments of the various methods provided herein, the one or more transcripts are selected from the group consisting of myristoylated alanine-rich C-kinase substrate, alpha-internexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of MBP, and VIM.

In certain embodiments of the various methods provided herein, the one or more transcripts are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1.

In other embodiments of the various methods provided herein, the one or more transcripts are selected from the group consisting of amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU).

In some embodiments of the various methods provided herein, the one or more transcripts are selected from the group consisting of proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN).

In some embodiments of the various methods provided herein, the one or more transcripts are selected from the group consisting of microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH).

In other embodiments of the various methods provided herein, the one or more transcripts are selected from the group consisting of neurofilament triplet L protein (NF-L), peroxiredoxin (EC 1.11.1.), aconitate hydratase (EC 4.2.1.3), enolase 2 (EC 4.2.1.11), and T-complex protein 1.

In certain embodiments of the various methods provided herein, the one or more transcripts are a transcript expressed in a heart.

In some embodiments of the various methods provided herein, the one or more transcripts are selected from the group consisting of myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2).

In other embodiments of the various methods provided herein, the one or more transcripts are selected from the group consisting of ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2).

In some embodiments of the various methods provided herein, the one or more transcripts are selected from the group consisting of ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the one or more transcripts is indicative of aging.

In certain embodiments of the various methods provided herein, the transcript is elongation factor 2 (Eef2) and an increase in the expression of Eef2 is indicative of aging.

In other embodiments of the various methods provided herein, the one or more transcripts are a transcript expressed in a kidney.

In some embodiments of the various methods provided herein, the one or more transcripts are selected from the group consisting of podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11 orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH).

In other embodiments of the various methods provided herein, the transcript is selected from the group consisting of transferrin, isocitrate dehydrogenase 1 (IDH), and 3-hydroxyisobutyrate dehydrogenase, wherein an increase in the expression of the one or more transcripts is indicative of an aging.

In certain embodiments of the various methods provided herein, the one or more transcripts are selected from the group consisting of afenopin, phosphoglycerate mutase, and glutathione (GSH), wherein a decrease in the expression of the one or more transcripts is indicative of aging.

In some embodiments of the various methods provided herein, the increase in expression of the one or more transcripts is gender specific. In other embodiments of the various methods provided herein, the transcript is ATP synthase and the expression of the ATP synthase in up-regulated in aging males. In some embodiments of the various methods provided herein, the transcript is catalase and the expression of the catalase is down-regulated in aging males. In certain embodiments of the various methods provided herein, the transcript is ATP synthase and the expression of ATP synthase is down-regulated in aging females. In other embodiments of the various methods provided herein, the transcript is ornithine aminotransferase and the expression of the ornithine aminotransferase is up-regulated in aging females. In some embodiments of the various methods provided herein, the transcript is glutamate dehydrogenase and the expression of the glutamate dehydrogenase is down-regulated in aging females.

In some embodiments of the various methods provided herein, the one or more transcripts are a transcript expressed in a liver.

In other embodiments of the various methods provided herein, the one or more transcripts are selected from the group consisting of apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase.

In certain embodiments of the various methods provided herein, the one or more transcripts are selected from the group consisting of epoxide hydroxylase, 3-ketoacyl-CoA thiolase A, sarcosine oxidase, and 2,4-dienoyl reductase, wherein an increase in expression of the one or more transcripts is indicative of aging.

In some embodiments of the various methods provided herein, the one or more transcripts are a transcript expressed in bone marrow.

In other embodiments of the various methods provided herein, the one or more transcripts are selected from the group consisting of defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterogeneous nuclear ribonucleoprotein A1, myosin light chain, regulatory B (Mrlcb), transgelin, similar to purine-nucleoside phosphorylase (punA), heterogeneous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5).

In certain embodiments of the various methods provided herein, the one or more transcripts are selected from the group consisting of fatty acid-binding protein 5, galectin-3, c-synuclein, heterogeneous nuclear ribonucleoprotein A1, myosin light chain, regulatory B, peroxiredoxin 5 precursor, and transgelin.

In other embodiments of the various methods provided herein, the one or more transcripts are selected from the group consisting of beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterogeneous nuclear ribonucleoprotein A1 isoform a (HNRPA1), heterogeneous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln).

In some embodiments of the various methods provided herein, the one or more transcripts are selected from the group consisting of transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5).

In some embodiments of the various methods provided herein, the one or more transcripts are a transcript expressed in the skin.

In other embodiments of the various methods provided herein, the one or more transcripts are selected from the group consisting of collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteacertain non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1).

In other embodiments of the various methods provided herein, the one or more transcripts are selected from the group consisting of mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the one or more transcripts is indicative of aging.

In some embodiments of the various methods provided herein, the one or more transcripts are selected from the group consisting of annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteacertain non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteaother non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1).

In some embodiments of the various methods provided herein, the one or more transcripts are selected from the group consisting of annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the one or more transcripts is indicative of aging.

In some embodiments of the various methods provided herein, the one or more transcripts are selected from the group consisting of twinfilin-2 (TWF2), 40S ribosomal protein S5 (RPS5), 26S proteaother non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteacertain non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the one or more transcripts is indicative of aging.

In some embodiments of the various methods provided herein, the aging cell is from a muscle. In other embodiments of the various methods provided herein, the aging cell is a muscle cell. In some embodiments of the various methods provided herein, the muscle cell is a skeletal muscle cell. In other embodiments of the various methods provided herein, the muscle cell is a striated muscle cell. In certain embodiments of the various methods provided herein, the aging cell is from a brain. In some embodiments of the various methods provided herein, the aging cell is a brain cell. In other embodiments of the various methods provided herein, the aging cell is from a heart. In some embodiments of the various methods provided herein, the aging cell is a heart cell. In certain embodiments of the various methods provided herein, the aging cell is from a kidney. In other embodiments of the various methods provided herein, the aging cell is a kidney cell. In some embodiments of the various methods provided herein, the aging cell is from a liver. In some embodiments of the various methods provided herein, the aging cell is a liver cell. In other embodiments of the various methods provided herein, the aging cell is from bone marrow. In certain embodiments of the various methods provided herein, the aging cell is a bone marrow cell. In some embodiments of the various methods provided herein, the aging cell is from skin. In other embodiments of the various methods provided herein, the aging cell is a skin cell.

### 5. Examples

### 5.1 Example 1

### Detection and Quantification of Stem Cell Niches: Phase I Project in Aging

The purpose of phase I experimentation was as follows:

1) To determine the age-related changes in the stem cells of skeletal muscle, liver, skin, kidney, brain, cardiac tissue, and bone marrow; and

2) To correlate the age-related decline in skeletal muscle and cardiac muscle stem cells to functional outcomes (ventricular function via “echo”) as well as skeletal muscle strength (endurance using a Rotarod).

Materials and Methods:

Fisher 344 rats were purchased from a commercial laboratory (Harlan Laboratories, Indianapolis, Ind.) and were allowed to acclimate in the animal housing facility for at least one week prior to experimentation. During acclimation, animals were provided standard rodent chow (24% protein, 58% CHO, 18% fat; Teklad Global #2018 Diet, Harlan Laboratories) and water ad libitum in a maintained ambient temperature and constant 12 h light: 12 h dark cycle.

The morning of experimentation, animals were transported to the Molecular and Applied Sciences Laboratory and were subjected to a muscular endurance test using a Rotarod (Med Associates Inc., St. Albans, Vt.). Rats were placed on the Rotarod whereby a progressive speed regimen (4-40 rpm) was applied. Rats were only allowed one turn on the Rotarod and time spent on the device prior to falling was recorded.

Following the muscular endurance testing, animals were allowed to acclimate for approximately two hours. Thereafter, echocardiogram assessment occurred whereby animals were anesthetized with isoflurane, the chest cavity was shaven, and a rat-specific probe interfaced with a high resolution ultrasound (Logiq™ S7 R2 Expert; General Electric, Fairfield, Conn.) was placed over the chest cavity to record beat-by-beat heart beat cardiac function data over a 10-second period. Afterwards, rats were placed back in their home cage and were allowed to recover from isoflurance anesthesia.

Approximately 30 min following recovery from anaesthesia, animals were euthanized under CO₂ gas in a 2 L induction chamber (VetEquip, Inc., Pleasanton, Calif.). Following euthanasia, whole blood was removed using multiple 3 ml syringes with 23 gauge needles via heart puncture. An aliquot of blood was placed in a K-EDTA tube for peripheral blood mononuclear cells (PMBC) isolation and subsequent flow cytometry for circulating endothelial progenitor cell (EPC) quantitation described below. A second aliquot was placed in a serum seperator tube, spun at 3,000×g for five minutes at room temperature, and serum was aliquoted for metabolomics.

Following blood collection, different tissues (i.e., right triceps, left ventricle, hippocampus, right kidney, liver, right femur) were dissected out, weighed, and processed for stem cell isolation described below. Moreover, additional right triceps and left ventricle tissue was placed in OCT media and slow-frozen in liquid nitrogen-cooled isopentane and stored at −80° C. until cryosectioning described below.

Eight groups of seven age-matched (3, 6, 9, 12, 15, 18, 21 and 24 months) rats were euthanized for stem cell quantification. Each group consisted of 9-10 animals.

General Aging Characteristics Measurements:

Body Mass Measurements

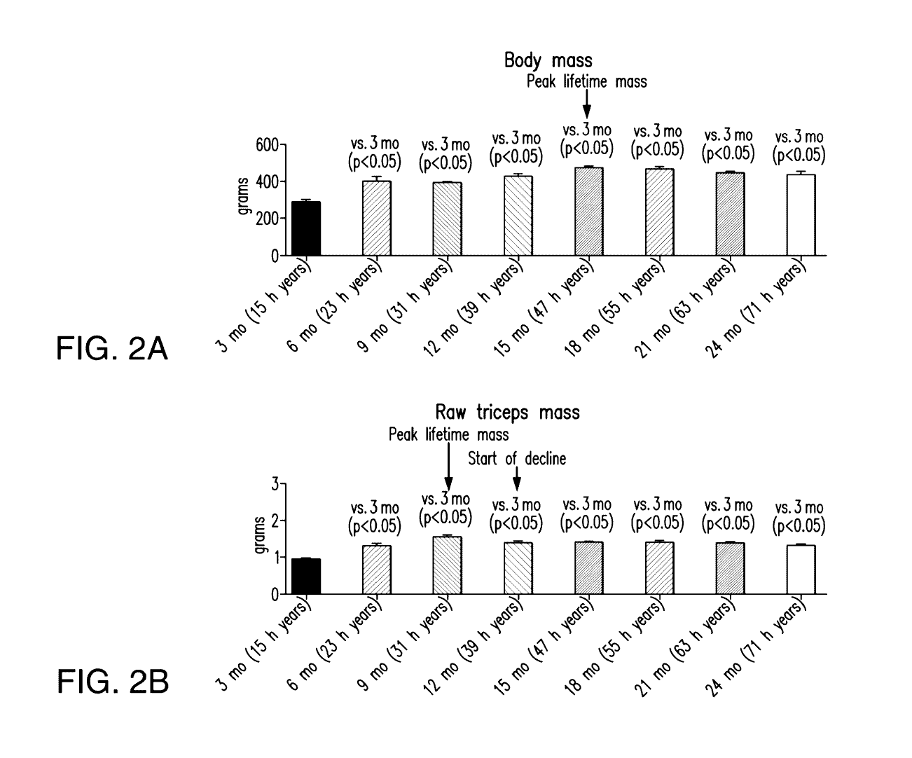

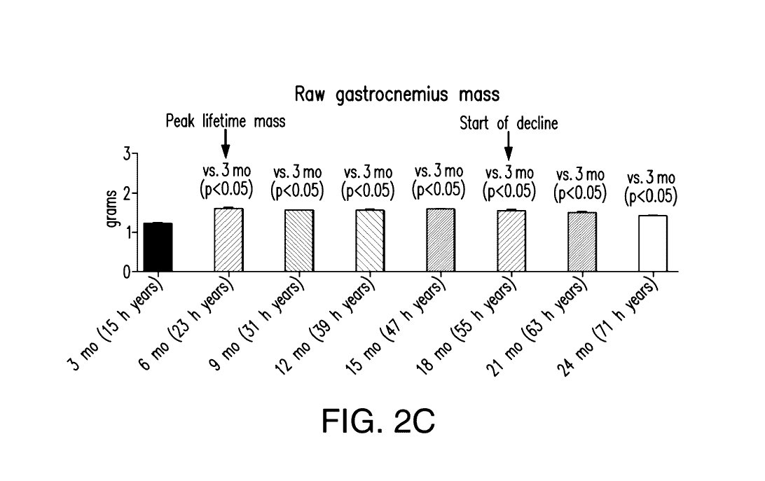

FIG. 2A shows a precipitous growth spurt from 3-6 months and lifetime peak mass at 15 months.

Raw Triceps Muscle Mass Measurements

FIG. 2B shows that peak lifetime triceps masses were realized at 9 months, and that age-related declines began at 12 months. This is likely due to the forelimb not being utilized much in rats (i.e., feeding and drinking occurs with hind limb action) resulting in age-related atrophy occurring at a much greater rate in the forelimb muscle.

Raw Gastrocnemius Muscle Mass Measurements

FIG. 2C shows that peak lifetime gastrocnemius masses were realized at 6 months, and that age-related decline started at 18 months. Again, this is because the rats use these muscles routinely for eating and drinking and, thus, neuromuscular activation likely protects this muscle from age-related atrophy.

Skeletal Muscle Stem Cell Measurements:

NCAM (CD56)-Positive Skeletal Muscle Satellite Cell Flow Cytometry

Because age-related atrophy affected the forelimb muscles more than the hind limb muscles (see above), stem cell quantification in the triceps using flow cytometry was chosen to be evaluated. NCAM (CD56) was chosen as a marker because this is a cell surface marker that is expressed on muscle satellite cells (Trapecar et al., J Muscle Res Cell Motil. 2014, 35(5-6):249-257).

Following right triceps extraction, the muscle was weighed, and a portion (˜200 mg) from the long head was removed, rinsed in ice-cold PBS, and placed in 20 volumes of digestion solution (1% collagenase II in phosphate buffered saline (PBS)). Tissue was then minced and subsequently incubated on a rocking platform (150 rpm) at 37° C. for 30 min. The resultant slurry was passed through a 100 μm cell strainer and the effluent was collected in a 50 mL conical tube. Tubes were centrifuged for 5 min. at 2,500×g, supernatant was siphoned off and the resultant pellet was washed with 5 ml of PBS. Tubes were again centrifuged for 5 min. at 2,500×g, the resultant pellet was resuspended in 200 μL of flow cytometry (FC) buffer (eBiosciences), 50 μL of the resuspended cell slurry was placed in a new 1.7 mL microcentrifuge tube, and samples were resuspended in primary antibody solution (2 μL mouse anti-rat NCAM IgG1 (Abcam)+48 μL of FC buffer) at room temperature for 60 min. Cells were not fixed in paraformaldehyde prior to primary antibody incubation due to fixation drastically reducing cell yield by the end of the assay during pilot experiments.

Following primary antibody incubation, tubes were centrifuged for 5 min. at 2,500×g and the resultant pellet was resuspended and washed in 500 μL of FC buffer. Tubes were again centrifuged for 5 min. at 2,500×g and the resultant pellet was resuspended in secondary antibody solution (2 μL FITC-conjugated anti-mouse IgG1 antibody (eBiosciences)+98 μL of FC buffer) in the dark at room temperature for 60 min. Tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and FITC-labelled cells were detected using a flow cytometer (BD Accuri C6). Specifically, 10,000 events were quantified using a pre-defined gate and the proportion of cells that emitted a fluorescent intensity above background fluorescence (detected in unstained samples) were considered to be FITC-labelled NCAM-positive muscle satellite cells.

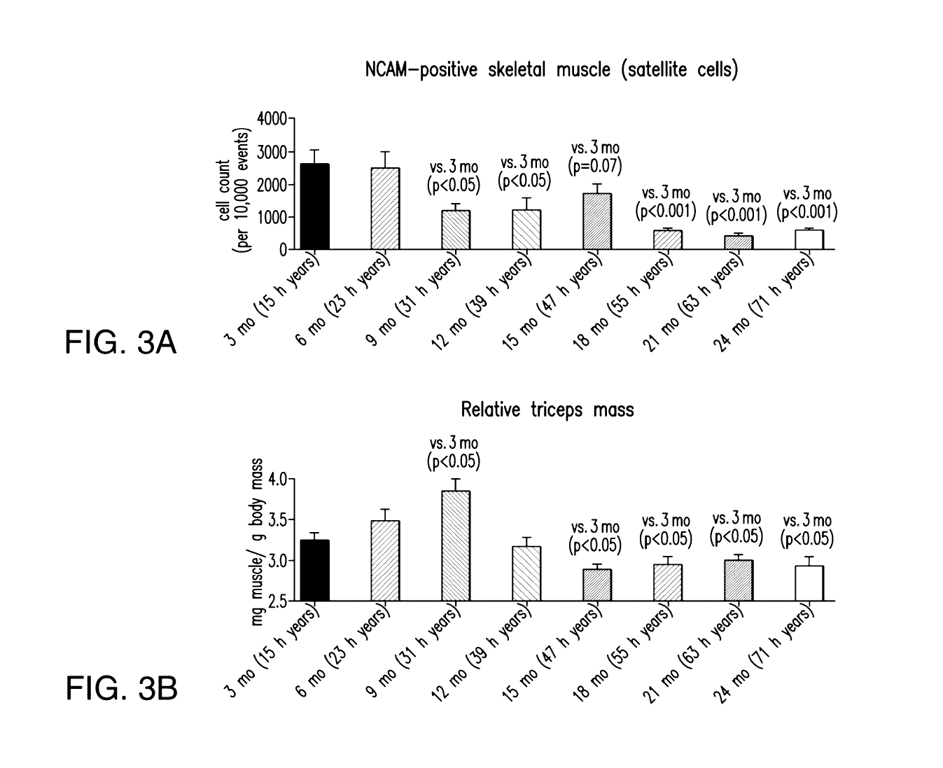

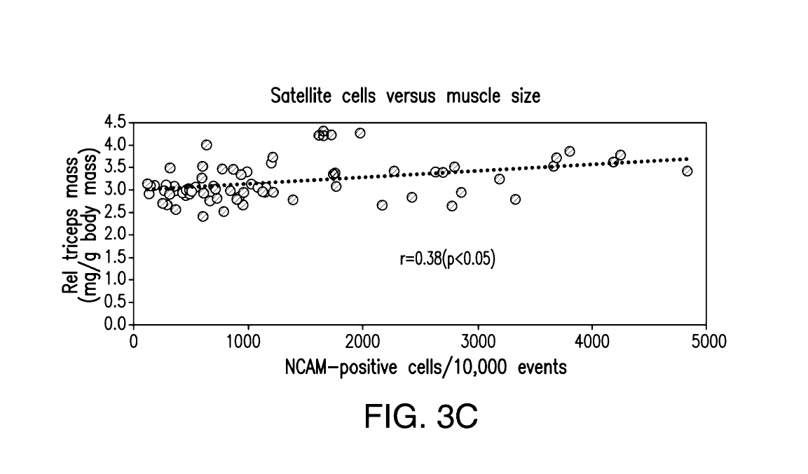

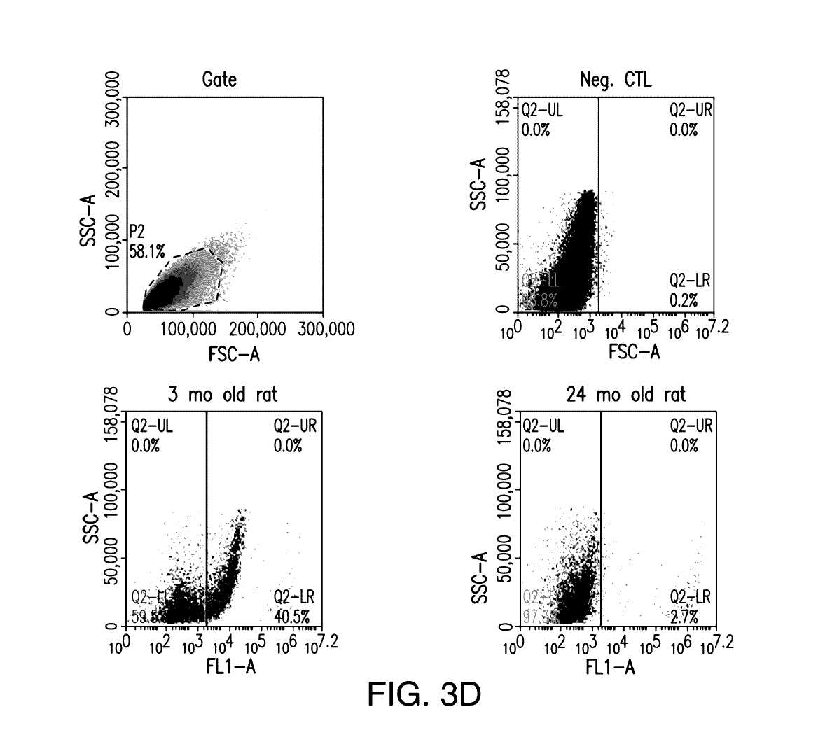

The NCAM (CD56)-positive skeletal muscle satellite cell counts (FIG. 3A) were in agreement with Day et al., who reported that mice aged 19-25 months had half of the satellite cells as mice aged 3-4 months (Dev Biol. 2010, 340(2): 330-343). FIG. 3B shows that peak lifetime relative triceps masses (mg muscle/g body mass) were realized at 9 months, and that age-related decline started at 12 months. FIG. 3C demonstrates that there may be a predictive relationship between satellite cell content and relative triceps muscle mass. FIG. 3D shows representative flow cytometry data, including gate, negative control, 3 month old rat, and 24 month old rat.

Pax7 immunofluorescence Measurements of Skeletal Muscle Satellite Cells

In addition, Pax7 immunofluorescence was performed. Triceps sections from OCT-preserved samples were cut at a thickness of 10 μm using a cryotome (HM 525 Cryostat; Thermo Fisher Scientific, Waltham, Mass.) and were adhered to positively-charged histology slides. Once all samples were sectioned, batch processing occurred for Pax7 immunofluoresence. Briefly, sections were dried at room temperature for 30 min. and incubated in permeabilization solution (0.5% Triton X-100 in PBS). Sections were rinsed in PBS and were immunostained for 60 min. at room temperature with a cocktail of rabbit anti-dystrophin IgG (1:100; Abcam) and mouse anti-Pax7 IgG (1:100; Developmental Studies Hybridoma Bank, University of Iowa, Iowa City, Iowa) in PBS containing 5% blocking solution (Super Blocker; Thermo Fisher Scientific, Waltman, Mass.). Thereafter, slides were rinsed in PBS and incubated for 60 min. with a cocktail containing goat anti-rabbit IgG (Texas Red-conjugated) (1:100; Vector Laboratories, Burlingame, Calif.) and anti-mouse IgG (FITC-conjugated; Santa Cruz Biotech, Dallas, Tex.). Thereafter, slides were rinsed in PBS, mounted with glass coverslips and DAPI media (Vector Laboratories, Burlingame, Calif.), and stored in the dark until imaging.

A 40× image of each respective fluorescent filter (cell membrane, Texas Red; satellite cell, FITC; nuclei, DAPI) was obtained using a fluorescent microscope (Nikon Eclipse Ti-U; Nikon Instruments, Melville, N.Y.). Images were merged using the NIS Elements software (Nikon) and the number of Pax-7-positive cells with nuclei were quantified per 40× image.

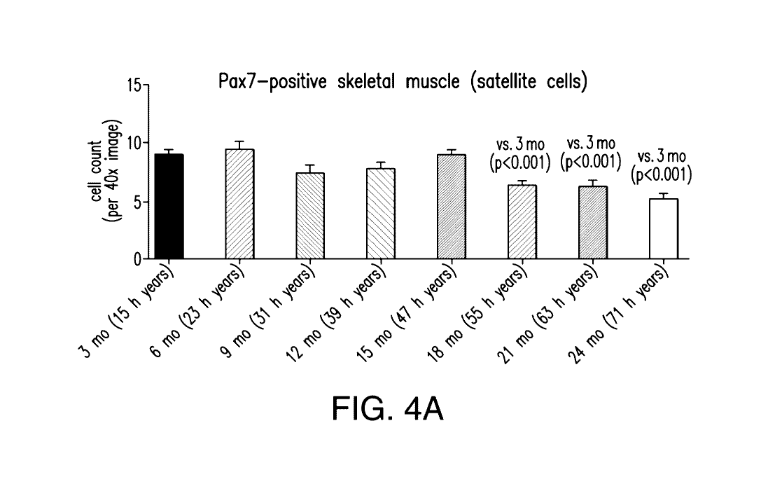

FIG. 4A shows Pax7-positive skeletal muscle satellite cell counts over rats lifespan (3-24 months), and FIG. 4B are exemplary Pax7 staining images of muscle satellite cells from 3, 9, 18, or 24 month old rats. The Pax7 immunofluorescence data generally agree with the NCAM (CD56) flow cytometry data, but are likely different due to NCAM being used as a cell surface marker for flow cytometry, and Pax7 being used for immunofluorescence.

Triceps Fibrosis Measurements

Further, triceps fibrosis was studied by using Trichrome™ staining. Triceps sections from OCT-preserved samples were cut at a thickness of 10 μm using a cryotome (HM 525 Cryostat; Thermo Fisher Scientific, Waltham, Mass.) and were adhered to positively-charged histology slides. Once all samples were sectioned, batch processing occurred for Trichrome staining using a commercially-available kit per the manufacturer's instructions (Abcam). Following staining, slides were mounted and imaged using bright field imaging (Nikon Eclipse Ti-U; Nikon Instruments, Melville, N.Y.).

A 20× image of stained triceps tissue was obtained. Images were then analyzed via ImageJ (National Institutes of Health, Bethesda, Md.) whereby the percent of each image that was fibrotic was quantified.

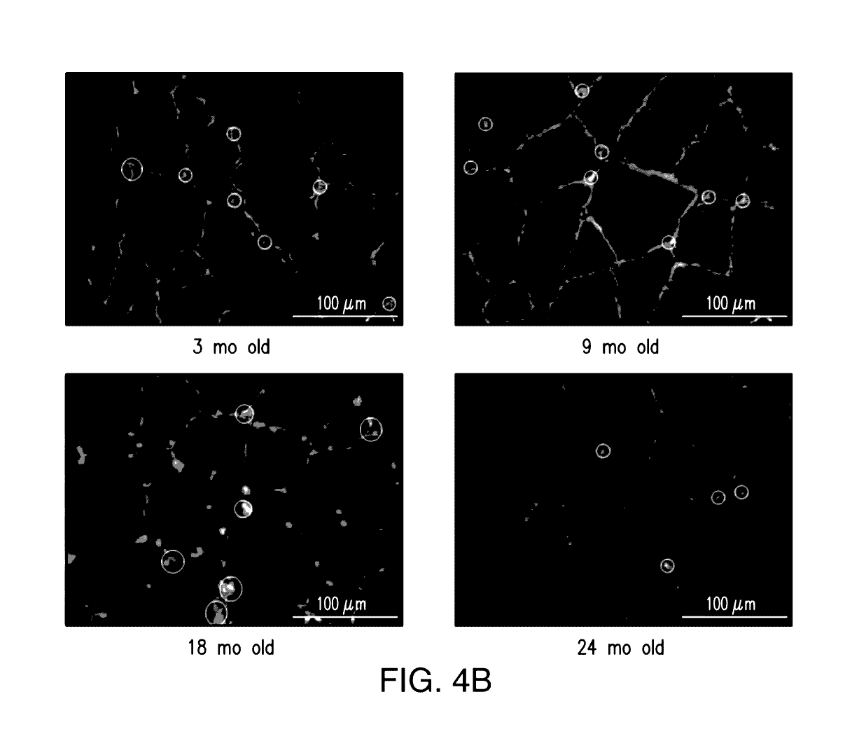

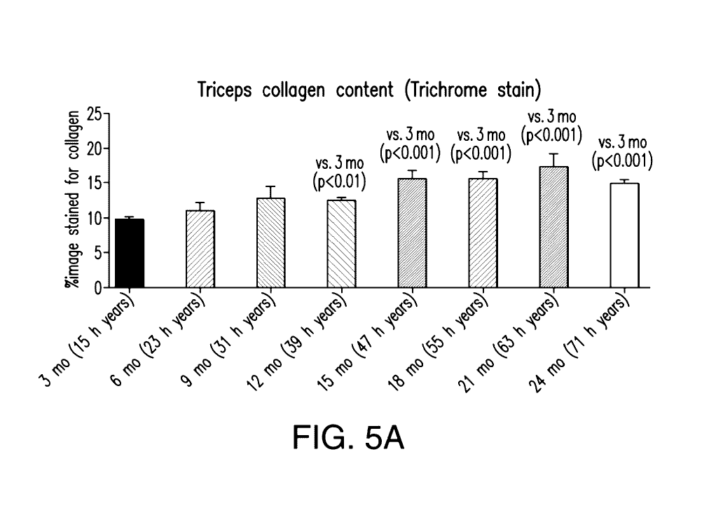

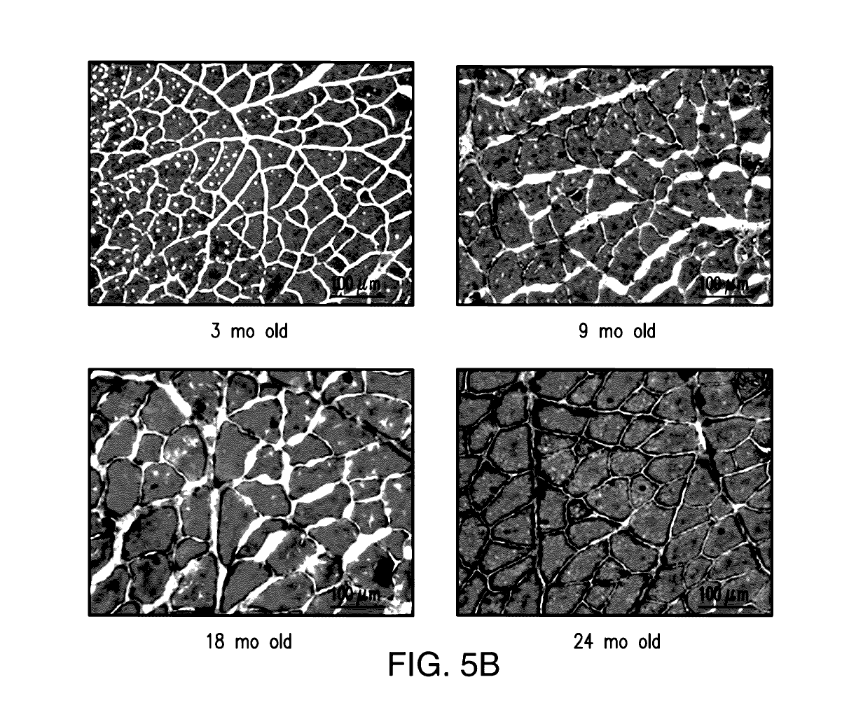

An increase in triceps fibrosis was observed beginning at 12 months of age and subsequently stabilized from 15 to 24 months of age. FIG. 5A shows tricep collagen content by Trichrome™ staining over rats lifespan (3-24 months), and FIG. 5B are exemplary Trichrome™ staining images of triceps from 3, 9, 18, or 24 month old rats.

Muscle Performance Measurements

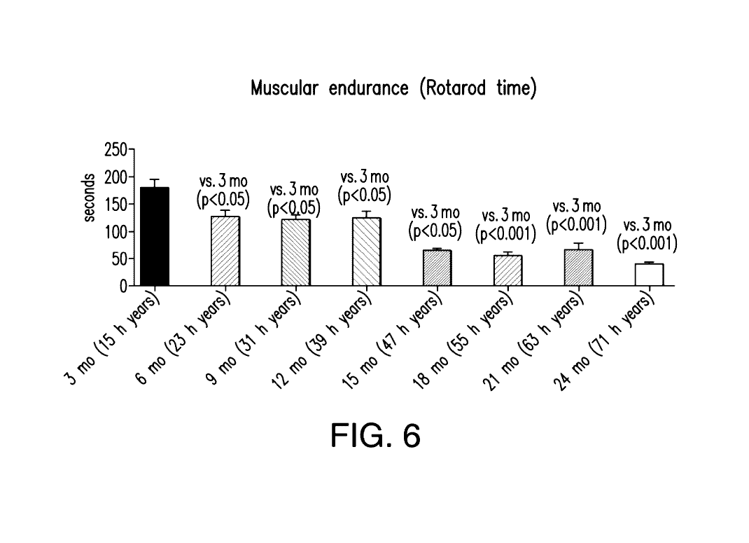

As with muscle quality (reduced mass with aging as well as satellite cell content), muscle performance also decreases with aging. Next, a muscle performance variable, muscular endurance (Rotarod time), was assessed (FIG. 6). Specifically, muscle endurance decreased after 3 months and again after 12 months.

Cardiac Muscle Stem Cell Measurements:

C-Kit-Positive Cardiac Muscle Stem Cell Flow Cytometry

Cardiac stem cells were quantified using flow cytometry. c-kit was chosen as a marker for cardiac stem cells because this is a cell surface marker that is expressed on cardiac stem cells (Magenta et al., Circ Res. 2013, 112:1202-1204).

Following heart extraction, whole hearts were weighed and the left ventricle was dissected apart from the remainder of the heart. Tissue was rinsed in ice-cold PBS and placed in 20 volumes of digestion solution (0.13% collagenase II in phosphate buffered saline (PBS)). Tissue was then minced and subsequently incubated on a rocking platform (150 rpm) at 37° C. for 30 min. The resultant slurry was passed through a 40 μm cell strainer and the effluent was collected in a 50 mL conical tube. Tubes were centrifuged for 5 min. at 2,500×g, supernatant was siphoned off and the resultant pellet was washed with 5 mL of PBS. Tubes were again centrifuged for 5 min. at 2,500×g, supernatant was siphoned off and the resultant pellet was resuspended in 1 mL of fixation solution (4% paraformaldehyde in PBS) at 37° C. for 10 min. followed by a one-minute incubation on ice. Tubes were again centrifuged for 5 min. at 2,500×g, the resultant pellet was resuspended in 200 μL of flow cytometry (FC) buffer (eBiosciences), 50 μL of the resuspended cell slurry was placed in a new 1.7 mL microcentrifuge tube, and samples were resuspended in primary antibody solution (2 μl biotin-labeled mouse anti-rat c-kit IgG1 (Abcam)+48 μL of FC buffer) at room temperature for 60 min.

Following primary antibody incubation, tubes were centrifuged for 5 min. at 2,500×g and the resultant pellet was resuspended and washed in 500 μL of FC buffer. Tubes were again centrifuged for 5 min. at 2,500×g and the resultant pellet was resuspended in secondary solution (2 μL FITC-conjugated streptavidin (Abcam)+98 μL of FC buffer) in the dark at room temperature for 60 min. Tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and FITC-labelled cells were detected using a flow cytometer (BD Accuri C6). Specifically, 10,000 events were quantified using a pre-defined gate and the proportion of cells that emitted a fluorescent intensity above background fluorescence (detected in unstained samples) were considered to be FITC-labelled c-kit-positive ventricular stem cells.

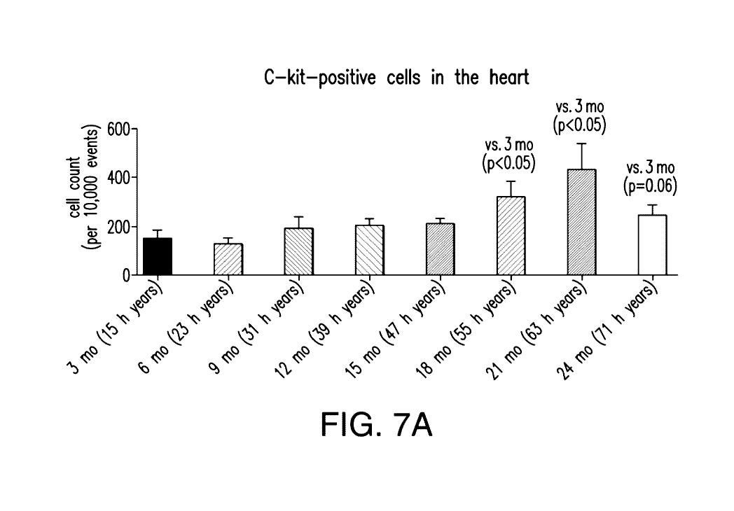

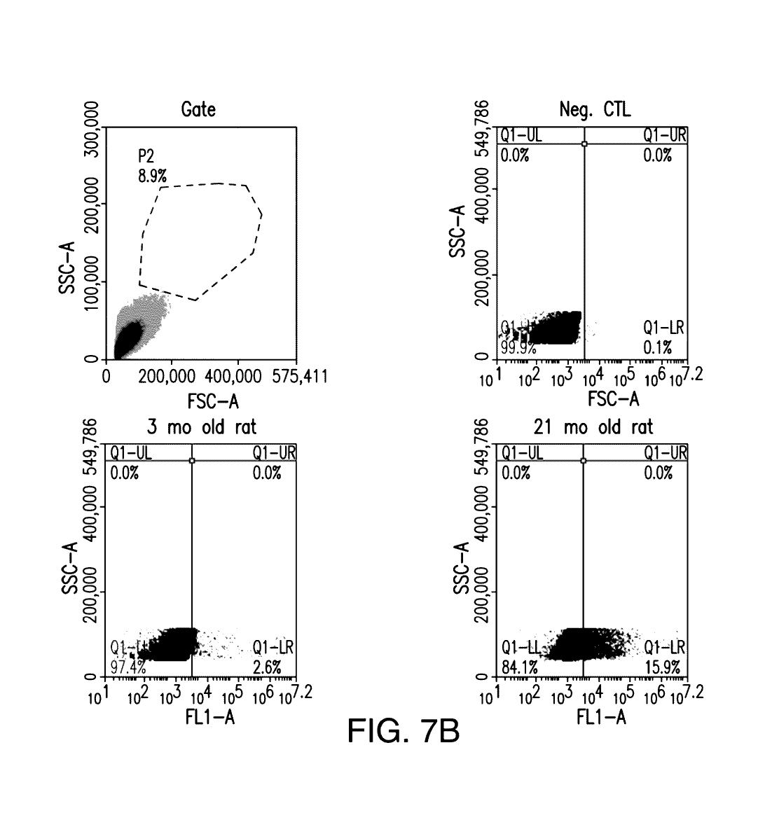

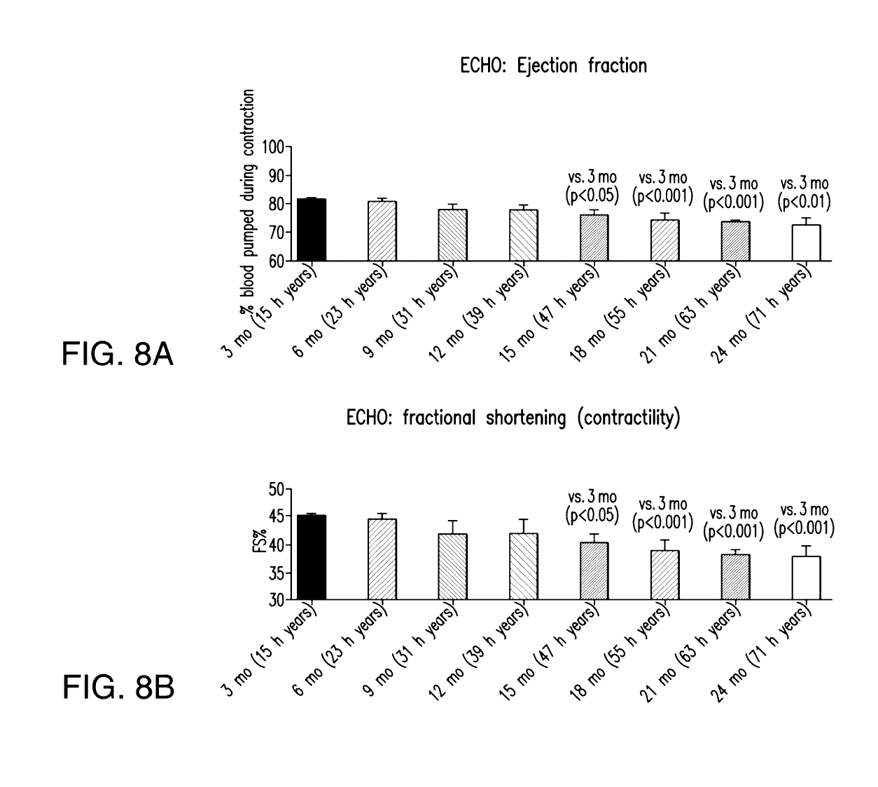

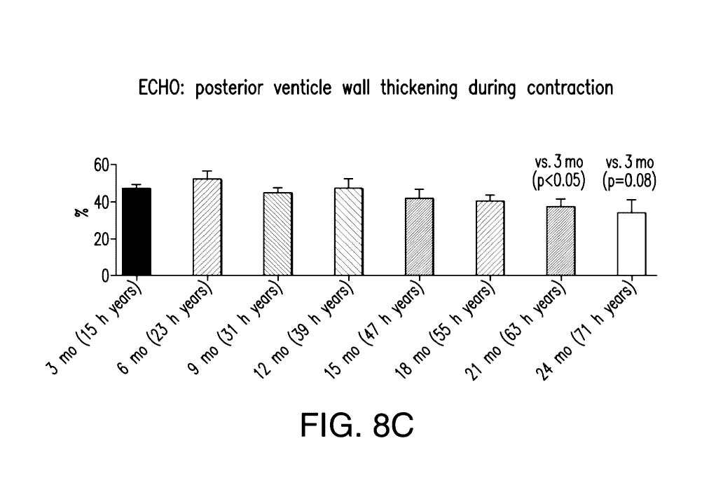

C-kit-positive cardiac stem cells increased with aging (FIG. 7A), which is in agreement with Torella et al., which reported that mice aged 20-22 months had more c-kit-positive cardiac stem cells compared to mice aged 4 months (Circ Res. 2004, 94(4):514-524 at, e.g., FIG. 8E). However, Torella et al. demonstrated that stem cell aging was also accelerated in older mice, making these stem cells less physiologically-relevant (i.e., unable to contribute to ventricular repair). FIG. 7B shows representative flow cytometry data, including gate, negative control, 3 month old rat, and 21 month old rat.

Cardiac Functions Measurements

Cardiac functions, including ejection fraction (FIG. 8A), fraction shortening (fractional shortening, FIG. 8B), and posterior ventricle wall thickening during contraction (FIG. 8C), were assessed using a small animal probe for echocardiogram. As data shown, all these cardiac functions declined with aging. Our results are in agreement with Hacker et al. (AJP—Heart 2006, 290(1):H304-H311), who reported that fractional shortening and ejection fraction decreased with aging. In addition, a negative correlation was observed between fractional shortening and cardiac stem cell content (r=−0.39, data not shown). This may illustrate a negative link between cardiac stem cell content and cardiac functions, which is opposite of our skeletal muscle stem cell findings (i.e., positive correlation between muscle stem cell content and muscle performance). A hypothetical model could be: 1. Decrease in functional ventricle mass occurs with aging due to fibrosis, which leads to a decrease in FS % and ejection fraction; or 2. Stem cells proliferate in an attempt to rescue this age-related deficit (likely unsuccessfully due to “stem cell quality/senescence”).

Cardiac Fibrosis Measurements

Fibrosis staining via Trichrome™ was then conducted. Left ventricle sections from OCT-preserved samples were cut at a thickness of 10 μm using a cryotome (HM 525 Cryostat; Thermo Fisher Scientific, Waltham, Mass.) and were adhered to positively-charged histology slides. Once all samples were sectioned, batch processing occurred for Trichrome staining using a commercially-available kit per the manufacturer's instructions (Abcam). Following staining, slides were mounted and imaged using bright field imaging (Nikon Eclipse Ti-U; Nikon Instruments, Melville, N.Y.).

A 10× image of stained ventricle tissue was obtained. Images were then analyzed via ImageJ (National Institutes of Health, Bethesda, Md.) whereby the percent of each image that was fibrotic was quantified.

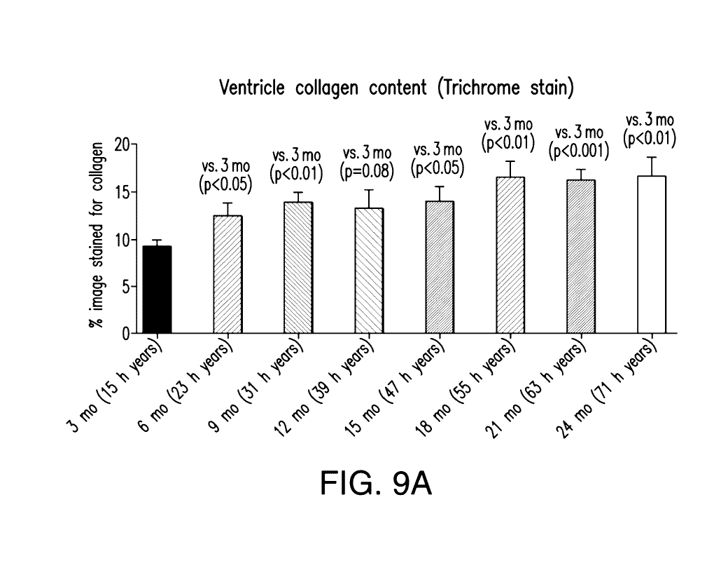

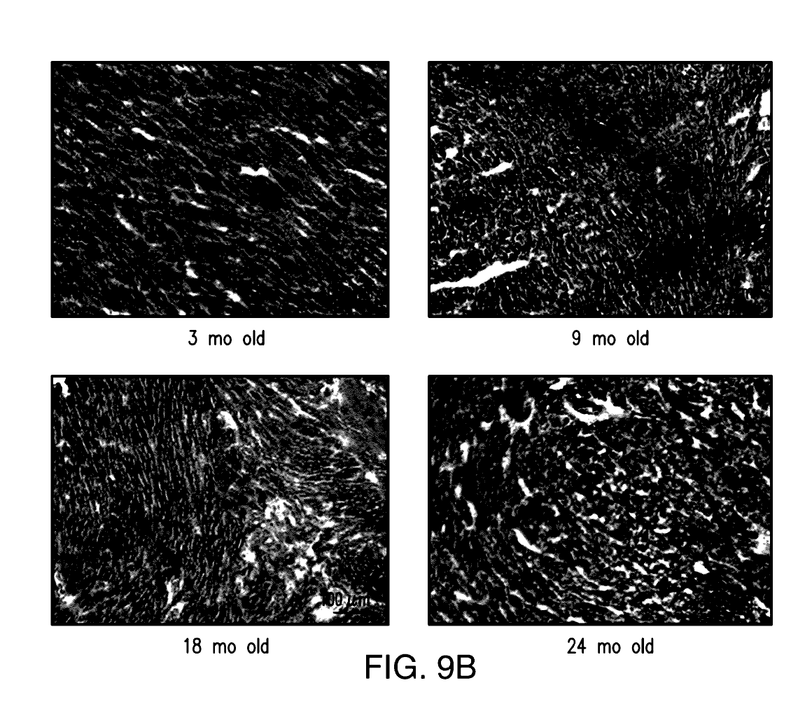

A consistent increase in left ventricle fibrosis was seen beginning at 6 months of age and a steady increase with aging. FIG. 9A shows ventricle collagen content over rats lifespan (3-24 months), and FIG. 9B are exemplary Trichrome™ staining images of left ventricle from 3, 9, 18, or 24 month old rats.

Bone Stem Cell Measurements:

Bone stem cells were quantified using flow cytometry. CD44 was chosen as a marker for bone stem cells because it is a cell surface marker that is expressed in mesenchymal stem cells derived from bone marrow (Kern et al., Stem Cells 2006, 24(5):1294-1301).

Following right femur extraction, the bone was weighed, two holes were drilled into the intercondylar fossa and femur head, respectively, and 1 ml of PBS with 1% heparin salt was flushed through the bone. Effluent was passed through a 70 μm cell strainer collected in a 50 mL conical tube. Tubes were centrifuged for 5 min. at 2,500×g, supernatant was siphoned off and the resultant pellet was washed with 5 mL of PBS. Tubes were again centrifuged for 5 min. at 2,500×g, supernatant was siphoned off and the resultant pellet was resuspended in 1 mL of fixation solution (4% paraformaldehyde in PBS) at 37° C. for 10 min. followed by a one-minute incubation on ice. Tubes were again centrifuged for 5 min. at 2,500×g and the resultant pellet was resuspended in 500 μL of permeabilization solution (0.1% Tween20 in PBS) for 15 min. at room temperature. Tubes were again centrifuged for 5 min. at 2,500×g, the resultant pellet was resuspended in 200 μL of flow cytometry (FC) buffer (eBiosciences), 50 μL of the resuspended cell slurry was placed in a new 1.7 mL microcentrifuge tube, and samples were resuspended in primary antibody solution (2 μL mouse anti-rat CD44 IgG2b (Abcam, Cambridge, Mass.)+48 μL of FC buffer) at room temperature for 60 min.

Following primary antibody incubation, tubes were centrifuged for 5 min. at 2,500×g and the resultant pellet was resuspended and washed in 500 μL of FC buffer. Tubes were again centrifuged for 5 min. at 2,500×g and the resultant pellet was resuspended in secondary antibody solution (2 μL PE-conjugated anti-mouse IgG2b antibody (eBiosciences)+98 μL of FC buffer) in the dark at room temperature for 60 min. Tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and PE-labelled cells were detected using a flow cytometer (BD Accuri C6). Specifically, 10,000 events were quantified using a pre-defined gate and the proportion of cells that emitted a fluorescent intensity above background fluorescence (detected in unstained samples) were considered to be PE-labelled CD44-positive bone stem cells.

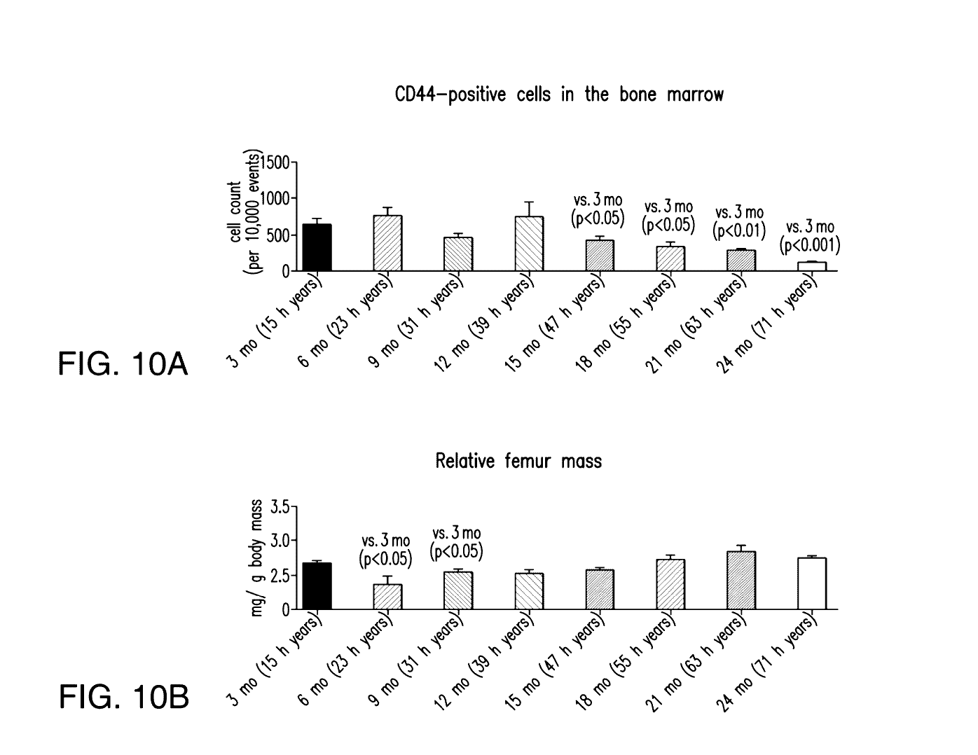

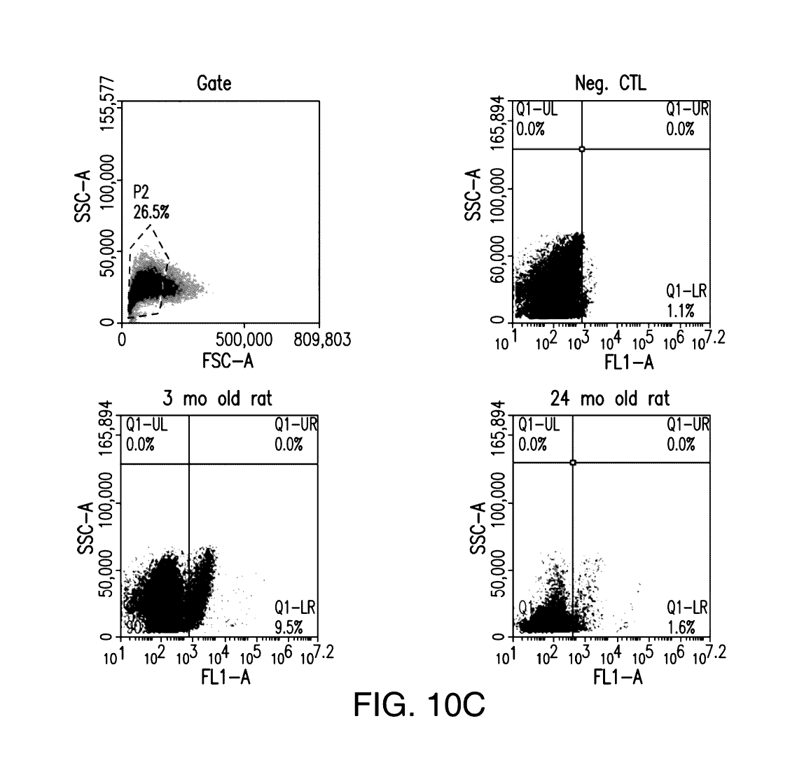

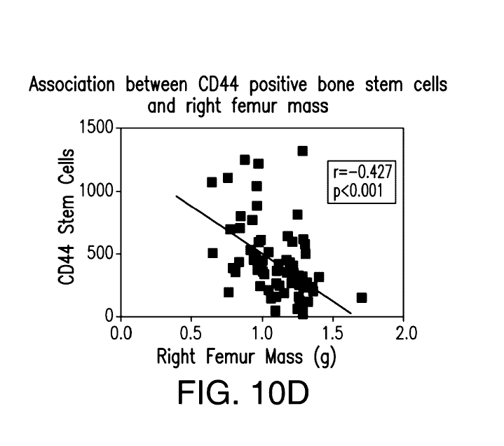

The counts of CD44-positive cells in the bone marrow (FIG. 10A) are in agreement with Stolzing et al., who reported that bone marrow-derived stem cells linearly decreased as a function of age (Mech Aging and Dev 2008, 129:163-173). FIG. 10C shows representative flow cytometry data, including gate, negative control, 3 month old rat, and 24 month old rat. On the other hand, relative femur masses increased with age (FIG. 10B). A negative relationship between relative right femur mass and bone stem cells was observed (FIG. 10D). Trabecular and cortical bone morphometrics and functional outcomes in femurs (using animal peripheral CT technology) are measured to determine specific relationships.

Brain Stem Cell Measurements:

Hippocampal (brain) stem cells were quantified using flow cytometry. NCAM (CD56) was chosen as a marker for hippocampal stem cells because this is a cell surface marker that is expressed in neuronal progenitor cells in the hippocampus (Schwartz et al., J Neuroscience Res. 2003, 74(6):838-851).

Following brain extraction, brains rinsed in ice-cold PBS and were placed in a rat-specific brain mold. The hippocampus from both hemispheres was removed using landmarks provided by Paxinos (Paxinos, The rat brain in stereotaxic coordinates, San Diego Academic (1998)). Extracted brain tissue was then placed in 20 volumes of digestion solution (2% papain in phosphate buffered saline (PBS)). Tissue was then minced and subsequently incubated on a rocking platform (150 rpm) at 37° C. for 30 min. The resultant slurry was passed through a 40 μm cell strainer and the effluent was collected in a 50 mL conical tube. Tubes were centrifuged for 5 min. at 2,500×g, supernatant was siphoned off and the resultant pellet was washed with 5 ml of PBS. Tubes were again centrifuged for 5 min. at 2,500×g, supernatant was siphoned off and the resultant pellet was resuspended in 1 ml of fixation solution (4% paraformaldehyde in PBS) at 37° C. for 10 min. followed by a one-minute incubation on ice. Tubes were again centrifuged for 5 min. at 2,500×g, the resultant pellet was resuspended in 200 μL of flow cytometry (FC) buffer (eBiosciences), 50 μL of the resuspended cell slurry was placed in a new 1.7 mL microcentrifuge tube, and samples were resuspended in primary antibody solution (2 μL mouse anti-rat NCAM IgG1 (Abcam)+48 μL of FC buffer) at room temperature for 60 min.

Following primary antibody incubation, tubes were centrifuged for 5 min. at 2,500×g and the resultant pellet was resuspended and washed in 500 μL of FC buffer. Tubes were again centrifuged for 5 min. at 2,500×g and the resultant pellet was resuspended in secondary antibody solution (2 μL FITC-conjugated anti-mouse IgG1 antibody (eBiosciences)+98 μL of FC buffer) in the dark at room temperature for 60 min. Tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and FITC-labelled cells were detected using a flow cytometer (BD Accuri C6). Specifically, 10,000 events were quantified using a pre-defined gate and the proportion of cells that emitted a fluorescent intensity above background fluorescence (detected in unstained samples) were considered to be FITC-labelled NCAM-positive neurons.

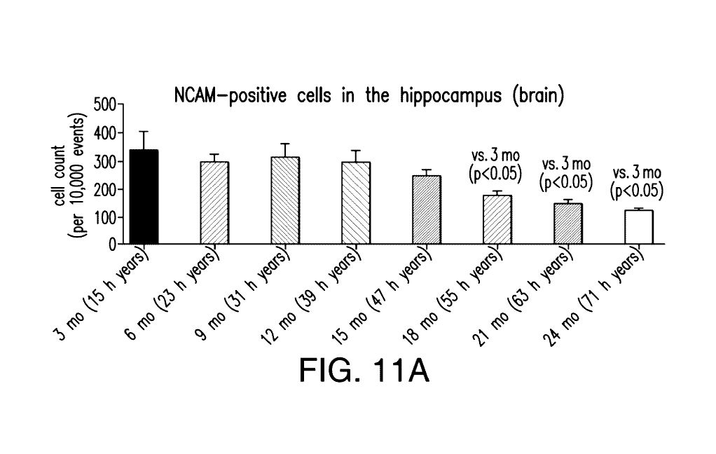

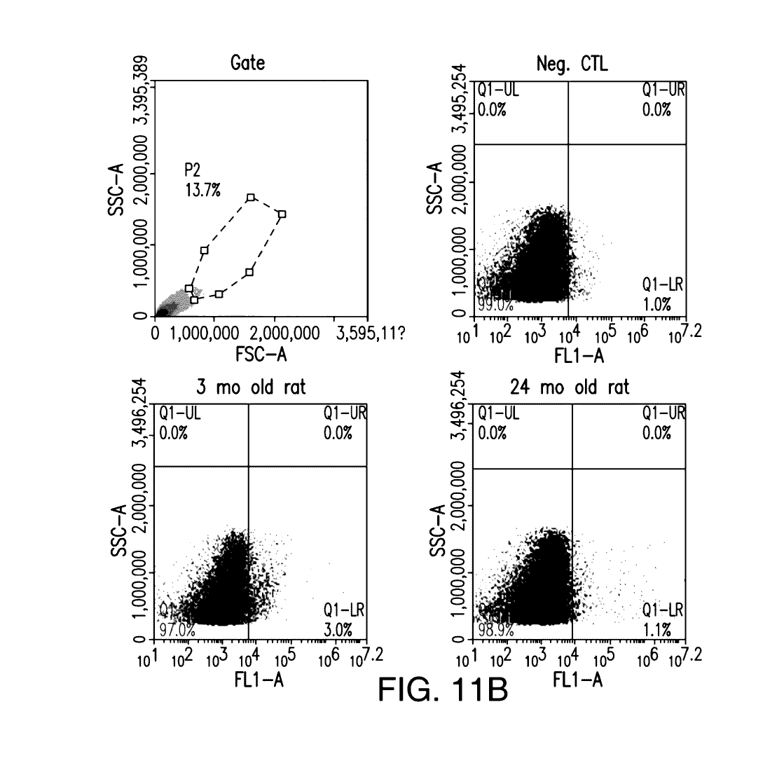

The counts of NCAM-positive cells in the hippocapal (FIG. 11A) are in agreement with Encinas et al., who reported that hippocampal stem cells linearly decreased as a function of age (Cell Stem Cell 2011, 8(5):566-579). FIG. 11B shows representative flow cytometry data, including gate, negative control, 3 month old rat, and 24 month old rat.

Other Stem Cell Measurements:

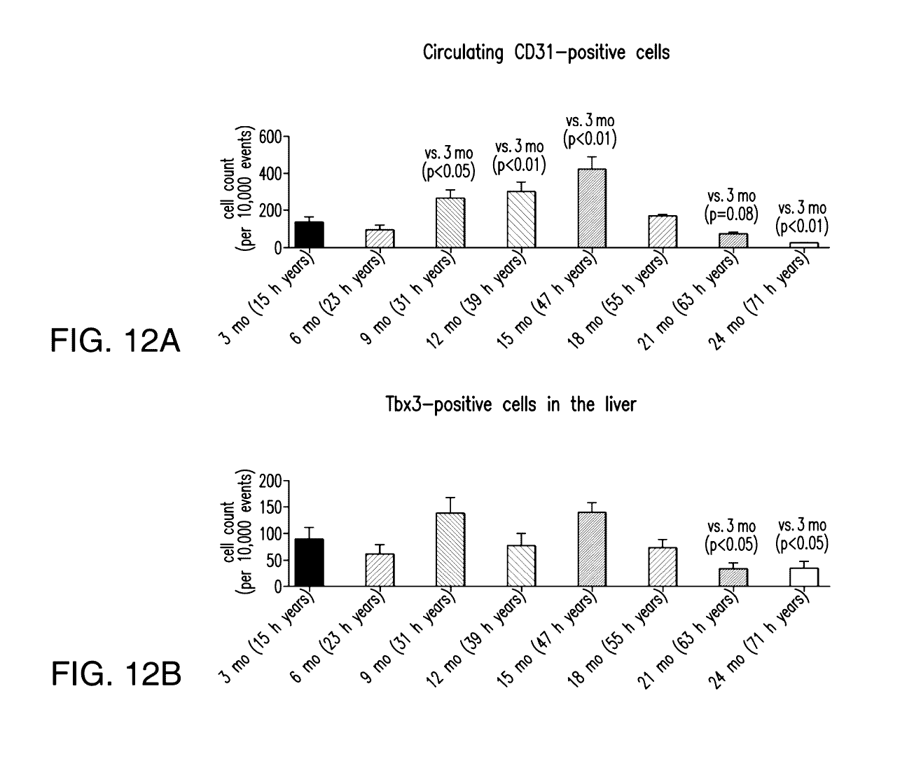

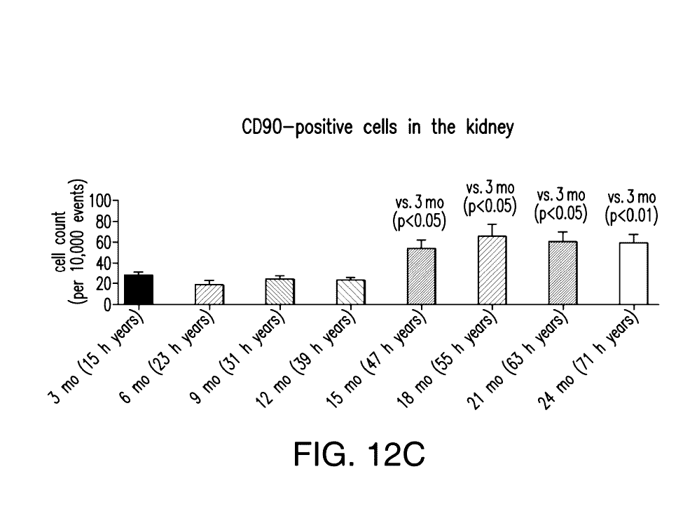

Circulating endothelial stem cells, liver stem cells, and kidney stem cells were further analyzed. CD31 was chosen as a marker for circulating endothelial stem cells (FIG. 12A); Tbx3 was chosen as a marker for liver stem cells (FIG. 12B); and CD90 was chosen as a marker for kidney stem cells (FIG. 12C).

CD31-Positive Circulating Endothelial Stem Cell Measurements

Whole blood collected in 3 ml K-EDTA tubes (described above) were first subjected to Ficoll® density gradients by placing 3 ml of whole blood and 3 mL of phosphate-buffered saline (PBS) in a 15 mL Falcon tube. Three milliliters of Ficoll®-Paque PLUS (GE Healthcare, Atlanta, Ga.) was then under-laid using a 25 mL syringe without disrupting the whole blood/PBS mixture. Thereafter, tubes were centrifuged at 400×g for 20 min. at room temperature with a slow acceleration and deceleration to prevent gradient disruption. Approximately 1 mL of the resultant PMBC layer was placed in a new 15 mL Falcon tube, washed with 5 mL of PBS, and centrifuged at 400×g for 5 min. at room temperature. The resultant supernatant was discarded, the PMBC pellet was resuspended with 200 μL FC buffer, 50 μL of the resuspended cell slurry was placed in a new 1.7 mL microcentrifuge tube, and samples were resuspended in primary antibody solution (2 μL mouse anti-rat CD31 IgG2a (Abcam)+48 μL of FC buffer) at room temperature for 60 min. CD31 was used as a circulating endothelial stem cell marker per the report by Yoder and Ingram suggesting that circulating endothelial stem cells are CD31-positive (Yoder and Ingram, Biochim Biophys Acta 2009, 1796:50-54).

Following primary antibody incubation, tubes were centrifuged for 5 min. at 2,500×g and the resultant pellet was resuspended and washed in 500 μL of FC buffer. Tubes were again centrifuged for 5 min. at 2,500×g and the resultant pellet was resuspended in secondary antibody solution (2 μL FITC-conjugated anti-mouse IgG2a antibody (eBiosciences)+98 μL of FC buffer) in the dark at room temperature for 60 min. Tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and FITC-labelled cells were detected using a flow cytometer (BD Accuri C6). Specifically, 10,000 events were quantified using a pre-defined gate and the proportion of cells that emitted a fluorescent intensity above background fluorescence (detected in unstained samples) were considered to be FITC-labelled CD31-positive circulating endothelial cells.

FIG. 12A shows that counts of circulating CD31-positive cells increased at 9 months, peaked at 15 months, and then decreased.

Tbx3-Positive Liver Stem Cell Measurements

Following liver extraction, the whole liver was weighed, and a portion of the liver (˜200-300 mg) in close proximity to the central vein was removed, rinsed in ice-cold PBS, and placed in 20 volumes of digestion solution (0.2% collagenase I in phosphate buffered saline (PBS)). Tissue was then minced and subsequently incubated on a rocking platform (150 rpm) at 37° C. for 30 min. The resultant slurry was passed through a 70 μm cell strainer and the effluent was collected in a 50 mL conical tube. Tubes were centrifuged for 5 min. at 2,500×g, supernatant was siphoned off. and the resultant pellet was washed with 5 mL of PBS. Tubes were again centrifuged for 5 min. at 2,500×g, supernatant was siphoned off. and the resultant pellet was resuspended in 1 ml of fixation solution (4% paraformaldehyde in PBS) at 37° C. for 10 min. followed by a one-minute incubation on ice. Tubes were again centrifuged for 5 min. at 2,500×g. and the resultant pellet was resuspended in 500 μL of permeabilization solution (0.1% Tween20 in PBS) for 15 min. at room temperature. Tubes were again centrifuged for 5 min. at 2,500×g, the resultant pellet was resuspended in 200 μL of flow cytometry (FC) buffer (eBiosciences, San Diego, Calif.), 50 μL of the resuspended cell slurry was placed in a new 1.7 mL microcentrifuge tube, and samples were resuspended in primary antibody solution (2 μL mouse anti-rat Tbx3 IgG1 (Abcam, Cambridge, Mass., USA)+48 μL of FC buffer) at room temperature for 60 min. Tbx3 was used as a liver stem cell marker per the findings of Wang et al. suggesting that liver stem cells are Tbx3-positive (Wang et al., Nature 2015, 524:180-185).

Following primary antibody incubations, tubes were centrifuged for 5 min. at 2,500×g and the resultant pellet was resuspended and washed in 500 μL of FC buffer. Tubes were again centrifuged for 5 min. at 2,500×g, and the resultant pellet was resuspended in secondary antibody solution (2 μL FITC-conjugated anti-mouse IgG1 antibody (eBiosciences)+98 μL of FC buffer) in the dark at room temperature for 60 min. Tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and FITC-labelled cells were detected using a flow cytometer (BD Accuri C6, San Jose, Calif.). Specifically, 10,000 events were quantified using a pre-defined gate and the proportion of cells that emitted a fluorescent intensity above background fluorescence (detected in unstained samples) were considered to be FITC-labelled Tbx3-positive liver stem cells.

FIG. 12B shows that counts of Tbx3-positive cells in the liver increased at 9 months, and decreased after 15 months.

CD90-Positive Kidney Stem Cell Measurements

Following right kidney extraction, the whole kidney was weighed, and a portion of the kidney (˜200-300 mg) was removed, rinsed in ice-cold PBS, and placed in 20 volumes of digestion solution (0.1% collagenase I in phosphate buffered saline (PBS)). Tissue was then minced and subsequently incubated on a rocking platform (150 rpm) at 37° C. for 30 min. The resultant slurry was passed through a 40 μm cell strainer and the effluent was collected in a 50 mL conical tube. Tubes were centrifuged for 5 min. at 2,500×g, supernatant was siphoned off, and the resultant pellet was washed with 5 mL of PBS. Tubes were again centrifuged for 5 min. at 2,500×g, supernatant was siphoned off, and the resultant pellet was resuspended in 1 mL of fixation solution (4% paraformaldehyde in PBS) at 37° C. for 10 min. followed by a one-minute incubation on ice. Tubes were again centrifuged for 5 min. at 2,500×g, and the resultant pellet was resuspended in 500 μL of permeabilization solution (0.1% Tween20 in PBS) for 15 min. at room temperature. Tubes were again centrifuged for 5 min. at 2,500×g, the resultant pellet was resuspended in 200 μL of flow cytometry (FC) buffer (eBiosciences), 50 μL of the resuspended cell slurry was placed in a new 1.7 mL microcentrifuge tube, and samples were resuspended in primary antibody solution (2 μL mouse anti-rat FITC-conjugated CD90 (Abcam, Cambridge, Mass.)+48 μL of FC buffer) at room temperature for 60 min. CD90 was used as a kidney stem cell marker per the report by Gupta et al. suggesting that kidney stem cells are CD90-positive (Gupta et al., J Am Soc Nephrol 2006, 17:3028-3040).

Following primary antibody incubation, tubes were centrifuged for 5 min. at 2,500×g and the resultant pellet was resuspended and washed in 500 μL of FC buffer. Tubes were again centrifuged for 5 min. at 2,500×g, pellets were resuspended in 100 μL of FC buffer, and FITC-labelled cells were detected using a flow cytometer (BD Accuri C6). Specifically, 10,000 events were quantified using a pre-defined gate and the proportion of cells that emitted a fluorescent intensity above background fluorescence (detected in unstained samples) were considered to be FITC-labelled CD90-positive kidney stem cells.

FIG. 12C shows that counts of CD90-positive cells in the kidney increased at 15 month and maintained the level through 24 month.

Statistical Comparison of all Age Group Versus 24 Month Old Rats:

Statistical analysis was conducted by comparing data of each individual age group to data of the 24 month old group. Standard errors and T-test values are summarized in the following tables.

TABLE 1
Effects of aging on body mass, organ mass, and performance

| | | | | Rel. Triceps | |
|---|---|---|---|---|---|
| | | Rotarod | | (g/kg), | |
| | Body mass | time | Triceps | corr. for | Liver |
| Age group | (g) | (sec) | (grams) | body mass | (grams) |
| 3 mo old mean | 285 | 180 | 0.93 | 3.26 | 11.50 |
| Std Err | 10 | 15 | 0.03 | 0.07 | 0.34 |
| T-test to 24 mo old | 0.00000002 | 0.00000003 | 0.00000531 | 0.01933144 | 0.00130218 |
| 6 mo old mean | 399 | 129 | 1.31 | 3.49 | 13.50 |
| Std Err | 24 | 10 | 0.05 | 0.15 | 0.57 |
| T-test to 24 mo old | 0.15254065 | 0.00000016 | 0.85907676 | 0.00749284 | 0.22753125 |
| 9 mo old mean | 393 | 123 | 1.51 | 3.85 | 13.69 |
| Std Err | 3 | 7 | 0.06 | 0.15 | 0.26 |
| T-test to 24 mo old | 0.00528909 | 0.00000001 | 0.01321761 | 0.00013206 | 0.22725827 |
| 12 mo old mean | 429 | 126 | 1.36 | 3.17 | 15.32 |
| Std Err | 7 | 13 | 0.04 | 0.12 | 0.23 |
| T-test to 24 mo old | 0.52513775 | 0.00000399 | 0.40311933 | 0.15226166 | 0.54913332 |
| 15 mo old mean | 474 | 66 | 1.37 | 2.89 | 16.17 |
| Std Err | 6 | 5 | 0.04 | 0.07 | 0.25 |
| T-test to 24 mo old | 0.02432816 | 0.000954985 | 0.284271597 | 0.694624978 | 0.115931819 |
| 18 mo old mean | 466 | 55 | 1.38 | 2.96 | 17.02 |
| Std Err | 14 | 8 | 0.06 | 0.09 | 0.91 |
| T-test to 24 mo old | 0.166825293 | 0.091945224 | 0.327474599 | 0.882162296 | 0.080800462 |
| 21 mo old mean | 445 | 69 | 1.34 | 3.00 | 14.51 |
| Std Err | 6 | 9 | 0.04 | 0.07 | 0.33 |
| T-test to 24 mo old | 0.652636022 | 0.008174369 | 0.577315957 | 0.645279172 | 0.760560069 |
| 24 mo old mean | 439 | 40 | 1.30 | 2.94 | 14.79 |
| Std Err | 13 | 4 | 0.05 | 0.10 | 0.80 |

| | | | | Femur | Rel femur |
|---|---|---|---|---|---|
| | Rel. Liver | Heart | Rel Heart | mass | mass |
| Age group | (g/kg) | (grams) | (g/kg) | (grams) | (g/kg) |
| 3 mo old mean | 40.35 | 0.89 | 3.14 | 0.76 | 2.66 |
| Std Err | 0.51 | 0.03 | 0.07 | 0.02 | 0.04 |
| T-test to 24 mo old | 0.00282063 | 0.00001705 | 0.00235036 | 0.00000000 | 0.17626808 |
| 6 mo old mean | 35.77 | 1.14 | 2.92 | 0.95 | 2.38 |
| Std Err | 1.04 | 0.08 | 0.26 | 0.01 | 0.11 |
| T-test to 24 mo old | 0.40134465 | 0.75260586 | 0.37637449 | 0.00001769 | 0.00433028 |
| 9 mo old mean | 34.82 | 1.03 | 2.62 | 1.00 | 2.55 |
| Std Err | 0.47 | 0.02 | 0.04 | 0.01 | 0.03 |
| T-test to 24 mo old | 0.62335726 | 0.00476542 | 0.70492623 | 0.00003229 | 0.00243629 |
| 12 mo old mean | 35.80 | 1.22 | 2.86 | 1.08 | 2.53 |
| Std Err | 0.67 | 0.03 | 0.10 | 0.03 | 0.06 |
| T-test to 24 mo old | 0.34359178 | 0.21722557 | 0.22411314 | 0.01373215 | 0.01385334 |
| 15 mo old mean | 34.16 | 1.23 | 2.59 | 1.21 | 2.56 |
| Std Err | 0.63 | 0.02 | 0.04 | 0.03 | 0.04 |
| T-test to 24 mo old | 0.867193592 | 0.127194614 | 0.514888517 | 0.792532149 | 0.006457671 |
| 18 mo old mean | 37.07 | 1.18 | 2.54 | 1.26 | 2.72 |
| Std Err | 3.05 | 0.03 | 0.08 | 0.03 | 0.07 |
| T-test to 24 mo old | 0.374219078 | 0.702799447 | 0.37690033 | 0.188767438 | 0.731271384 |
| 21 mo old mean | 32.56 | 1.14 | 2.57 | 1.27 | 2.85 |
| Std Err | 0.46 | 0.03 | 0.05 | 0.03 | 0.07 |
| T-test to 24 mo old | 0.525179174 | 0.712974928 | 0.452702354 | 0.173103302 | 0.240346164 |
| 24 mo old mean | 33.84 | 1.16 | 2.67 | 1.20 | 2.75 |
| Std Err | 1.81 | 0.03 | 0.12 | 0.03 | 0.04 |

TABLE 2
Effects of aging on tissue-specific stem cell markers

| | | | Brain | | | | Circulating |
|---|---|---|---|---|---|---|---|
| | Muscle NCAM- | Bone CD44- | (hippocampus) | Heart c-kit- | Kidney CD90- | Liver Tbx3- | progenitor cells |
| | positive cells | positive cells | NCAM-positive | positive cells | positive cells | positive cells | (CD31-positive) |
| | per 10,000 | per 10,000 | cells per 10,000 | per 10,000 | per 10,000 | per 10,000 | per 10,000 |
| Age group | gated events | gated events | gated events | gated events | gated events | gated events | gated events |
| 3 mo old mean | 2618 | 629 | 335 | 153 | 27 | 88 | 133 |
| Std Err | 394 | 97 | 67 | 27 | 3 | 11 | 30 |
| T-test to 24 mo old | 0.00008369 | 0.00006921 | 0.00738734 | 0.04797990 | 0.00140559 | 0.00174812 | 0.00122845 |
| 6 mo old mean | 2469 | 757 | 296 | 133 | 19 | 61 | 99 |
| Std Err | 401 | 114 | 27 | 20 | 4 | 9 | 19 |
| T-test to 24 mo old | 0.00015122 | 0.00002177 | 0.00001441 | 0.01689147 | 0.00058966 | 0.06351425 | 0.00038869 |
| 9 mo old mean | 1202 | 455 | 286 | 192 | 20 | 117 | 234 |
| Std Err | 194 | 67 | 46 | 60 | 2 | 18 | 45 |
| T-test to 24 mo old | 0.006792069 | 0.000128592 | 0.002579499 | 0.405614311 | 0.000434992 | 0.000697346 | 0.000102828 |
| 12 mo old mean | 1196 | 752 | 288 | 210 | 23 | 77 | 308 |
| Std Err | 382 | 203 | 27 | 25 | 3 | 23 | 49 |
| T-test to 24 mo old | 0.12497094 | 0.00306499 | 0.00002584 | 0.39080522 | 0.00110998 | 0.09552810 | 0.00000892 |
| 15 mo old mean | 1684 | 417 | 249 | 211 | 54 | 140 | 423 |
| Std Err | 298 | 59 | 22 | 17 | 8 | 16 | 59 |
| T-test to 24 mo old | 0.00225765 | 0.00018015 | 0.00012110 | 0.34719767 | 0.66190351 | 0.00001843 | 0.00000201 |
| 18 mo old mean | 582 | 336 | 176 | 326 | 65 | 73 | 170 |
| Std Err | 79 | 42 | 17 | 56 | 12 | 16 | 9 |
| T-test to 24 mo old | 0.81112539 | 0.00025312 | 0.02850855 | 0.28480646 | 0.69290446 | 0.05816650 | 0.00000000 |
| 21 mo old mean | 385 | 279 | 149 | 434 | 60 | 33 | 67 |
| Std Err | 94 | 29 | 15 | 108 | 10 | 11 | 8 |
| T-test to 24 mo old | 0.05737144 | 0.00048719 | 0.28283698 | 0.11503539 | 0.95858171 | 0.90533781 | 0.00003615 |
| 24 mo old mean | 606 | 117 | 130 | 251 | 59 | 35 | 19 |
| Std Err | 58 | 24 | 10 | 38 | 8 | 9 | 4 |

TABLE 3
Effects of aging on muscle and heart fibrosis
and muscle Pax-7 positive cells

| | # Pax-7- | | |
|---|---|---|---|
| | positive cells | % fibrotic | % fibrotic |
| Age group | per 40x image | (ventricle) | (triceps) |
| 3 mo old mean | 8.90 | 9.12 | 9.61 |
| Std Err | 0.43 | 0.59 | 0.51 |
| T-test to 24 mo old | 0.00001492 | 0.00063030 | 0.00000027 |
| 6 mo old mean | 9.30 | 12.36 | 11.00 |
| Std Err | 0.60 | 1.24 | 1.00 |
| T-test to 24 mo old | 0.00003374 | 0.05884418 | 0.00191279 |
| 9 mo old mean | 7.67 | 14.27 | 12.78 |
| Std Err | 0.76 | 0.89 | 1.60 |
| T-test to 24 mo old | 0.00987616 | 0.25527109 | 0.18403839 |
| 12 mo old mean | 7.78 | 13.16 | 12.41 |
| Std Err | 0.57 | 1.77 | 0.50 |
| T-test to 24 mo old | 0.00218998 | 0.17778908 | 0.00117922 |
| 15 mo old mean | 8.90 | 13.94 | 15.61 |
| Std Err | 0.43 | 1.61 | 1.15 |
| T-test to 24 mo old | 0.00001492 | 0.26812054 | 0.60839549 |
| 18 mo old mean | 6.30 | 16.40 | 15.57 |
| Std Err | 0.26 | 1.44 | 0.91 |
| T-test to 24 mo old | 0.041861106 | 0.918810946 | 0.558724895 |
| 21 mo old mean | 6.11 | 16.14 | 17.37 |
| Std Err | 0.63 | 0.98 | 1.59 |
| T-test to 24 mo old | 0.215723553 | 0.811682841 | 0.144838643 |
| 24 mo old mean | 5.10 | 16.63 | 14.97 |
| Std Err | 0.48 | 1.72 | 0.44 |

TABLE 4
Effects of aging on functional echocardiogram measures

| | Ejection fraction (% | Fractional |
|---|---|---|
| Age group | blood pumped) | shortening (FS %) |
| 3 mo old mean | 81.38 | 45.01 |
| Std Err | 0.62 | 0.62 |
| T-test to 24 mo old | 0.00160 | 0.00210 |
| 6 mo old mean | 80.80 | 44.58 |
| Std Err | 0.91 | 0.92 |
| T-test to 24 mo old | 0.00370 | 0.00510 |
| 9 mo old mean | 78.15 | 42.09 |
| Std Err | 2.05 | 1.82 |
| T-test to 24 mo old | 0.09510 | 0.12500 |
| 12 mo old mean | 77.83 | 42.07 |
| Std Err | 2.15 | 2.00 |
| T-test to 24 mo old | 0.12300 | 0.14000 |
| 15 mo old mean | 76.17 | 40.30 |
| Std Err | 1.83 | 1.59 |
| T-test to 24 mo old | 0.26100 | 0.32730 |
| 18 mo old mean | 74.13 | 38.79 |
| Std Err | 2.34 | 1.93 |
| T-test to 24 mo old | 0.69330 | 0.71770 |
| 21 mo old mean | 73.70 | 38.04 |
| Std Err | 1.22 | 1.04 |
| T-test to 24 mo old | 0.73730 | 0.9101 |
| 24 mo old mean | 72.84 | 37.79 |
| Std Err | 2.21 | 1.92 |

Molecular Analysis:

The same tissues (e.g., skin, kidney, liver, brain, heart, muscle, etc.) are also collected for further molecular analysis.

Preservation of Samples for mRNA:

RNA stabilization—RNA in harvested animal tissue is not protected until the tissue is completely submerged in a sufficient volume of RNAlater® RNA Stabilization Reagent (Qiagen). After harvesting, the tissue is immediately placed in at least 10 volumes of the reagent (or approximately 10 μl reagent per 1 mg tissue). Larger volumes can be used if necessary or desired; whereas smaller volumes could possibly lead to RNA degradation during storage. The storage containers used are wide enough so that the reagent covers the entire tissue. Procedures for tissue harvesting and RNA stabilization is carried out as quickly as possible.

Tissue size should be optimized to ensure successful RNA stabilization with RNAlater® RNA Stabilization per manufacturer's instructions.

Immediately upon contact, the reagent diffuses into the surface layer and outer portions of solid tissues. To ensure rapid and reliable stabilization of RNA (e.g., in the inner parts of solid tissues), the sample is cut into slices less than 0.5 cm thick. The slices can be any convenient size, provided one dimension of the sample is <0.5 cm. If the slices are thicker than 0.5 cm, it is possible that the reagent can diffuse too slowly into the interior of the sample and RNA degradation can occur. Small organs such as rat kidney and spleen or most mouse organs (except liver) do not require slicing and the entire organ can be placed in RNAlater® RNA Stabilization Reagent.

The following guide can be used to determine the amount of RNAlater®RNA Stabilization Reagent required for RNA stabilization:

A cube of rat kidney with a 5 mm edge length ((5 mm)³=125 mm³=125 μL) weighs 150-175 mg and requires at least 1.5-1.75 mL of the reagent. A 3 mm cube ((3 mm)³=27 mm³=27 μL) of most animal tissues weighs 30-35 mg and requires at least 300-350 μL of the reagent.

Although weighing tissues is generally more accurate, RNA in unstabilized tissues can degrade during weighing. In some cases, however, it may be more convenient to quickly estimate the weight of tissue pieces. Average weights of various entire adult mouse organs and the corresponding amounts of RNAlater® RNA Stabilization Reagent used are provided in the table below. RNA in tissues weighing up to 150 mg can be stabilized in 1.5 mL RNAlater® TissueProtect™ Tubes. For tissue pieces weighing more than 150 mg and less than 500 mg, 5 mL RNAlater® Tissue Protect Tubes can be used.

| Organ/Tissue | Weight (mg) | Reagent (mL) | Protect Tube |
|---|---|---|---|
| Kidney | 180-250 | 1.8-2.5 | 5 mL |
| Spleen | 100-160 | 1-1.6 | 1.5 mL or 5 mL |
| Lung | 190-210 | 1.9-2.1 | 5 mL |
| Heart | 100-170 | 1-1.7 | 1.5 mL or 5 mL |
| Liver | 1000-1800 | 10-18 | Use other container |

Tissues to be preserved—Brain, heart and major blood vessels, lungs, liver, kidney, bone marrow, skeletal muscle, and skin are preserved. Other tissues that can be preserved include spinal cord, lungs, eyes, adipose tissue, pancreas, lymph nodes, testis, prostate, ovary, endometrium, thyroid glands, spleen, GI tract, and serum proteins.

Biomarker Discovery:

The brain, heart and major blood vessels, lungs, liver, kidney, bone marrow, skeletal muscle, skin are analyzed for biomarkers.

Gene expression analysis—Gene expression analysis is performed by TaqMan® Low Density Arrays on 7900HT Real-Time PCR Systems. The general protocol is summarized below:

First, DNA is synthesized from total RNA samples using the High Capacity™ cDNA Archive Kit (PN 4322171). Use random primers to generate cDNA from total RNA samples. The cDNA sample can be stored at −15 to −25° C.

Next, cDNA is amplified, and a sample-specific PCR mix is prepared. For each cDNA sample, a 1.5 mL microcentrifuge tube is labeled. If the cDNA samples were stored at −15 to −25° C., then the samples are thawed. The samples are vortexed, and the tubes are centrifuged. For each sample, the following components are added to the labeled 1.5 mL microcentrifuge tube:

| | Volume (μL) |
|---|---|
| Component | per Fill Reservoir |
| cDNA sample (30 to 1000 ng) + RNase-free water | 50.0 |
| TaqMan ® Universal PCR Master Mix (2X) | 50.0 |
| Total Volume | 100.0 |

The microcentrifuge tubes are capped, and the solution is thoroughly mixed by gently vortexing. The tubes are centrifuged to eliminate air bubbles from the mixtures. The TaqMan® Arrays are then loaded as described below.

The sample-specific PCR Reaction mix is then loaded in to fill reservoirs. When the original packaging (plastic tubs) has reached room temperature, a TaqMan® Array is removed from its packaging. The TaqMan® Array is placed on a lab bench, with the foil side down.

One hundred microliters of the desired sample-specific PCR reaction mix is loaded into a 100 μL micropipette. The micropipette is held in an angled position and place the tip in the fill port. The sample-specific PCR reaction mix is then dispensed so that it sweeps in and around the fill reservoir toward the vent port.

Next, the TaqMan® Array is centrifuged. The TaqMan® Arrays are placed into buckets. An empty Sorvall/Heraeus Custom Bucket and array holder is obtained. The bucket is placed on a lab bench. Then the TaqMan® Arrays are placed into the array holding, making sure that the fill reservoir projects upwards out of the array holder and the reaction wells face the same direction as the “This Side Out” label. Blank balance arrays are used to fill any remaining positions in the array holder. A filled array holder is filled in the bucket so that the “This Side Out” label faces the front of the bucket, which may have the Sorvall® emblem on it.

The Centrifuge settings are set using the following operating parameters:

| Parameter | EASYSet (touchpad) | QUIKSet (knob-operated) |
|---|---|---|
| Up Ramp rate | 9 | 3 |
| Down Ramp rate | 9 | N/A |
| Rotational speed | 1,200 rpm (331 × g) | 1200 rpm |
| Centrifugation time | 2 × 1 min | 2 × 1 min |

The buckets are then placed in the centrifuge and the centrifuge is started.

The TaqMan® Array is then sealed. The sealer is placed on a sturdy lab bench, and the TaqMan Array® is inserted into the sealer. The TaqMan Array® is oriented in the proper direction over the sealer's insert plate. The TaqMan Array's fill reservoir end is the end closest to the arrows etched in the base of the sealer. The Array's rear pin groves, foil side up, are lined up to the stylus pins on the sealer. The Array is gently placed on top of the insert plate and it is ensured that the front end of the array is held securely in place by the spring clips. The TaqMan® Array is gently pulled out until it is seated securely in the insert plate. The carriage across the base of the sealer is pushed in the direction of the arrows. The sealed TaqMan® Array is then removed by grasping its sides and lifting it off the sealer's insert plate. The TaqMan® Array is inspected for proper sealing. The indentations from the stylus assembly are matched up with the TaqMan® Array's main channels.

Next, the fill reservoirs are trimmed off. Using scissors, the fill reservoirs are trimmed from the TaqMan® Array. The edge of the TaqMan® Array's carrier is used as a guide.

Finally, real-time data analysis is performed. SDS plate documents store data collected from a run including sample names and detectors. On the computer, the SDS software is started, and the setup file is imported into a new plate document and save the plate document. To perform the run, the plate document in the SDS software is opened, and the Instrument tab of the plate document and then the Real-Time tab are selected. The “Connected to Plate Name” is verified to be displayed in the status bar. The TaqMan® Array thermal cycling block is then verified to be installed in the instrument tray. The prepared array is then placed in the instrument tray with Well A1 at the top left corner of the tray, the notched corner at the top right, and the bar code toward the front of the instrument. When the run is complete and the Run Complete dialog box appears, the dialog box is closed.

Protein expression quantification—Protein expression quantification is performed by Two-Dimensional Differential Gel Electrophoresis (2D-DIGE) (Aberdeen Proteomics, www.abdn.ac.uk/ims/proteomics). The analytical workflow is summarized below:

First, samples are prepared using standard protocols for protein analysis by 2D-DIGE and the protein concentration determined. The proteins are precipitated and resuspended, at a concentration of approximately 6 g/μL, in a compatible buffer for labeling.

Next, individual protein samples are labeled using the Cy3 or Cy5 dyes in a minimal labelling protocol; 50 μg of protein are labelled according to the manufacturer's protocol. A pooled sample, prepared by mixing equal amounts of protein for all samples in the experiment, is labelled with the Cy2 dye.

Following labelling, the samples are then loaded to immobilized pH gradient (IPG) gel strips. Specifically, Cy3- and Cy5-labelled samples along with an aliquot of the Cy2-labelled pool sample are loaded to each IPG gel.

The IPG gel strips are then focused on an IPGPhor® instrument (GE Healthcare) before being transferred to a Criterion precast slab gel (Bio-Rad Ltd) for the second dimension electrophoresis step using standard equilibration buffers. (Cash et al., Methods Mol Bio 2009, 519:131-144).

Finally, the gels are scanned using an Ettan™ DIGE Imager (GE Healthcare) to image each gel to detect the Cy2-, Cy3- and Cy5-labelled proteins. The image files are transferred to Progenesis SameSpots, Version 4.2 (Nonlinear Dynamics) for gel alignment, spot detection and statistical analysis. Data processing uses the in-built routines available within the Progenesis SameSpots software.

Protein identification—Protein identification is performed by mass spectrometry.

### 5.2 Example 2

### Longevity Study

This study was conducted to evaluate the effect of administering exogenous stem cells on the lifespan of experimental animals and determine how those cells impact the quality and quantity of stem cell reservoirs in various organs and tissues.

Materials and Methods

Animals

F344 Fisher Rats, aged 11, 17 and 21 months were housed under a 12-hour light/12-hour dark cycle and provided standard rat chow and water ad libitum. Animals were weighed and assigned an ID number. Animals were randomly divided into groups using a random number generator. All procedures were approved through the Institutional Animal Care and Use Committee.

Rats were administered subcutaneously or intravenously with PDSC at age of 11, 17, or 21 months. In all the data presented herein, the age of rats refers to the age when the rats were treated, not the age when they were sacrificed.

Blood Sample Preparation and Analysis

Venous blood samples were collected from the saphenous vein and collected into ethylene-diamine-tetra acetic acid (EDTA) (for whole blood count) and Lithium Heparin (for plasma) tubes. Approximately 400 μL total was collected from each animal before and after the experimental period. Whole blood counts (WBC) were determined using a VestScanII Blood Panel. Lithium Heparin tubes were centrifuged for 10 min at 1200×g. Plasma was transferred to a fresh microtube and immediately frozen at −80° C. for later analysis.

Preparation and Transplantation of PSC-100

PSC-100 cells were quickly thawed in a 37° C. water bath until a small amount of frozen material remained. Cells were resuspended approximately 10-fold in 2.5% bovine serum albumin (BSA) in phosphate buffered saline (PBS) and a cell count aliquot was taken to determine total cells obtained. Cells were then centrifuged at 500×g for 5 min. An appropriate volume of 2.5% BSA in PBS was used to resuspend the cell pellet to a concentration yielding a 0.5 mL dose per animal. A correction factor (18.74%) was utilized to account for the cell lysis occurring during injection with a 26 gauge needle and was experimentally determined from trials of sample cell preparation cell counts before and after passing through a 26 gauge needle. F344 rats were subcutaneously or intravenously (through tail vein) administered 0.5 mL of placebo (negative control), 1 million PSC-100 (low dose), or 10 million PSC-100 (high dose).

Rotarod Evaluation

An IITC Rotarod (IITC Life Science Inc., Woodlands Hills, Calif.) equipped with 9.5 cm diameter drums was used. All animals completed one training period in which repeated bouts on the rotarod were performed until 2 consecutive successful 10 second duration trials were able to be performed. Approximately 2 hours after this training period, the actual test was performed with a starting RPM of 2, constant acceleration to 30 RPM over a period of 100 seconds. Total test time was 100 seconds. Animals performed 3 consecutive trials for data acquisition.

RNAseq Analysis

RNA extraction of tissue samples was carried out using a combination of Trizol (Thermo Fisher Scientific Inc., San Jose, Calif.) extraction followed by on column purification using an RNeasy® Mini Column (Qiagen Inc., Valencia, Calif.). Samples were homogenized in Trizol using a 5 mm stainless bead with a TissuLyser® II set at 50 Hz for a 3 min period. Aqueous extractions were applied to RNeasy® Mini Columns and purified according to the manufacturer's instructions. Sample concentrations were determined using a Nanodrop spectrophotometer and RNA quality was evaluated using the Bioanalyzer.

Poly(A) RNA was isolated using the kit from New England Biolabs (Ipswich, Mass.), NEBNext® Poly(A) mRNA Magnetic Isolation Module, and barcoded libraries were made using the NEBNext® Ultra™ Directional RNA Library Prep Kit for Illumina® (NEB, Ipswich, Mass.). Libraries were pooled and single end sequenced (1X75) on the Illumina® NextSeq 500 using the High output V2 kit (Illumina Inc., San Diego, Calif.) producing about 30 million reads per sample. Read data was processed in BaseSpace (basespace.illumina.com). Reads were aligned to Rattus norvegicus genome (rn5) using STAR aligner (https://code.google.com/p/rna-star/) with default settings. Differential transcript expression was determined using the Cufflinks Cuffdiff package (http://cufflinks.cbcb.umd.edu/) and result lists were generated using a 2-fold difference cutoff with a False Discovery Rate of less than 0.05. Lists of differentially expressed genes were input into Ingenuity Pathway Analysis (IPA) to identify networks and determine putative upstream regulators.

Skeletal Muscle Preparation

Soleus and plantaris muscles from the left leg were quickly dissected and flash frozen in liquid nitrogen (N₂) and stored at −80° C. for RNA and proteomic analyses. Right leg soleus and plantaris were dissected, embedded in cryomolds, and frozen in liquid nitrogen chilled isopentane and stored at −80° C. Total gastrocnemius was removed, weighed, and flash frozen in liquid N₂.

Cortex Preparation

The brain was quickly removed from sacrificed rats and hemispheres were separated. The right hemisphere was used to extract the hippocampus which was flash frozen in liquid N₂. The left hemisphere was placed into a 22 mm cryomold, embedded with OCT, and frozen in liquid N₂-chilled isopentane.

Bone Marrow Preparation, CFU Assay and Flow Cytometry

Cells were isolated from rat bone marrow by removing the ends of the femurs and flushing with a 21 gauge needle with 5 mL alpha-MEM with 10% FBS for a total of three times. The crude marrow suspension was then passed through a 70 μm filter to obtain a cell suspension. Cell suspensions were then spun at 300×g for 5 mins at 4° C. The supernatant was aspirated and the pellet was resuspended in 5 mL 1×RBC lysis buffer (BioLegend Cat#420301). Cells in RBC lysis buffer were kept on ice and agitated once per minute for 5 mins. After 5 mins, the reaction was quenched by adding 25 mL PBS. For the colony-forming unit (CFU) assay, the cells were pelleted at 300×g for 5 mins. The supernatant was discarded and the pellet was resuspended in 20 mL PBS and spun at 300×g for 5 mins. The supernatant was discarded and the pellet was resuspended in 5-10 mL alpha-MEM+10% FBS media. The cells were then counted via trypan blue exclusion using a Countess machine (Cat# C10277) and appropriate amounts of media were added to make a final concentration of 1-2 million cells/mL. For the CFU assay, two dilutions were plated, 1 million cells in a T75 flask and 250,000 cells in a T75 flask.

Ten million cells were removed from the prepared bone marrow and fixed with 2% PFA at room temperature with agitation for 10 mins. Cells were then pelleted by centrifugation to remove PFA and washed twice with PBS. Pre-conjugated antibodies were used for flow cytometry for identification and quantification of cell types.

Immunofluorescence Detection of Endogenous Stem Cells

Anti-Pax7 antibody from Developmental Studies Hybridoma Bank (Iowa City, Iowa) was used to detect satellite cells. Fresh frozen muscle samples were sectioned at 10 microns at −20° C. and fixed in 2% PFA for 10 min. Following fixation, antigen retrieval was performed using HistoVT™ One (70° C. for 20 min). Slides were then washed with PBS and blocked in 5% normal donkey serum with 0.3% Triton-X 100 for 20 min. Anti-Pax7 antibody at 1:20 dilution was used in an overnight incubation. Slides were then washed 3 times 10 min each with PBS and then incubated with donkey anti-mouse antibody #555 (from Abcam Inc., Cambridge UK) for 1 to 2 hours. Slides were washed once and incubated for 5 min with DAPI, followed by washing 3 times in PBS. Slides were then mounted with Fluorsave™ (EMD Millipore, Darmstadt, Germany) and coverslipped with #1½ coverslips. Imaging was carried out using a Zeiss Axiovision upright fluorescence microscope and images were obtained using the Zeiss software package. Images were then imported to NIH ImageJ for analysis.

Anti-Ki67 antibody from Abcam Inc. (Cambridge UK) was used to detect subventricular zone stem cells. Fresh frozen cortex samples were sagittally sectioned at 10 microns at −20° C. and fixed in ice cold acetone for 5 min. Following fixation, slides were washed with PBS and blocked in 5% normal donkey serum with 0.3% Triton-X 100 for 20 min. Anti-Ki67 antibody at 1:200 dilution was used in an overnight incubation. Slides were then washed 3 times 10 min each with PBS and then incubated with donkey anti-rabbit antibody #555 (from Abcam) for 1 to 2 hours. Slides were washed once and incubated for 5 min with DAPI, followed by washing 3 times in PBS. Slides were then mounted with Fluorsave and coverslipped with #1-½ coverslips. Imaging was carried out using a Zeiss Axiovision upright fluorescence microscope and images were obtained using the Zeiss software package. Images were then imported to NIH ImageJ for analysis.

Anti-laminin antibody from Developmental Studies Hybridoma Bank (Iowa City, Iowa) was used to detect muscle fiber boundaries. Fresh frozen muscle samples were sectioned at 10 microns at −20° C. and fixed in 2% PFA for 10 min. Slides were then washed with PBS and blocked in 5% normal donkey serum with 0.3% Triton-X 100 for 20 min. Anti-Laminin antibody at 1:200 dilution was used in an overnight incubation. Slides were then washed 3 times 10 min each with PBS and then incubated with donkey anti-mouse antibody #555 (from Abcam) for 1 to 2 hours. Slides were washed once and incubated for 5 min with DAPI, followed by washing 3 times in PBS. Slides were then mounted with Fluorsave and coverslipped with #1½ coverslips. Imaging was carried out using a Zeiss Axiovision upright fluorescence microscope and images were obtained using the Zeiss software package. Images were then imported to NIH ImageJ for analysis.

Image Analysis and Quantification

Stem cell populations identified by Pax7 in muscle or Ki67 in hippocampus were quantified via the particle counting function in ImageJ software after appropriate thresholding. Parameters were adjusted to be consistent with independent manual counting measurements.

Determination of Muscle Fiber Cross-Sectional Area (CSA)

Anti-laminin muscle section images were analyzed with NIH ImageJ and threshold was adjusted to select the fiber entities. Pixel size limits were set between 400 and 60,000 and particle counts were performed in which the output was the number of fibers and the respective CSA for each counted entity.

CSA Frequency Distribution Analysis

CSA data were imported to GraphPad™ Prism program and a frequency analysis was used in which the bin setting was 0.05. The output file provided the number of fibers for each bin.

Statistical Analysis

A one way analysis of variance (ANOVA) with multiple comparisons was used to determine significant differences among means and a Tukey's post hoc test was used to identify the significantly different groups.

Results

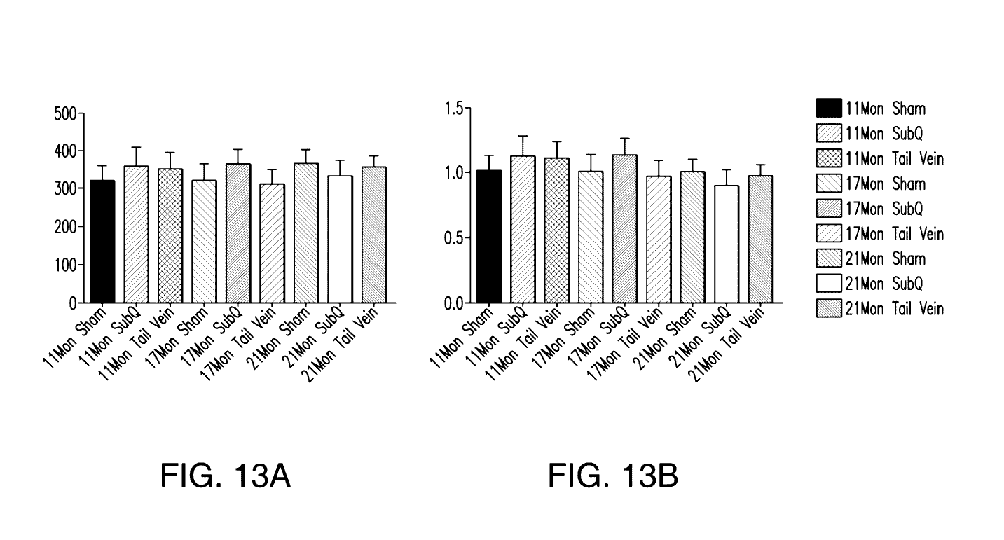

Total body mass of rats was maintained throughout aging (FIGS. 13A-13B). However, changes in body composition were observed (data not shown), so was tissue replacement by fibrosis, which leads to a decline in tissue functionality parallel to a decline in stem cell reservoir.

Skeletal Muscle Performance Test

The rotarod test measures skeletal muscle endurance. Some of the functions of the test include balance, grip strength, and neuromuscular coordination. It is used to mimic the 2-6 min walk test in humans, gait speed, and balance, which have been associated with survival in older adults.

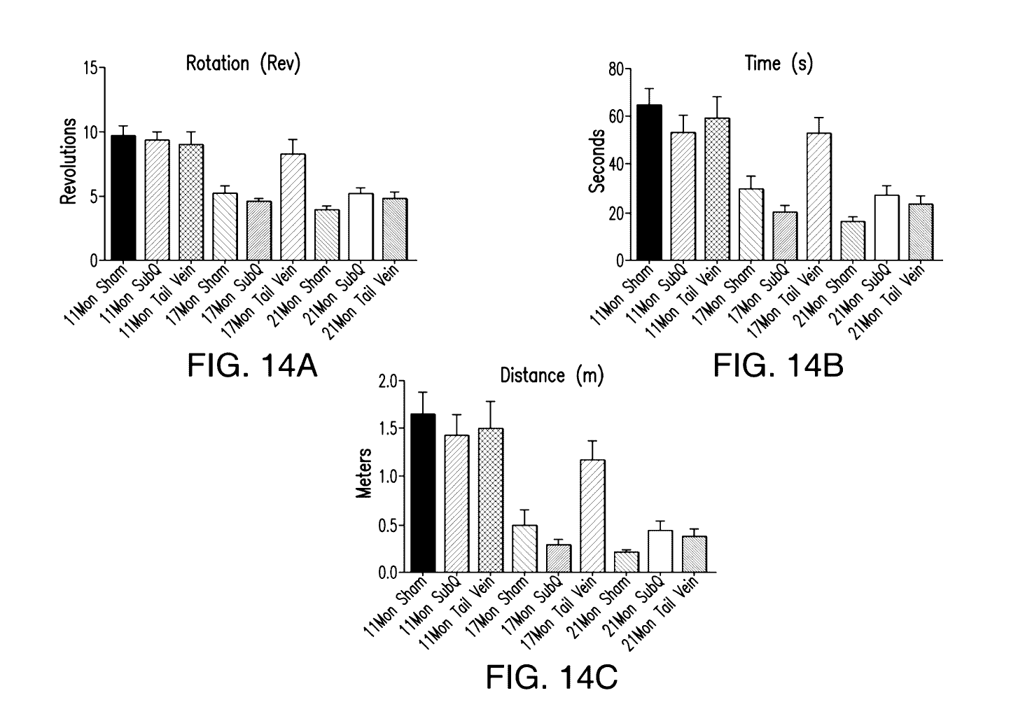

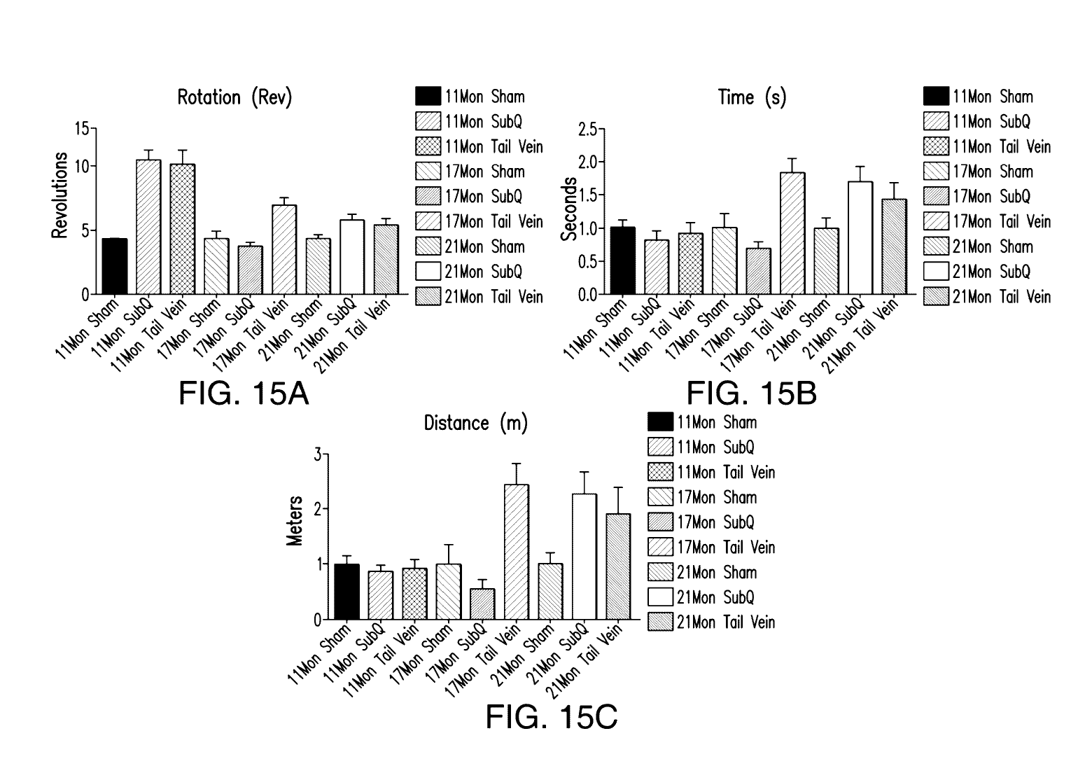

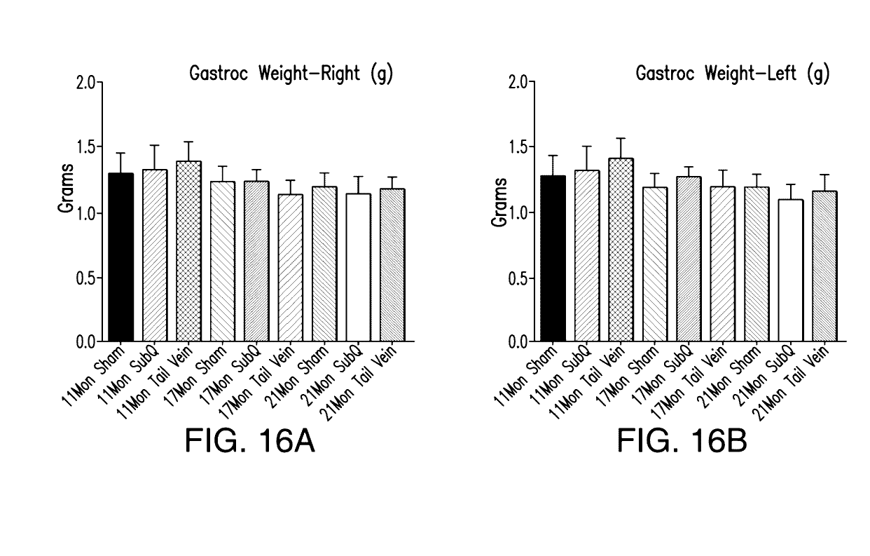

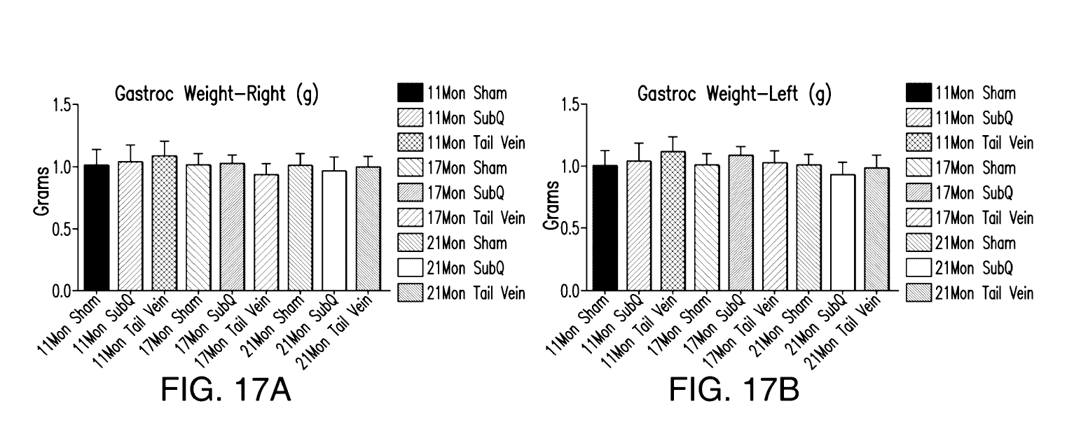

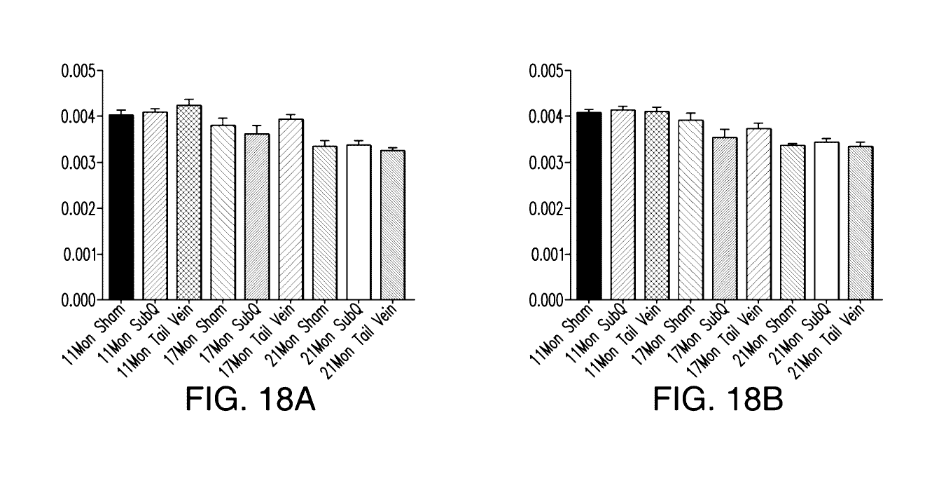

FIGS. 14A-14C (absolute data) and 15A-15C (data normalized to sham of same age group) show end of study rotarod evaluation. Animals performed 3 consecutive trials for data acquisition. The measurement was triggered when the animals fell to the base and triggered the end of measurement. The 17 and 21 month old animals that received injections of PSC-100 demonstrated improved performance compared to age matched controls. This is particularly evident in the 17 month old tail vein injected animals, which stayed on for 60% more revolutions (FIGS. 14A and 15A), 250% longer time (FIGS. 14B and 15B) and 120% greater distance (FIGS. 14C and 15C).

Mesenchymal Stem Cell (MSC) Flow Cytometry

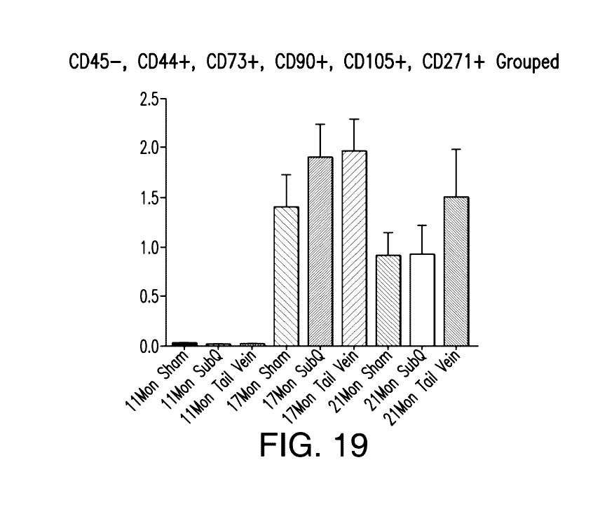

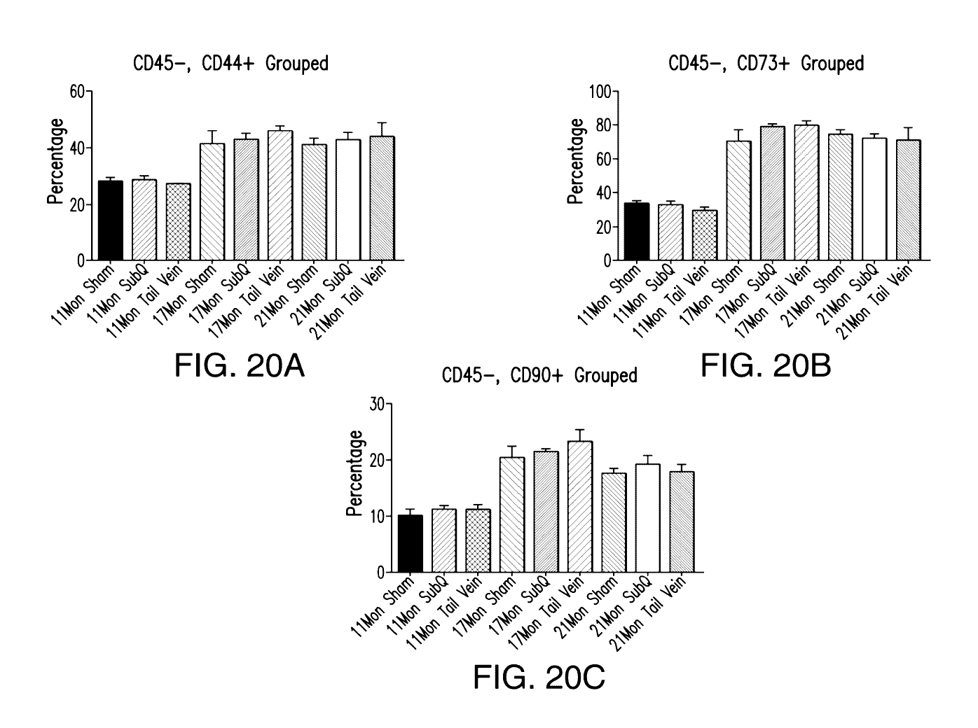

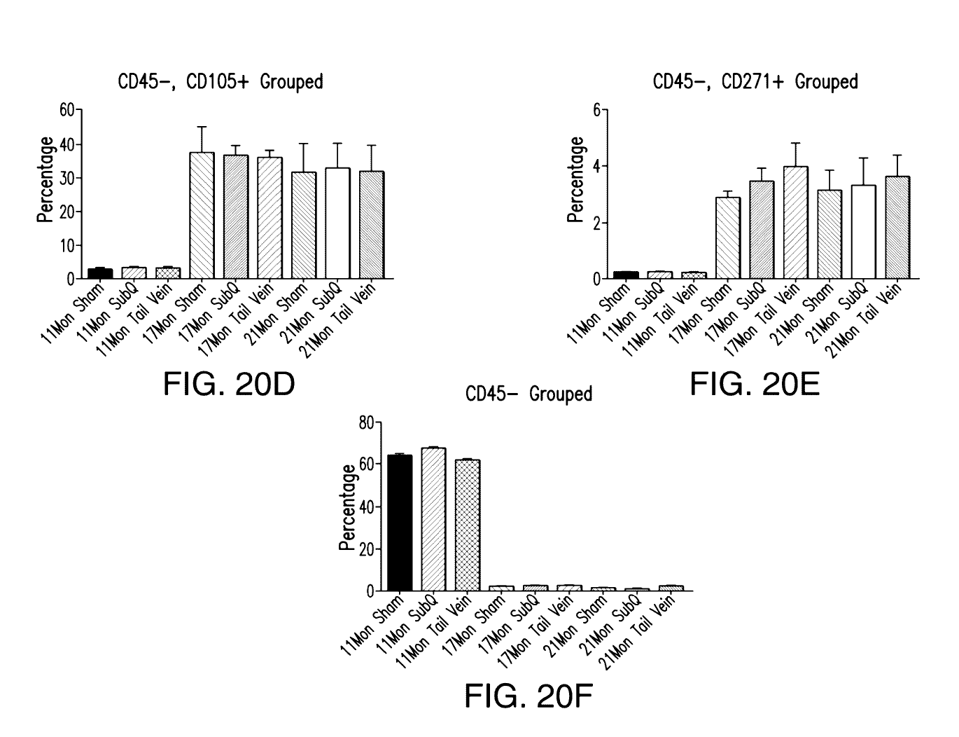

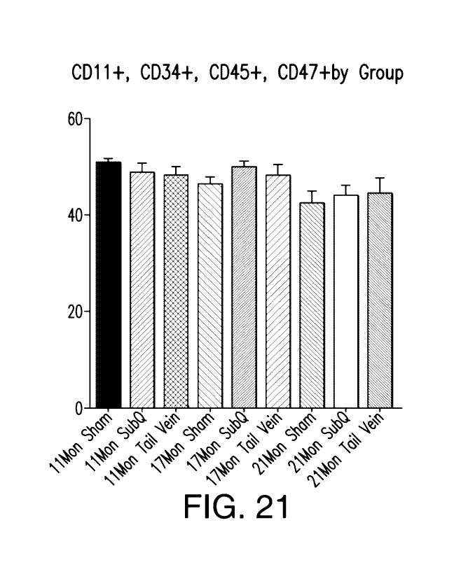

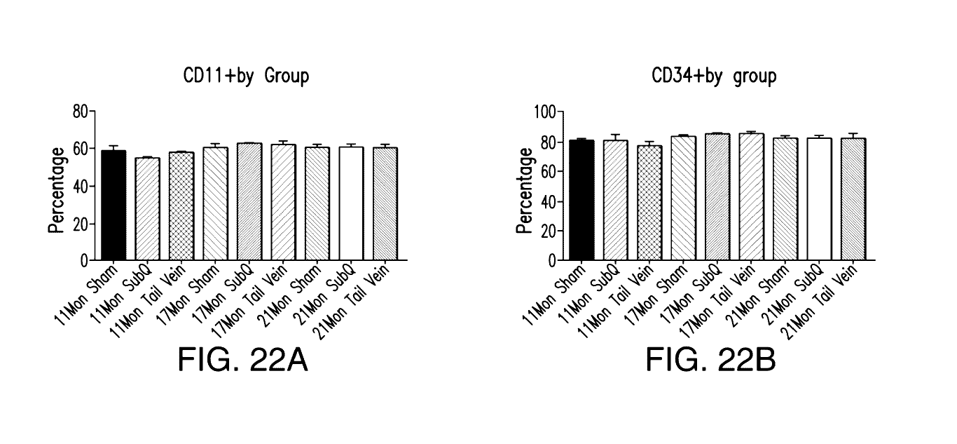

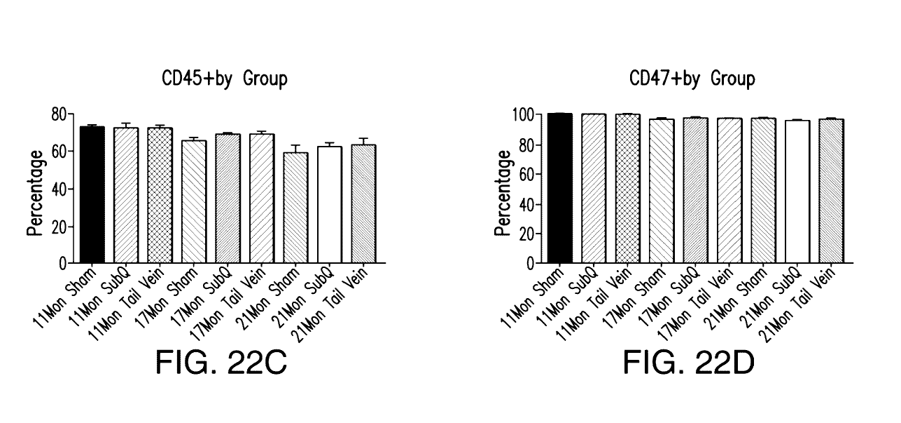

Mesenchymal stem cell (MSC)-like cells identified by flow cytometry are negative for expression of CD45 but positive for CD44, CD73, CD90, CD105, and CD271. Rats receiving injections of PSC-100 for 4 weeks demonstrated increased percentage of MSC-like stem cells (FIGS. 19 and 20A-20F). This was most evident in the 17 month old rats receiving PSC-100 by either tail vein or subcutaneous injections. In the older 21 month rats, the increase in MSC-like cells was most evident in the tail vein injected rats.

Quantification of Endogenous Pax7+ Stem Cells in the Muscle

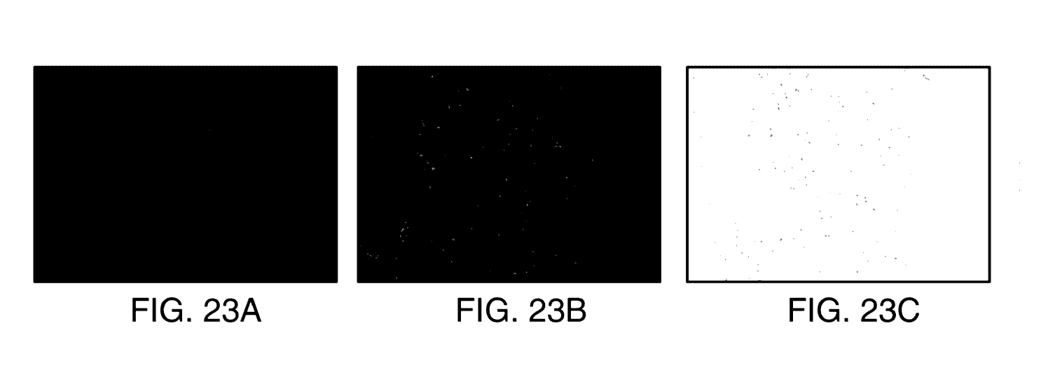

Pax7 is a marker expressed on the surface of muscle stem cells (also known as satellite cells). FIGS. 23A-23C and 24B demonstrate that the injection of PSC-100 in aging rats increased the percentage of Pax7+ muscle stem cells in the soleus muscle of 17 month old rats by both subcutaneous and tail vein injection

Quantification of Endogenous Ki67+ Stem Cells in the Brain

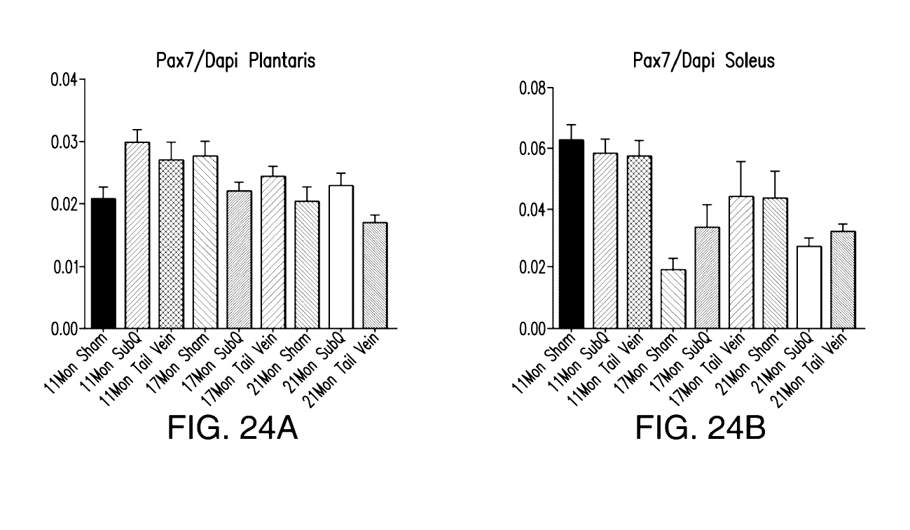

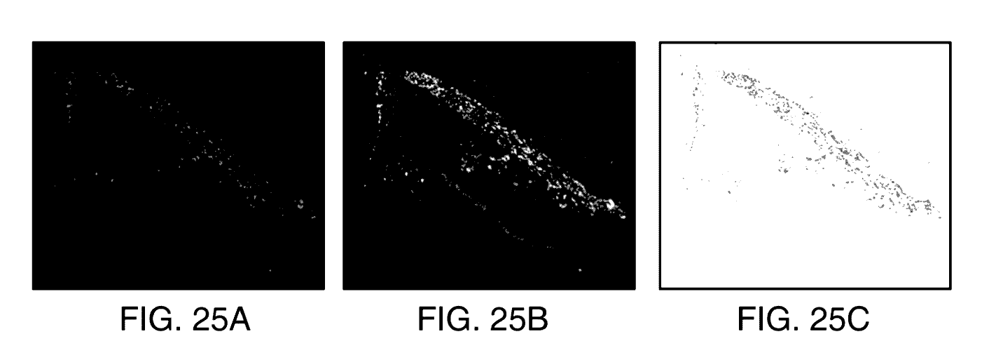

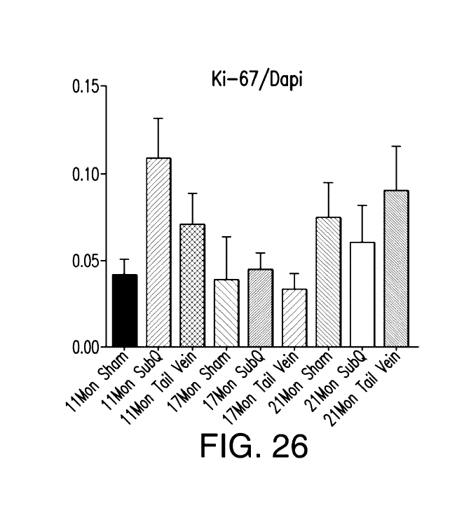

Stem cells are known to reside in the subventricular zone of the brain. These cells can proliferate and migrate to regenerate brain cells. The anti-Ki67 antibody binds only to proliferating cells, in all tissues including brain. FIGS. 25A-25C and 26 demonstrate that injections of PSC-100 increased the number of Ki67-positive brain stem cells in the subventricular zone in 11 month old rats and 21 month old rats.

Muscle Fiber Size Determination

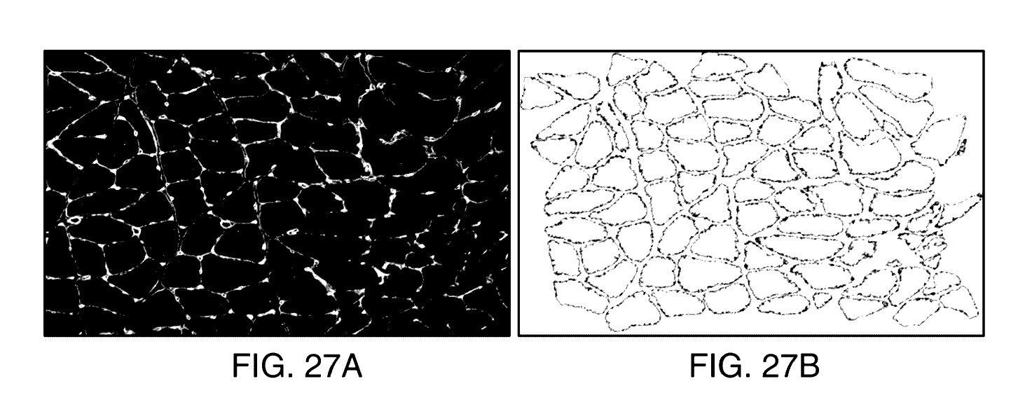

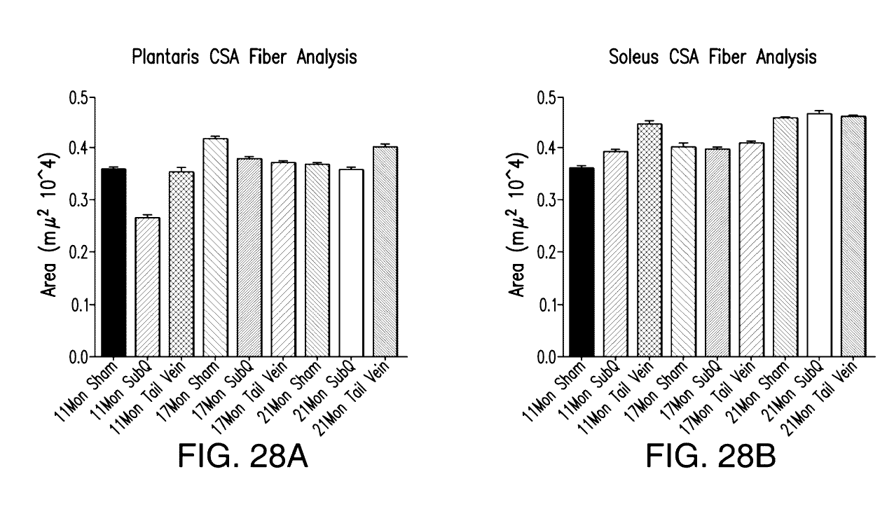

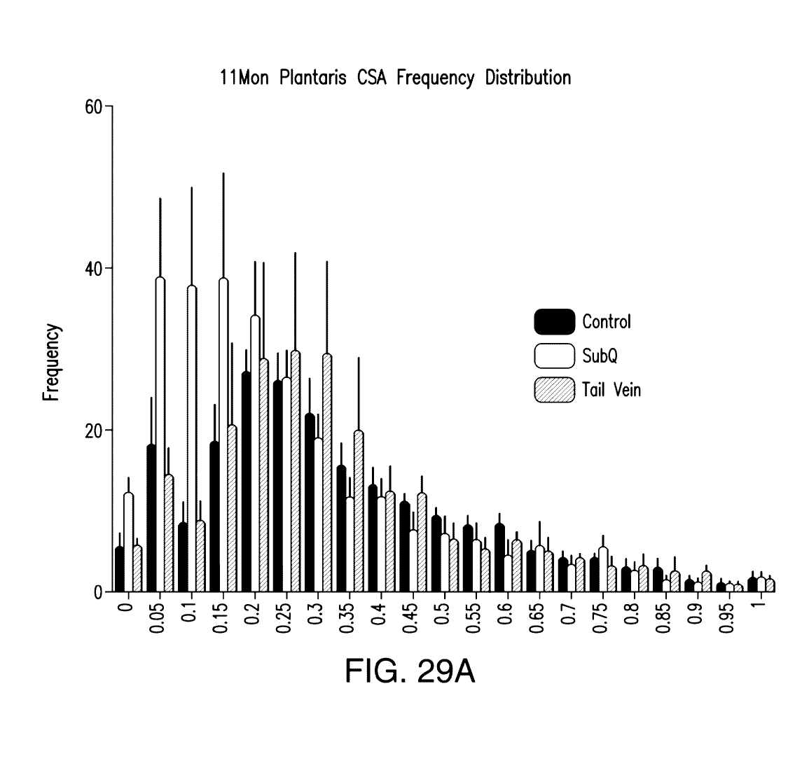

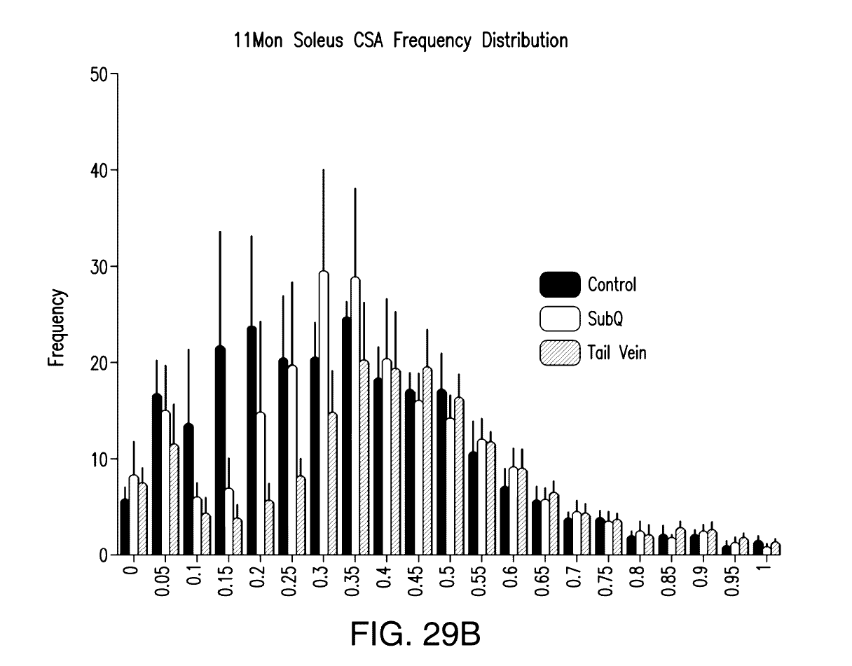

Muscle strength is related to muscle fiber size, which decreases with aging, thus causing the decrease in strength, balance, and walking duration in aging humans. To examine muscle fiber size in aging rats treated with PSC-100, anti-laminin antibody from Developmental Studies Hybridoma Bank was used to detect muscle fiber boundaries (FIGS. 27A-27B).

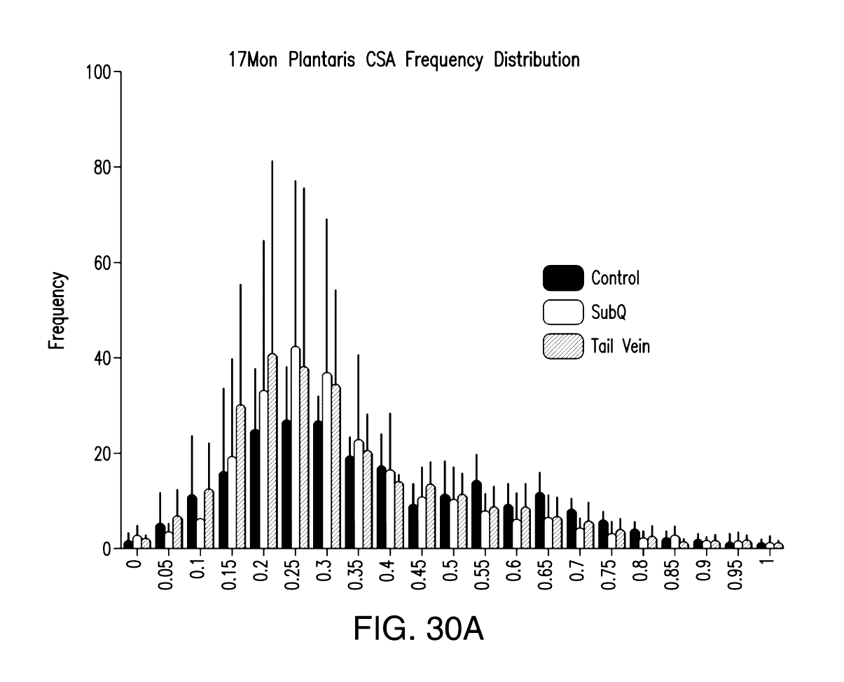

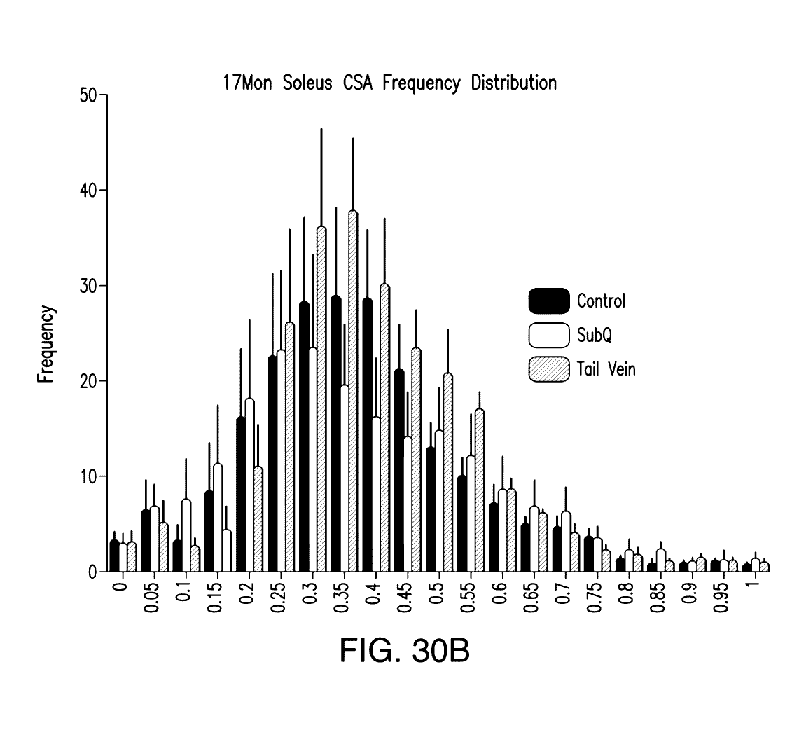

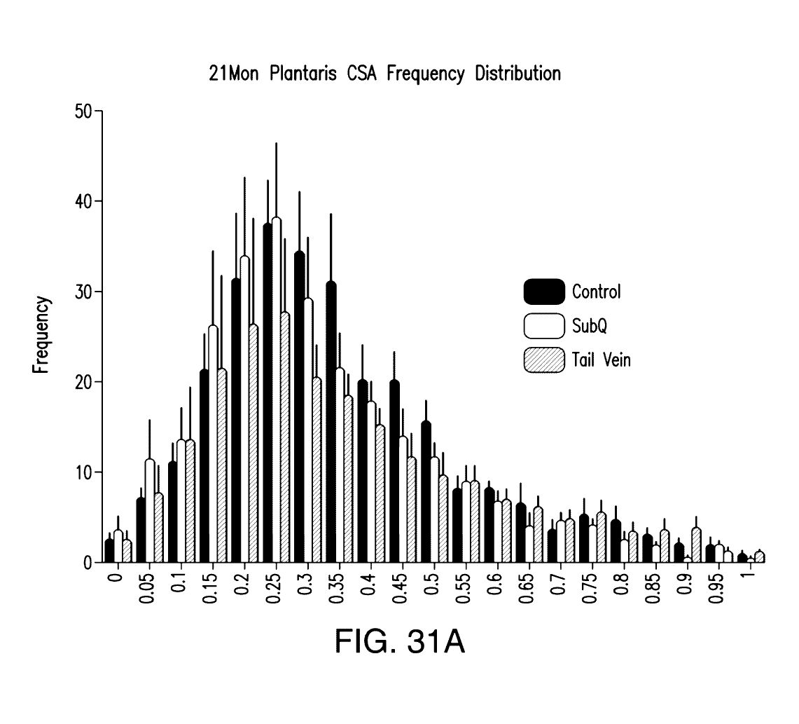

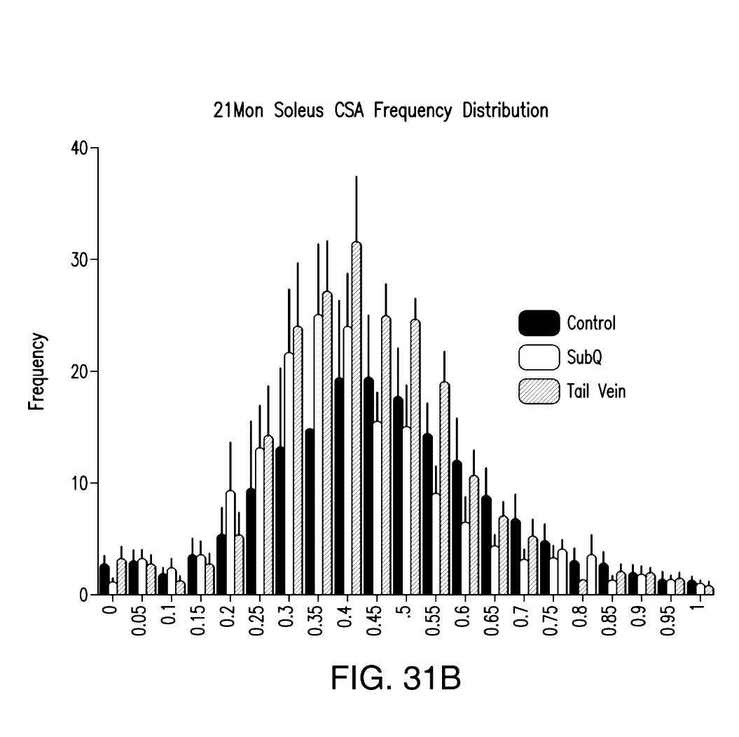

FIGS. 30B (soleus muscle in 17 month old rats) and 31B (soleus muscle in 21 month old rats) demonstrate that the PSC-100 injected rats have an improved (increased) fiber size distribution, and that the tail vein injections showed more improvement than the subcutaneous injections. This improvement in muscle fiber size could translate into increased strength with PSC-100 treatment and is likely, at least in part, associated with the improved rotarod performance shown in FIGS. 14A-14C and 15A-15C.

Colony Forming Unit (CFU) Assay

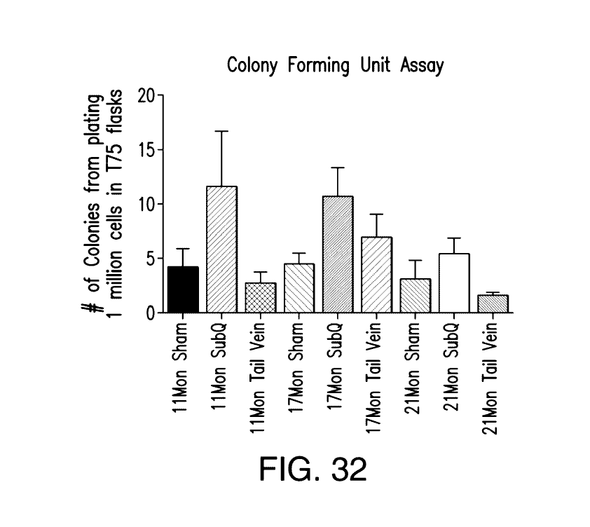

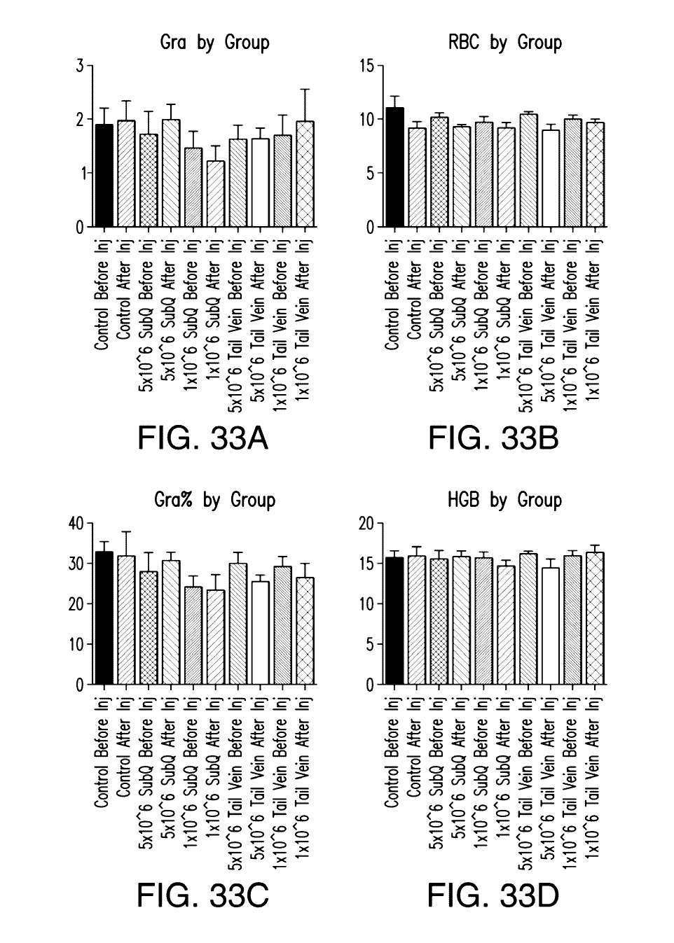

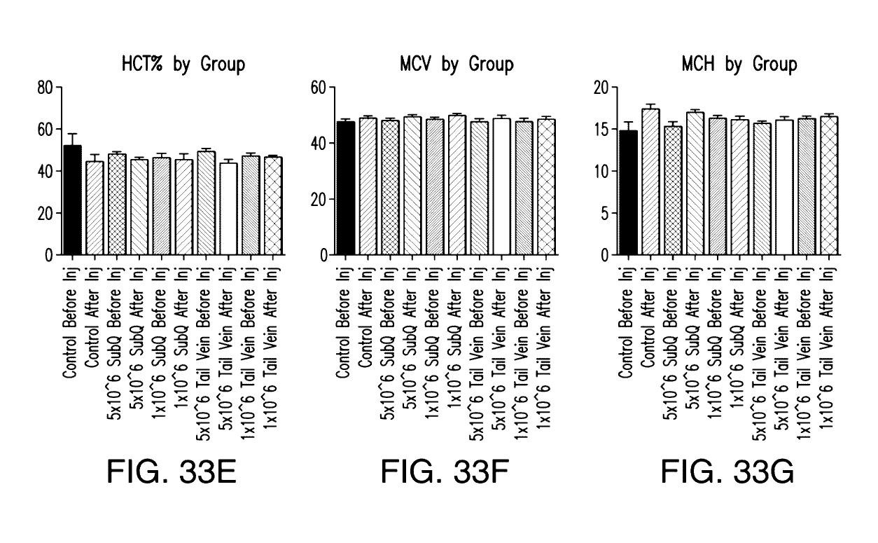

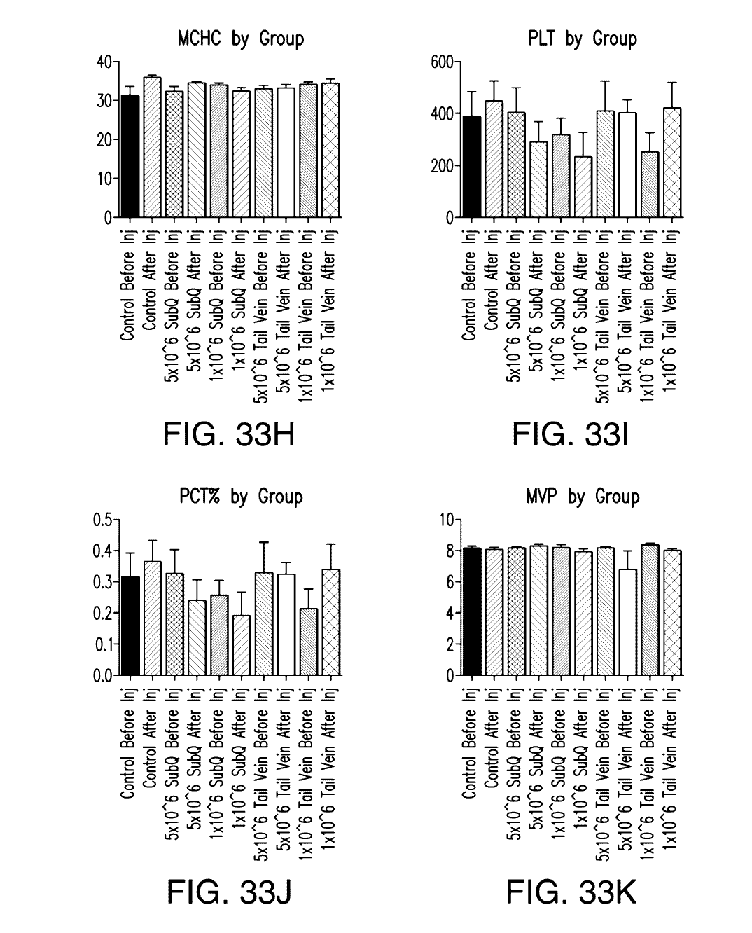

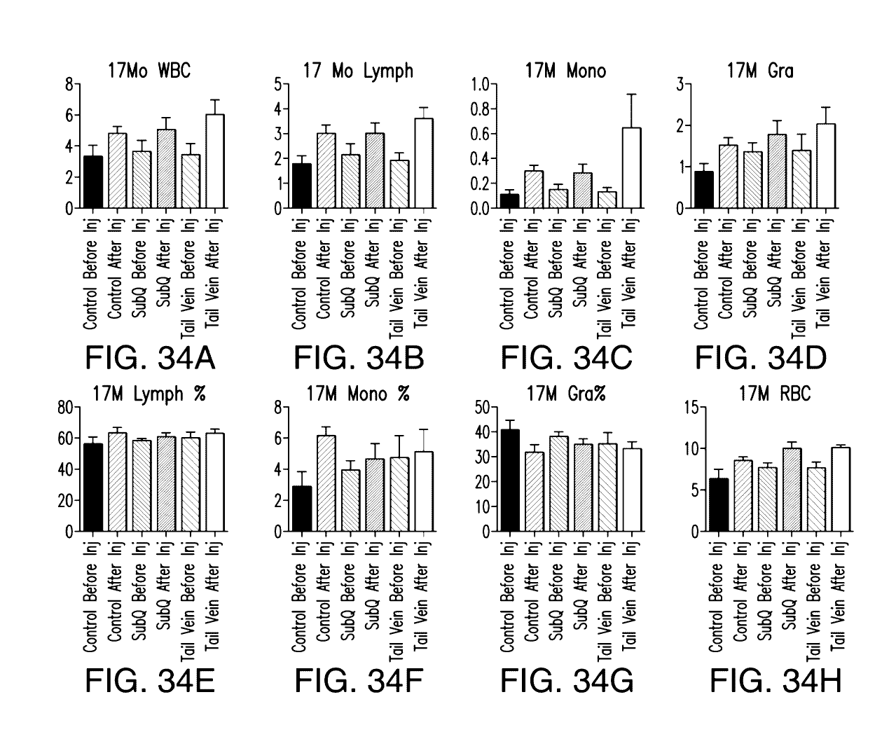

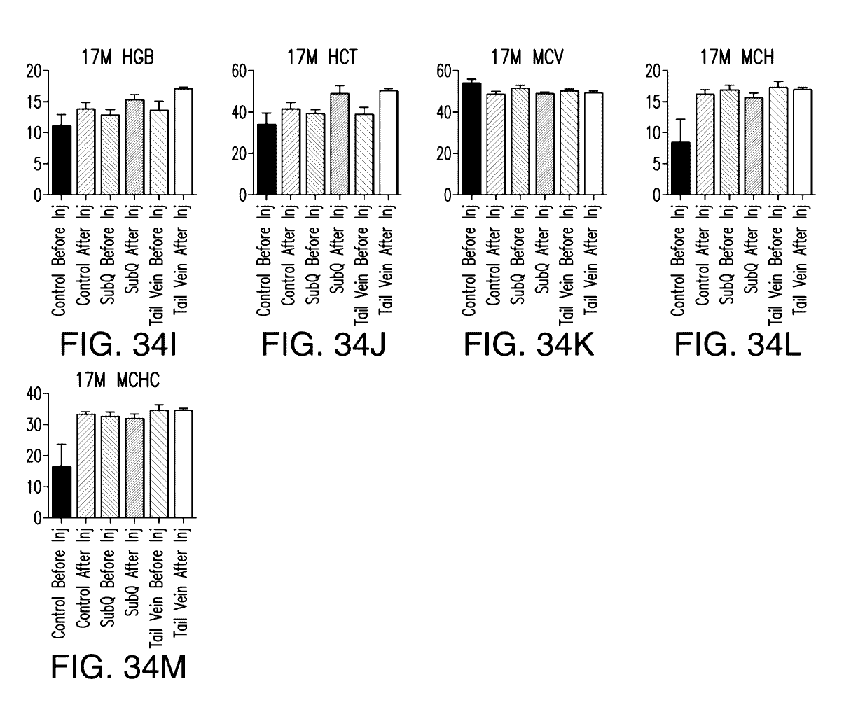

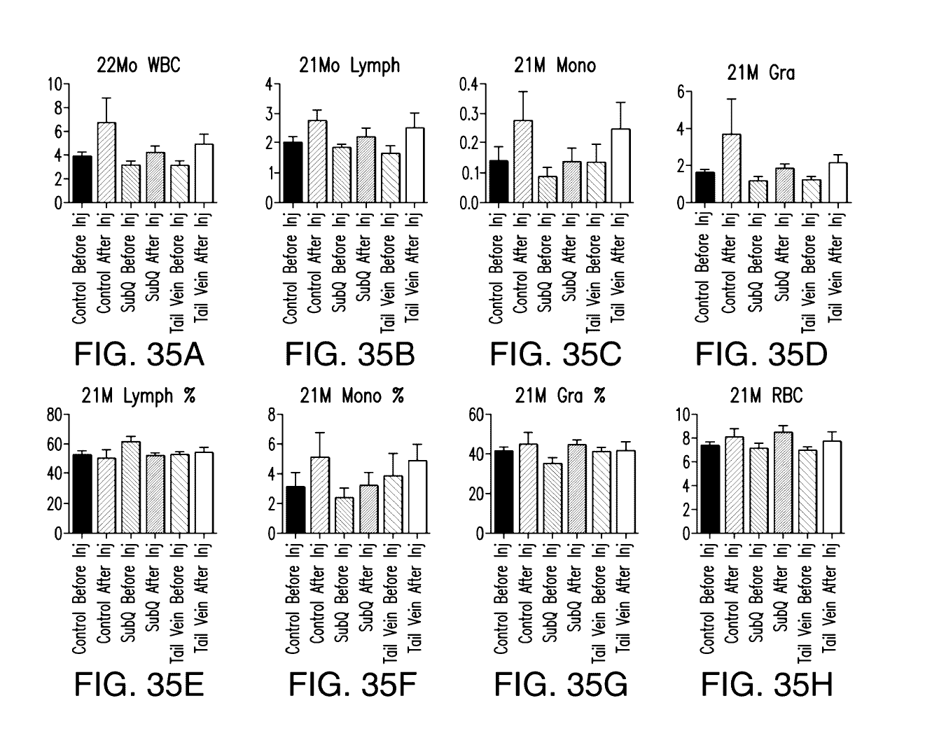

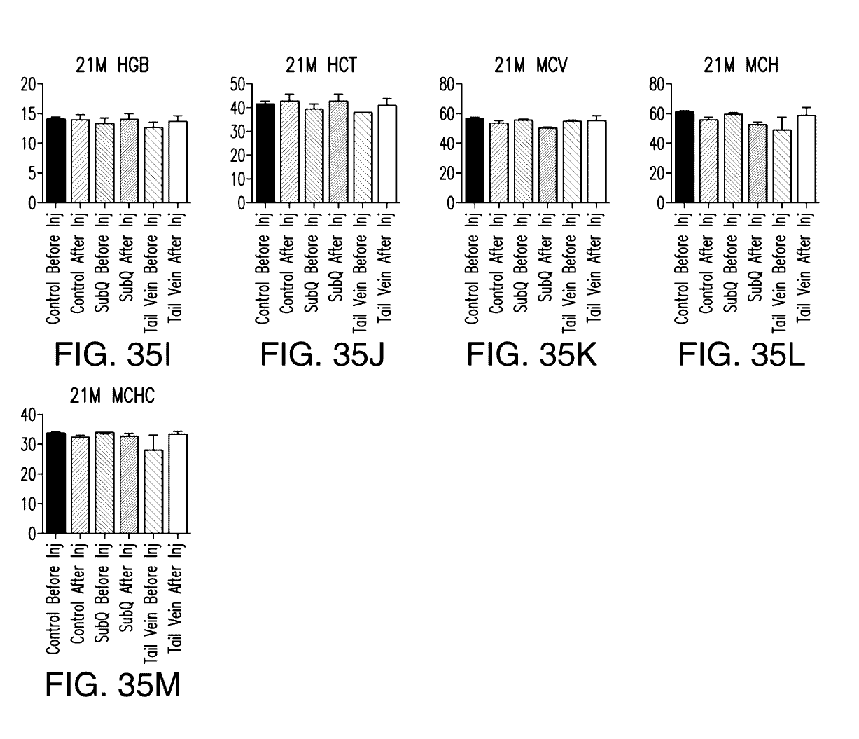

The CFU-F assay is used to determine the number of MSC-like cells present in a population of cells isolated from a tissue. The stem cells proliferate to form colonies which can then be counted, each colony representing a stem cell present in the starting population. As described in the methods, the femur bone marrow CFU assay was performed. FIG. 32 demonstrates that PSC-100 injections increased the number of colonies formed from the 11 month subcutaneously treated rats, 17 month subcutaneously treated rats, 17 month intravenously treated rats and 21 month subcutaneously treated rats but not 11 or 21 month intravenously treated rats. More colonies formed in the assay indicate more stem cells in the femur bone marrow. Thus, in summary, four of the six treatment groups demonstrated an increase in this important stem cell population.

RNAseq Analysis Results

The analysis of RNA expression after administration of PSC-100 in the aging F344 rats demonstrated significant changes in genes involved in several pathways. These genes provide potential new therapeutic targets for treating various diseases, either individually or in combination.

The global analysis of RNA expression demonstrated that the gene expression profile of 21 month rats treated intravenously with PSC-100 was closer to the gene expression profile of 17 month old sham control animals than to that of 21 month old sham control animals (data not shown). This suggests that the treatment of PSC-100 made the PSC-100 recipient animals resemble their younger counterparts.

The gene expression change is summarized in the following Tables 5-9, with a 2-fold cutoff. The value in the first column is the log 2 value of expression ratio between treated and sham control rats. Thus, a log 2 value of −1.0 means a 2-fold decrease, whereas a log 2 value of 1.0 means a 2-fold increase.

TABLE 5
Gene expression change in the hippocampus of 17
month old rat subcutaneously administered with
PDSC compared to 17 month old control rats

| | Exp False | | |
|---|---|---|---|
| Exp | Discovery | | |
| Log2 | Rate (q- | | |
| Ratio | value) | ID | Entrez Gene Name |
| −1.500 | 1.06E−02 | Alas2 | 5′-aminolevulinate synthase 2 |
| −3.140 | 1.06E−02 | Alox15 | arachidonate 15-lipoxygenase |
| 1.630 | 1.06E−02 | Cd74 | CD74 molecule |
| 2.520 | 1.06E−02 | Clic6 | chloride intracellular channel 6 |
| 11.210 | 1.06E−02 | Gngt1 | guanine nucleotide binding protein |
| | | | (G protein), gamma transducing |
| | | | activity polypeptide 1 |
| −1.200 | 1.06E−02 | Hba1 | hemoglobin, alpha 1 |
| −1.100 | 1.06E−02 | Hba2 | hemoglobin, alpha 1 |
| −1.530 | 1.06E−02 | Hbb | hemoglobin subunit beta |
| −1.310 | 1.06E−02 | LOC689064 | hemoglobin subunit beta |
| 1.820 | 1.06E−02 | RT1-Ba | major histocompatibility complex, |
| | | | class II, DQ alpha 1 |
| 1.930 | 1.06E−02 | RT1-Bb | major histocompatibility complex, |
| | | | class II, DQ beta 1 |
| 1.860 | 1.06E−02 | RT1-Da | major histocompatibility complex, |
| | | | class II, DR alpha |
| 1.760 | 1.06E−02 | RT1-Db1 | major histocompatibility complex, |
| | | | class II, DR beta 5 |
| 2.270 | 1.06E−02 | Mfrp | membrane frizzled-related protein |
| 9.920 | 1.06E−02 | Pdc | phosducin |
| 1.260 | 4.94E−02 | Ptpn14 | protein tyrosine phosphatase, non- |
| | | | receptor type 14 |
| 1.120 | 1.06E−02 | RGD1359290 | Ribosomal_L22 domain contain- |
| | | | ing protein RGD1359290 |
| −1.100 | 1.06E−02 | Rn45s | 45S pre-ribosomal RNA |
| 1.570 | 2.69E−02 | Slc18a2 | solute carrier family 18 (vesicular |
| | | | monoamine transporter), member 2 |
| −2.700 | 2.05E−02 | Slc4a1 | solute carrier family 4 (anion |
| | | | exchanger), member 1 (Diego |
| | | | blood group) |
| 2.650 | 1.06E−02 | Slc4a5 | solute carrier family 4 (sodium |
| | | | bicarbonate cotransporter), member |
| | | | 5 |
| −1.650 | 1.06E−02 | Snhg4 | small nucleolar RNA host gene 4 |
| | | | (non-protein coding) |
| 1.050 | 2.69E−02 | Sv2c | synaptic vesicle glycoprotein 2C |
| 1.630 | 1.06E−02 | Zic1 | Zic family member 1 |

TABLE 6
Gene expression change in the soleus of 17 month
old rat subcutaneously administered with PDSC
compared to 17 month old control rats

| | Exp False | | |
|---|---|---|---|
| Exp | Discovery | | |
| Log | Rate (q- | | |
| Ratio | value) | ID | Entrez Gene Name |
| −1.470 | 9.34E−03 | Alas2 | 5′-aminolevulinate synthase 2 |
| −4.300 | 9.34E−03 | Alox15 | arachidonate 15-lipoxygenase |
| −1.100 | 9.34E−03 | Angptl4 | angiopoietin like 4 |
| −1.030 | 9.34E−03 | Apod | apolipoprotein D |
| 1.180 | 9.34E−03 | Atf3 | activating transcription factor 3 |
| −1.760 | 9.34E−03 | Chac1 | ChaC glutathione-specific gamma- |
| | | | glutamylcyclotransferase 1 |
| −1.140 | 9.34E−03 | Fasn | fatty acid synthase |
| 1.110 | 9.34E−03 | Fst | follistatin |
| −1.000 | 9.34E−03 | Hba1 | hemoglobin, alpha 1 |
| −1.050 | 9.34E−03 | LOC689064 | hemoglobin subunit beta |
| −1.020 | 9.34E−03 | Mpz | myelin protein zero |
| 1.260 | 9.34E−03 | Myocd | myocardin |
| 1.450 | 9.34E−03 | Nr4a3 | nuclear receptor subfamily 4 group |
| | | | A member 3 |
| −1.350 | 9.34E−03 | Scd1 | stearoyl-CoA desaturase (delta-9- |
| | | | desaturase) |
| −1.440 | 3.63E−02 | Spag8 | sperm associated antigen 8 |
| −1.000 | 4.22E−02 | Thrsp | thyroid hormone responsive |
| −1.390 | 9.34E−03 | Zfyve28 | zinc finger, FYVE domain containing |
| | | | 28 |

TABLE 7
Gene expression change in the soleus of 17 month old rat intravenously
administered with PDSC compared to 17 month old control rats

| | Exp False | | |
|---|---|---|---|
| Exp | Discovery | | |
| Log | Rate (q- | | |
| Ratio | value) | ID | Entrez Gene Name |
| 1.140 | 2.57E−02 | Actn3 | actinin alpha 3 |
| −4.840 | 2.57E−02 | Alox15 | arachidonate 15-lipoxygenase |
| 1.610 | 2.57E−02 | Cxcl13 | chemokine (C-X-C motif) ligand 13 |
| 1.110 | 2.57E−02 | Fos | FBJ murine osteosarcoma viral oncogene |
| | | | homolog |
| 1.500 | 2.57E−02 | Fosb | FBJ murine osteosarcoma viral oncogene |
| | | | homolog B |
| 3.530 | 2.57E−02 | Myh4 | myosin, heavy chain 4, skeletal muscle |
| 3.040 | 2.57E−02 | Nr4a2 | nuclear receptor subfamily 4 group A |
| | | | member 2 |
| 1.130 | 2.57E−02 | Nr4a3 | nuclear receptor subfamily 4 group A |
| | | | member 3 |
| 1.770 | 2.57E−02 | Pvalb | parvalbumin |
| −1.320 | 2.57E−02 | Scd1 | stearoyl-CoA desaturase (delta-9- |
| | | | desaturase) |

TABLE 8
Gene expression change in the soleus of 21 month
old rat subcutaneously administered with PDSC
compared to 21 month old control rats

| | Exp False | | |
|---|---|---|---|
| Exp | Discovery | | |
| Log | Rate (q- | | |
| Ratio | value) | ID | Entrez Gene Name |
| −1.580 | 3.41E−03 | Actn3 | actinin alpha 3 |
| −1.520 | 3.41E−03 | Alas2 | 5′-aminolevulinate synthase 2 |
| −4.270 | 3.41E−03 | Alox15 | arachidonate 15-lipoxygenase |
| −2.810 | 3.41E−03 | Angptl4 | angiopoietin like 4 |
| −1.890 | 3.41E−03 | Apold1 | apolipoprotein L domain |
| | | | containing 1 |
| −1.270 | 3.41E−03 | Arc | activity-regulated cytoskeleton- |
| | | | associated protein |
| 1.560 | 3.41E−03 | Arntl | aryl hydrocarbon receptor nuclear |
| | | | translocator like |
| 1.050 | 3.41E−03 | Asb5 | ankyrin repeat and SOCS box |
| | | | containing 5 |
| 1.020 | 3.41E−03 | Bcl6 | B-cell CLL/lymphoma 6 |
| 1.570 | 3.41E−03 | Bdnf | brain-derived neurotrophic factor |
| −2.460 | 3.41E−03 | Ccl5 | chemokine (C-C motif) ligand 5 |
| −1.550 | 3.41E−03 | Chac1 | ChaC glutathione-specific gamma- |
| | | | glutamylcyclotransferase 1 |
| −1.080 | 3.41E−03 | Ciart | circadian associated represser of |
| | | | transcription |
| −1.740 | 3.41E−03 | Cish | cytokine inducible SH2-containing |
| | | | protein |
| −1.080 | 3.41E−03 | Dbp | D site of albumin promoter |
| | | | (albumin D-box) binding protein |
| 1.380 | 3.41E−03 | Dyrk2 | dual specificity tyrosine-(Y)- |
| | | | phosphorylation regulated kinase 2 |
| −2.350 | 3.41E−03 | Egr1 | early growth response 1 |
| −2.380 | 4.09E−02 | Egr3 | early growth response 3 |
| 1.500 | 3.41E−03 | Fam46a | family with sequence similarity 46 |
| | | | member A |
| −1.550 | 3.41E−03 | Fosb | FBJ murine osteosarcoma viral |
| | | | oncogene homolog B |
| −1.080 | 3.41E−03 | Hba1 | hemoglobin, alpha 1 |
| −1.250 | 3.41E−03 | Hba2 | hemoglobin, alpha 1 |
| −1.010 | 3.41E−03 | Hbb | hemoglobin subunit beta |
| −1.370 | 3.41E−03 | Hbb-b1 | hemoglobin subunit beta |
| −1.690 | 3.41E−03 | LOC689064 | hemoglobin subunit beta |
| 1.370 | 3.41E−03 | Hbegf | heparin-binding EGF-like growth |
| | | | factor |
| −1.640 | 6.08E−03 | Il2rb | interleukin 2 receptor subunit beta |
| 1.200 | 3.41E−03 | Irs1 | insulin receptor substrate 1 |
| 1.340 | 3.41E−03 | Irs2 | insulin receptor substrate 2 |
| −1.060 | 3.41E−03 | Junb | jun B proto-oncogene |
| 1.110 | 3.41E−03 | Kcnk5 | potassium channel, two pore |
| | | | domain subfamily K, member 5 |
| 1.010 | 3.93E−02 | Kirrel2 | kin of IRRE like 2 (Drosophila) |
| 1.340 | 3.41E−03 | RGD1564428 | zinc finger protein 474-like |
| 1.460 | 3.41E−03 | Lonrf3 | LON peptidase N-terminal |
| | | | domain and ring finger 3 |
| 1.070 | 3.41E−03 | Lrrc38 | leucine rich repeat containing 38 |
| 1.010 | 3.41E−03 | Lrrc52 | leucine rich repeat containing 52 |
| −2.730 | 3.41E−03 | Mybpc2 | myosin binding protein C, fast type |
| −2.120 | 3.41E−03 | Myh1 | myosin, heavy chain 1, skeletal |
| | | | muscle, adult |
| 1.490 | 3.41E−03 | Myocd | myocardin |
| 1.080 | 3.41E−03 | Nedd9 | neural precursor cell expressed, |
| | | | developmentally down-regulated 9 |
| 2.160 | 3.41E−03 | Nfil3 | nuclear factor, interleukin 3 |
| | | | regulated |
| −2.640 | 3.41E−03 | Nr4a2 | nuclear receptor subfamily 4 group |
| | | | A member 2 |
| 1.860 | 3.41E−03 | Nr4a3 | nuclear receptor subfamily 4 group |
| | | | A member 3 |
| −1.110 | 3.41E−03 | Parp16 | poly(ADP-ribose) polymerase |
| | | | family member 16 |
| −1.580 | 3.41E−03 | Plk3 | polo-like kinase 3 |
| −2.890 | 3.41E−03 | Prf1 | perforin 1 (pore forming protein) |
| −3.260 | 3.41E−03 | Pvalb | parvalbumin |
| 1.240 | 3.41E−03 | Rcan1 | regulator of calcineurin 1 |
| 1.430 | 3.41E−03 | Rp1 | retinitis pigmentosa 1 (autosomal |
| | | | dominant) |
| 1.190 | 3.41E−03 | Rrad | Ras-related associated with |
| | | | diabetes |
| −1.140 | 6.08E−03 | Siglec5 | sialic acid binding Ig-like lectin 8 |
| −1.090 | 3.41E−03 | Slc2a5 | solute carrier family 2 (facilitated |
| | | | glucose/fructose transporter), |
| | | | member 5 |
| 1.180 | 3.41E−03 | Slc30a4 | solute carrier family 30 (zinc |
| | | | transporter), member 4 |
| −1.650 | 3.41E−03 | Slpi | secretory leukocyte peptidase |
| | | | inhibitor |
| 2.230 | 3.41E−03 | Tnfrsf12a | tumor necrosis factor receptor |
| | | | superfamily member 12A |
| −1.170 | 3.41E−03 | Txnip | thioredoxin interacting protein |
| 1.770 | 3.41E−03 | Zfp474 | zinc finger protein 474 |

TABLE 9
Gene expression change in the soleus of 21 month old rat intravenously
administered with PDSC compared to 21 month old control rats

| | Exp False | | |
|---|---|---|---|
| Exp | Discovery | | |
| Log | Rate (q- | | |
| Ratio | value) | ID | Entrez Gene Name |
| 1.080 | 1.35E−03 | Abcg1 | ATP binding cassette subfamily G |
| | | | member 1 |
| 1.010 | 1.35E−03 | Abra | actin binding Rho activating protein |
| 1.360 | 1.35E−03 | Alox15 | arachidonate 15-lipoxygenase |
| −2.930 | 1.35E−03 | Angptl4 | angiopoietin like 4 |
| −1.110 | 1.35E−03 | Apod | apolipoprotein D |
| 1.020 | 1.35E−03 | Arhgap24 | Rho GTPase activating protein 24 |
| 1.290 | 1.35E−03 | Arl4c | ADP-ribosylation factor like GTPase 4C |
| 2.120 | 1.35E−03 | Arntl | aryl hydrocarbon receptor nuclear |
| | | | translocator like |
| −1.380 | 1.35E−03 | Arrdc2 | arrestin domain containing 2 |
| 1.900 | 1.35E−03 | Asb5 | ankyrin repeat and SOCS box containing |
| | | | 5 |
| 1.510 | 1.35E−03 | Atf3 | activating transcription factor 3 |
| 1.050 | 1.35E−03 | Bag2 | BCL2 associated athanogene 2 |
| 1.760 | 1.35E−03 | Bcl11a | B-cell CLL/lymphoma 11A |
| 1.060 | 1.35E−03 | Bdh1 | 3-hydroxybutyrate dehydrogenase, type |
| | | | 1 |
| 1.500 | 1.35E−03 | Bdnf | brain-derived neurotrophic factor |
| 1.030 | 1.35E−03 | Best3 | bestrophin 3 |
| −1.180 | 1.35E−03 | Bhlhe40 | basic helix-loop-helix family member |
| | | | e40 |
| −1.430 | 1.35E−03 | RGD1307461 | chromosome 3 open reading frame 18 |
| 1.420 | 1.35E−03 | Car12 | carbonic anhydrase XII |
| 1.040 | 1.91E−02 | Calhm1 | calcium homeostasis modulator 1 |
| 2.330 | 1.35E−03 | Calml3 | calmodulin like 3 |
| −2.770 | 1.35E−03 | Ccl5 | chemokine (C-C motif) ligand 5 |
| 1.420 | 1.26E−02 | Cdc42se1 | CDC42 small effector 1 |
| −2.010 | 1.35E−03 | Chac1 | ChaC glutathione-specific gamma- |
| | | | glutamylcyclotransferase 1 |
| 1.300 | 1.03E−02 | Chst5 | carbohydrate (N-acetylglucosamine 6-O) |
| | | | sulfotransferase 6 |
| 1.130 | 1.35E−03 | Cidec | cell death-inducing DFFA-like effector c |
| −1.430 | 1.35E−03 | Cish | cytokine inducible SH2-containing |
| | | | protein |
| −1.070 | 1.35E−03 | Cited4 | Cbp/p300-interacting transactivator, |
| | | | with Glu/Asp rich carboxy-terminal |
| | | | domain, 4 |
| 1.150 | 1.35E−03 | Ckap4 | cytoskeleton-associated protein 4 |
| 1.390 | 8.87E−03 | Cldn2 | claudin 2 |
| −1.100 | 1.35E−03 | Cpt1a | carnitine palmitoyltransferase 1A (liver) |
| 1.330 | 1.35E−03 | Csrnp1 | cysteine-serine-rich nuclear protein 1 |
| −1.570 | 8.87E−03 | Cxcl13 | chemokine (C-X-C motif) ligand 13 |
| −1.470 | 1.35E−03 | Dbp | D site of albumin promoter (albumin D- |
| | | | box) binding protein |
| 1.120 | 1.35E−03 | Dnajb5 | DnaJ heat shock protein family (Hsp40) |
| | | | member B5 |
| 1.050 | 1.35E−03 | Dynll1 | dynein, light chain, LC8-type 1 |
| 1.940 | 1.35E−03 | Dyrk2 | dual specificity tyrosine-(Y)- |
| | | | phosphorylation regulated kinase 2 |
| 1.090 | 3.82E−02 | Edn1 | endothelin 1 |
| −1.100 | 1.35E−03 | Egr1 | early growth response 1 |
| 1.020 | 9.61E−03 | Elfn1 | extracellular leucine-rich repeat and |
| | | | fibronectin type III domain containing 1 |
| 1.050 | 4.59E−03 | Emb | embigin |
| 1.440 | 1.35E−03 | Enah | enabled homolog (Drosophila) |
| 1.160 | 1.35E−03 | Fam107b | family with sequence similarity 107 |
| | | | member B |
| 1.760 | 1.35E−03 | Fam110a | family with sequence similarity 110 |
| | | | member A |
| 1.540 | 1.35E−03 | Fam134b | family with sequence similarity 134 |
| | | | member B |
| 1.490 | 9.61E−03 | Fam167a | family with sequence similarity 167 |
| | | | member A |
| 1.170 | 1.35E−03 | RGD1309676 | family with sequence similarity 213 |
| | | | member A |
| 1.730 | 1.35E−03 | Fam46a | family with sequence similarity 46 |
| | | | member A |
| 1.170 | 1.35E−03 | Fgfr3 | fibroblast growth factor receptor 3 |
| 1.090 | 1.35E−03 | Fhl2 | four and a half LIM domains 2 |
| 1.370 | 1.35E−03 | Fosb | FBJ murine osteosarcoma viral |
| | | | oncogene homolog B |
| 1.010 | 1.35E−03 | Frk | fyn-related Src family tyrosine kinase |
| 1.300 | 1.35E−03 | Fst | follistatin |
| 1.270 | 1.57E−02 | Gdf15 | growth differentiation factor 15 |
| 1.360 | 1.35E−03 | Gem | GTP binding protein overexpressed in |
| | | | skeletal muscle |
| 1.080 | 1.35E−03 | Gnl3 | guanine nucleotide binding protein-like |
| | | | 3 (nucleolar) |
| 2.400 | 1.35E−03 | Hbegf | heparin-binding EGF-like growth factor |
| 1.010 | 1.35E−03 | Hmox1 | heme oxygenase 1 |
| −1.030 | 1.26E−02 | Hpdl | 4-hydroxyphenylpyruvate dioxygenase- |
| | | | like |
| 2.240 | 3.73E−02 | Hspa1b | heat shock protein family A (Hsp70) |
| | | | member 1A |
| 1.200 | 1.35E−03 | Id4 | inhibitor of DNA binding 4, dominant |
| | | | negative helix-loop-helix protein |
| −1.720 | 6.41E−03 | Il2rb | interleukin 2 receptor subunit beta |
| 1.400 | 1.35E−03 | Irs1 | insulin receptor substrate 1 |
| 1.590 | 1.35E−03 | Irs2 | insulin receptor substrate 2 |
| 1.000 | 1.35E−03 | Jund | jun D proto-oncogene |
| 1.040 | 1.35E−03 | Kbtbd8 | kelch repeat and BTB (POZ) domain |
| | | | containing 8 |
| 1.190 | 1.35E−03 | Kcnk5 | potassium channel, two pore domain |
| | | | subfamily K, member 5 |
| −1.280 | 1.70E−02 | Kctd7 | potassium channel tetramerization |
| | | | domain containing 7 |
| 1.040 | 3.17E−02 | Kirrel2 | kin of IRRE like 2 (Drosophila) |
| −1.290 | 1.35E−03 | Ky | kyphoscoliosis peptidase |
| 1.520 | 1.35E−03 | Lamc2 | laminin subunit gamma 2 |
| 1.300 | 1.35E−03 | Lipg | lipase, endothelial |
| 2.130 | 1.35E−03 | RGD1564428 | zinc finger protein 474-like |
| 2.570 | 1.35E−03 | Lonrf3 | LON peptidase N-terminal domain and |
| | | | ring finger 3 |
| 1.230 | 1.35E−03 | Lrrc38 | leucine rich repeat containing 38 |
| 1.730 | 1.35E−03 | Lrrc52 | leucine rich repeat containing 52 |
| −1.800 | 3.58E−03 | Lrrn2 | leucine rich repeat neuronal 2 |
| 1.210 | 1.48E−02 | Lsr | lipolysis stimulated lipoprotein receptor |
| 1.300 | 1.35E−03 | Maff | v-maf avian musculoaponeurotic |
| | | | fibrosarcoma oncogene homolog F |
| −1.140 | 4.07E−02 | Mchr1 | melanin concentrating hormone receptor |
| | | | 1 |
| 1.920 | 2.52E−03 | Mllt11 | myeloid/lymphoid or mixed-lineage |
| | | | leukemia; translocated to, 11 |
| 1.400 | 1.35E−03 | Mns1 | meiosis specific nuclear structural 1 |
| 1.140 | 3.41E−02 | Mogat1 | monoacylglycerol O-acyltransferase 1 |
| 1.140 | 1.35E−03 | Mphosph6 | M-phase phosphoprotein 6 |
| 1.540 | 1.48E−02 | Muc20 | mucin 20, cell surface associated |
| −2.240 | 1.35E−03 | Mybpc2 | myosin binding protein C, fast type |
| 1.010 | 1.35E−03 | Myf6 | myogenic factor 6 |
| −1.290 | 1.35E−03 | Myh1 | myosin, heavy chain 1, skeletal muscle, |
| | | | adult |
| −1.060 | 1.35E−03 | Myh2 | myosin, heavy chain 2, skeletal muscle, |
| | | | adult |
| 1.130 | 1.35E−03 | Myocd | myocardin |
| 1.270 | 1.35E−03 | Nedd9 | neural precursor cell expressed, |
| | | | developmentally down-regulated 9 |
| 2.490 | 1.35E−03 | Nfil3 | nuclear factor, interleukin 3 regulated |
| −2.740 | 2.88E−02 | Nkg7 | natural killer cell granule protein 7 |
| −1.720 | 2.52E−03 | Nr1d1 | nuclear receptor subfamily 1 group D |
| | | | member 1 |
| 4.000 | 1.35E−03 | Nr4a3 | nuclear receptor subfamily 4 group A |
| | | | member 3 |
| −1.020 | 4.53E−02 | Ntf4 | neurotrophin 4 |
| 1.100 | 1.35E−03 | Nuak1 | NUAK family, SNF1-like kinase, 1 |
| −1.030 | 1.35E−03 | Parp16 | poly(ADP-ribose) polymerase family |
| | | | member 16 |
| 1.140 | 1.35E−03 | Pde7a | phosphodiesterase 7A |
| 1.000 | 1.35E−03 | Pfkfb2 | 6-phosphofructo-2-kinase/fructose-2,6- |
| | | | biphosphatase 2 |
| −1.390 | 1.35E−03 | Pfkfb3 | 6-phosphofructo-2-kinase/fructose-2,6- |
| | | | biphosphatase 3 |
| 1.320 | 1.35E−03 | Pgam1 | phosphoglycerate mutase 1 |
| 1.000 | 1.35E−03 | Phlda1 | pleckstrin homology-like domain, family |
| | | | A, member 1 |
| −1.460 | 1.35E−03 | Pik3ip1 | phosphoinositide-3-kinase interacting |
| | | | protein 1 |
| 1.090 | 1.35E−03 | Postn | periostin, osteoblast specific factor |
| 1.010 | 1.35E−03 | Ppargc1a | peroxisome proliferator-activated |
| | | | receptor gamma, coactivator 1 alpha |
| 1.210 | 2.47E−02 | Ppp1r14c | protein phosphatase 1 regulatory |
| | | | inhibitor subunit 14C |
| −2.460 | 1.35E−03 | Prf1 | perforin 1 (pore forming protein) |
| 1.120 | 1.35E−03 | Rab23 | RAB23, member RAS oncogene family |
| 1.000 | 1.35E−03 | Rab30 | RAB30, member RAS oncogene family |
| 1.100 | 1.35E−03 | Rbm20 | RNA binding motif protein 20 |
| 2.020 | 1.35E−03 | Rcan1 | regulator of calcineurin 1 |
| 1.210 | 1.35E−03 | Rell1 | RELT-like 1 |
| 1.230 | 1.35E−03 | Rfx1 | regulatory factor X1 |
| 2.120 | 1.35E−03 | Rhpn2 | rhophilin, Rho GTPase binding protein 2 |
| 1.450 | 1.35E−03 | Rnd1 | Rho family GTPase 1 |
| 1.890 | 1.35E−03 | Rp1 | retinitis pigmentosa 1 (autosomal |
| | | | dominant) |
| 1.870 | 1.35E−03 | Rrad | Ras-related associated with diabetes |
| −1.000 | 6.41E−03 | Rtn4rl1 | reticulon 4 receptor-like 1 |
| 1.390 | 1.35E−03 | Scd1 | stearoyl-CoA desaturase (delta-9- |
| | | | desaturase) |
| 1.130 | 1.35E−03 | Sdc4 | syndecan 4 |
| −1.060 | 1.35E−03 | Sec14l5 | SEC14-like lipid binding 5 |
| 1.030 | 1.35E−03 | Pragmin | homolog of rat pragma of Rnd2 |
| 1.050 | 1.35E−03 | Sik1 | salt inducible kinase 1 |
| −1.780 | 1.35E−03 | Slc2a5 | solute carrier family 2 (facilitated |
| | | | glucose/fructose transporter), member 5 |
| 1.740 | 1.35E−03 | Slc30a4 | solute carrier family 30 (zinc |
| | | | transporter), member 4 |
| 1.230 | 4.59E−03 | Slc4a1 | solute carrier family 4 (anion |
| | | | exchanger), member 1 (Diego blood |
| | | | group) |
| 1.000 | 1.35E−03 | Smad7 | SMAD family member 7 |
| −1.120 | 2.73E−02 | Spag8 | sperm associated antigen 8 |
| 1.160 | 2.73E−02 | Stc1 | stanniocalcin 1 |
| 1.020 | 1.35E−03 | Terf2ip | telomeric repeat binding factor 2, |
| | | | interacting protein |
| −1.420 | 1.77E−02 | Tmc8 | transmembrane channel like 8 |
| 1.990 | 1.35E−03 | Tmem171 | transmembrane protein 171 |
| 1.060 | 1.35E−03 | Tmx4 | thioredoxin-related transmembrane |
| | | | protein 4 |
| 3.280 | 1.35E−03 | Tnfrsf12a | tumor necrosis factor receptor |
| | | | superfamily member 12A |
| −1.010 | 1.35E−03 | Tnni2 | troponin I type 2 (skeletal, fast) |
| −1.150 | 1.35E−03 | Ttc30b | tetratricopeptide repeat domain 30B |
| −1.200 | 1.35E−03 | Txnip | thioredoxin interacting protein |
| −1.370 | 1.35E−03 | Ucp3 | uncoupling protein 3 (mitochondrial, |
| | | | proton carrier) |
| 1.230 | 1.35E−03 | Unc5b | unc-5 netrin receptor B |
| −1.360 | 1.35E−03 | Zfyve28 | zinc finger, FYVE domain containing 28 |
| −1.060 | 2.52E−03 | Zmynd10 | zinc finger, MYND-type containing 10 |
| −1.010 | 2.52E−03 | Zfp112 | zinc finger protein 112 |
| −1.260 | 1.35E−03 | Zfp13 | zinc finger protein 205 |
| 1.010 | 1.35E−03 | Zfp385b | zinc finger protein 385B |
| 2.590 | 1.35E−03 | Zfp474 | zinc finger protein 474 |

Changes in gene expression of treated rats compared to control rats can guide new therapeutic development for treating various diseases. For example, as shown in Table 7, Alox15 gene is significantly downregulated, whereas Myh4 gene and Nr4a2 gene are drastically upregulated in 17 month rats intravenously administered with PSC-100 compared to 17 month controls.

Alox15 encodes arachidonate 15-lipoxygenase, a protein associated with artery disease, leukomalacia, and apoptotic death of synovial cells.

Myh4 encoded myosin heavy chain. The increased expression of Myh4 in the intravenously treated animals compared to the sham controls is indicative of improved muscle maintenance by the treatment of PSC-100 and may explain the increased performance of the treated animals in the rotarod test.

Nr4a2 encodes a member of the steroid-thyroid hormone-retinoid receptor superfamily. This protein may act as a transcription factor for the increased expression of other genes. Mutations in Nr4a2 have been associated with disorders related to dopaminergic dysfunction, including Parkinson disease, schizophrenia, and manic depression. Misregulation of this gene may be associated with rheumatoid arthritis. Alternatively spliced transcript variants have been described, but their biological validity has not been determined.

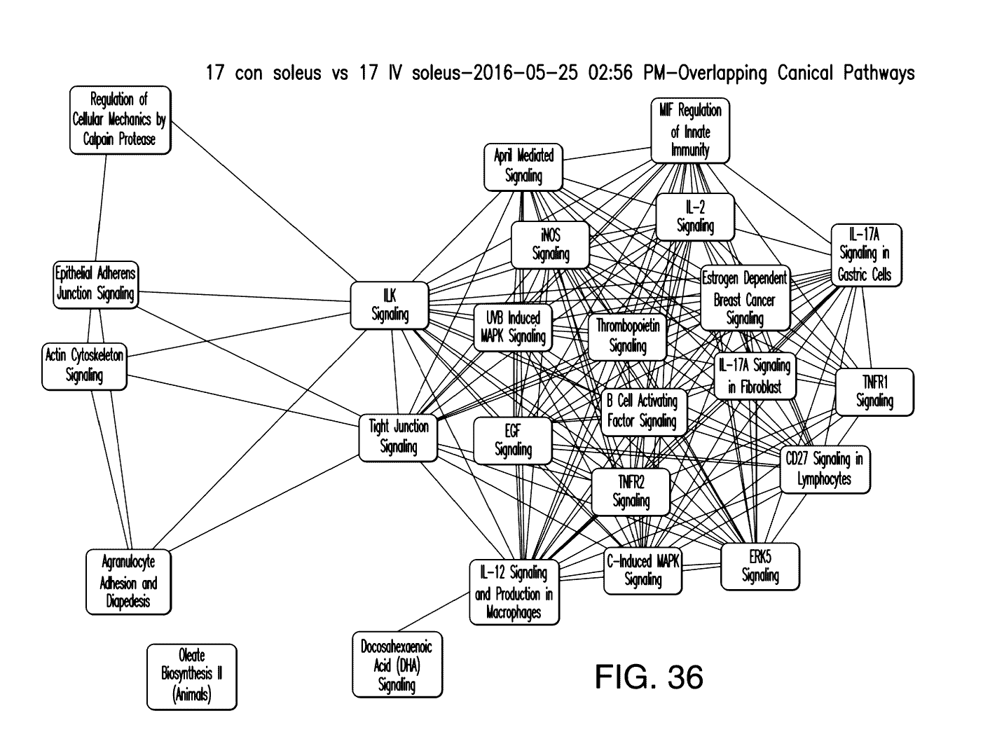

An example of pathways on which changed gene expression can impact is illustrated in FIG. 36.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

What is claimed:

1. A method, wherein:
(i) the method is a method for maintaining or increasing the ratio of the number of stem cells to the number of differentiated cells in a tissue of a subject over time, comprising administering to the subject an effective amount of a population of stem cells, wherein the ratio is maintained or increased over time as compared to the ratio of the number of stem cells to the number of differentiated cells in a tissue of a control subject over time;
(ii) the method is a method of maintaining or increasing the number of stem cells in a tissue of a subject over time, comprising administering to the subject an effective amount of a population of stem cells, wherein the number of stem cells in the tissue of the subject is maintained or increased over time as compared to the number of stem cells in the same tissue of a control subject;
(iii) the method is a method of altering the phenotype of an aging stem cell resident in a tissue of a subject, comprising administering to the subject an effective amount of a population of stem cells, wherein the amount is effective to alter the environmental niche of the aging stem cell such that the phenotype of the aging stem cell is altered as compared to the phenotype of the aging stem cell resident in the tissue of a control subject;
(iv) the method is a method of altering the proteome of an aging cell in a tissue of a subject, comprising administering to the subject an effective amount of a population of stem cells, wherein the amount is effective to alter the proteome of the aging cell, wherein the altered proteome comprises one or more biomarkers found in a younger cell in the tissue of a control subject; or
(v) the method is a method of altering the transcriptome of an aging cell in a tissue of a subject, comprising administering to the subject an effective amount of a population of stem cells, wherein the amount is effective to alter the transcriptome of the aging cell, wherein the altered transcriptome comprises one or more transcripts found in a younger cell in the tissue of a control subject.

2. The method of claim 1, wherein:
(i) the tissue is selected from the group consisting of muscle, brain, heart, kidney, liver, bone marrow, and skin; or
(ii) the aging cell is a somatic cell;
wherein optionally the aging cell is selected from the group consisting of a muscle cell, a brain cell, a heart cell, a liver cell, a kidney cell, a bone marrow cell, and a skin cell; and
wherein optionally the muscle cell is a skeletal muscle cell or a striate muscle cell.

3. The method of claim 1 to 2, wherein the control subject is the same subject before administration of the population of stem cells or a subject that has not received the population of stem cells.

4. The method of any one of claims 1 to 3, wherein:
(i) the population of stem cells is administered systemically, locally to the tissue, parenterally, intravenously, intramuscularly, subcutaneously, subdermally, intracompartmentally, by continuous drip, or as a bolus;
(ii) the population of stem cells is prepared to be administered in an injectable liquid suspension or other biocompatible medium;
(iii) the population of stem cells is administered using a catheter, a controlled-release system, or an implantable substrate or matrix;
(iv) the population of stem cells is administered at a dose of between 1×10⁵ cells and 1×10⁹ cells, between 1×10⁵ cells and 1×10⁷ cells, or between 1×10⁶ cells and 1×10⁷ cells;
(v) the population of stem cells is administered as a single dose or multiple doses;
(vi) the population of stem cells is the first administration to the subject;
(vii) the population of stem cells is administered when the subject is 10-15 years of age, 15-20 years of age, 20-25 years of age, 25-30 years of age, 30-35 years of age, 35-40 years of age, 40-45 years of age, 45-50 years of age, 50-55 years of age, 55-60 years of age, 60-65 years of age, 65-70 years of age, 70-75 years of age, 75-80 years of age, 80-85 years of age, 85-90 years of age, 90-95 years of age, 95-100 years of age, or over 100 years of age; or
(viii) the population of stem cells is serially administered over the lifetime of the subject.

5. The method of any one claims 1 to 4, wherein the population of stem cells:
(i) comprises a population of stem cells selected from the group consisting of: embryonic stem cells, adult stem cells, and induced pluripotent stem cells;
(ii) consists essentially of a population of stem cells selected from the group consisting of: embryonic stem cells, adult stem cells, and induced pluripotent stem cells; or
(iii) consists of a population of stem cells selected from the group consisting of: embryonic stem cells, adult stem cells, and induced pluripotent stem cells.

6. The method of any one claims 1 to 5, wherein the population of stem cells:
(i) comprises a population of stem cells selected from the group consisting of: bone marrow mesenchymal stem cells, amniotic membrane-derived mesenchymal stem cells, adipose tissue-derived mesenchymal stem cells, stem cells from human exfoliated deciduous teeth, skeletal muscle-derived stem cells, blood stem cells, skin stem cells, cord blood stem cells, limbal stem cells, hematopoietic stem cells, neural stem cells, heart-derived stem cells, intestinal stem cells, endothelial stem cells, epithelial stem cells, olfactory adult stem cells, neural crest stem cells, testicular stem cells, placental derived stem cells, and amniotic fluid-derived stem cells;
(ii) consists essentially of a population of stem cells selected from the group consisting of: bone marrow mesenchymal stem cells, amniotic membrane-derived mesenchymal stem cells, adipose tissue-derived mesenchymal stem cells, stem cells from human exfoliated deciduous teeth, skeletal muscle-derived stem cells, blood stem cells, skin stem cells, cord blood stem cells, limbal stem cells, hematopoietic stem cells, neural stem cells, heart-derived stem cells, intestinal stem cells, endothelial stem cells, epithelial stem cells, olfactory adult stem cells, neural crest stem cells, testicular stem cells, placental derived stem cells, and amniotic fluid-derived stem cells; or
(iii) consists of a population of stem cells selected from the group consisting of: bone marrow mesenchymal stem cells, amniotic membrane-derived mesenchymal stem cells, adipose tissue-derived mesenchymal stem cells, stem cells from human exfoliated deciduous teeth, skeletal muscle-derived stem cells, blood stem cells, skin stem cells, cord blood stem cells, limbal stem cells, hematopoietic stem cells, neural stem cells, heart-derived stem cells, intestinal stem cells, endothelial stem cells, epithelial stem cells, olfactory adult stem cells, neural crest stem cells, testicular stem cells, placental derived stem cells, and amniotic fluid-derived stem cells;
wherein optionally the population of stem cells does not comprise one or more stem cells selected from the group consisting of: bone marrow mesenchymal stem cells, amniotic membrane-derived mesenchymal stem cells, adipose tissue-derived mesenchymal stem cells, stem cells from human exfoliated deciduous teeth, skeletal muscle-derived stem cells, blood stem cells, skin stem cells, cord blood stem cells, limbal stem cells, hematopoietic stem cells, neural stem cells, heart-derived stem cells, intestinal stem cells, endothelial stem cells, epithelial stem cells, olfactory adult stem cells, neural crest stem cells, testicular stem cells, placental derived stem cells, and amniotic fluid-derived stem cells.

7. The method of any one of claims 1 to 6, wherein the population of stem cells (i) comprises placental-derived stem cells (PDSC), (ii) consists essentially of PDSC, or (iii) consists of PDSC.

8. The method of any one of claims 1 to 7, wherein:
(i) the population of stem cells has previously been cryopreserved;
(ii) the population of stem cells has been passaged at least three times;
(iii) the population of stem cells has been passaged no more than ten times;
(iv) the population of stem cells are cells from a placental stem cell bank;
(v) the stem cells are embryonic-like stem cells;
(vi) the stem cells are pluripotent or multipotent stem cells;
(vii) the population of stem cells comprises cells obtained from a placenta that has been drained of cord blood; or
(viii) the population of stem cells comprises cells obtained from a placenta that has been perfused to remove residual blood.

9. The method of any one of claims 1 to 8, wherein:
(i) the population of stem cells is autologous to the subject;
(ii) the population of stem cells is allogeneic to the subject;
(iii) the population of stem cells is syngeneic to the subject;
wherein optionally the population of stem cells is a homogeneous cell population or a mixed cell population;
(iv) the population of stem cells is an enriched stem cells population;
(v) the population of stem cells comprises PSC-100 cells;
wherein optionally the population of stem cells is an enriched population of PSC-100 cells; or
(vi) the population of stem cells is obtained from multiple donors;
wherein optionally the population of stem cells is obtained from multiple donors without use of HLA typing.

10. The method of any one of claims 1 to 9, wherein:
(i) the population of stem cells comprises cells that are CD34⁻, CD10⁺, SH2⁺, and CD90⁺ placental multipotent cells;
(ii) the population of stem cells comprises cells that are CD34⁻, CD38⁻, CD45⁻, CD10⁺, CD29⁺, CD44⁺, CD54⁺, CD90⁺, SH2⁺, SH3⁺, SH4⁻ and OCT-4⁺;
(iii) the population of stem cells comprises cells that are CD34⁻, CD10⁺, CD105⁺, and CD200⁺;
(iv) the population of stem cells comprises cells that are CD73⁺;
(v) the population of stem cells comprises cells that are CD73⁺ and CD105⁺;
(vi) the population of stem cells comprises cells that are CD200⁺;
(vii) the population of stem cells comprises cells that are CD34, CD38⁻, CD45⁻, OCT-4⁻, and CD200⁺;
(viii) the population of stem cells comprises cells that are CD34⁻, CD38⁻, CD45⁻, CD73⁺, OCT-4⁺, and CD200⁺;
(ix) the population of stem cells comprises cells that are OCT-4⁺;
(x) the population of stem cells comprises cells that are CD73⁺, CD105⁺, and OCT-4⁺;
(xi) the population of stem cells comprises cells that are CD73⁺, CD105⁺, and CD200⁺;
(xii) the population of stem cells comprises cells that are CD200⁺ and OCT-4⁺;
(xiii) the population of stem cells comprises cells that are CD73⁺, CD105⁺, and HLA-G⁺;
(xiv) the population of stem cells comprises cells that are CD73⁺, CD105⁺, CD200⁺, and HLA-G⁺;
(xv) the population of stem cells comprises cells that are CD34, CD38⁻, CD45⁻, and HLA-G⁺; or
(xvi) the population of stem cells comprises cells that are CD34⁻; CD38⁻; CD45⁻; CD34⁻ and CD38⁻; CD34⁻ and CD45⁻; CD38⁻ and CD45⁻; or CD34⁻, CD38⁻ and CD45⁻.

11. The method of any one of claims 1 to 10, wherein the method further comprises
(i) determining the number of stem cells and/or differentiated cells in the tissue before administration of the population of stem cells to the subject, and
(ii) determining the number of stem cells and/or differentiated cells in the tissue after administration of the population of stem cells to the subject;
wherein optionally
(A) the method increases the number of stem cells in the tissue after administration as compared to before administration of the population of stem cells; or
(B) the subject has an increased number of stem cells as compared to a subject that has not received an administration of population of stem cells;
and wherein optionally
(1) the increase in the number of stem cells persists over time;
(2) the increase in the number of stem cells is the result of an expansion of stem cells resident in the tissue or the result of an expansion of the stem cells in the tissue; or
(3) the increase in the number of stem cells results in the remodeling, renewal, renovation, rejuvenation, repair and/or restoration of the tissue of the subject;
and wherein the number of stem cells is assessed by stem cell colony forming units.

12. The method of any one of claims 1 to 11, wherein the method further comprises contacting the population of stem cells with
(i) young stem cells, young progenitor cells, or young precursor cells; or
(ii) one or more additional factors isolated from young stem cells, young progenitor cells, or young precursor cells;
wherein optionally the one or more additional factors are selected from the group consisting of cytokines, hormones, promoters, repressors, proteins, nucleic acids, viruses, immunogens, angiogenic factors, growth factors, anti-apoptotic factors, and anti-oxidative factors.

13. The method of any one of claims 1 to 12, wherein the method further comprises culturing and/or expanding the population of stem cells prior to administration to the subject, wherein
(i) the culturing and/or expanding is in vitro or in situ;
(ii) the population of stem cells is cultured and/or expanded in an extracorporeal device; or
(iii) the population of stem cells is cultured and/or expanded in the presence of
(A) young stem cells, young progenitor cells, or young precursor cells; or
(B) additional factors isolated from young stem cells, young progenitor cells, or young precursor cells;
wherein optionally the one or more additional factors are selected from the group consisting of cytokines, hormones, promoters, repressors, proteins, nucleic acids, viruses, immunogens, angiogenic factors, growth factors, anti-apoptotic factors, and anti-oxidative factors.

14. The method of any one of claims 1 to 13, wherein the method further comprises
(i) characterizing the genome of the stem cells, wherein the genomic characterization is conducted
(A) prior to administration of the population of stem cells to the subject;
(B) after administration of the population of stem cells to the subject; or
(C) prior to administration of the population of stem cells to the subject, and after administration of the population of stem cells to the subject;
(ii) characterizing the proteome of the stem cells, wherein the proteome characterization is conducted
(A) prior to administration of the population of stem cells to the subject;
(B) after administration of the population of stem cells to the subject; or
(C) prior to administration of the population of stem cells to the subject, and after administration of the population of stem cells to the subject;
(iii) characterizing the genome of the stem cells and/or differentiated cells in the tissue, wherein the genomic characterization is conducted
(A) prior to administration of the population of stem cells to the subject;
(B) after administration of the population of stem cells to the subject; or
(C) prior to administration of the population of stem cells to the subject, and after administration of the population of stem cells to the subject; or
(iv) characterizing the proteome of the stem cells and/or differentiated cells in the tissue, wherein the proteome characterization is conducted
(A) prior to administration of the population of stem cells to the subject;
(B) after administration of the population of stem cells to the subject; or
(C) prior to administration of the population of stem cells to the subject, and after administration of the population of stem cells to the subject.

15. The method of any one of claims 1 to 14, wherein the one or more biomarkers are proteins expressed in a skeletal muscle cell, a striated muscle cell, a brain cell, a heart cell, a kidney cell, a liver cell, a bone marrow cell, or a skin cell.

16. The method of any one of claims 1 to 15, wherein the one or more biomarkers are selected from the group consisting of
(i) myosin light chain 3 (MLCF3), myosin light polypeptide 2 (slow), myosin light chain 1 (MLC1F), myosin binding protein C (MYBPC1), myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), heat shock 27 kDa protein (Hsp27), disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, myosin heavy chain 2 (MYH2), troponin T type 1 (TNNT1), ryanodine receptor 1 (skeletal) (RYR1), calsequestrin 1 (fast-twitch, skeletal muscle) (CASQ1), junctophilin 1 (JPH1), adenosine monosphosphate deaminase (AMPD1), phosphorylase glycogen muscle (PYGM), and enolase 3 (beta, muscle) (ENO3);
(ii) MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the one or more biomarkers is indicative of aging;
(iii) troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the one or more biomarkers is indicative of aging;
(iv) myristoylated alanine-rich C-kinase substrate, alpha-intemexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of myelin basic protein (MBP), and vimentin (VIM);
(v) amyloid beta (A4) precursor protein (APP), myristoylated alanine-rich protein kinase C substrate (MARCKS), internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hid (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1;
(vi) amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU);
(vii) proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN);
(viii) microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH);
(ix) neurofilament triplet L protein (NF-L), peroxiredoxin (EC 1.11.1.), aconitate hydratase (EC 4.2.1.3), enolase 2 (EC 4.2.1.11), and T-complex protein 1;
(x) myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2);
(xi) ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2);
wherein optionally the biomarker is elongation factor 2 (Eef2) and an increase in the expression of Eef2 is indicative of aging;
(xii) ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the one or more biomarkers is indicative of aging;
(xiii) podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH);
(xiv) transferrin, isocitrate dehydrogenase 1 (IDH), and 3-hydroxyisobutyrate dehydrogenase, wherein an increase in the expression of the one or more biomarkers is indicative of an aging;
(xv) afenopin, phosphoglycerate mutase, and glutathione (GSH), wherein a decrease in the expression of the one or more biomarkers is indicative of aging;
(xvi) apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase;
(xvii) epoxide hydroxylase, 3-ketoacyl-CoA thiolase A, sarcosine oxidase, and 2,4-dienoyl reductase, wherein an increase in expression of the one or more biomarkers is indicative of aging;
(xviii) defensin, alpha 1 (DEFA1), defensin, alpha 1B (DEFA1B), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterobiomarkerous nuclear ribonucleoprotein A1, myosin light chain, regulatory B (Mrlcb), transgelin, similar to purine-nucleoside phosphorylase (punA), heterobiomarkerous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5);
(xix) fatty acid-binding protein 5, galectin-3, c-synuclein, heterobiomarkerous nuclear ribonucleoprotein A1, myosin light chain, regulatory B, peroxiredoxin 5 precursor, and transgelin;
(xx) beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterobiomarkerous nuclear ribonucleoprotein A1 isoform a (HNRPA1), heterobiomarkerous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln);
(xxi) transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5);
(xxii) collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1);
(xxiii) mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the one or more biomarkers is indicative of aging;
(xxiv) annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1);
(xxv) annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the one or more biomarkers is indicative of aging;
(xxvi) twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the one or more biomarkers is indicative of aging; or
(xxv) Abcg1, Abra, Actn3, Alas2, Alox15, Angpt14, Apod, Apold1, Arc, Arhgap24, Arl4c, Amt1, Arrdc2, Asb5, Atf3, Bag2, Bcl11a, Bcl6, Bdh1, Bdnf, Best3, Bhlhe40, Calhm1, Calm13, Car12, Ccl5, Cd74, Cdc42se1, Chac1, Chst5, Ciart, Cidec, Cish, Cited4, Ckap4, Cldn2, Clic6, Cpt1a, Csrnp1, Cxcl13, Dbp, Dnajb5, Dynl11, Dyrk2, Edn1, Egr1, Egr3, Elfn1, Emb, Enah, Fam107b, Fam110a, Fam134b, Fam167a, Fam46a, Fasn, Fgfr3, Fhl2, Fos, Fosb, Frk, Fst, Gdf15, Gem, Gngt1, Gnl3, Hba1, Hba2, Hbb, Hbb-b1, Hbegf, Hmox1, Hpd1, Hspa1b, Id4, Il2rb, Irs1, Irs2, Junb, Jund, Kbtbd8, Kcnk5, Kctd7, Kirrel2, Ky, Lamc2, Lipg, LOC689064, Lonrf3, Lrrc38, Lrrc52, Lrrn2, Lsr, Maff, Mchr1, Mfrp, Mllt11, Mns1, Mogat1, Mphosph6, Mpz, Muc20, Mybpc2, Myf6, Myh1, Myh2, Myh4, Myocd, Nedd9, Nfil3, Nkg7, Nrld1, Nr4a2, Nr4a3, Ntf4, Nuak1, Parp16, Pdc, Pde7a, Pfkfb2, Pfkfb3, Pgam1, Phlda1, Pik3ip1, Plk3, Postn, Ppargc1a, Ppp1r14c, Pragmin, Prf1, Ptpn14, Pva1b, Rab23, Rab30, Rbm20, Rcan1, Rel11, Rfx1, RGD1307461, RGD1309676, RGD1359290, RGD1564428, Rhpn2, Rn45s, Rnd1, Rp1, Rrad, RT1-Ba, RT1-Bb, RT1-Da, RT1-Db1, Rtn4rl1, Scd1, Sdc4, Sec1415, Siglec5, Sik1, Slc18a2, Slc2a5, Slc30a4, Slc4a1, Slc4a5, Slpi, Smad7, Snhg4, Spag8, Stc1, Sv2c, Terf2ip, Thrsp, Tmc8, Tmem171, Tmx4, Tnfrsfl2a, Tnni2, Ttc30b, Txnip, Txnip, Ucp3, Unc5b, Zfp112, Zfp13, Zfp385b, Zfp474, Zfyve28, Zic1 and Zmynd10.

17. The method of any one of claims 1 to 16, wherein
(i) the increase in expression of the one or more biomarkers is gender specific;
(ii) the biomarker is ATP synthase and the expression of the ATP synthase in up-regulated in aging males;
(iii) the biomarker is catalase and the expression of the catalase is down-regulated in aging males;
(iv) the biomarker is ATP synthase and the expression of ATP synthase is down-regulated in aging females;
(v) the biomarker is ornithine aminotransferase and the expression of the ornithine aminotransferase is up-regulated in aging females; or
(vi) the biomarker is glutamate dehydrogenase and the expression of the glutamate dehydrogenase is down-regulated in aging females.

18. The method of any one of claims 1 to 17, wherein the one or more transcripts are
(i) identified using a transcript array analysis;
(ii) expressed in the skeletal muscle, the brain, the heart, the kidney, the liver, the bone marrow, or the skin; or
(iii) selected from the group consisting of:
(A) myosin light chain 3 (MLCF3), myosin light polypeptide 2 (slow), myosin light chain 1 (MLC1F), myosin binding protein C (MYBPC1), myosin binding protein H, alpha actin (fragment), actin (skeletal muscle), actin alpha (cardiac), troponin T class Ia alpha-1, troponin T class IIa beta-1, troponin T beta/alpha, capZ beta, desmin, gelsolin (cytosolic), beta-tubulin, p23, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, ferritin heavy chain (H-ferritin), aldehyde dehydrogenase (mitochondrial), glutathione transferase (omega 1), heat shock 20 kDa protein (Hsp20), heat shock 27 kDa protein (Hsp27), disulfide isomerase ER60 (ERp57), 14-3-3 protein, guanine deaminase (guanase), Rho-GDI (alpha), phosphohistidine phosphatase, mRNA capping enzyme, similar to apobec2 protein, galectin 1, albumin, vitamin D binding protein prepeptide, protein kinase C interacting protein-1, RIKEN cDNA 1700012G19, myosin heavy chain 2 (MYH2), troponin T type 1 (TNNT1), ryanodine receptor 1 (skeletal) (RYR1), calsequestrin 1 (fast-twitch, skeletal muscle) (CASQ1), junctophilin 1 (JPH1), adenosine monosphosphate deaminase (AMPD1), phosphorylase glycogen muscle (PYGM), and enolase 3 (beta, muscle) (ENO3);
(B) MLCF3, myosin light polypeptide 2 (slow), MLC1F, myosin binding protein C, myosin binding protein H, alpha actin (fragment), actin (skeletal muscle), actin alpha (cardiac), troponin T class IIa beta-1, troponin T beta/alpha, capZ beta, triosephosphate isomerase 1, glycosylase I, glyoxalase I, enolase 3 (beta muscle), glycerol 3-P dehydrogenase, isocitrate dehydrogenase 3 (NAD+), cytochrome c oxidase (polypeptide Va), creatine kinase (muscle form), Cu/Zn superoxide dismutase, phosphohistidine phosphatase, protein kinase C interacting protein-1, and RIKEN cDNA 1700012G19, wherein a decrease in expression in the one or more transcripts is indicative of aging;
(C) troponin T class Ia alpha-1, troponin T class IIa beta-1, desmin, gelsolin (cytosolic), beta-tubulin, p23, ferritin heavy chain (H-ferritin), aldehyde dehydrogenase (mitochondrial), glutathione transferase (omega 1), Hsp20, Hsp20, disulfide isomerase ER60 (ERp57), 14-3-3 protein, guanine deaminase (guanase), Rho-GDI (alpha), mRNA capping enzyme, similar to apobec2 protein, galectin 1, albumin, vitamin D binding protein prepeptide, wherein an increase in expression in the one or more transcripts is indicative of aging;
(D) myristoylated alanine-rich C-kinase substrate, alpha-intemexin, isoform B of methyl-CpG-binding protein 2, histone H1.4, isoform 1 of serum albumin, guanine nucleotide-binding protein (G(1)/G(S)/G(T) subunit beta-1, adenylate kinase 1, fructose-biphosphate aldolase A, tenascin-R, isoform 2 of clusterin, synaptic transmission, cation transport, isoform 1 of myeline proteolipid protein, neuromodulin, dihydropyrimidinase-related protein 2, dihydropteridine reductase, matrin-3, alpha-enolase, isoform 1 of gelsolin, APP isoform of APP714 of amyloid beta A4 protein (fragment), annexin A6, isoform tau-E of microtubule-associated protein tau, MAP1A 331 kDa protein, neuroblast differentiation-associated protein AH NAK, cell cycle exit and neuronal differentiation protein 1, glyceraldehyde-3-phosphate dehydrogenase, HIST1H1D, isoform KGA of glutaminase kidney isoform, superoxide dismutase (Mn) (SOD2), isoform 1 of myelin basic protein (MBP), and vimentin (VIM);
(E) amyloid beta (A4) precursor protein (APP), myristoylated alanine-rich protein kinase C substrate (MARCKS), internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR), clusterin (CLU), synapsin 1 (SYN1), ATP synthase, H+ transporting, mitochondrial F1 complex, alpha subunit 1, cardiac musle (ATP5A1), proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), gelsolin (GSN), annexin A6 (ANXA6), microtubule associated protein tau (MAPT), microtuble-associated protein 1A (MAP1A), AHNAK nucleoprotein, cell cycle exit and neuronal differentiation 1 (CEND1), glyceraldehyde-3-phosphate dehydrogenase (GAPDH), histone cluster 1, Hld (HIST1H1D), glutaminase (GLS), superoxide dismutase (SOD2), MBP, VIM, ELAV-like protein 3 (ELAVL3), neurogranin (NRGN), receptor expression enhancing protein 2 (REEP2), glutamate decarboxylase 1 (GAD1), protocadherin alpha-1 (PCDHA1), glial fibrillary acidic protein (GFAP), S100 calcium binding protein (S100B), family with sequence similarity 19 (chemokine (C-C-motif)-like), member A1 (FAM19A1), aquaporin 4 (AQP4), c-type lectin domain family 2, member L (CLEC2L), neurofilament triplet L protein (NF-L), peroxiredoxin (EC 1.11.1.), aconitate hydratase (EC 4.2.1.3), enolase 2 (EC 4.2.1.11), and T-complex protein 1;
(F) amyloid beta (A4) precursor protein (APP), marcks, internexin neuronal intermediate filament protein alpha (INA), methyl CpG binding protein (MECP), histone cluster 1 H1e (HIST1H1E), albumin (ALB), guanine nucleotide binding protein (G protein) beta polypeptide (GNB1), adenylate kinase 1 (AK1), aldose A fructose-biphosphate (ALDOA), tenascin R (TNR) and clusterin (CLU);
(G) proteolipid protein 1 (PLP1), growth associated protein 43 (GAP43), dihydropyrimidinase-like 2 (DPYSL2), quinoid dihydropteridine reductase (QDPR), matrin 3 (MATR3), enolase 1 (alpha) (ENO1), and gelsolin (GSN);
(H) microtubule associated protein tau (MAPT), microtuble-associated protein 1A (MAP1A), AHNAK nucleoprotein, cell cycle exit and neuronal differentiation 1 (CEND1) and glyceraldehyde-3-phosphate dehydrogenase (GAPDH);
(I) neurofilament triplet L protein (NF-L), peroxiredoxin (EC 1.11.1.), aconitate hydratase (EC 4.2.1.3), enolase 2 (EC 4.2.1.11), and T-complex protein 1;
(J) myosin, heavy chain 6, cardiac muscle, alpha (MYH6), actin, alpha, cardiac muscle 1 (ACTC1), troponin I type 3 (cardiac) (TNNI3), natriuretic peptide A (NPPA), A kinase (PRKA) anchor protein 6 (AKAP6), nestin (NES), ATPase, Na+, K+ transporting, alpha 3 polypeptide (ATP1A3), cadherin 2, type 1, N-cadherin (neuronal) (CDH2), plakophilin 2 (PKP2), ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2);
(K) ATP synthase subunit d (Atp5h), ATP synthase subunit o (Atp5o), ATP synthase subunit delta (Atp5d), ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), 60 kDa heat shock protein (Hspd1), desmin (Desm), troponin T2 (Tnnt2), tropomyosin alpha 1 (Tpm1), voltage dependent anion channel-1 (Vdac1), and elongation factor 2 (Eef2);
wherein optionally the biomarker is elongation factor 2 (Eef2) and an increase in the expression of Eef2 is indicative of aging;
(L) ATP synthase subunit alpha (Atp5a1), ATP synthase subunit beta (Atp5b), cytochrome c (Cycs), mito, pyruvate dehydgrenase E1 component subunit beta (Pdhb), phosphoglycerate kinase 1 (Pgk1), heat shock protein 70 (Hspa9), desmin (Desm), troponin T2 (Tnnt2), tropomyosin alpha 1 (Tpm1), voltage dependent anion channel-1 (Vdac1), wherein a decrease in the expression of the one or more transcripts is indicative of aging;
(M) podocin (NPHS2), nephrin (NPHS1), kin of IRRE like (NEPH1 or KIRREL), podocalyxin-like (PODXL), fibroblast growth factor 1 (FGF1), crumbs family member 2 (CRB2), solute carrier family 22 (organic anion transporter), member 8 (SLC22A8), solute carrier family 22 (organic anion transporter), member 13 (SLC22A13), aminocarboxymuconate semialdehyde decarboxylase (ACMSD), agmatine ureohydrolase (agmatinase) (AGMAT), betaine-homocysteine S-methyltransferase (BHMT), chromosome 11 open reading frame 54 (C11orf54), cadherin 6, type 2, K-cadherin (fetal kidney) (CDH6), dihycropyrimidinase (DPYS), gamma-glutamyltransferase 1 (GGT1), 4-hydroxyphenylpyruvate dioxygenase (HPD), heat-responsive protein 12 (HRSP12), low density lipoprotein receptor-related protein 2 (LRP2), pyruvate kinase, liver and RBC (PKLR), X-prolyl aminopeptidase (aminopeptidase P)2, membrane-bound (XPNPEP2), uromodulin (UMOD), calbindin (CALB1), solute carrier family 12 (sodium/potassium/chloride transporter), member 1 (SLC12A1), solute carrier family 12 (sodium/chloride transporter), member 3 (SLC12A3), calcium-sensing receptor (CASR), aquaporin (AQP2), ATPase, H+ transporting, lysosomal 38 kDa, V0 subunit d2 (ATP6V0D2), parvalbumin (PVALB), transmembrane protein 213 (TMEM213), transferrin, isocitrate dehydrogenase 1 (IDH), 3-hydroxyisobutyrate dehydrogenase, afenopin, heat shock protein (HSP) 9A, ATP synthase, ornithine aminotransferase, glutamate dehydrogenase, phosphoglycerate mutase, catalase, and glutathione (GSH);
(N) transferrin, isocitrate dehydrogenase 1 (IDH), and 3-hydroxyisobutyrate dehydrogenase, wherein an increase in the expression of the one or more transcripts is indicative of aging;
(O) afenopin, phosphoglycerate mutase, and glutathione (GSH), wherein a decrease in the expression of the one or more transcripts is indicative of aging;
(P) apolipoprotein B (APOB), apolipoprotein A-I (APOA1), fibrinogen gamma chain (FGG), complement component 2 (C2), kininogen 1 (KNG1), fibrinogen alpha chain (FGA), hydroxyacid oxidase (glycolate oxidase) 1 (HAO1), retinol dehydrogenase 16 (all-trans) (RDH16), aldolase B, fructose-bisphosphate (ALDOB), bile acid CoA: amino acid N-acyltransferase (glycine N-choloyltransferase) (BAAT), aldo-keto reductase family 1, member C4 (AKR1C4), solute carrier family 27 (fatty acid transporter), member 5 (SLC27A5), epoxide hydrolase, 3-ketoacyl-CoA thiolase A, sarcosine oxidase, and 2,4-dienoyl reductase;
(Q) epoxide hydroxylase, 3-ketoacyl-CoA thiolase A, sarcosine oxidase, and 2,4-dienoyl reductase, wherein an increase in expression of the one or more transcripts is indicative of aging;
(R) defensin, alpha 1 (DEFA1), defensin, alpha 1B (DEFAIB), defensin, alpha 3 (DEFA3), defensin, alpha 4 (DEFA4), cathepsin G (CTSG), myeloperoxidase (MPO), hemoglobin, beta (HBB), hemoglobin, alpha 1 (HBA1), hemoglobin, alpha 2 (HBA2), S100 calcium binding protein 12 (S100A12), chromosome 19 open reading frame 59 (C19orf59), pyruvate dehydrogenase (lipoamide) beta, fatty acid-binding protein 5, galectin-3, c-synuclein, heterobiomarkerous nuclear ribonucleoprotein A1, myosin light chain, regulatory B (Mrlcb), transgelin, similar to purine-nucleoside phosphorylase (punA), heterobiomarkerous nuclear ribonucleoprotein A2/B1 isoform A2 (Hnrpa2b1), Huntingtin interacting protein K (HYPK), beta-actin FE-3 (Actg1), caldesmon 1 (Cald1, calponin-1 (Cnn1), E-FABP (C-FABP) (Fabp5), capping protein (actin filament), gelsolin-like (CAPG), similar to coactosin-like 1 (Cotl1), calponin-1 (calponin H1, smooth muscle; basic calponin) (Cnn1), vinculin (VCL), VIM, beta-tropomyosin (TPM2), transgelin 2 (Tagln2), tropomyosin 1, alpha isoform c (TPM1), calponin 3, acidic (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5);
(S) fatty acid-binding protein 5, galectin-3, c-synuclein, heterobiomarkerous nuclear ribonucleoprotein A1, myosin light chain, regulatory B, peroxiredoxin 5 precursor, and transgelin;
(T) beta-actin FE-3 (Actg1), caldesmon 1 (Cald1, calponin-1 (Cnn1), E-FABP (C-FABP) (Fabp5), galectin-3 (LGALS3), gamma synuclein (Sncg), heterobiomarkerous nuclear ribonucleoprotein A1 isoform a (HNRPA1), heterobiomarkerous nuclear ribonucleoprotein A2/B1 isoform A2 (Hnrpa2b1), Huntingtin interacting protein K (HYPK), myosin light chain, regulatory B (Mrlcb), peroxiredoxin 5 precursor (Prdx5), similar to purine-nucleoside phosphorylase (punA), pyruvate dehydrogenase (lipoamide) beta (PDHB), and transgelin (Tagln);
(U) transgelin (Tagln), capping protein (actin filament), gelsolin-like (CAPG), caldesmon 1 (Cald1), beta-actin FE-3 (Actg1), similar to coactosin-like 1 (Cotl1), calphonin-1 (calphonin H1, smooth muscle; basic calponin) (Cnn1), vinculin (VCL), VIM, beta-tropomyosin (TPM2), myosin light chain, regulatory B (Mrlcb), transgelin 2 (Tagln2), tropomyosin 1, alpha isoform c (TPM1), calponin 3, acidid (CNN3), calponin 2 isoform a (Calponin 2), F-actin capping protein beta subunit (Capzb), alpha-globulin (Hba1), alpha-actin (aa 40-375) (Acta2), smooth muscle protein SM22 homolog-bovine (fragments) (Tagln2), thioredoxin 2 (Txn1), peroxideroxin 2 (Prdx2), peroxiderodoxin 5 precursor (Prdx5), and Cu—Zn superoxide dismutase A5 (GSTA5);
(V) collagen, type XVII, alplha 1 (COL17A1), tumor protein p73 (TP73), keratin 10 (KRT10), caspase 14, apoptosis-related cysteine peptidase (CASP14), filaggrin (FLG), keratinocyte proline-rich protein (KPRP), corneodesmosin (CDSN), kallikrein-related peptidase 5 (KLK5), melan-A (MLANA), dopachrome tautomerase (DCT), tyrosinase (TYR), CD1a molecule (CD1A), CD207 molecule, langerin, (CD207), annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1);
(W) mitochondrially encoded cytochrome c oxidase II (MTCO2), NADH dehydrogenase (ubiquinone) 1 alpha subcomplex, 5 (NDUFA5), NADH dehydrogenase (ubiquinone) 1 alpha subcomplex, 9 (NDUFA9), NADH dehydrogenase (ubiquinone) 1 alpha subcomplex, 10 (NDUFA10) and NADH dehydrogenase (ubiquinone) Fe—S protein 6, 13 kDa (NADH-coenzyme Q reductase) (NDUFS6), wherein a decrease in expression of the one or more transcripts is indicative of aging;
(X) annexin A6 (ANXA6), glutaminyl-tRNA synthetase (QARS), cation-independent mannose-6-phosphate (IGF2R), twinfilin-2 (TWF2), 40S ribosomal protein S5 (RPS5), putative pre-mRNA-splicing factor ATP-dependent RNA helicase DHX15 (DHX15), 26S proteasome non-ATPase regulatory subunit 1 (PSMD1), 40S ribosomal protein S29 (RPS29), synaptopodin-2 (SYNPO2), T-complex protein 1 subunit zeta (CCT6A), annexin 5 (ANXA5), tRNA-splicing ligase RtcB homolog (C22orf28), serine/arginine-rich splicing factor 9 (SRSF9), myosin light polypeptide 6 (MYL6), protein phosphatase 1 regulatory subunit 7 (PPP1R7), UPF0568 protein C14orfl66 (C14orfl66), 26 proteasome non-ATPase regulatory subunit 14 (PSMD14), serine hydroxymethyltransferase, mitochondrial (SHMT2), heat shock 70 kDa protein 1A/1B (HSPA1A), ATP-dependent RNA helicase DDX1 (DDX1), calmodulin (CALM1), AP-2 complex subunit alpha-2 (AP2A2), Rho guanine nucleotide exchange factor 2 (ARHGEF2), annexin A4 (ANXA4), erythrocyte band 7 integral membrane protein (STOM), ATP-dependent RNA helicase DDX3X (DDX3X), calpain small subunit 1 (CAPNS1), NAD(P)H dehydrogenase [quinone] 1 (NQO1), Protein S100-A16 (S100A16), clathrin light chain B (CLTB), brain acid soluble protein 1 (BASP1), DnaJ homolog subfamily C member 3 (DNAJC3), AP-2 complex subunit alpha-1 (AP2A1), 40S ribosomal protein (RPS6), glycyl-tRNA synthetase (GARS), EH domain-containing protein 2 (EHD2), oligoribonuclease, mitochondrial (REXO2), thrombospondin-1 (THBS1), glycylpeptide N-tetradecanoyltransferase 1 (NMT1), adenylyl cyclase-associated protein 1 (CAP1), heat shock-related 70 kDa protein 2 (HSPA2), histone H2A type 1-A (HIST1H2AA), and T-complex protein 1 subunit alpha (TCP1);
(Y) annexin A6 (ANXA6), glutaminyl-tRNA synthetase (QARS), cation-independent mannose-6-phosphate (IGF2R), putative pre-mRNA-splicing factor ATP-dependent RNA helicase DHX15 (DHX15), 40S ribosomal protein S29 (RPS29), synaptopodin-2 (SYNPO2), annexin 5 (ANXA5), serine/arginine-rich splicing factor 9 (SRSF9), myosin light polypeptide 6 (MYL6), heat shock 70 kDa protein 1A/1B (HSPA1A), calmodulin (CALM1), annexin A4 (ANXA4), erythrocyte band 7 integral membrane protein (STOM), NAD(P)H dehydrogenase [quinone] 1 (NQO1), clathrin light chain B (CLTB), brain acid soluble protein 1 (BASP1), 40S ribosomal protein (RPS6), EH domain-containing protein 2 (EHD2), thrombospondin-1 (THBS1), heat shock-related 70 kDa protein 2 (HSPA2), wherein an increase in expression of the one or more transcripts is indicative of aging;
(Z) twinfilin-2 (TWF2), 40S ribosomal protein S5 (RPS5), 26S proteasome non-ATPase regulatory subunit 1 (PSMD1), T-complex protein 1 subunit zeta (CCT6A), tRNA-splicing ligase RtcB homolog (C22orf28), protein phosphatase 1 regulatory subunit 7 (PPP1R7), UPF0568 protein C14orf166 (C14orf166), 26 proteasome non-ATPase regulatory subunit 14 (PSMD14), serine hydroxymethyltransferase, mitochondrial (SHMT2), ATP-dependent RNA helicase DDX1 (DDX1), AP-2 complex subunit alpha-2 (AP2A2), Rho guanine nucleotide exchange factor 2 (ARHGEF2), ATP-dependent RNA helicase DDX3X (DDX3X), calpain small subunit 1 (CAPNS1), Protein S100-A16 (S100A16), DnaJ homolog subfamily C member 3 (DNAJC3), AP-2 complex subunit alpha-1 (AP2A1), glycyl-tRNA synthetase (GARS), oligoribonuclease, mitochondrial (REXO2), glycylpeptide N-tetradecanoyltransferase 1 (NMT1), adenylyl cyclase-associated protein 1 (CAP1), histone H2A type 1-A (HIST1H2AA), and T-complex protein 1 subunit alpha (TCP1), wherein a decrease in the expression of the one or more transcripts is indicative of aging; or
(AA) Abcg1, Abra, Actn3, Alas2, Alox15, Angpt14, Apod, Apold1, Arc, Arhgap24, Arl4c, Amt1, Arrdc2, Asb5, Atf3, Bag2, Bcl11a, Bcl6, Bdh1, Bdnf, Best3, Bhlhe40, Calhm1, Calm13, Car12, Ccl5, Cd74, Cdc42se1, Chac1, Chst5, Ciart, Cidec, Cish, Cited4, Ckap4, Cldn2, Clic6, Cpt1a, Csrnp1, Cxcl13, Dbp, Dnajb5, Dynl11, Dyrk2, Edn1, Egr1, Egr3, Elfn1, Emb, Enah, Fam107b, Fam110a, Fam134b, Fam167a, Fam46a, Fasn, Fgfr3, Fhl2, Fos, Fosb, Frk, Fst, Gdf15, Gem, Gngt1, Gnl3, Hba1, Hba2, Hbb, Hbb-b1, Hbegf, Hmox1, Hpd1, Hspa1b, Id4, Il2rb, Irs1, Irs2, Junb, Jund, Kbtbd8, Kcnk5, Kctd7, Kirrel2, Ky, Lamc2, Lipg, LOC689064, Lonrf3, Lrrc38, Lrrc52, Lrrn2, Lsr, Maff, Mchr1, Mfrp, Mllt1, Mns1, Mogat1, Mphosph6, Mpz, Muc20, Mybpc2, Myf6, Myh1, Myh2, Myh4, Myocd, Nedd9, Nfil3, Nkg7, Nrld1, Nr4a2, Nr4a3, Ntf4, Nuak1, Parp16, Pdc, Pde7a, Pfkfb2, Pfkfb3, Pgam1, Phlda1, Pik3ip1, Plk3, Postn, Ppargc1a, Ppp1r14c, Pragmin, Prf1, Ptpn14, Pva1b, Rab23, Rab30, Rbm20, Rcan1, Rel11, Rfx1, RGD1307461, RGD1309676, RGD1359290, RGD1564428, Rhpn2, Rn45s, Rnd1, Rp1, Rrad, RT1-Ba, RT1-Bb, RT1-Da, RT1-Db1, Rtn4rl1, Scd1, Sdc4, Sec1415, Siglec5, Sik1, Slc18a2, Slc2a5, Slc30a4, Slc4a1, Slc4a5, Slpi, Smad7, Snhg4, Spag8, Stc1, Sv2c, Terf2ip, Thrsp, Tmc8, Tmem171, Tmx4, Tnfrsfl2a, Tnni2, Ttc30b, Txnip, Txnip, Ucp3, Unc5b, Zfp112, Zfp13, Zfp385b, Zfp474, Zfyve28, Zic1 and Zmynd10.
